SEQUENCE LISTING (139 sequences)

SEQ ID NO: 1 - protein, 11 aa, Hepatitus B virus
  VARIANT 1..3: note = X = any amino acid
  VARIANT 6..8: note = X = any amino acid
  VARIANT 10: note = X = any amino acid
XXXTCXXXAXG

SEQ ID NO: 2 - protein, 11 aa, Hepatitus B Virus
  VARIANT 1: note = X = P, T or S,
  VARIANT 2: note = X = C or S,
  VARIANT 3: note = X = R, K, D or I,
  VARIANT 6: note = X = M or T
  VARIANT 7: note = X = T, A or I
  VARIANT 8: note = X = T, P or L
  VARIANT 10: note = X = Q, H or L.
XXXTCXXXAXG

SEQ ID NO: 3 - protein, 226 aa, Hepatitis B Virus
  REGION 1..226: note = S domain of HBsAg (GenBank acc. no. J02203)
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 4 - protein, 226 aa, Hepatits B Virus
  REGION 1..226: note = S domain of HBsAg (GenBank acc. no. FJ899792)
MENVTSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTGTGPCRTCTTPAQGTSMYPSCCCTKPSDGNCTCIPIPSSWAFGKFLWEWASARFSWLSLLVPFVQWFVGLSPTVWLSVIWMMWYWGPSLYSTLSPFLPLLPIFFCLWVYI

SEQ ID NO: 5 - protein, 72 aa, Hepatitis B Virus
  REGION 1..72: note = J02203 (D, ayw3)
QGMLPVCPLIPGSSTTSTGPCRTCMTTAQGTSMYPSCCCTKPSDGNCTCIPIPSSWAFGKFLWEWASARFSW

SEQ ID NO: 6 - protein, 72 aa, Hepatitis B Virus
  REGION 1..72: note = FJ899792 (D, adw2)
QGMLPVCPLIPGSSTTGTGPCRTCTTPAQGTSMYPSCCCTKPSDGNCTCIPIPSSWAFGKFLWEWASARFSW

SEQ ID NO: 7 - protein, 72 aa, Hepatitis B Virus
  REGION 1..72: note = AM282986 (A)
QGMLPVCPLIPGTTTTSTGPCKTCTTPAQGNSMFPSCCCTKPSDGNCTCIPIPSSWAFAKYLWEWASVRFSW

SEQ ID NO: 8 - protein, 72 aa, Hepatitis B Virus
  REGION 1..72: note = D23678 (B1)
QGMLPVCPLIPGSSTTSTGPCKTCTTPAQGTSMFPSCCCTKPTDGNCTCIPIPSSWAFAKYLWEWASVRFSW

SEQ ID NO: 9 - protein, 72 aa, Hepatitis B Virus
  REGION 1..72: note = AB117758 (C1)
QGMLPVCPLLPGTSTTSTGPCKTCTIPAQGTSMFPSCCCTKPSDGNCTCIPIPSSWAFARFLWEWASVRFSW

SEQ ID NO: 10 - protein, 72 aa, Hepatitis B Virus
  REGION 1..72: note = AB205192 (E)
QGMLPVCPLIPGSSTTSTGPCRTCTTLAQGTSMFPSCCCSKPSDGNCTCIPIPSSWAFGKFLWEWASARFSW

SEQ ID NO: 11 - protein, 72 aa, Hepatitis B Virus
  REGION 1..72: note = X69798 (F4)
QGMLPVCPLLPGSTTTSTGPCKTCTTLAQGTSMFPSCCCSKPSDGNCTCIPIPSSWALGKYLWEWASARFSW

SEQ ID NO: 12 - protein, 72 aa, Hepatitis B Virus
  REGION 1..72: note = AF160501 (G)
QGMLPVCPLIPGSSTTSTGPCKTCTTPAQGNSMYPSCCCTKPSDGNCTCIPIPSSWAFAKYLWEWASVRFSW

SEQ ID NO: 13 - protein, 72 aa, Hepatitis B Virus
  REGION 1..72: note = AY090454 (H)
QGMLPVCPLLPGSTTTSTGPCKTCTTLAQGTSMFPSCCCTKPSDGNCTCIPIPSSWAFGKYLWEWASARFSW

SEQ ID NO: 14 - protein, 72 aa, Hepatitis B Virus
  REGION 1..72: note = AF241409 (I)
QGMLPVCPLIPGSSTTSTGPCKTCTTPAQGNSMYPSCCCTKPSDGNCTCIPIPSSWAFAKYLWEWASARFSW

SEQ ID NO: 15 - protein, 72 aa, Hepatitis B Virus
  REGION 1..72: note = AB486012 (J)
QGMLPVCPLLPGSTTTSTGPCRTCTITAQGTSMFPSCCCTKPSDGNCTCIPIPSSWAFAKFLWEWASVRFSW

SEQ ID NO: 16 - protein, 73 aa, Hepatitis B Virus
  REGION 1..73: note = HBsAg Y100C/P120T
CQGMLPVCPLIPGSSTTGTGTCRTCTTPAQGTSMYPSCCCTKPSDGNCTCIPIPSSWAFGKFLWEWASARFSW

SEQ ID NO: 17 - protein, 72 aa, Hepatitis B Virus
  REGION 1..72: note = HBsAg P120T
QGMLPVCPLIPGSSTTGTGTCRTCTTPAQGTSMYPSCCCTKPSDGNCTCIPIPSSWAFGKFLWEWASARFSW

SEQ ID NO: 18 - protein, 72 aa, Hepatitis B Virus
  REGION 1..72: note = HBsAg P120T/S143L
QGMLPVCPLIPGSSTTGTGTCRTCTTPAQGTSMYPSCCCTKPLDGNCTCIPIPSSWAFGKFLWEWASARFSW

SEQ ID NO: 19 - protein, 72 aa, Hepatitis B Virus
  REGION 1..72: note = HBsAg C121S
QGMLPVCPLIPGSSTTGTGPSRTCTTPAQGTSMYPSCCCTKPSDGNCTCIPIPSSWAFGKFLWEWASARFSW

SEQ ID NO: 20 - protein, 72 aa, Hepatitis B Virus
  REGION 1..72: note = HBsAg R122D
QGMLPVCPLIPGSSTTGTGPCDTCTTPAQGTSMYPSCCCTKPSDGNCTCIPIPSSWAFGKFLWEWASARFSW

SEQ ID NO: 21 - protein, 72 aa, Hepatitis B Virus
  REGION 1..72: note = HBsAg R122I
QGMLPVCPLIPGSSTTGTGPCITCTTPAQGTSMYPSCCCTKPSDGNCTCIPIPSSWAFGKFLWEWASARFSW

SEQ ID NO: 22 - protein, 72 aa, Hepatitis B Virus
  REGION 1..72: note = HBsAg T123N
QGMLPVCPLIPGSSTTGTGPCRNCTTPAQGTSMYPSCCCTKPSDGNCTCIPIPSSWAFGKFLWEWASARFSW

SEQ ID NO: 23 - protein, 72 aa, Hepatitis B Virus
  REGION 1..72: note = HBsAg Q129H
QGMLPVCPLIPGSSTTGTGPCRTCTTPAHGTSMYPSCCCTKPSDGNCTCIPIPSSWAFGKFLWEWASARFSW

SEQ ID NO: 24 - protein, 72 aa, Hepatitis B Virus
  REGION 1..72: note = HBsAg Q129L
QGMLPVCPLIPGSSTTGTGPCRTCTTPALGTSMYPSCCCTKPSDGNCTCIPIPSSWAFGKFLWEWASARFSW

SEQ ID NO: 25 - protein, 72 aa, Hepatitis B Virus
  REGION 1..72: note = HBsAg M133H
QGMLPVCPLIPGSSTTGTGPCRTCTTPAQGTSHYPSCCCTKPSDGNCTCIPIPSSWAFGKFLWEWASARFSW

SEQ ID NO: 26 - protein, 72 aa, Hepatitis B Virus
  REGION 1..72: note = HBsAg M133L
QGMLPVCPLIPGSSTTGTGPCRTCTTPAQGTSLYPSCCCTKPSDGNCTCIPIPSSWAFGKFLWEWASARFSW

SEQ ID NO: 27 - protein, 72 aa, Hepatitis B Virus
  REGION 1..72: note = HBsAg M133T
QGMLPVCPLIPGSSTTGTGPCRTCTTPAQGTSTYPSCCCTKPSDGNCTCIPIPSSWAFGKFLWEWASARFSW

SEQ ID NO: 28 - protein, 72 aa, Hepatitis B Virus
  REGION 1..72: note = HBsAg K141E
QGMLPVCPLIPGSSTTGTGPCRTCTTPAQGTSMYPSCCCTEPSDGNCTCIPIPSSWAFGKFLWEWASARFSW

SEQ ID NO: 29 - protein, 72 aa, Hepatitis B Virus
  REGION 1..72: note = HBsAg P142S
QGMLPVCPLIPGSSTTGTGPCRTCTTPAQGTSMYPSCCCTKSSDGNCTCIPIPSSWAFGKFLWEWASARFSW

SEQ ID NO: 30 - protein, 72 aa, Hepatitis B Virus
  REGION 1..72: note = HBsAg S143K
QGMLPVCPLIPGSSTTGTGPCRTCTTPAQGTSMYPSCCCTKPKDGNCTCIPIPSSWAFGKFLWEWASARFSW

SEQ ID NO: 31 - protein, 72 aa, Hepatitis B Virus
  REGION 1..72: note = HBsAg D144A
QGMLPVCPLIPGSSTTGTGPCRTCTTPAQGTSMYPSCCCTKPSAGNCTCIPIPSSWAFGKFLWEWASARFSW

SEQ ID NO: 32 - protein, 72 aa, Hepatitis B Virus
  REGION 1..72: note = HBsAg G145R
QGMLPVCPLIPGSSTTGTGPCRTCTTPAQGTSMYPSCCCTKPSDRNCTCIPIPSSWAFGKFLWEWASARFSW

SEQ ID NO: 33 - protein, 72 aa, Hepatitis B Virus
  REGION 1..72: note = HBsAg N146A
QGMLPVCPLIPGSSTTGTGPCRTCTTPAQGTSMYPSCCCTKPSDGACTCIPIPSSWAFGKFLWEWASARFSW

SEQ ID NO: 34 - protein, 8 aa, synthetic construct
  REGION 1..8: note = Synthetic sequence HBC34 CDRH1
GRIFRSFY

SEQ ID NO: 35 - protein, 7 aa, synthetic construct
  REGION 1..7: note = Synthetic sequence HBC34 CDRH2
NQDGSEK

SEQ ID NO: 36 - protein, 12 aa, synthetic construct
  REGION 1..12: note = Synthetic sequence HBC34 CDRH3
AAWSGNSGGMDV

SEQ ID NO: 37 - protein, 6 aa, synthetic construct
  REGION 1..6: note = Synthetic sequence HBC34 CDRL1
KLGNKN

SEQ ID NO: 38 - skipped

SEQ ID NO: 39 - protein, 9 aa, synthetic construct
  REGION 1..9: note = Synthetic sequence HBC34 CDRL2 long
VIYEVKYRP

SEQ ID NO: 40 - protein, 9 aa, synthetic construct
  REGION 1..9: note = Synthetic sequence HBC34 CDRL3
QTWDSTTVV

SEQ ID NO: 41 - protein, 119 aa, synthetic construct
  REGION 1..119: note = Synthetic sequence HBC34, HBC34-V7, HBC34-V34, HBC34-V35 VH
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSS

SEQ ID NO: 42 - protein, 106 aa, synthetic construct
  REGION 1..106: note = Synthetic sequence HBC34 VL
SYELTQPPSVSVSPGQTVSIPCSGDKLGNKNVCWFQHKPGQSPVLVIYEVKYRPSGIPERFSGSNSGNTATLTISGTQAMDEAAYFCQTWDSTTVVFGGGTRLTVL

SEQ ID NO: 43 - DNA, 24 bp, synthetic construct
  misc_feature 1..24: note = Synthetic sequence HBC34 CDRH1
  source 1..24: mol_type = other DNA
ggacgcatctttagaagtttttac

SEQ ID NO: 44 - DNA, 24 bp, synthetic construct
  misc_feature 1..24: note = Synthetic sequence HBC34 CDRH2
  source 1..24: mol_type = other DNA
ataaaccaagatggaagtgagaaa

SEQ ID NO: 45 - DNA, 36 bp, synthetic construct
  misc_feature 1..36: note = Synthetic sequence HBC34 CDRH3
  source 1..36: mol_type = other DNA
gcggcttggagcggcaatagtgggggtatggacgtc

SEQ ID NO: 46 - DNA, 18 bp, synthetic construct
  misc_feature 1..18: note = Synthetic sequence HBC34 CDRL1
  source 1..18: mol_type = other DNA
aaattggggaataaaaat

SEQ ID NO: 47 - skipped

SEQ ID NO: 48 - DNA, 27 bp, synthetic construct
  misc_feature 1..27: note = Synthetic sequence HBC34 CDRL2 long
  source 1..27: mol_type = other DNA
gtcatctatgaggttaaataccgcccc

SEQ ID NO: 49 - DNA, 27 bp, synthetic construct
  misc_feature 1..27: note = Synthetic sequence HBC34 CDRL3
  source 1..27: mol_type = other DNA
cagacgtgggacagcaccactgtggtg

SEQ ID NO: 50 - DNA, 357 bp, synthetic construct
  misc_feature 1..357: note = Synthetic sequence HBC34 VH
  source 1..357: mol_type = other DNA
gaactgcagctggtggagtctgggggaggctgggtccagccgggggggtcccagagactgtcctgtgcagcctctggacgcatctttagaagtttttacatgagctgggtccgccaggccccagggaaggggctggagtgggtggccactataaaccaagatggaagtgagaaattatatgtggactctgtgaagggccgattcaccatctccagagacaacgccaagaactcactatttctgcaaatgaacaacctgagagtcgaggacacggccgtttattactgcgcggcttggagcggcaatagtgggggtatggacgtctggggccaggggaccacggtctccgtctcctca

SEQ ID NO: 51 - DNA, 318 bp, synthetic construct
  misc_feature 1..318: note = Synthetic sequence HBC34 VL
  source 1..318: mol_type = other DNA
tcctatgagctgactcagccaccctcagtgtccgtgtccccaggacagacagtcagcatcccctgctctggagataaattggggaataaaaatgtttgctggtttcagcataagccaggccagtcccctgtgttggtcatctatgaggttaaataccgcccctcggggattcctgagcgattctctggctccaactctgggaacacagccactctgaccatcagcgggacccaggctatggatgaggctgcctatttctgtcagacgtgggacagcaccactgtggtgttcggcggagggaccaggctgaccgtccta

SEQ ID NO: 52 - protein, 33 aa, Hepatitis B Virus
  VARIANT 33: note = X = Any Amino Acid
  VARIANT 1: note = X = Any Amino Acid
  VARIANT 17: note = X = Any Amino Acid
XGSSTTSTGPCRTCMTXPSDGNATAIPIPSSWX

SEQ ID NO: 53 - protein, 15 aa, Hepatitis B Virus
TSTGPCRTCMTTAQG

SEQ ID NO: 54 - protein, 26 aa, Hepatitis B Virus
GMLPVCPLIPGSSTTSTGPCRTCMTT

SEQ ID NO: 55 - protein, 33 aa, Hepatitis B Virus
  VARIANT 33: note = X = Any Amino Acid
  VARIANT 17: note = X = Any Amino Acid
  VARIANT 1: note = X = Any Amino Acid
XSMYPSASATKPSDGNXTGPCRTCMTTAQGTSX

SEQ ID NO: 56 - protein, 11 aa, Hepatitis B Virus
  REGION 1..11: note = amino acids 120 - 130 of the S domain of HBsAg (HBV-D J02203)
PCRTCMTTAQG

SEQ ID NO: 57 - protein, 11 aa, Hepatitis B Virus
  VARIANT 3: note = X = R or K
  VARIANT 6: note = X = M or T
  VARIANT 7: note = X = T or I
  VARIANT 8: note = X = T, P or L
PCXTCXXXAQG

SEQ ID NO: 58 - protein, 9 aa, synthetic construct
  REGION 1..9: note = Synthetic sequence HBC34-V7 CDRL3 and HBC34-V23 CDRL3
QTFDSTTVV

SEQ ID NO: 59 - protein, 106 aa, synthetic construct
  REGION 1..106: note = Synthetic sequence HBC34-V7 VL
SYELTQPPSVSVSPGQTVSIPCSGDKLGNKNVCWFQHKPGQSPVLVIYEVKYRPSGIPERFSGSNSGNTATLTISGTQAMDEAAYFCQTFDSTTVVFGGGTRLTVL

SEQ ID NO: 60 - DNA, 18 bp, synthetic construct
  misc_feature 1..18: note = Synthetic sequence HBC34v7 CDRL1 and HBC-V23 CDRL1
  source 1..18: mol_type = other DNA
aagctggggaacaaaaat

SEQ ID NO: 61 - skipped

SEQ ID NO: 62 - DNA, 27 bp, synthetic construct
  misc_feature 1..27: note = Synthetic sequence HBC34-V7 CDRL2 long and CDRL2 HBC34-V23 long
  source 1..27: mol_type = other DNA
gtcatctacgaggtgaaatatcggcct

SEQ ID NO: 63 - DNA, 27 bp, synthetic construct
  misc_feature 1..27: note = Synthetic sequence CDRL3 HBC34-V7 and CDRL3 HBC34-V23
  source 1..27: mol_type = other DNA
cagacattcgattccaccacagtggtc

SEQ ID NO: 64 - DNA, 318 bp, synthetic construct
  misc_feature 1..318: note = Synthetic sequence HBC34-V7, HBC34-V34, and HBC34-V35 VL
  source 1..318: mol_type = other DNA
tcttacgagctgacacagccacctagcgtgtccgtctctccaggacagaccgtgtccatcccttgctctggcgacaagctggggaacaaaaatgtctgttggttccagcacaagccagggcagagtcccgtgctggtcatctacgaggtgaaatatcggccttcaggaattccagaacggttcagcggatcaaacagcggcaatactgcaaccctgacaattagcgggacccaggccatggacgaagccgcttatttctgccagacattcgattccaccacagtggtctttggcgggggaactaggctgaccgtgctg

SEQ ID NO: 65 - protein, 106 aa, synthetic construct
  REGION 1..106: note = Synthetic sequence HBC34-V23 VL
SYELTQPPSVSVSPGQTASITCSGDKLGNKNACWYQQKPGQSPVLVIYEVKYRPSGIPERFSGSNSGNTATLTISGTQAMDEADYYCQTFDSTTVVFGGGTKLTVL

SEQ ID NO: 66 - protein, 8 aa, synthetic construct
  REGION 1..8: note = Synthetic sequence HBC34wt CDRH2
INQDGSEK

SEQ ID NO: 67 - protein, 119 aa, synthetic construct
  REGION 1..119: note = Synthetic sequence HBC34-V31, HBC34-V32 and HBC34-V33 VH
EVQLVESGGGLVQPGGSLRLSCAASGRIFRSFYMSWVRQAPGKGLEWVANINQDGSEKLYVDSVKGRFTISRDNAKNSLFLQMNNLRVEDTAVYYCAAWSGNSGGMDVWGQGTTVTVSS

SEQ ID NO: 68 - DNA, 357 bp, synthetic construct
  misc_feature 1..357: note = Synthetic sequence HBC34-V31, HBC34-V32 and HBC34-V33 VH
  source 1..357: mol_type = other DNA
gaggtgcagctggtggaatccggcgggggactggtgcagcctggcggctcactgagactgagctgtgcagcttctggaagaatcttcagatctttttacatgagttgggtgagacaggctcctgggaagggactggagtgggtcgcaaacatcaatcaggacggatcagaaaagctgtatgtggatagcgtcaaaggcaggttcactatttcccgcgacaacgccaaaaattctctgtttctgcagatgaacaatctgcgggtggaggataccgctgtctactattgtgcagcctggtctggcaacagtggaggcatggacgtgtggggacagggaaccacagtgacagtcagctcc

SEQ ID NO: 69 - DNA, 318 bp, synthetic construct
  misc_feature 1..318: note = Synthetic sequence HBC34v23 VL
  source 1..318: mol_type = other DNA
tcttacgagctgacacagccccctagcgtgtccgtctctccaggccagacagcatccatcacttgctctggcgacaagctggggaacaaaaatgcctgttggtatcagcagaagccagggcagagtcccgtgctggtcatctacgaggtgaaatatcggccttcaggaattccagaaagattcagtggatcaaacagcggcaatactgctaccctgacaattagcgggacccaggccatggacgaagctgattactattgccagacattcgattccaccacagtggtctttggcgggggaactaagctgaccgtgctg

SEQ ID NO: 70 - DNA, 357 bp, synthetic construct
  misc_feature 1..357: note = Synthetic sequence HBC34 wt VH codon optimized
  source 1..357: mol_type = other DNA
gaactgcagctggtcgaatcaggaggagggtgggtccagcccggagggagccagagactgtcttgtgccgcatcagggaggatcttcaggagcttctacatgtcctgggtgcgccaggcaccaggcaagggactggagtgggtcgccaccatcaaccaggacggatctgaaaagctgtatgtggatagtgtcaaaggccggttcacaattagcagagacaacgctaaaaattctctgtttctgcagatgaacaatctgcgagtggaggataccgccgtctactattgcgccgcttggtctggcaacagcggcgggatggatgtctgggggcagggcacaacagtgagcgtctcttcc

SEQ ID NO: 71 - DNA, 318 bp, synthetic construct
  misc_feature 1..318: note = Synthetic sequence HBC34 wt VL codon optimized
  source 1..318: mol_type = other DNA
tcatacgaactgactcagcctccctccgtctccgtctcacctggacagaccgtctcaatcccctgctccggcgataaactgggcaacaagaacgtgtgctggttccagcacaaacccggacagagtcctgtgctggtcatctacgaggtcaagtatcggccaagcggcattcccgaaagattcagcggctccaactctgggaataccgcaacactgactatctctggaacccaggcaatggacgaggcagcttacttttgccagacttgggattcaactactgtcgtgttcggcggcggaactagactgactgtcctg

SEQ ID NO: 72 - DNA, 24 bp, synthetic construct
  misc_feature 1..24: note = Synthetic sequence HBC34 wt CDRH1 codon optimized
  source 1..24: mol_type = other DNA
gggaggatcttcaggagcttctac

SEQ ID NO: 73 - DNA, 24 bp, synthetic construct
  misc_feature 1..24: note = Synthetic sequence HBC34 wt CDRH2 codon optimized
  source 1..24: mol_type = other DNA
atcaaccaggacggatctgaaaag

SEQ ID NO: 74 - DNA, 36 bp, synthetic construct
  misc_feature 1..36: note = Synthetic sequence HBC34 wt CDRH3 codon optimized
  source 1..36: mol_type = other DNA
gccgcttggtctggcaacagcggcgggatggatgtc

SEQ ID NO: 75 - DNA, 18 bp, synthetic construct
  misc_feature 1..18: note = Synthetic sequence HBC34 wt CDRL1 codon optimized
  source 1..18: mol_type = other DNA
aaactgggcaacaagaac

SEQ ID NO: 76 - skipped

SEQ ID NO: 77 - DNA, 27 bp, synthetic construct
  misc_feature 1..27: note = Synthetic sequence HBC34 wt CDRL2 long codon optimized
  source 1..27: mol_type = other DNA
gtcatctacgaggtcaagtatcggcca

SEQ ID NO: 78 - DNA, 27 bp, synthetic construct
  misc_feature 1..27: note = Synthetic sequence HBC34 wt CDRL3 codon optimized
  source 1..27: mol_type = other DNA
cagacttgggattcaactactgtcgtg

SEQ ID NO: 79 - protein, 5 aa, synthetic construct
  REGION 1..5: note = Synthetic linker sequence
GGSGG

SEQ ID NO: 80 - protein, 7 aa, Hepatitis B Virus
TGPCRTC

SEQ ID NO: 81 - protein, 7 aa, Hepatitis B Virus
GNCTCIP

SEQ ID NO: 82 - protein, 25 aa, Hepatitis B Virus
  MOD_RES 2: note = Cysteine coupled to acetamidomethyl
  MOD_RES 21: note = Cysteine coupled to acetamidomethyl
  MOD_RES 24: note = Cysteine coupled to acetamidomethyl
CCIPIPSSWAFGCSTTSTGPCRTCC

SEQ ID NO: 83 - protein, 28 aa, Hepatitis B Virus
  MOD_RES 4: note = Cys is coupled to acetamidomethyl
  MOD_RES 6: note = Cys is coupled to acetamidomethyl
  MOD_RES 24: note = Cys is coupled to acetamidomethyl
  MOD_RES 27: note = Cys is coupled to acetamidomethyl
CGNCTCIPIPSSWAFCSTTSTGPCRTCC

SEQ ID NO: 84 - protein, 17 aa, Hepatitis B Virus
  VARIANT 13: note = cys is coupled to acetamidomethyl
  MOD_RES 16: note = cys is coupled to acetamidomethyl
CGGGCSTTSTGPCRTCC

SEQ ID NO: 85 - protein, 11 aa, Hepatitis B virus
STTSTGPCRTC

SEQ ID NO: 86 - protein, 15 aa, Hepatitis B Virus
GNCTCIPIPSSWAFC

SEQ ID NO: 87 - protein, 14 aa, Hepatitis B Virus
GNCTCIPIPSSWAF

SEQ ID NO: 88 - protein, 5 aa, Hepatitis B Virus
  VARIANT 4: note = X = Any Amino Acid
PCRXC

SEQ ID NO: 89 - protein, 106 aa, synthetic construct
  REGION 1..106: note = Synthetic sequence HBC34-V35 VL
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVL

SEQ ID NO: 90 - protein, 106 aa, synthetic construct
  REGION 1..106: note = Synthetic sequence HBC34-V34 VL
SYELTQPPSVSVSPGQTVSIPCSGDKLGNKNVSWFQHKPGQSPVLVIYEVKYRPSGIPERFSGSNSGNTATLTISGTQAMDEAAYFCQTFDSTTVVFGGGTRLTVL

SEQ ID NO: 91 - protein, 449 aa, synthetic construct
  REGION 1..449: note = Synthetic sequence HC of HBC34-V35-MLNS-GAALIE and HBC34-V34-MLNS-GAALIE (g1M17, 1)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 92 - protein, 449 aa, synthetic construct
  REGION 1..449: note = Synthetic sequence HC of HBC34-V35-MLNS and HBC34-V34-MLNS (g1M17, 1)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 93 - protein, 212 aa, synthetic construct
  REGION 1..212: note = Synthetic sequence LC of HBC34-V35
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 94 - protein, 212 aa, synthetic construct
  REGION 1..212: note = Synthetic sequence LC of HBC34-V34
SYELTQPPSVSVSPGQTVSIPCSGDKLGNKNVSWFQHKPGQSPVLVIYEVKYRPSGIPERFSGSNSGNTATLTISGTQAMDEAAYFCQTFDSTTVVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - protein, 128 aa, synthetic construct
  REGION 1..128: note = Synthetic sequence HBC24 VH
EVQLLESGGGLVQPGGSLRLSCAASGSTFTKYAMSWVRQAPGKGLEWVASISGSVPGFGIDTYYADSVKGRFTISRDTSKNTLYLQMNSLRAEDTALYYCAKDVGVIGSYYYYAMDVWGQGTAVTVSS

SEQ ID NO: 96 - protein, 109 aa, synthetic construct
  REGION 1..109: note = Synthetic sequence HBC24 VL
EIVLTQSPGTLSLSPGERATLSCRASQGLSSSYLAWYQQKPGQAPRLLIYSASTRATGIPDRFSGSGSGTDFTLTISRLEPEDFAVYYCQQYAYSPRWTFGQGTKVEIK

SEQ ID NO: 97 - protein, 8 aa, synthetic construct
  REGION 1..8: note = Synthetic sequence CDRH1 of HBC24
GSTFTKYA

SEQ ID NO: 98 - protein, 8 aa, synthetic construct
  REGION 1..8: note = Synthetic sequence CDRH2 of HBC24
ISGSVPGF

SEQ ID NO: 99 - protein, 21 aa, synthetic construct
  REGION 1..21: note = Synthetic sequence CDRH3 of HBC24
LYYCAKDVGVIGSYYYYAMDV

SEQ ID NO: 100 - protein, 7 aa, synthetic construct
  REGION 1..7: note = Synthetic sequence CDRL1 of HBC24
QGLSSSY

SEQ ID NO: 101 - skipped

SEQ ID NO: 102 - protein, 10 aa, synthetic construct
  REGION 1..10: note = Synthetic sequence CDRL3 of HBC24
QQYAYSPRWT

SEQ ID NO: 103 - DNA, 357 bp, synthetic construct
  misc_feature 1..357: note = Synthetic sequence VH of HBC34-V7, HBC34-V35, and HBC34-V34 (codon optimized)
  source 1..357: mol_type = other DNA
gagctgcagctggtggagtccggcggcggctgggtgcagcctggcggctcccagaggctgagctgtgccgcttctggcaggatcttccggtccttttacatgtcttgggtgcggcaggctccaggcaagggcctggagtgggtggctaccatcaaccaggacggctccgagaagctgtatgtggatagcgtgaagggcagattcacaatctctcgcgacaacgccaagaactccctgtttctgcagatgaacaatctgagggtggaggataccgccgtgtactattgcgccgcttggtctggcaatagcggcggcatggacgtgtggggacagggcaccaccgtgtccgtgtccagc

SEQ ID NO: 104 - DNA, 318 bp, synthetic construct
  misc_feature 1..318: note = Synthetic sequence HBC34-V34 VL (codon optimized)
  source 1..318: mol_type = other DNA
agctacgagctgacacagcccccttccgtgtccgtgtcccctggacagaccgtgtccatcccatgcagcggcgacaagctgggcaacaagaacgtgtcctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctg

SEQ ID NO: 105 - DNA, 318 bp, synthetic construct
  misc_feature 1..318: note = Synthetic sequence HBC34-V35 VL (codon optimized)
  source 1..318: mol_type = other DNA
agctacgagctgacacagcccccttccgtgtccgtgtcccctggacagaccgtgtccatcccatgcagcggcgacaagctgggcaacaagaacgtggcctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctg

SEQ ID NO: 106 - DNA, 360 bp, synthetic construct
  misc_feature 1..360: note = Synthetic sequence HBC24 VH (wild type)
  source 1..360: mol_type = other DNA
gaggtgcagttgttggagtctgggggaggcttggtacagcctggggggtccctgagactctcctgtgcagcctctggatccacttttaccaaatatgccatgagctgggtccgtcaggctccagggaaggggctggagtgggtcgcaagtattagtggaagtgttcctggttttggtattgacacatactacgcagactccgttaagggccggttcaccatctccagagacacttccaagaacaccctgtatctgcaaatgaacagcctgagagccgaggacacggccttatattactgtgcgaaagatgtcggggttatcgggtcatactattactacgctatggacgtctggggtcaa

SEQ ID NO: 107 - DNA, 327 bp, synthetic construct
  misc_feature 1..327: note = Synthetic sequence HBC24 VL (wild type)
  source 1..327: mol_type = other DNA
aaattgtgttgacgcagtctccaggcaccctgtctttgtctccaggggaaagagccaccctctcctgcagggccagtcagggtcttagcagcagttacttagcctggtaccagcagaaacctggccaggctcccaggctcctcatctatagtgcgtccaccagggccactggcatcccagacaggttcagtggcagtgggtctgggacagacttcactctcaccatcagcagactggagcctgaagattttgcagtgtattactgtcaacagtatgcttactcacctcggtggacgttcggccaagggaccaaggtggagatcaaac

SEQ ID NO: 108 - DNA, 384 bp, synthetic construct
  misc_feature 1..384: note = Synthetic sequence HBC24 VH (codon optimized)
  source 1..384: mol_type = other DNA
gaggtgcagctgctggaaagcggcggcggcctggtgcagcccggcggctccctgaggctgtcttgcgccgcctctggcagcaccttcacaaagtatgcaatgtcttgggtgcgccaggcaccaggcaagggcctggagtgggtggcctccatctctggcagcgtgcctggcttcggcatcgacacctactatgccgattccgtgaagggccggtttacaatcagcagagacacctccaagaacacactgtatctgcagatgaattctctgcgggccgaggacaccgccctgtactattgtgccaaggatgtgggcgtgatcggcagctactattactatgcaatggacgtgtggggacagggaacagcagtgacagtgagctcc

SEQ ID NO: 109 - DNA, 327 bp, synthetic construct
  misc_feature 1..327: note = Synthetic sequence HBC24 VL (codon optimized)
  source 1..327: mol_type = other DNA
gagatcgtgctgacccagtctcctggcacactgtccctgtcccctggagagagagccaccctgtcctgcagagcctctcagggcctgagctcctcttacctggcctggtatcagcagaagcctggacaggcccctcggctgctgatctactctgcctccaccagagcaacaggcattcctgaccgcttctccggatctggaagcggcacagacttcaccctgacaatcagccggctggagcctgaggacttcgccgtgtactattgtcagcagtacgcctattccccaaggtggacctttggccagggcacaaaggtggagatcaag

SEQ ID NO: 110 - DNA, 318 bp, synthetic construct
  misc_feature 1..318: note = Synthetic sequence HBC34-V7 VL (codon optimized)
  source 1..318: mol_type = other DNA
agctacgagctgacacagcccccttccgtgtccgtgtcccctggacagaccgtgtccatcccatgcagcggcgacaagctgggcaacaagaacgtgtgctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctg

SEQ ID NO: 111 - protein, 106 aa, synthetic construct
  REGION 1..106: note = Synthetic sequence HBC34v23-L_C40S
SYELTQPPSVSVSPGQTASITCSGDKLGNKNASWYQQKPGQSPVLVIYEVKYRPSGIPERFSGSNSGNTATLTISGTQAMDEADYYCQTFDSTTVVFGGGTKLTVL

SEQ ID NO: 112 - protein, 106 aa, synthetic construct
  REGION 1..106: note = Synthetic sequence HBC34v23-L_C40A
SYELTQPPSVSVSPGQTASITCSGDKLGNKNAAWYQQKPGQSPVLVIYEVKYRPSGIPERFSGSNSGNTATLTISGTQAMDEADYYCQTFDSTTVVFGGGTKLTVL

SEQ ID NO: 113 - protein, 106 aa, synthetic construct
  REGION 1..106: note = Synthetic sequence HBC34v31-L_C40S
SYELTQPPSVSVSPGQTVSIPCSGDKLGNKNVSWFQHKPGQSPVLVIYEVKYRPSGIPERFSGSNSGNTATLTISGTQAMDEAAYFCQTWDSTTVVFGGGTRLTVL

SEQ ID NO: 114 - protein, 106 aa, synthetic construct
  REGION 1..106: note = Synthetic sequence HBC34v31-L_C40A
SYELTQPPSVSVSPGQTVSIPCSGDKLGNKNVAWFQHKPGQSPVLVIYEVKYRPSGIPERFSGSNSGNTATLTISGTQAMDEAAYFCQTWDSTTVVFGGGTRLTVL

SEQ ID NO: 115 - protein, 106 aa, synthetic construct; identical to SEQ ID NO: 90
  REGION 1..106: note = Synthetic sequence HBC34v32-L_C40S

SEQ ID NO: 116 - protein, 106 aa, synthetic construct; identical to SEQ ID NO: 114
  REGION 1..106: note = Synthetic sequence HBC34v32-L_C40A

SEQ ID NO: 117 - protein, 106 aa, synthetic construct; identical to SEQ ID NO: 111
  REGION 1..106: note = Synthetic sequence HBC34v33-L_C40S

SEQ ID NO: 118 - protein, 106 aa, synthetic construct; identical to SEQ ID NO: 112
  REGION 1..106: note = Synthetic sequence HBC34v33-L_C40A

SEQ ID NO: 119 - protein, 106 aa, synthetic construct; identical to SEQ ID NO: 113
  REGION 1..106: note = Synthetic sequence HBC34-L_C40S

SEQ ID NO: 120 - protein, 106 aa, synthetic construct; identical to SEQ ID NO: 114
  REGION 1..106: note = Synthetic sequence HBC34-L_C40A

SEQ ID NO: 121 - protein, 458 aa, synthetic construct
  REGION 1..458: note = Synthetic sequence HBC24-MLNS
EVQLLESGGGLVQPGGSLRLSCAASGSTFTKYAMSWVRQAPGKGLEWVASISGSVPGFGIDTYYADSVKGRFTISRDTSKNTLYLQMNSLRAEDTALYYCAKDVGVIGSYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 122 - protein, 458 aa, synthetic construct
  REGION 1..458: note = Synthetic sequence HBC24-MLNS-GAALIE
EVQLLESGGGLVQPGGSLRLSCAASGSTFTKYAMSWVRQAPGKGLEWVASISGSVPGFGIDTYYADSVKGRFTISRDTSKNTLYLQMNSLRAEDTALYYCAKDVGVIGSYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 123 - protein, 449 aa, synthetic construct
  REGION 1..449: note = Synthetic sequence HBC34-V7-mu (IgG2a) HC
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 124 - protein, 212 aa, synthetic construct
  REGION 1..212: note = Synthetic sequence HBC34-V7-mu (IgG2a) LC
SYELTQPPSVSVSPGQTVSIPCSGDKLGNKNVCWFQHKPGQSPVLVIYEVKYRPSGIPERFSGSNSGNTATLTISGTQAMDEAAYFCQTFDSTTVVFGGGTRLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 125 - protein, 449 aa, synthetic construct; identical to SEQ ID NO: 123
  REGION 1..449: note = Synthetic sequence HBC34-V35-mu (IgG2a) HC

SEQ ID NO: 126 - protein, 212 aa, synthetic construct
  REGION 1..212: note = Synthetic sequence HBC34-V35-mu (IgG2a) LC
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 127 - protein, 458 aa, synthetic construct
  REGION 1..458: note = Synthetic sequence HBC24-mu (IgG2a) HC
EVQLLESGGGLVQPGGSLRLSCAASGSTFTKYAMSWVRQAPGKGLEWVASISGSVPGFGIDTYYADSVKGRFTISRDTSKNTLYLQMNSLRAEDTALYYCAKDVGVIGSYYYYAMDVWGQGTAVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 128 - protein, 215 aa, synthetic construct
  REGION 1..215: note = Synthetic sequence HBC24-mu (IgG2a) LC
EIVLTQSPGTLSLSPGERATLSCRASQGLSSSYLAWYQQKPGQAPRLLIYSASTRATGIPDRFSGSGSGTDFTLTISRLEPEDFAVYYCQQYAYSPRWTFGQGTKVEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 129 - protein, 449 aa, synthetic construct
  REGION 1..449: note = Synthetic sequence HBC34-V7, HBC34-V34, HBC34-V35 HC (wild-type)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - DNA, 990 bp, synthetic construct
  misc_feature 1..990: note = Synthetic sequence HBC34-V7, HBC34-V34, HBC34-V35 CH1-hinge-CH2-CH3 (codon-optimized)
  source 1..990: mol_type = other DNA
gcctccacaaagggcccaagcgtgtttccactggctccctcttccaagtctacctccggcggcacagccgctctgggatgtctggtgaaggattacttcccagagcccgtgaccgtgtcttggaactccggcgccctgaccagcggagtgcatacatttccagctgtgctgcagagctctggcctgtactctctgtccagcgtggtgaccgtgccctcttccagcctgggcacccagacatatatctgcaacgtgaatcacaagccaagcaatacaaaggtggacaagaaggtggagcccaagtcttgtgataagacccatacatgccctccatgtccagctccagagctgctgggcggcccaagcgtgttcctgtttccacccaagcctaaggataccctgatgatctccagaacccccgaggtgacatgcgtggtggtggacgtgagccacgaggatcctgaggtgaagttcaactggtacgtggacggcgtggaggtgcataatgctaagaccaagcccagggaggagcagtacaactctacctatcgggtggtgtccgtgctgacagtgctgcaccaggattggctgaacggcaaggagtataagtgcaaggtgtctaataaggccctgcccgctcctatcgagaagaccatctccaaggccaagggccagcctagagagccacaggtgtacacactgcctccatctcgcgatgagctgaccaagaaccaggtgtccctgacatgtctggtgaagggcttctatccttccgacatcgctgtggagtgggagagcaatggccagccagagaacaattacaagaccacaccccctgtgctggacagcgatggctctttctttctgtatagcaagctgaccgtggacaagtctcgctggcagcagggcaacgtgtttagctgttctgtgatgcatgaggccctgcacaatcattatacacagaagtccctgagcctgtctcctggcaag

SEQ ID NO: 131 - DNA, 1417 bp, synthetic construct
  misc_feature 1..1417: note = Synthetic sequence HBC34-V7, HBC34-V34, HBC34-V35 VH-CH1-hinge-CH2-CH3 (codon-optimized)
  source 1..1417: mol_type = other DNA
gagctgcagctggtggagtccggcggcggctgggtgcagcctggcggctcccagaggctgagctgtgccgcttctggcaggatcttccggtccttttacatgtcttgggtgcggcaggctccaggcaagggcctggagtgggtggctaccatcaaccaggacggctccgagaagctgtatgtggatagcgtgaagggcagattcacaatctctcgcgacaacgccaagaactccctgtttctgcagatgaacaatctgagggtggaggataccgccgtgtactattgcgccgcttggtctggcaatagcggcggcatggacgtgtggggacagggcaccaccgtgtccgtgtccagcgcctccacaaagggcccaagcgtgtttccactggctccctcttccaagtctacctccggcggcacagccgctctgggatgtctggtgaaggattacttcccagagcccgtgaccgtgtcttggaactccggcgccctgaccagcggagtgcatacatttccagctgtgctgcagagctctggcctgtactctctgtccagcgtggtgaccgtgccctcttccagcctgggcacccagacatatatctgcaacgtgaatcacaagccaagcaatacaaaggtggacaagaaggtggagcccaagtcttgtgataagacccatacatgccctccatgtccagctccagagctgctgggcggcccaagcgtgttcctgtttccacccaagcctaaggataccctgatgatctccagaacccccgaggtgacatgcgtggtggtggacgtgagccacgaggatcctgaggtgaagttcaactggtacgtggacggcgtggaggtgcataatgctaagaccaagcccagggaggagcagtacaactctacctatcgggtggtgtccgtgctgacagtgctgcaccaggattggctgaacggcaaggagtataagtgcaaggtgtctaataaggccctgcccgctcctatcgagaagaccatctccaaggccaagggccagcctagagagccacaggtgtacacactgcctccatctcgcgatgagctgaccaagaaccaggtgtccctgacatgtctggtgaagggcttctatccttccgacatcgctgtggagtgggagagcaatggccagccagagaacaattacaagaccacaccccctgtgctggacagcgatggctctttctttctgtatagcaagctgaccgtggacaagtctcgctggcagcagggcaacgtgtttagctgttctgtgatgcatgaggccctgcacaatcattatacacagaagtccctgagcctgtctcctggcaagtgatgaggtaccgtgcgacggccggcaagcccccgctccccgggctctcgcggtcgtacgaggaaagctt

SEQ ID NO: 132 - DNA, 318 bp, synthetic construct
  misc_feature 1..318: note = Synthetic sequence HBC34-V7 CL (codon-optimized)
  source 1..318: mol_type = other DNA
ggacagccaaaggctgctccatctgtgaccctgtttccaccctcttccgaggagctgcaggccaacaaggccaccctggtgtgcctgatctctgacttctaccctggagctgtgacagtggcttggaaggctgatagctctcccgtgaaggctggcgtggagacaacaacccctagcaagcagtctaacaataagtacgccgcttccagctatctgtctctgacaccagagcagtggaagtcccaccgctcttattcctgccaggtgacccatgagggcagcaccgtggagaagacagtggcccccaccgagtgttct

SEQ ID NO: 133 - DNA, 636 bp, synthetic construct
  misc_feature 1..636: note = Synthetic sequence HBC34-V7 LC (VL-CL) (codon-optimized)
  source 1..636: mol_type = other DNA
agctacgagctgacacagcccccttccgtgtccgtgtcccctggacagaccgtgtccatcccatgcagcggcgacaagctgggcaacaagaacgtgtgctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctgggacagccaaaggctgctccatctgtgaccctgtttccaccctcttccgaggagctgcaggccaacaaggccaccctggtgtgcctgatctctgacttctaccctggagctgtgacagtggcttggaaggctgatagctctcccgtgaaggctggcgtggagacaacaacccctagcaagcagtctaacaataagtacgccgcttccagctatctgtctctgacaccagagcagtggaagtcccaccgctcttattcctgccaggtgacccatgagggcagcaccgtggagaagacagtggcccccaccgagtgttct

SEQ ID NO: 134 - DNA, 318 bp, synthetic construct; identical to SEQ ID NO: 132
  misc_feature 1..318: note = Synthetic sequence HBC34-V34, HBC34-V35 CL (codon-optimized)
  source 1..318: mol_type = other DNA

SEQ ID NO: 135 - DNA, 636 bp, synthetic construct
  misc_feature 1..636: note = Synthetic sequence HBC34-V34 LC (VL-CL) (codon-optimized)
  source 1..636: mol_type = other DNA
agctacgagctgacacagcccccttccgtgtccgtgtcccctggacagaccgtgtccatcccatgcagcggcgacaagctgggcaacaagaacgtgtcctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctgggacagccaaaggctgctccatctgtgaccctgtttccaccctcttccgaggagctgcaggccaacaaggccaccctggtgtgcctgatctctgacttctaccctggagctgtgacagtggcttggaaggctgatagctctcccgtgaaggctggcgtggagacaacaacccctagcaagcagtctaacaataagtacgccgcttccagctatctgtctctgacaccagagcagtggaagtcccaccgctcttattcctgccaggtgacccatgagggcagcaccgtggagaagacagtggcccccaccgagtgttct

SEQ ID NO: 136 - DNA, 636 bp, synthetic construct
  misc_feature 1..636: note = Synthetic sequence HBC34-V35 LC (VL-CL) (codon-optimized)
  source 1..636: mol_type = other DNA
agctacgagctgacacagcccccttccgtgtccgtgtcccctggacagaccgtgtccatcccatgcagcggcgacaagctgggcaacaagaacgtggcctggtttcagcataagcctggccagtcccccgtgctggtcatctacgaggtgaagtataggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctgggacagccaaaggctgctccatctgtgaccctgtttccaccctcttccgaggagctgcaggccaacaaggccaccctggtgtgcctgatctctgacttctaccctggagctgtgacagtggcttggaaggctgatagctctcccgtgaaggctggcgtggagacaacaacccctagcaagcagtctaacaataagtacgccgcttccagctatctgtctctgacaccagagcagtggaagtcccaccgctcttattcctgccaggtgacccatgagggcagcaccgtggagaagacagtggcccccaccgagtgttct

SEQ ID NO: 137 - protein, 217 aa, Homo sapiens
  REGION 1..217: note = IgG1
  REGION 1..217: note = WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, synthetic construct
  REGION 1..449: note = Synthetic sequence HBC34, HBC34v7, HBC34v23, HBC34v34, HBC34v35, HBC34_C40S, HBC34_C40A, HBC34v23_C40S, HBC34v23_C40A HC with GAALIE mutation in hIgG1 Fc
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 19 aa, synthetic construct
  REGION 1..19: note = Chimeric hinge sequence
ESKYGPPCPPCPAPPVAGP

(19) United States
(12) Patent Application Publication
(10) Pub. No.: US 2026/0201019 A1
(43) Pub. Date: Jul. 16, 2026

(54) ANTIBODY COMPOSITIONS AND METHODS FOR TREATING HEPATITIS B VIRUS INFECTION

(71) Applicant: VIR BIOTECHNOLOGY, INC., San Francisco, CA (US)

(72) Inventors: Phillip S. PANG, Menlo Park, CA (US); Erik MOGALIAN, San Francisco, CA (US); Lynn E. CONNOLLY, San Francisco, CA (US); Jonathan GALL, San Francisco, CA (US)

(21) Appl. No.: 19/429,547

(22) Filed: Dec. 22, 2025

Related U.S. Application Data
(63) Continuation of application No. 17/637,605, filed on Aug. 28, 2020, now abandoned.
(60) Provisional application No. 62/893,742, filed on Aug. 29, 2019.

(51) Int. Cl. C07K 16/082; A61K 39/00; A61P 31/20

(52) U.S. Cl. CPC C07K 16/082; A61P 31/20; A61K 2039/505; A61K 2039/545; C07K 2317/21; C07K 2317/524; C07K 2317/526; C07K 2317/72; C07K 2317/94

(57) ABSTRACT

The present disclosure relates to pharmaceutical compositions that comprise an antibody that neutralizes infection of hepatitis B virus (HBV). In addition, the present disclosure relates to the use of the pharmaceutical compositions in the treatment of HBV infection.

### STATEMENT REGARDING SEQUENCE LISTING

This application contains a Sequence Listing, which has been submitted electronically in xml format and is hereby incorporated by reference in its entirety. Said xml copy, created on Dec. 17, 2025, is named SeqList-368561-41211.xml and is 196,308 bytes in size.

The present disclosure relates to pharmaceutical antibody compositions and methods for prophylaxis and treatment of Hepatitis B Virus infection.

### BACKGROUND

HBV consists of (i) an envelope containing three related surface proteins (hepatitis B surface antigen, HBsAg) and lipid and (ii) an icosahedral nucleocapsid enclosing the viral DNA genome and DNA polymerase. The HBV capsid is formed in the cytosol of the infected cell during packaging of an RNA pregenome replication complex and gains the ability to bud during synthesis of the viral DNA genome by reverse transcription of the pregenome in the lumen of the particle. The three HBV envelope proteins S-HBsAg, M-HBsAg, and L-HBsAg shape a complex transmembrane fold at the endoplasmic reticulum, and form disulfide-linked homo- and heterodimers. During budding at an intracellular membrane, a short linear domain in the cytosolic preS region interacts with binding sites on the capsid surface. The virions are subsequently secreted into the blood. In addition, the surface proteins can bud in the absence of capsids and form subviral particles (SVPs) which are also secreted in 3-4 log excess over virions. High level of HBsAg can exhaust HBsAg-specific T-cell response, and is proposed as an important factor for viral immunotolerance in patients with chronic hepatitis B (CHB) (Chisari F V, Isogawa M, Wieland S F, Pathologie Biologie, 2010; 58:258-66).

Hepatitis B virus causes potentially life-threatening acute and chronic liver infections. Acute hepatitis B is characterized by viremia, with or without symptoms, with the risk of fulminant hepatitis occurrence (Liang T J, Block T M, McMahon B J, Ghany M G, Guo J T, Locarnini S, Zoulim F, Chang K M, Lok A S. Present and future therapies of hepatitis B: From discovery to cure. Hepatology. 2015 Aug. 3. doi: 10.1002/hep.28025. [Epub ahead of print]). Despite an efficacious vaccine against hepatitis B being available since 1982, WHO reports that 240 million people are chronically infected with hepatitis B and more than 780 000 people die every year due to hepatitis B complications. Approximately one third of chronic hepatitis B (CHB) patients develop cirrhosis, liver failure and hepatocellular carcinoma, accounting for 600,000 deaths per year (Liang T J, Block T M, McMahon B J, Ghany M G, Urban S, Guo J T, Locarnini S, Zoulim F, Chang K M, Lok A S. Present and future therapies of hepatitis B: From discovery to cure. Hepatology. 2015 Aug. 3. doi: 10.1002/hep.28025. [Epub ahead of print]).

For patients infected with HBV, severe complications can develop as a result of coinfection or superinfection with HDV. According to the WHO, hepatitis D infects about 15 million people worldwide. HDV is considered a subviral satellite because it can propagate only in the presence of HBV. HDV is one of the smallest known animal viruses (40 nm), whereby its genome is only 1.6 kb and encodes for S and L HDAg. All other proteins needed for genome replication of HDV, including the RNA polymerase, are provided by the host cell, and the HDV envelope is provided by HBV. When introduced into permissive cells, the HDV RNA genome replicates and associates with multiple copies of the HDV-encoded proteins to assemble a ribonucleoprotein (RNP) complex. The RNP is exported from the cell by the HBV envelope proteins, which are able to assemble lipoprotein vesicles that bud into the lumen of a pre-Golgi compartment before being secreted. Moreover, the HBV envelope proteins also provide a mechanism for the targeting of HDV to an uninfected cell, thereby ensuring the spread of HDV.

Complications caused by HDV include a greater likelihood of experiencing liver failure in acute infections and a rapid progression to liver cirrhosis, with an increased chance of developing liver cancer in chronic infections. In combination with hepatitis B virus, hepatitis D has the highest fatality rate of all the hepatitis infections, at 20% (Fattovich G, Giustina G, Christensen E, Pantalena M, Zagni I, Realdi G, Schalm S W. Influence of hepatitis delta virus infection on morbidity and mortality in compensated cirrhosis type B. Gut. 2000 March; 46 (3): 420-6). The only approved therapy for chronic HDV infection is interferon-alpha. However, treatment of HDV with interferon-alpha is relatively inefficient and not well-tolerated. Treatment with interferon-alpha results in sustained virological response six months post-treatment in one fourth of the patients. Also, nucleos(t)ide analogs (NAs) have been widely tested in hepatitis delta, but they appear to be ineffective. Combination treatment of NAs with interferon also proved to be disappointing (Zaigham Abbas, Minaam Abbas Management of hepatitis delta: Need for novel therapeutic Options. World J Gastroenterol 2015 Aug. 28; 21 (32): 9461-9465). Accordingly, new therapeutic options are needed.

### BRIEF DESCRIPTION OF THE DRAWINGS

The figures provided herein are intended to illustrate subject matter included in the present disclosure in more detail. The figures are not intended to limit the disclosure in any way. Throughout the disclosure, exemplary antibody HBC34v35 (with or without Fc mutations such as MLNS and GAALIE) is also referred-to as HBC34-v35 and HBC34-V35. Accordingly, it will be understood that HBC34v35, HBC34-v35, and HBC34-V35 have the same meaning. Similarly, exemplary antibody HBC34v34 is also referred-to as HBC34-v34 and HBC34-V34, and exemplary antibody HBC34v7 is also referred-to as HBC34-v7 and HBC34-V7. Further, it will be understood “MLNS-GAALIE” has the same meaning as “MLNS_GAALIE” (i.e., M428L+N434S+G236A+A330L+1332E mutations (EU numbering) in a Fc moiety).

FIGS. 1A-1B show binding of HBC34-v7 and two engineered antibodies of the present disclosure (“HBC34-v34”; “HBC34-v35”) at the indicated concentrations to HBsAg adw (1A) and HBsAg adr (1B), as determined in direct antigen-based ELISA assays. All antibodies were produced as IgG1 (glm17, 1 allotype).

FIGS. 2A-2K show binding of HBC34-v7, HBC34-v34, and HBC34-v35 to all known HBsAg genotypes ((A)-(J), respectively) and to mock control (K). Genotype-representative sequences representing the HBsAg antigenic outer loop, as shown in Example 5 of PCT Publication No. WO 2017/060504, were used. Staining was performed by FACS. Antibody concentrations were as indicated on the y-axis of the graphs.

FIGS. 3A and 3B show binding of HBC34-v7 and HBC34-v35 with wild type or variant Fc regions to HBsAg adw in a direct antigen-based ELISA assay (2 experiments; data from “Experiment 1” is shown in FIG. 3A, and data from “Experiment 2” is shown in FIG. 3B). Antigen-binding curves are shown in the top panel of each Figure. EC50 values (determined by fitting the curves using Graphpad prism) are shown in the middle panel of each Figure. Binding to uncoated plates (control) is shown in the bottom panel of each Figure. Fc regions: “HBC34v7” and “HBC34-v35”=wild-type; “HBC34-v35-MLNS”=M428L/N434S. “HBC34-v35-MLNS-GAALIE”=M428L/N434S/G236A/A330L/1332E. Three lots of HBC34-v35 were tested. Two lots of HBC34-v35-MLNS and two lots of HBC34-v35-MLNS-GAALIE were tested. One lot of HBC34-v7 was used.

FIGS. 4-7 show the effect of HBC34-v35 on serum HBAg levels in an in vivo mouse model of HBV infection. AAV/HBV-infected SCID mice were transplanted with primary human hepatocytes and administered HBC34-v35 at 1, 5, or 15 mg/kg, or PBS (control), as described in Example 5. FIG. 4 shows serum HBV DNA concentration before and after treatment. FIG. 5 shows serum HBsAg concentration before and after treatment. FIG. 6 shows serum HBeAg concentration before and after treatment. FIG. 7 shows serum HBcrAg concentration before and after treatment.

FIGS. 8A-8E show binding of HBC34-v35-MLNS and HBC34-v35-MLNS-GAALIE to human FcγRs as assessed by biolayer interferometry (BLI). His-tagged human FcγRs ((A) FcγRIIa allele H131; (B) FcγRIla allele R131; (C) FcγRIIIa allele F158; (D) FcγRIIIa allele V158; (E) FcγRIIb) at 2 μg/ml were captured onto anti-penta-His sensors for 6 minutes. FcγRs-loaded sensors were then exposed for 5 minutes to a solution of kinetics buffer (pH 7.1) containing 2 μg/ml of each mAb (left part of the plot) in the presence 1 μg/ml of affiniPure F(ab′)₂ Fragment Goat Anti-Human IgG, F(ab′)₂ fragment specific (to cross-link human mAbs through the Fab fragment), followed by a dissociation step in the same buffer for additional 4 minutes (right part of the plot). Association and dissociation profiles were measured in real time as change in the interference pattern using an Octet RED96 (FortéBio).

FIG. 9 shows binding of HBC34-v35-MLNS and HBC34-V35-MLNS-GAALIE to human C1q as measured by Octet. Anti-human Fab (CH1) sensors were used to capture, through the Fab fragment, the full IgG1 of HBC34-v35-MLNS and HBC34-v35-MLNS-GAALIE mAbs at 10 μg/ml for 10 minutes. IgG-loaded sensors were then exposed for 4 minutes to a solution of kinetics buffer (pH 7.1) containing 3 μg/ml of purified human C1q (left part of the plot), followed by a dissociation step in the same buffer for additional 4 minutes (right part of the plot). Association and dissociation profiles were measured in real time as change in the interference pattern using an Octet RED96 (FortéBio).

FIGS. 10A and 10B show in vitro activation of human FcγRIIIa using receptor-linked activation of a NFAT-mediated Luciferase reporter in engineered Jurkat cells. FcγRIIIa activation was tested using a validated, commercially available bioreporter assay in which recombinant HBsAg (Engerix B) is used as target antigen. Serial dilutions of HBC34v35-MLNS and HBC34-v35-MLNS-GAALIE and a control (Ctr) mAb were incubated with 0.2 μg/ml of HBsAg at 37° C. for 25 min. Jurkat effector cells (Promega) expressing either FcγRIIIa low affinity allele F158 (A) or FcγRIIIa high affinity allele V158 (B) were resuspended in assay buffer and then added to assay plates. After incubation at 37° C. for 24 hours, Bio-Glo-™ Luciferase Assay Reagent (Promega) was added, and luminescence was quantified using luminometer (Bio-Tek).

FIGS. 11A and 11B show in vitro activation of human FcγRIla using receptor-linked activation of a NFAT-mediated luciferase reporter in engineered Jurkat cells. Activation of human FcγRIIa using a validated, commercially available bioreporter assay in which recombinant HBsAg (Engerix B) is used as target antigen. Serial dilutions of HBC34-v35-MLNS and HBC34-v35-MLNS-GAALIE and a control mAb (Ctr) were incubated with 2 (A) or 0.2 μg/ml (B) of HBsAg at 37° C. for 25 min. Jurkat effector cells (Promega) expressing FcγRIIa high affinity allele H131 were resuspended in assay buffer and then added to assay plates. After incubation at 37° C. for 23 hours, Bio-Glo-™ Luciferase Assay Reagent (Promega) was added, and luminescence was quantified using luminometer (Bio-Tek).

FIG. 12 shows in vitro activation of human FcγRIIb using receptor-linked activation of a NFAT-mediated luciferase reporter in engineered Jurkat cells. Activation of human FcγRIIb was tested using a validated, commercially available bioreporter assay in which recombinant HBsAg (Engerix B) is used as target antigen. Serial dilutions of HBC34-v35-MLNS and HBC34-v35-MLNS-GAALIE and a control mAb (Ctr) were incubated with 1 μg/ml of HBsAg at 37° C. for 15 min. Jurkat effector cells (Promega) expressing FcγRIIb were resuspended in assay buffer and then added to assay plates. After incubation at 37° C. for 20 hours, Bio-Glo-™ Luciferase Assay Reagent (Promega) was added, and luminescence was quantified using luminometer (Bio-Tek).

FIGS. 13A and 13B show in vitro killing of PLC/PRF/5 human hepatoma cells by human primary NK cells in the presence of HBC34-v35-MLNS and HBC34-v35-MLNS-GAALIE. (A) ADCC was tested using freshly isolated NK cells from one donor previously genotyped for expressing heterozygous high (V158) and low (F158) affinity FcγRIIIa (F/V). Serial dilutions of HBC34-v35, HBC34-v35-MLNS, HBC34-v35-MLNS-GAALIE, 17.1.41, and a control mAb were added to the HBsAg-secreting hepatoma cell line PLC/PRF/5 (also referred to as Alexander cells). PLC/PRF/5 cells were incubated together with antibodies at room temperature for 10 min. NK cells were added to assay plates (effector cells to target cells ratio of 10:1) and incubated at 37° C. for 4 hours. Cell death was determined by measuring lactate dehydrogenase (LDH) release. (B) Staining of PLC/PRF/5 human hepatoma cells by HBC34v35 and 17.1.41 mAbs as assessed by flow cytometry. Cells were extensively washed, fixed with formaldehyde (4%) or fixed and permeabilized (saponin 0.5%) before staining with different concentrations of HBC34-v35 and 17.1.41 mAbs. Binding of these human mAbs was detected by flow-cytometry using an Alexa Fluor® 647 AffiniPure F(ab′)₂ Fragment Goat Anti-Human IgG, Fcγ Fragment Specific antibody.

FIGS. 14A and 14B show in vitro activation of primary human NK cells in the presence of HBC34v35-MLNS and HBC34-v35-MLNS-GAALIE and HBsAg. Activation of NK cells was tested using freshly isolated cells from two donors previously genotyped for expressing (A) homozygous high (V158) or (B) low (F158) affinity FcγRIIIa. Serial dilutions of HBC34-V35, HBC34-v35-MLNS-GAALIE, and HBC34-v35-LALA mAbs were incubated with NK cells for 4 hours. Activation of NK cells was measured by flow cytometry by staining NK cells with anti-CD107a mAb, as a functional marker for the identification of NK cell activity. CD107a, also known as LAMP-1, is a marker for degranulation of NK cells.

FIGS. 15A-15C show a Schedule of Assessments for healthy adult subjects in an exemplary single ascending dose (SAD) clinical study of an exemplary a pharmaceutical composition comprising antibody HBC34-v35-MLNS-GAALIE, as described in Example 9.

FIGS. 16A-16E show a Schedule of Assessments for subjects with chronic HBV infection without cirrhosis and on nucleoside reverse transcriptase inhibitor (NRTI) therapy, in the exemplary clinical study described in Example 9.

FIGS. 17A-17C show timepoints for taking pharmacokinetic measurements of subjects according to the exemplary clinical study described in Example 9.

FIG. 18 shows a dosing schedule according to the exemplary clinical study described in Example 9.

FIG. 19 shows clinical laboratory assessments according to the exemplary clinical study described in Example 9.

FIG. 20 shows upregulation of activation and co-stimulatory markers on monocyte-derived dendritic cells (moDCs) stimulated via immune complexes of: HBC34-v35-MLNS+HBsAg; or HBC34-v35-MLNS-GAALIE+HBsAg, as described in Example 10.

FIG. 21 shows secretion of cytokines by moDCs stimulated via immune complexes of: HBC34-v35-MLNS+HBsAg; or HBC34-v35-MLNS-GAALIE+HBsAg, as described in Example 10.

FIGS. 22A and 22B show release of IFN-γ in whole blood cultures stimulated via immune complexes with: HBC34-v35-MLNS and HBsAg; or HBC34-v35-MLNS-GAALIE and HBsAg, as described in Example 10. (A) IFN-γ concentration (log 10); (B) IFN-γ-fold change (log 10), normalized, as described in Example 10.

FIGS. 23A and 23B show release of IL-2 in whole blood cultures stimulated via immune complexes with: HBC34-v35-MLNS and HBsAg; or HBC34-v35-MLNS-GAALIE and HBsAg, as described in Example 10. (A) IL-2 concentration (log 10); (B) IL-2-fold change (log 10), normalized, as described in Example 10.

FIGS. 24A and 24B show IFN-γ and IL-2 in whole blood cultures stimulated via immune complexes with: HBC34-v35-MLNS and HBsAg; or HBC34-v35-MLNS-GAALIE and HBsAg, as described in Example 10. (A) IFN-γ; 100 μg/ml mAb; (B). IL-2; IL-2 μg/ml mAb.

### DETAILED DESCRIPTION

The present disclosure provides pharmaceutical compositions including antibodies that neutralize a Hepatitis B virus (HBV) infection and methods of using those compositions. In certain embodiments, the antibodies bind an HBsAg of a genotype selected from A, B, C, D, E, F, G, H, I, and J, or any combination thereof. In certain embodiments, the antibodies include mutations in the heavy chain that extend in vivo half-life of the antibodies (e.g., in a human) and mutations in the heavy chain that increase binding affinity to a FcγR (e.g., a human FcγRIla, a human FcγRIIIa, or both).

In some embodiments, the antibody and the pharmaceutical composition are well-tolerated by the subject when administered in amounts that are therapeutically effective. In some embodiments, the methods described herein include administering an antibody or pharmaceutical composition according to the present description to a subject infected by HBV.

Though antibodies that neutralize HBV, pharmaceutical compositions including those antibodies, and methods for using such pharmaceutical compositions are described in detail below, it is to be understood that this disclosure is not limited to the particular methodologies, protocols and reagents described herein as these may vary. It is also to be understood that the terminology used herein is not intended to limit the scope of the present disclosure.

Unless defined otherwise, all technical and scientific terms used herein have the same meaning as commonly understood by one of ordinary skill in the art.

In the following, aspects of the present disclosure are described. Certain embodiments are provided, however, it should be understood that embodiments of the disclosure may be combined in any manner and in any number to create additional embodiments. The variously described examples and embodiments should not be construed to limit the present disclosure to only explicitly described embodiments. This description should be understood to support and encompass embodiments which combine explicitly described embodiments with any disclosed subject-matter. Furthermore, any permutations and combinations of all described subject-matter in this application should be considered disclosed by the description of the present application unless the context indicates otherwise.

Throughout this disclosure, unless the context requires otherwise, the term “comprise,” and variations thereof, such as “comprises,” and “comprising,” is used synonymously with, e.g. “having,” “has,” “including,” “includes,” or the like, and will be understood to imply the inclusion of a stated member, ratio, integer (including, where appropriate, a fraction thereof; e.g., one tenth and one hundredth of an integer), concentration, or step but not the exclusion of any other non-stated member, ratio, integer, concentration, or step. The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “consist of” is a particular embodiment of the term “comprise”, wherein any other non-stated member, integer or step is excluded. In the context of the present disclosure, the term “comprise” encompasses the term “consist of”. The term “comprising” thus encompasses “including” as well as “consisting” e.g., a composition “comprising” X may consist exclusively of X or may include something additional e.g., X+Y.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The terms “a” and “an” and “the” and similar reference used in the context of describing the disclosure (including in the context of the claims) are to be construed to cover both the singular and the plural, unless otherwise indicated herein or clearly contradicted by context. The use of the alternative (e.g., “or”) should be understood to mean either one, both, or any combination of the alternatives. Recitation of ranges of values herein is intended to serve as a shorthand method of referring individually to each separate value falling within the range. Unless otherwise indicated herein, each individual value is incorporated into the disclosure as if it were individually recited herein. No language in the specification should be construed as indicating any non-claimed element as essential to the practice of the subject matter disclosed herein.

The word “substantially” does not exclude “completely”; e.g., a composition which is “substantially free” from Y may be completely free from Y. In certain embodiments, “substantially” refers to a given amount, effect, or activity of a composition, method, or use of the present disclosure as compared to that of a reference composition, method, or use, and describes a reduction in the amount, effect, or activity of no more than 50%, such as no more than 40%, 30%, 25%, 20%, 15%, 10%, 5%, or 1%, or less, of the amount, effect, or activity of the reference composition, method, or use.

The term “about” in relation to a numerical value x means x±20%, for example, x±5%, or x±7%, or x±10%, or x±12%, or x±15%, or x±20%. For example, in certain embodiments, “about” means±20% of the indicated range, value, or structure.

“Optional” or “optionally” means that the subsequently described element, component, event, or circumstance may or may not occur, and that the description includes instances in which the element, component, event, or circumstance occurs and instances in which they do not.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the affected human or animal to have a reduced duration or quality of life.

As used herein, the term “therapeutically effective” refers to the nature or amount of a pharmaceutical composition or antibody as described herein that is sufficient to provide a benefit to the subject. In the context of the present disclosure, the benefit provided to the subject is treatment of Hepatitis B virus infection. As used herein, reference to “treatment” of a subject or patient is intended to include prevention, prophylaxis, attenuation, amelioration and therapy. Benefits of treatment include improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof. The terms “subject” or “patient” are used interchangeably herein to mean humans that are susceptible to infection by HBV or have already been infected by HBV.

Doses are often expressed in relation to bodyweight (i.e., of a subject). Thus, a dose which is expressed as [g, mg, or other unit]/kg (or g, mg etc.) can refer to [g, mg, or other unit] “per kg (or g, mg etc.) bodyweight”, even if the term “bodyweight” is not explicitly mentioned.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, the terms “peptide,” “polypeptide,” and “protein,” and variations of these terms, refer to a molecule that comprises at least two amino acids joined to each other by a (normal or modified) peptide bond. For example, a peptide, polypeptide or protein may be composed of a plurality of amino acids selected from the 20 amino acids defined by the genetic code, each being linked to at least one other by a peptide bond. A peptide, polypeptide or protein can be composed of L-amino acids and/or D-amino acids. The terms “peptide”, “polypeptide,” “protein” also include “peptidomimetics” which are defined as peptide analogs containing non-peptidic structural elements, which peptides are capable of mimicking or antagonizing the biological action(s) of a natural parent peptide. In certain embodiments, a peptidomimetic lacks characteristics such as enzymatically scissile peptide bonds.

A peptide, polypeptide or protein may comprise amino acids other than the 20 amino acids defined by the genetic code in addition to these amino acids, or it can be composed of amino acids other than the 20 amino acids defined by the genetic code. In certain embodiments, a peptide, polypeptide or protein in the context of the present disclosure can comprise amino acids that are modified by natural processes, such as post-translational maturation processes, or by chemical processes (e.g., synthetic processes), which are known in the art and include those described herein. Such modifications can appear anywhere in the polypeptide; e.g., in the peptide skeleton; in the amino acid chain; or at the carboxy- or amino-terminal ends. A peptide or polypeptide can be branched, such as following an ubiquitination, or may be cyclic, with or without branching. The terms “peptide”, “polypeptide”, “protein” also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications have been described in the literature (see Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663:48-62). Accordingly, the terms “peptide”, “polypeptide”, “protein” can include for example lipopeptides, lipoproteins, glycopeptides, glycoproteins and the like. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

As used herein, “(poly)peptide” and “protein” may be used interchangeably in reference to a polymer of amino acid residues, such as a plurality of amino acid monomers linked by peptide bonds.

“Nucleic acid molecule” or “polynucleotide” or “nucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, or the like.

Nucleic acid molecules include polyribonucleic acid (RNA), polydeoxyribonucleic acid (DNA), which includes cDNA, genomic DNA, and synthetic DNA, any of which may be single or double-stranded. If single-stranded, the nucleic acid molecule may be the coding strand or non-coding (anti-sense strand). Polynucleotides (including oligonucleotides), and fragments thereof may be generated, for example, by polymerase chain reaction (PCR) or by in vitro translation, or generated by any of ligation, scission, endonuclease action, or exonuclease action.

A nucleic acid molecule encoding an amino acid sequence includes all nucleotide sequences that encode the same amino acid sequence. Some versions of the nucleotide sequences may also include intron(s) to the extent that the intron(s) may be removed through co- or post-transcriptional mechanisms. In other words, different nucleotide sequences may encode the same amino acid sequence as the result of the redundancy or degeneracy of the genetic code, or by splicing, or both.

Variants of nucleic acid molecules of this disclosure are also contemplated. Variant nucleic acid molecules are at least 70%, 75%, 80%, 85%, 90%, 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

As used herein, the term “sequence variant” refers to any sequence having one or more alterations in comparison to a reference sequence, whereby a reference sequence is any published sequence and/or of the sequences listed in the “Table of Sequences and SEQ ID Numbers” (sequence listing), i.e. SEQ ID NO: 1 to SEQ ID NO: 120. Thus, the term “sequence variant” includes nucleotide sequence variants and amino acid sequence variants. In certain embodiments, a sequence variant in the context of a nucleotide sequence, the reference sequence is also a nucleotide sequence, whereas in certain embodiments for a sequence variant in the context of an amino acid sequence, the reference sequence is also an amino acid sequence. A “sequence variant” as used herein can be at least 80%, at least 85%, at least 90%, at least 91%, at least 92%, at least 93%, at least 94%, at least 95%, at least 96%, at least 97%, at least 98%, or at least 99% identical to the reference sequence.

“Percent sequence identity” refers to a relationship between two or more sequences, as determined by comparing the sequences. Methods to determine sequence identity can be designed to give the best match between the sequences being compared. For example, the sequences may be aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e. in an amino acid “sequence variant” or not. In certain embodiments, a nucleotide sequence variant does not result in an amino acid sequence variant (e.g., a silent mutation). In some embodiments, a nucleotide sequence variant that results in a to “non-silent” mutations is contemplated. In some embodiments, a nucleotide sequence variant of the present disclosure encodes an amino acid sequence that is at least 80%, at least 85%, at least 90%, at least 91%, at least 92%, at least 93%, at least 94%, at least 95%, at least 96%, at least 97%, at least 98%, or at least 99% identical to a reference amino acid sequence. Nucleotide and amino sequences as disclosed herein refer also to codon-optimized versions of a reference or wild-type nucleotide or amino acid sequence. In any of the embodiments described herein, a polynucleotide of the present disclosure may be codon-optimized for a host cell containing the polynucleotide (see, e.g., Scholten et al., Clin. Immunol. 119:135-145 (2006).

A “sequence variant” in the context of an amino acid sequence has an altered sequence in which one or more of the amino acids is deleted, substituted, or inserted in comparison to a reference amino acid sequence. As a result of the alterations, such a sequence variant has an amino acid sequence which is at least 80%, at least 85%, at least 90%, at least 91%, at least 92%, at least 93%, at least 94%, at least 95%, at least 96%, at least 97%, at least 98%, or at least 99% identical to the reference amino acid sequence. For example, per 100 amino acids of the reference sequence a variant sequence that has no more than 10 alterations, i.e. any combination of deletions, insertions or substitutions, is “at least 90% identical” to the reference sequence.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Amino acid sequence insertions can include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In general, alterations in the sequence variants do not abolish or significantly reduce a desired functionality of the respective reference sequence. For example, it is preferred that a variant sequence of the present disclosure does not significantly reduce or completely abrogate the functionality of a sequence of an antibody, or antigen binding fragment thereof, to bind to the same epitope and/or to sufficiently neutralize infection of HBV and HDV as compared to antibody or antigein binding fragment having (or encoded by) the reference sequence. Guidance in determining which nucleotides and amino acid residues, respectively, may be substituted, inserted or deleted without abolishing a desired structure or functionality can be found by using known computer programs.

As used herein, a nucleic acid sequence or an amino acid sequence “derived from” a designated nucleic acid, peptide, polypeptide or protein refers to the origin of the nucleic acid, peptide, polypeptide or protein. A nucleic acid sequence or amino acid sequence which is derived from a particular sequence may have an amino acid sequence that is essentially identical to that sequence or a portion thereof, from which it is derived, whereby “essentially identical” includes sequence variants as defined above. A nucleic acid sequence or amino acid sequence which is derived from a particular peptide or protein, may be derived from the corresponding domain in the particular peptide or protein. In this context, “corresponding” refers to possession of a same functionality or characteristic of interest. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art. Likewise, a sequence “derived from” another (e.g., “source”) sequence can be identified by one of ordinary skill in the art as having its origin in the source sequence.

A nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein, one or more amino acid residues may be substituted with other amino acid residues, or one or more amino acid residue insertions or deletions may occur.

As used herein, the term “mutation” relates to a change in a nucleic acid sequence and/or in an amino acid sequence in comparison to a reference sequence, e.g. a corresponding genomic, wild type, or reference sequence. A mutation, e.g. in comparison to a reference genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, for example, by altering (e.g., by site-directed mutagenesis) a codon (e.g., by alterning one, two, or three nucleotide bases therein) of a nucleic acid molecule encoding one amino acid to provide a codon that encodes a different amino acid, or that encodes a same amino acid, or by synthesizing a sequence variant.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

The term “recombinant”, as used herein (e.g. a recombinant antibody, a recombinant protein, a recombinant nucleic acid, or the linke., refers to any molecule (antibody, protein, nucleic acid, or the like) which is prepared, expressed, created or isolated by recombinant means, and which is not naturally occurring. “Recombinant” can be used synonymously with “engineered” or “non-natural” and can refer to to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject. As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same or substantially the same function, phenotype, or biological activity as screened for in the originally transformed cell are included. Where distinct designations are intended, it will be clear from the context.

The present disclosure is based, in part, on the design of antibodies and antigen binding fragments that are capable of neutralizing hepatitis B and hepatitis delta viruses. Embodiments of the antibodies and antigen binding fragments, according to the present description may be used in methods of preventing, treating, or attenuating HBV and HDV. In particular embodiments, the antibodies and antigen binding fragments described herein bind to two or more different genotypes of hepatitis B virus surface antigen and to two or more different infectious mutants of hepatitis B virus surface antigen. In specific embodiments, the antibodies and antigen binding fragments described herein bind to currently all known genotypes of hepatitis B virus surface antigen and to all currently known infectious mutants of hepatitis B virus surface antigen.

### Antibodies and Antigen-Binding Fragments Thereof

In one aspect, the present disclosure provides an isolated antibody, or an antigen binding fragment thereof, for use in a pharmaceutical composition and method as disclosed herein, that binds to the antigenic loop region of HBsAg and neutralizes infection with hepatitis B virus and hepatitis delta virus.

As used herein, and unless the context clearly indicates otherwise, “antibody” refers to an intact antibody comprising at least two heavy (H) chains and two light (L) chains inter-connected by disulfide bonds (though it will be understood that heavy chain antibodies, which lack light chains, are still encompassed by the term “antibody”), as well as any antigen-binding portion or fragment of an intact antibody that has or retains the ability to bind to the antigen target molecule recognized by the intact antibody, such as, for example, a scFv, Fab, or F(ab′)2 fragment. Thus, the term “antibody” herein is used in the broadest sense and includes polyclonal and monoclonal antibodies, including intact antibodies and functional (antigen-binding) antibody fragments thereof, including fragment antigen-binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class thereof, including IgG and sub-classes thereof, IgM, IgE, IgA, and IgD.

Accordingly, antibodies of the disclosure can be of any isotype (e.g., IgA, IgG, IgM, also referred to as α, γ and μ heavy chain, respectively). For example, in certain embodiments, antibody is of the IgG type. Within the IgG isotype, antibodies may be IgG1, IgG2, IgG3 or IgG4 subclass, for example IgG1. In some embodiments, an antibody comprises an amino acid sequence from two different isotypes (e.g., exchange of constant domain amino acid sequence), such as, for example, an antibody comprising a constant region that comprises amino acid sequence from an IgA antibody and amino acid sequence from an IgG antibody. Antibodies of the disclosure may comprise a κ or a λ light chain. In some embodiments, the antibody is of IgG1 type and comprises a κ light chain.

As used herein, the terms “antigen binding fragment,” “fragment,” and “antibody fragment” are used interchangeably to refer to any fragment of an antibody of the disclosure that retains the antigen-binding activity of the antibody. Examples of antibody fragments include, but are not limited to, a single chain antibody, Fab, Fab′, F(ab′)₂, Fv or scFv. Further, the term “antibody” as used herein, includes both antibodies and antigen binding fragments thereof. Antibodies and antigen binding fragments are discussed further herein.

Human antibodies are known (van Dijk, M. A., and van de Winkel, J. G., Curr. Opin. Chem. Biol. 5 (2001) 368-374). Human antibodies can be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). Human monoclonal antibodies may be prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. The term “human antibody” as used herein also comprises such antibodies which are modified, e.g., in the variable region, to generate properties according to the antibodies and antibody fragments of the present disclosure. As used herein, the term “variable region” (variable region of a light chain (V_{L}), variable region of a heavy chain (V_{H})) denotes each of the pair of light and heavy chains which is involved directly in binding the antibody to the antigen.

As used herein, the term “variable region” (e.g., variable region of a light chain (V_{L}), variable region of a heavy chain (V_{H})) refers to the variable region of an antibody light chain or an antibody heavy chain, which is involved directly in binding the antibody to the antigen. In other words, the terms “V_{L}” or “V_{L}” and “V_{H}” or “V_{H}” refer to the variable binding region from an antibody light chain and an antibody heavy chain, respectively.

The variable binding regions are made up of discrete, well-defined sub-regions known as “complementarity determining regions” (CDRs) and “framework regions” (FRs). The terms “complementarity determining region” and “CDR” are synonymous with “hypervariable region” or “HVR,” and are known in the art to refer to non-contiguous sequences of amino acids within antibody variable regions, which, in general, confer antigen specificity and/or binding affinity. In general, there are three CDRs in each variable region of anantibody; the VH and VL regions together comprise six CDRs HCDR1, HCDR2, HCDR3; LCDR1, LCDR2, LCDR3; also referred to herein as CDRH1, CDRH2, CDRH3, CDRL1, CDRL2, and CDRL3, respectively). The CDRs on the heavy and/or light chain may be separated in primary amino acid sequence by framework regions, whereby a framework region (FR) is a region in the variable domain which is less variable (i.e., from one antibody to another (e.g., from one antibody to another encoded by a same allele or alleles)) than the CDR. For example, a chain (or each chain, respectively) may be composed of four framework regions, separated by three CDRs. In certain embodiments, an antibody VH comprises four FRs and three CDRs arranged as follows: FR1-CDRH1-FR2-CDRH2-FR3-CDRH3-FR4; and an antibody VL comprises four FRs and three CDRs as follows: FR1-CDRL1-FR2-CDRL2-FR3-CDRL3-FR4. In general, the VH and the VL together form the antigen-binding site through their respective CDRs, though it will be understood that in some cases, a binding site can be formed by or comprise one, two, three, four, or five of the CDRs.

As used herein, a “variant” of a CDR refers to a functional variant of a CDR sequence having up to 1-3 amino acid substitutions, deletions, or combinations thereof. Immunoglobulin sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300). It will be understood that in certain embodiments, an antibody or antigen binding fragment of the present disclosure can comprise all or part of a heavy chain (HC), a light chain (LC), or both. For example, a full-length intact IgG antibody monomer typically includes a VH, a CH1, a CH2, a CH3, a VL, and a CL. Fc components are described further herein.

In the present disclosure, the position of the CDR amino acids are defined according to the IMGT numbering system (IMGT: www_imgt.org; cf. Lefranc, M.-P. et al. (2009) Nucleic Acids Res. 37, D1006-D1012).

Table 1 shows the amino acid sequences of heavy chain variable
regions (VH), light chain variable regions (VL), CDRs, heavy
chains (HC), and light chains (LC) of certain exemplary
antibodies according to the present disclosure.

| Antibody | SEQ ID | |
|---|---|---|
| sequence description | NO: | Amino acid sequence |
| HBC34-V35 VH; | 41 | ELQLVESGGGWVQPGGSQRLSCAAS |
| HBC34-V34 VH; | | GRIFRSFYMSWVRQAPGKGLEWVATI |
| HBC23-LC40A VH; | | NQDGSEKLYVDSVKGRFTISRDNAKN |
| HBC23-LC40S VH; | | SLFLQMNNLRVEDTAVYYCAAWSGN |
| HBC34-LC40A VH; | | SGGMDVWGQGTTVSVSS |
| HBC34-LC40S VH | | |
| HBC34v31_LC40A VH | 67 | EVQLVESGGGLVQPGGSLRLSCAASG |
| HBC34v31_LC40S VH | | RIFRSFYMSWVRQAPGKGLEWVANIN |
| HBC34v32_LC40A VH | | QDGSEKLYVDSVKGRFTISRDNAKNS |
| HBC34v32_LC40S VH | | LFLQMNNLRVEDTAVYYCAAWSGNS |
| HBC34v33_LC40A VH | | GGMDVWGQGTTVTVSS |
| HBC34v32_LC40S VH | | |
| HBC34-V35 VL | 89 | SYELTQPPSVSVSPGQTVSIPCSGDKL |
| | | GNKNVAWFQHKPGQSPVLVIYEVKY |
| | | RPSGIPERFSGSNSGNTATLTISGTQA |
| | | MDEAAYFCQTFDSTTVVFGGGTRLTV |
| | | L |
| HBC34-V34 VL | 90 | SYELTQPPSVSVSPGQTVSIPCSGDKL |
| | | GNKNVSWFQHKPGQSPVLVIYEVKY |
| | | RPSGIPERFSGSNSGNTATLTISGTQA |
| | | MDEAAYFCQTFDSTTVVFGGGTRLTV |
| | | L |
| HBC34-V23-VL_C40S | 110 | SYELTQPPSVSVSPGQTASITCSGDKL |
| | | GNKNASWYQQKPGQSPVLVIYEVKY |
| | | RPSGIPERFSGSNSGNTATLTISGTQA |
| | | MDEADYYCQTFDSTTVVFGGGTKLT |
| | | VL |
| HBC34-V23-VL_C40A | 111 | SYELTQPPSVSVSPGQTASITCSGDKL |
| | | GNKNAAWYQQKPGQSPVLVIYEVKY |
| | | RPSGIPERFSGSNSGNTATLTISGTQA |
| | | MDEADYYCQTFDSTTVVFGGGTKLT |
| | | VL |
| HBC34-V31-VL_C40S | 112 | SYELTQPPSVSVSPGQTVSIPCSGDKL |
| | | GNKNVSWFQHKPGQSPVLVIYEVKY |
| | | RPSGIPERFSGSNSGNTATLTISGTQA |
| | | MDEAAYFCQTWDSTTVVFGGGTRLT |
| | | VL |
| HBC34-V31-VL_C40A | 113 | SYELTQPPSVSVSPGQTVSIPCSGDKL |
| | | GNKNVAWFQHKPGQSPVLVIYEVKY |
| | | RPSGIPERFSGSNSGNTATLTISGTQA |
| | | MDEAAYFCQTWDSTTVVFGGGTRLT |
| | | VL |
| HBC34-V32-VL_C40S | 114 | SYELTQPPSVSVSPGQTVSIPCSGDKL |
| | | GNKNVSWFQHKPGQSPVLVIYEVKY |
| | | RPSGIPERFSGSNSGNTATLTISGTQA |
| | | MDEAAYFCQTFDSTTVVFGGGTRLTV |
| | | L |
| HBC34-V32-VL_C40A | 115 | SYELTQPPSVSVSPGQTVSIPCSGDKL |
| | | GNKNVAWFQHKPGQSPVLVIYEVKY |
| | | RPSGIPERFSGSNSGNTATLTISGTQA |
| | | MDEAAYFCQTFDSTTVVFGGGTRLTV |
| | | L |
| HBC34-V33-VL_C40S | 116 | SYELTQPPSVSVSPGQTASITCSGDKL |
| | | GNKNASWYQQKPGQSPVLVIYEVKY |
| | | RPSGIPERFSGSNSGNTATLTISGTQA |
| | | MDEADYYCQTFDSTTVVFGGGTKLT |
| | | VL |
| HBC34-V33-VL_C40A | 117 | SYELTQPPSVSVSPGQTASITCSGDKL |
| | | GNKNAAWYQQKPGQSPVLVIYEVKY |
| | | RPSGIPERFSGSNSGNTATLTISGTQA |
| | | MDEADYYCQTFDSTTVVFGGGTKLT |
| | | VL |
| HBC34-VL_C40S | 118 | SYELTQPPSVSVSPGQTVSIPCSGDKL |
| | | GNKNVSWFQHKPGQSPVLVIYEVKY |
| | | RPSGIPERFSGSNSGNTATLTISGTQA |
| | | MDEAAYFCQTWDSTTVVFGGGTRLT |
| | | VL |
| HBC34-VL_C40A | 119 | SYELTQPPSVSVSPGQTVSIPCSGDKL |
| | | GNKNVAWFQHKPGQSPVLVIYEVKY |
| | | RPSGIPERFSGSNSGNTATLTISGTQA |
| | | MDEAAYFCQTWDSTTVVFGGGTRLT |
| | | VL |
| HBC34-V35 CDRH1; | 34 | GRIFRSFY |
| HBC34-V34 CDRH1; | | |
| HBC34-V23_LC40S CDRH1; | | |
| HBC34-V23_LC40A CDRH1; | | |
| HBC34-V31_LC40S CDRH1; | | |
| HBC34-V31_LC40A CDRH1; | | |
| HBC34-V32_LC40S CDRH1; | | |
| HBC34-V32_LC40A CDRH1; | | |
| HBC34-V33_LC40S CDRH1; | | |
| HBC34-V33_LC40A CDRH1; | | |
| HBC34_LC40S CDRH1; | | |
| HBC34_LC40A CDRH1 | | |
| HBC34-V35 CDRH2; | 35 | NQDGSEK |
| HBC34-V34 CDRH2; | | |
| HBC34-V23_LC40S CDRH2; | | |
| HBC34-V23_LC40A CDRH2; | | |
| HBC34-V31_LC40S CDRH2; | | |
| HBC34-V31_LC40A CDRH2; | | |
| HBC34-V32_LC40S CDRH2; | | |
| HBC34-V32_LC40A CDRH2; | | |
| HBC34-V33_LC40S CDRH2; | | |
| HBC34-V33_LC40A CDRH2; | | |
| HBC34_LC40S CDRH2; | | |
| HBC34_LC40A CDRH2 | | |
| (short CDRH2) | | |
| HBC34-V35 CDRH2; | 66 | INQDGSEK |
| HBC34-V34 CDRH2; | | |
| HBC34-V23_LC40S CDRH2; | | |
| HBC34-V23_LC40A CDRH2; | | |
| HBC34-V31_LC40S CDRH2; | | |
| HBC34-V31_LC40A CDRH2; | | |
| HBC34-V32_LC40S CDRH2; | | |
| HBC34-V32_LC40A CDRH2; | | |
| HBC34-V33_LC40S CDRH2; | | |
| HBC34-V33_LC40A CDRH2; | | |
| HBC34_LC40S CDRH2; | | |
| HBC34_LC40A CDRH2 | | |
| (long CDRH2) | | |
| HBC34-V35 CDRH3; | 36 | AAWSGNSGGMDV |
| HBC34-V34 CDRH3; | | |
| HBC34-V23_LC40S CDRH3; | | |
| HBC34-V23_LC40A CDRH3; | | |
| HBC34-V31_LC40S CDRH3; | | |
| HBC34-V31_LC40A CDRH3; | | |
| HBC34-V32_LC40S CDRH3; | | |
| HBC34-V32_LC40A CDRH3; | | |
| HBC34-V33_LC40S CDRH3; | | |
| HBC34-V33_LC40A CDRH3; | | |
| HBC34_LC40S CDRH3; | | |
| HBC34_LC40A CDRH3 | | |
| HBC34-V35 CDRL1; | 37 | KLGNKN |
| HBC34-V34 CDRL1; | | |
| HBC34-V23_LC40S CDRL1; | | |
| HBC34-V23_LC40A CDRL1; | | |
| HBC34-V31_LC40S CDRL1; | | |
| HBC34-V31_LC40A CDRL1; | | |
| HBC34-V32_LC40S CDRL1; | | |
| HBC34-V32_LC40A CDRL1; | | |
| HBC34-V33_LC40S CDRL1; | | |
| HBC34-V33_LC40A CDRL1; | | |
| HBC34_LC40S CDRL1; | | |
| HBC34_LC40A CDRL1 | | |
| HBC34-V35 CDRL2; | 38 | EVK |
| HBC34-V34 CDRL2; | | |
| HBC34-V23_LC40S CDRL2; | | |
| HBC34-V23_LC40A CDRL2; | | |
| HBC34-V31_LC40S CDRL2; | | |
| HBC34-V31_LC40A CDRL2; | | |
| HBC34-V32_LC40S CDRL2; | | |
| HBC34-V32_LC40A CDRL2; | | |
| HBC34-V33_LC40S CDRL2; | | |
| HBC34-V33_LC40A CDRL2; | | |
| HBC34_LC40S CDRL2; | | |
| HBC34_LC40A CDRL2 | | |
| (short CDRL2) | | |
| HBC34-V35 CDRL2; | 39 | VIYEVKYRP |
| HBC34-V34 CDRL2; | | |
| HBC34-V23_LC40S CDRL2; | | |
| HBC34-V23_LC40A CDRL2; | | |
| HBC34-V31_LC40S CDRL2; | | |
| HBC34-V31_LC40A CDRL2; | | |
| HBC34-V32_LC40S CDRL2; | | |
| HBC34-V32_LC40A CDRL2; | | |
| HBC34-V33_LC40S CDRL2; | | |
| HBC34-V33_LC40A CDRL2; | | |
| HBC34_LC40S CDRL2; | | |
| HBC34_LC40A CDRL2 | | |
| (long LCDR2) | | |
| HBC34-V35 CDRL3; | 58 | QTFDSTTVV |
| HBC34-V34 CDRL3; | | |
| HBC34-V23_LC40S CDRL3; | | |
| HBC34-V23_LC40A CDRL3; | | |
| HBC34-V32_LC40S CDRL3; | | |
| HBC34-V32_LC40A CDRL3; | | |
| HBC34-V33_LC40S CDRL3; | | |
| HBC34-V33_LC40A CDRL3; | | |
| HBC34_LC40S CDRL3; | 40 | QTWDSTTVV |
| HBC34_LC40A CDRL3; | | |
| HBC34-V31_LC40S CDRL3; | | |
| HBC34-V31_LC40A CDRL3; | | |
| HC of HBC34-V35-MLNS- | 91 | ELQLVESGGGWVQPGGSQRLSCAAS |
| GAALIE and HBC34-V34- | | GRIFRSFYMSWVRQAPGKGLEWVATI |
| MLNS-GAALIE (g1M17, 1) | | NQDGSEKLYVDSVKGRFTISRDNAKN |
| | | SLFLQMNNLRVEDTAVYYCAAWSGN |
| | | SGGMDVWGQGTTVSVSSASTKGPSV |
| | | FPLAPSSKSTSGGTAALGCLVKDYFPE |
| | | PVTVSWNSGALTSGVHTFPAVLQSSG |
| | | LYSLSSVVTVPSSSLGTQTYICNVNHK |
| | | PSNTKVDKKVEPKSCDKTHTCPPCPA |
| | | PELLAGPSVFLFPPKPKDTLMISRTPEV |
| | | TCVVVDVSHEDPEVKFNWYVDGVEV |
| | | HNAKTKPREEQYNSTYRVVSVLTVLH |
| | | QDWLNGKEYKCKVSNKALPLPEEKTI |
| | | SKAKGQPREPQVYTLPPSRDELTKNQ |
| | | VSLTCLVKGFYPSDIAVEWESNGQPE |
| | | NNYKTTPPVLDSDGSFFLYSKLTVDK |
| | | SRWQQGNVFSCSVLHEALHSHYTQK |
| | | SLSLSPGK |
| HC of HBC34-V35-MLNS and | 92 | ELQLVESGGGWVQPGGSQRLSCAAS |
| HBC34-V34-MLNS | | GRIFRSFYMSWVRQAPGKGLEWVATI |
| | | NQDGSEKLYVDSVKGRFTISRDNAKN |
| | | SLFLQMNNLRVEDTAVYYCAAWSGN |
| | | SGGMDVWGQGTTVSVSSASTKGPSV |
| | | FPLAPSSKSTSGGTAALGCLVKDYFPE |
| | | PVTVSWNSGALTSGVHTFPAVLQSSG |
| | | LYSLSSVVTVPSSSLGTQTYICNVNHK |
| | | PSNTKVDKKVEPKSCDKTHTCPPCPA |
| | | PELLGGPSVFLFPPKPKDTLMISRTPEV |
| | | TCVVVDVSHEDPEVKFNWYVDGVEV |
| | | HNAKTKPREEQYNSTYRVVSVLTVLH |
| | | QDWLNGKEYKCKVSNKALPAPIEKTI |
| | | SKAKGQPREPQVYTLPPSRDELTKNQ |
| | | VSLTCLVKGFYPSDIAVEWESNGQPE |
| | | NNYKTTPPVLDSDGSFFLYSKLTVDK |
| | | SRWQQGNVFSCSVLHEALHSHYTQK |
| | | SLSLSPGK |
| LC of HBC34-V35 | 93 | SYELTQPPSVSVSPGQTVSIPCSGDKL |
| | | GNKNVAWFQHKPGQSPVLVIYEVKY |
| | | RPSGIPERFSGSNSGNTATLTISGTQA |
| | | MDEAAYFCQTFDSTTVVFGGGTRLTV |
| | | LGQPKAAPSVTLFPPSSEELQANKATL |
| | | VCLISDFYPGAVTVAWKADSSPVKAG |
| | | VETTTPSKQSNNKYAASSYLSLTPEQ |
| | | WKSHRSYSCQVTHEGSTVEKTVAPTE |
| | | CS |
| LC of HBC34-V34 | 94 | SYELTQPPSVSVSPGQTVSIPCSGDKL |
| | | GNKNVSWFQHKPGQSPVLVIYEVKY |
| | | RPSGIPERFSGSNSGNTATLTISGTQA |
| | | MDEAAYFCQTFDSTTVVFGGGTRLTV |
| | | LGQPKAAPSVTLFPPSSEELQANKATL |
| | | VCLISDFYPGAVTVAWKADSSPVKAG |
| | | VETTTPSKQSNNKYAASSYLSLTPEQ |
| | | WKSHRSYSCQVTHEGSTVEKTVAPTE |
| | | CS |
| HBC24 VH | 95 | EVQLLESGGGLVQPGGSLRLSCAASG |
| | | STFTKYAMSWVRQAPGKGLEWVASI |
| | | SGSVPGFGIDTYYADSVKGRFTISRDT |
| | | SKNTLYLQMNSLRAEDTALYYCAKD |
| | | VGVIGSYYYYAMDVWGQGTAVTVSS |
| HBC24 VL | 96 | EIVLTQSPGTLSLSPGERATLSCRASQ |
| | | GLSSSYLAWYQQKPGQAPRLLIYSAS |
| | | TRATGIPDRFSGSGSGTDFTLTISRLEP |
| | | EDFAVYYCQQYAYSPRWTFGQGTKV |
| | | EIK |
| HBC24 CDRH1 | 97 | GSTFTKYA |
| HBC24 CDRH2 | 98 | ISGSVPGF |
| HBC24 CDRH3 | 99 | LYYCAKDVGVIGSYYYYAMDV |
| HBC24 CDRL1 | 100 | QGLSSSY |
| HBC24 CDRL2 | 101 | SAS |
| HBC24 CDRL3 | 102 | QQYAYSPRWT |
| HBC34-V7, | 129 | ELQLVESGGGWVQPGGSQRLSCAAS |
| HBC34-V34, | | GRIFRSFYMSWVRQAPGKGLEWVATI |
| HBC34-V35 | | NQDGSEKLYVDSVKGRFTISRDNAKN |
| HC (VH-hinge-CH1-CH2-CH3) | | SLFLQMNNLRVEDTAVYYCAAWSGN |
| (wild-type) | | SGGMDVWGQGTTVSVSSASTKGPSV |
| | | FPLAPSSKSTSGGTAALGCLVKDYFPE |
| | | PVTVSWNSGALTSGVHTFPAVLQSSG |
| | | LYSLSSVVTVPSSSLGTQTYICNVNHK |
| | | PSNTKVDKKVEPKSCDKTHTCPPCPA |
| | | PELLGGPSVFLFPPKPKDTLMISRTPEV |
| | | TCVWDVSHEDPEVKFNWYVDGVEV |
| | | HNAKTKPREEQYNSTYRVVSVLTVLH |
| | | QDWLNGKEYKCKVSNKALPAPIEKTI |
| | | SKAKGQPREPQVYTLPPSRDELTKNQ |
| | | VSLTCLVKGFYPSDIAVEWESNGQPE |
| | | NNYKTTPPVLDSDGSFFLYSKLTVDK |
| | | SRWQQGNVFSCSVMHEALHNHYTQK |
| | | SLSLSPGK |
| WT hIgG1 Fc | 137 | APELLGGPSVFLFPPKPKDTLMISRTPE |
| | | VTCVVVDVSHEDPEVKFNWYVDGVE |
| | | VHNAKTKPREEQYNSTYRVVSVLTVL |
| | | HQDWLNGKEYKCKVSNKALPAPIEK |
| | | TISKAKGQPREPQVYTLPPSRDELTKN |
| | | QVSLTCLVKGFYPSDIAVEWESNGQP |
| | | ENNYKTTPPVLDSDGSFFLYSKLTVD |
| | | KSRWQQGNVFSCSVMHEALHNHYTQ |
| | | KSLSLSPGK |
| HBC34v7, | 138 | ELQLVESGGGWVQPGGSQRLSCAAS |
| HBC34v23, | | GRIFRSFYMSWVRQAPGKGLEWVATI |
| HBC34v34, | | NQDGSEKLYVDSVKGRFTISRDNAKN |
| HBC34v35, | | SLFLQMNNLRVEDTAVYYCAAWSGN |
| HBC34_C40S, | | SGGMDVWGQGTTVSVSSASTKGPSV |
| HBC34_C40A, | | FPLAPSSKSTSGGTAALGCLVKDYFPE |
| HBC34v23_C40S, | | PVTVSWNSGALTSGVHTFPAVLQSSG |
| HBC34v23_C40A | | LYSLSSVVTVPSSSLGTQTYICNVNHK |
| HC with GAALIE mutation in | | PSNTKVDKKVEPKSCDKTHTCPPCPA |
| hIgG1 Fc | | PELLAGPSVFLFPPKPKDTLMISRTPEV |
| | | TCVWDVSHEDPEVKFNWYVDGVEV |
| | | HNAKTKPREEQYNSTYRVVSVLTVLH |
| | | QDWLNGKEYKCKVSNKALPLPEEKTI |
| | | SKAKGQPREPQVYTLPPSRDELTKNQ |
| | | VSLTCLVKGFYPSDIAVEWESNGQPE |
| | | NNYKTTPPVLDSDGSFFLYSKLTVDK |
| | | SRWQQGNVFSCSVMHEALHNHYTQK |
| | | SLSLSPGK |

Fragments of the antibodies described herein can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Antibody “fragments” include Fab, Fab′, F(ab′)2 and Fv fragments. The present disclosure also encompasses single-chain Fv fragments (scFv) derived from the heavy and light chains of an antibody as described herein, including, for example, an scFv comprising the CDRs from an antibody according to the present description, heavy or light chain monomers and dimers, single domain heavy chain antibodies, single domain light chain antibodies, as well as single chain antibodies, in which the heavy and light chain variable domains are joined by a peptide linker.

In certain embodiments, an antibody according to the present disclosure, or an antigen binding fragment thereof, comprises a purified antibody, a single chain antibody, Fab, Fab′, F(ab′)2, Fv or scFv.

Antibodies and antigen binding fragments of the present disclosure may, in embodiments, be multispecific (e.g., bispecific, trispecific, tetraspecific, or the like), and may be provided in any multispecific format, as disclosed herein. In certain embodiments, an antibody or antigen-binding fragment of the present disclosure is a multispecific antibody, such as a bispecific or trispecific antibody. Formats for bispecific antibodies are disclosed in, for example, Spiess et al., Mol. Immunol. 67(2):95 (2015), and in Brinkmann and Kontermann, mAbs 9(2):182-212 (2017), which bispecific formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, KA-bodies, orthogonal Fabs, DVD-IgGs, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one). A bispecific or multispecific antibody may comprise a HBV- and/or HDV-specific binding domain of the instant disclosure in combination with another HBV- and/or HDV-specific binding domain of the instant disclosure, or in combination with a different binding domain that specifically binds to HBV and/or HDV (e.g., at a same or a different epitope), or with a binding domain that specifically binds to a different antigen.

Antibody fragments of the disclosure may impart monovalent or multivalent interactions and be contained in a variety of structures as described above. For instance, scFv molecules may be synthesized to create a trivalent “triabody” or a tetravalent “tetrabody”. The scFv molecules may include a domain of the Fc region resulting in bivalent minibodies. In addition, the sequences of the disclosure may be a component of multispecific molecules in which the sequences of the disclosure target the epitopes of the disclosure and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9:1126-1136).

In some embodiments, an antibody may be present in a pharmaceutical composition that is substantially free of other polypeptides e.g., where less than 90% (by weight), usually less than 60% and more usually less than 50% of the pharmaceutical composition is made up of other polypeptides.

Antibodies according to the present disclosure may be immunogenic in human and/or in non-human (or heterologous) hosts; e.g., in mice. For example, an antibody may have an idiotope that is immunogenic in non-human hosts, but not in a human host. Antibodies of the disclosure for human use include those that are not typically isolated from hosts such as mice, goats, rabbits, rats, non-primate mammals, or the like, and in some instances are not obtained by humanization or from xeno-mice. In certain embodiments, an antibody according to the present disclosure is non-immunogenic or is substantially non-immunogenic in a human.

Also contemplated herein are variant forms of the disclosed antibodies, which are engineered so as to reduce known or potential immunogenicity and/or other potential liabilities.

As used herein, a “neutralizing antibody” is one that can neutralize, i.e., prevent, inhibit, reduce, impede or interfere with, the ability of a pathogen to initiate and/or perpetuate an infection in a host (e.g., host organism or host cell). The terms “neutralizing antibody” and “an antibody that neutralizes” or “antibodies that neutralize” are used interchangeably herein. These antibodies can be used alone, or in combination (e.g., two or more of the presently disclosed antibodies in a combination, or an antibody of the present disclosure in combination with another agent, which may or may not be an antibody agent, including an antibody that is capable of neutralizing an HBV B and/or D infection), as prophylactic or therapeutic agents upon appropriate formulation, in association with active vaccination.

As used herein, “specifically binds” or “specific for” refers to an association or union of a binding protein (e.g., an antibody or antigen binding fragment thereof) or a binding domain to a target molecule with an affinity or Ka (i.e., an equilibrium association constant of a particular binding interaction with units of 1/M) equal to or greater than 10⁵ M⁻¹ (which equals the ratio of the on-rate [Kₒₙ] to the off rate [Koff] for this association reaction), while not significantly associating or uniting with any other molecules or components in a sample. Antibodies or binding domains may be classified as “high-affinity” binding proteins or binding domains or as “low-affinity” binding proteins or binding domains. “High-affinity” binding proteins or binding domains refer to those binding proteins or binding domains having a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Ka of up to 10⁷ M⁻¹, up to 10⁶ M⁻¹, or up to 10⁵ M⁻¹. Alternatively, affinity may be defined as an equilibrium dissociation constant (Kd) of a particular binding interaction with units of M (e.g., 10⁻⁵ M to 10⁻¹³ M). The terms “binding” and “specifically binding” and similar references do not encompass non-specific sticking.

In certain embodiments, antibodies according to the present disclosure can bind to the antigenic loop region of HBsAg. The envelope of the hepatitis B virus generally contains three “HBV envelope proteins” (also known as “HBsAg”, “hepatitis B surface antigen”): S protein (for “small”, also referred to as S-HBsAg), M protein (for “middle”, also referred to as M-HBsAg) and L protein (for “large”, also referred to as L-HBsAg). S-HBsAg, M-HBsAg and L-HBsAg share the same C-terminal extremity (also referred to as “S domain”, 226 amino acids), which corresponds to the S protein (S-HBsAg) and which is crucial for virus assembly and infectivity. S-HBsAg, M-HBsAg and L-HBsAg are synthesized in the endoplasmic reticulum (ER), assembled, and secreted as particles through the Golgi apparatus. The S domain comprises four predicted transmembrane (TM) domains, whereby both the N-terminus as well as the C-terminus of the S domain are exposed to the lumen. The transmembrane domains TM1 and TM2 are both believed necessary for cotranslational protein integration into the ER membrane and the transmembrane domains TM3 and TM4 are located in the C-terminal third of the S domain. The “antigenic loop region” of HBsAg is located between the predicted TM3 and TM4 transmembrane domains of the S domain of HBsAg, whereby the antigenic loop region comprises amino acids 101-172 of the S domain, which contains 226 amino acids in total (Salisse J. and Sureau C., 2009, Journal of Virology 83:9321-9328). A determinant of infectivity resides in the antigenic loop region of HBV envelope proteins. In particular, residues between 119 and 125 of the HBsAg contain a CXXC motif, which is considered to be important for the infectivity of HBV and HDV (Jaoude G A, Sureau C, Journal of Virology, 2005; 79:10460-6).

When positions in the amino acid sequence of the S domain of HbsAg are referred to herein, such positions are made with reference to the amino acid sequence as set forth in SEQ ID NO: 3 (shown below) or to natural or artificial sequence variants thereof.

MENITSGFLGPLLVLQAGFFLLTRILTIPQSLDSWWTSLNFLGGTTVCLG
QNSQSPTSNHSPTSCPPTCPGYRWMCLRRFIIFLFILLLCLIFLLVLLDY
QGMLPVCPLIPGSSTTSTGPCRTCMTTAQGTSMYPSCCCTKPSDGNCTCI
PIPSSWAFGKFLWEWASARFSWLSLLVPFVQWFVGLSPTVWLSVIWMMWY
WGPSLYSILSPFLPLLPIFFCLWVYI (SEQ ID NO: 3; amino
acids 101-172 are shown underlined)

For example, the expression “amino acids 101-172 of the S domain” refers to the amino acid residues from positions 101-172 of the polypeptide according to SEQ ID NO: 3. However, a person skilled in the art understands that mutations or variations (including, but not limited to, substitution, deletion and/or addition, for example, HBsAg of a different genotype or a different HBsAg mutant as described herein) may occur naturally in the amino acid sequence of the S domain of HBsAg or be introduced artificially into the amino acid sequence of the S domain of HBsAg without affecting its biological properties. Therefore, as used herein, the term “S domain of HBsAg” encompasses all such polypeptides including, for example, the polypeptide according to SEQ ID NO: 3 and its natural or artificial mutants. In addition, when sequence fragments of the S domain of HBsAg are described herein (e.g. amino acids 101-172 or amino acids 120-130 of the S domain of HBsAg), they include not only the corresponding sequence fragments of SEQ ID NO: 3, but also the corresponding sequence fragments of its natural or artificial mutants.

For example, the phrase “amino acid residues from positions 101-172 of the S domain of HBsAg” encompasses amino acid residues from positions 101-172 of SEQ ID NO: 3 and the corresponding fragments of its mutants (natural or artificial mutants). As used herein, the phrases “corresponding sequence fragments” and “corresponding fragments” referto fragments that are located in equal positions of sequences when the sequences are subjected to optimized alignment, namely, the sequences are aligned to obtain a highest percentage of identity.

The M protein (M-HBsAg) corresponds to the S protein extended by an N-terminal domain of 55 amino acids called “pre-S2”. The L protein (L-HBsAg) corresponds to the M protein extended by an N-terminal domain of 108 amino acids called “pre-S1” (genotype D). The pre-S1 and pre-S2 domains of the L protein can be present either at the inner face of viral particles (on the cytoplasmic side of the ER), and is believed to play a crucial role in virus assembly, or on the outer face (on the luminal side of the ER), available for the interaction with target cells and important for viral infectivity. Moreover, HBV surface proteins (HBsAgs) are not only incorporated into virion envelopes but also can spontaneously bud from ER-Golgi intermediate compartment membranes to form empty “subviral particles” (SVPs) that are released from the cell by secretion.

In some embodiments, an antibody or antigen binding fragment binds to the antigenic loop region of HBsAg, and is capable of binding to all of S-HBsAg, M-HBsAg and L-HBsAg.

In some embodiments, an antibody or antigen binding fragment neutralizes infection with hepatitis B virus and hepatitis delta virus. In some embodiments, the antibody or antigen binding fragment, reduces viral infectivity of hepatitis B virus and hepatitis delta virus.

To study and quantitate virus infectivity (or “neutralization”) in the laboratory, standard “neutralization assays” may be utilized. For a neutralization assay, animal viruses are typically propagated in cells and/or cell lines. A neutralization assay wherein cultured cells are incubated with a fixed amount of HBV or HDV in the presence (or absence) of the antibody (or antigen-binding fragment) to be tested may be used. In such an assay, the levels of hepatitis B surface antigen (HBsAg) or hepatitis B e antigen (HBeAg) secreted into the cell culture supernatant may be used and/or HBcAg staining may be assessed to provide a readout. For HDV, for example, delta antigen immunofluorescence staining may be assessed.

In a particular embodiment of an HBV neutralization assay, cultured cells, for example HepaRG cells, such as differentiated HepaRG cells, are incubated with a fixed amount of HBV in the presence or absence of the antibody to be tested. In such and embodiment, incubation may be carried out, for example, for 16 hours at 37° C. That incubation may be performed in a medium (e.g. supplemented with 4% PEG 8000). After incubation, cells may be washed and further cultivated. To measure virus infectivity, the levels of hepatitis B surface antigen (HBsAg) and hepatitis B e antigen (HBeAg) secreted into the culture supernatant, e.g. from day 7 to day 11 post-infection, may be determined by enzyme-linked immunosorbent assay (ELISA). Additionally, HBcAg staining may be assessed in an immunofluorescence assay. In an embodiment of a HDV neutralization assay, essentially the same assay as for HBV may be used, with the difference that sera from HDV carriers may be used as HDV infection inoculum on differentiated HepaRg cells (instead of HBV). For detection, delta antigen immunofluorescence staining may be used as a readout.

Embodiments of the antibodies of the disclosure have high neutralizing potency (e.g., in vitro). For example, certain embodiments, the concentration of an antibody as described herein required for 50% neutralization of hepatitis B virus (HBV) and hepatitis delta virus (HDV), is, for example, about 10 μg/ml or less. In other embodiments, the concentration of an antibody required for 50% neutralization of HBV and HDV is about 5 μg/ml. In other embodiments, the concentration of an antibody as described herein required for 50% neutralization of HBV and HDV is about 1 μg/ml. In still other embodiments, the concentration of an antibody required for 50% neutralization of HBV and HDV is about 750 ng/ml. In yet further embodiments, the concentration of an antibody as described herein required for 50% neutralization of HBV and HDV (e.g., in vitro) is 500 ng/ml or less. In such embodiments, the concentration of an antibody as described herein required for 50% neutralization of HBV and HDV may be selected from 450 ng/ml or less, 400 ng/ml or less, 350 ng/ml or less, 300 ng/ml or less, 250 ng/ml or less, 200 ng/ml or less, 175 ng/ml or less, 150 ng/ml or less, 125 ng/ml or less, 100 ng/ml or less, 90 ng/ml or less, 80 ng/ml or less, 70 ng/ml or less, 60 ng/ml or less or 50 ng/ml or less.

Antibodies or antigen binding fragments according to the present disclosure, which can neutralize both HBV and HDV, are useful in the prevention and treatment of hepatitis B and hepatitis D. Infection with HDV typically occurs simultaneously with or subsequent to infection by HBV (e.g., inoculation with HDV in the absence of HBV does not cause hepatitis D since HDV requires the support of HBV for its own replication) and hepatitis D is typically observed in chronic HBV carriers.

Embodiments of the disclosed antibodies promote clearance of HBsAg and HBV. In particular embodiments, antibodies promote clearance of both HBV and subviral particles of hepatitis B virus (SVPs). Clearance of HBsAg or of subviral particles may be assessed by measuring the level of HBsAg for example in a blood sample, e.g. from a hepatitis B patient. Similarly, clearance of HBV may be assessed by measuring the level of HBV for example in a blood sample, e.g. from a hepatitis B patient.

In the sera of patients infected with HBV, in addition to infectious particles (HBV), there is typically an excess (typically 1,000- to 100,000-fold) of empty subviral particles (SVP) composed solely of HBV envelope proteins (HBsAg) in the form of relatively smaller spheres and filaments of variable length. Subviral particles have been shown to strongly enhance intracellular viral replication and gene expression of HBV (Bruns M. et al. 1998 J Virol 72 (2): 1462-1468). This is also relevant in the context of infectivity of sera containing HBV, since the infectivity depends not only on the number of viruses but also on the number of SVPs (Bruns M. et al. 1998 J Virol 72 (2): 1462-1468). Moreover, an excess of subviral particles can serve as a decoy by absorbing neutralizing antibodies and therefore delay the clearance of infection. Achievement of hepatitis B surface antigen (HBsAg) loss is considered in some instances to be an endpoint of treatment and the closest outcome to cure chronic hepatitis B (CHB).

Embodiments of antibodies of the present disclosure may promote clearance of HbsAg. In certain embodiments, the antibodies promote clearance of subviral particles of hepatitis B virus. In some embodiments, the antibodies (e.g., in a presently disclosed pharmaceutical composition) may be used to treat chronic hepatitis B.

In any of the presently disclosed embodiments, an antibody or the antigen binding fragment binds an HBsAg of a genotype selected from the HBsAg genotypes A, B, C, D, E, F, G, H, I, and J, or any combination thereof.

In certain embodiments, an antibody or antigen binding fragment of the present disclosure binds to 1, 2, 3, 4, 5, 6, 7, 8, 9 or 10 of the HBsAg genotypes A, B, C, D, E, F, G, H, I, and J. Examples of different HBsAg genotypes of include the following: GenBank accession number J02203 (HBV-D, ayw3); GenBank accession number FJ899792.1 (HBV-D, adw2); GenBank accession number AM282986 (HBV-A); GenBank accession number D23678 (HBV-B1 Japan); GenBank accession number AB117758 (HBV-C1 Cambodia); GenBank accession number AB205192 (HBV-E Ghana); GenBank accession number X69798 (HBV-F4 Brazil); GenBank accession number AF160501 (HBV-G USA); GenBank accession number AY090454 (HBV-H Nicaragua); GenBank accession number AF241409 (HBV-I Vietnam); and GenBank accession number AB486012 (HBV-J Borneo). Exemplary amino acid sequences of the antigenic loop region of the S domain of HBsAg of different genotypes are described herein (e.g., SEQ ID NOs: 5-15).

In some embodiments, an antibody or antigen binding fragment binds to at least 6 of the 10 HBsAg genotypes A, B, C, D, E, F, G, H, I, and J. In certain embodiments, an antibody or antigen binding fragment binds to at least 8 of the 10 HBsAg genotypes A, B, C, D, E, F, G, H, I, and J. In some embodiments, an antibody or antigen binding fragment binds to all 10 of the 10 HBsAg genotypes A, B, C, D, E, F, G, H, I, and J. HBV is differentiated into several genotypes, according to genome sequence. To date, eight well-known genotypes (A-H) of the HBV genome have been defined. Moreover, two other genotypes, I and J, have also been identified (Sunbul M., 2014, World J Gastroenterol 20 (18): 5427-5434). The genotype is known to affect the progression of the disease and differences between genotypes in response to antiviral treatment have been determined.

In some embodiments, an antibody or antigen binding fragment according to the present disclosure binds to 1, 2, 3, 4, 5, 6, 7, 8, 9, 10, 11, 12, 13, 14, 15, 16, 17 or 18 of the HBsAg mutants having mutations in the antigenic loop region, with such mutant(s) being selected from one ore more of HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. These mutants are naturally occurring mutants based on the S domain of HBsAg Genotype D, Genbank accession no. FJ899792 (SEQ ID NO: 4). The mutated amino acid residue(s) in each of the mutants noted herein are indicated in the name.

SEQ ID NO: 4:
MENVTSGFLGPLLVLQAGFFLLTRILTIPQSLDSWWTSLNFLGGTTVCLG
QNSQSPTSNHSPTSCPPTCPGYRWMCLRRFIIFLFILLLCLIFLLVLLDY
QGMLPVCPLIPGSSTTGTGPCRTCTTPAQGTSMYPSCCCTKPSDGNCTCI
PIPSSWAFGKFLWEWASARFSWLSLLVPFVQWFVGLSPTVWLSVIWMMWY
WGPSLYSTLSPFLPLLPIFFCLWVYI (the antigenic loop
region, i.e. amino acids 101-172, is shown
underlined).

Amino acid sequences of the antigenic loop region of the S domain of HBsAg of different mutants are shown in SEQ ID NOs: 16-33.

In certain embodiments, an antibody or antigen binding fragment binds to at least 12 infectious HBsAg mutants selected from HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. In some such embodiments, an antibody according to the present disclosure, or an antigen binding fragment thereof, binds to at least 15 infectious HBsAg mutants selected from HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. In some embodiments, an antibody or antigen binding fragment binds to each of the following infectious HBsAg mutants: HBsAg Y100C/P120T; HBsAg P120T; HBsAg P120T/S143L; HBsAg C121S; HBsAg R122D; HBsAg R122I; HBsAg T123N; HBsAg Q129H; HBsAg Q129L; HBsAg M133H; HBsAg M133L; HBsAg M133T; HBsAg K141E; HBsAg P142S; HBsAg S143K; HBsAg D144A; HBsAg G145R; and HBsAg N146A.

In certain embodiments, the antibody or pharmaceutical composition comprising the same reduces a serum concentration of HBV DNA in a mammal having an HBV infection. In certain embodiments, the antibody or pharmaceutical composition comprising the same reduces a serum concentration of HBsAg in a mammal having an HBV infection. In certain embodiments, the antibody pharmaceutical composition comprising the same reduces a serum concentration of HBeAg in a mammal having an HBV infection. In certain embodiments, the antibody or pharmaceutical composition comprising the same reduces a serum concentration of HBcrAg in a mammal having an HBV infection.

The term “epitope” or “antigenic epitope” includes any molecule, structure, amino acid sequence, or protein determinant that is recognized and specifically bound by a cognate binding molecule, such as an immunoglobulin, chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

In some embodiments, an antibody or antigen binding fragment binds to an epitope comprising at least one, at least two, at least three, or at least four amino acids of the antigenic loop region of HbsAg. In certain embodiments, an antibody or antigen binding fragment binds at least two amino acids selected from amino acids 115-133 of the S domain of HbsAg, amino acids 120-133 of the S domain of HbsAg, or amino acids 120-130 of the S domain of HbsAg. In certain embodiments, an antibody or antigen binding fragment binds at least three amino acids selected from amino acids 115-133 of the S domain of HbsAg, amino acids 120-133 of the S domain of HbsAg, or amino acids 120-130 of the S domain of HbsAg. In some embodiments, an antibody or antigen binding fragment binds at least four amino acids selected from amino acids 115-133 of the S domain of HbsAg, amino acids 120-133 of the S domain of HbsAg, or amino acids 120-130 of the S domain of HbsAg. As used herein, the position of the amino acids (e.g. 115-133, 120-133, 120-130) refers to the S domain of HBsAg as described above, which is present in all three HBV envelope proteins S-HBsAg, M-HBsAg, and L-HBsAg, whereby S-HBsAg typically corresponds to the S domain of HBsAg.

The term “formed by” as used herein in the context of an epitope, means that the epitope to which an antibody, or an antigen binding fragment thereof, binds to may be linear (continuous) or conformational (discontinuous). A linear or a sequential epitope is an epitope that is recognized by an antibody according to its linear sequence of amino acids, or primary structure. A conformational epitope may be recognized according to a three-dimensional shape and protein structure. Accordingly, if the epitope is a linear epitope and comprises more than one amino acid located at positions selected from amino acid positions 115-133 or from amino acid positions 120-133 of the S domain of HBsAg, the amino acids comprised by the epitope may be located in adjacent positions of the primary structure (e.g., are consecutive amino acids in the amino acid sequence). In the case of a conformational epitope (3D structure), the amino acid sequence typically forms a 3D structure as epitope and, thus, the amino acids forming the epitope may be or may be not located in adjacent positions of the primary structure (i.e. maybe or may be not consecutive amino acids in the amino acid sequence).

In certain embodiments, an epitope to which an antibody or antigen binding fragment binds to a conformational epitope. In some embodiments, an antibody or antigen binding fragment binds to an epitope comprising at least two amino acids of the antigenic loop region of HBsAg, wherein the at least two amino acids are selected from amino acids 120-133 or from from amino acids 120-130, of the S domain of HbsAg, and wherein the at least two amino acids are not located in adjacent positions (of the primary structure). In certain embodiments, an antibody or antigen binding fragment binds to an epitope comprising at least three amino acids of the antigenic loop region of HBsAg, wherein the at least three amino acids are selected from amino acids 120-133 or from from amino acids 120-130, of the S domain of HbsAg, and wherein at least two of the three amino acids are not located in adjacent positions (of the primary structure). In some embodiments, a binding protein binds to an epitope comprising at least four amino acids of the antigenic loop region of HBsAg, wherein the at least four amino acids are selected from amino acids 120-133 or from from amino acids 120-130, of the S domain of HbsAg, and wherein at least two of the four amino acids are not located in adjacent positions (of the primary structure).

Amino acids to which a presently disclosed antibody or antigen binding fragment binds (i.e. the amino acids forming the epitope), which are not located in adjacent positions of the primary structure, are in some cases spaced apart by one or more amino acids, to which the antibody or antigen binding fragment does not bind. In some embodiments, at least one, at least two, at least three, at least four, or at least five amino acids may be located between two of the amino acids not located in adjacent positions comprised by the epitope.

In certain embodiments, an antibody or antigen binding fragment binds to an epitope comprising at least amino acids P120, C121, R122 and C124 of the S domain of HBsAg. In other embodiments, an antibody or antigen binding fragment of the present disclosure binds to an epitope comprising an amino acid sequence according to SEQ ID NO: 88:
PCRXC
wherein X is any amino acid or no amino acid; X is any amino acid; X is T, Y, R, S, or F; X is T, Y or R; or X is T or R.

In other embodiments, an antibody or antigen binding fragment of the present disclosure binds to an epitope comprising an amino acid sequence according to SEQ ID NO: 80:
TGPCRTC
or to an amino acid sequence sharing at least 80%, at least 90%, or at least 95% sequence identity with SEQ ID NO: 80.

In other embodiments, an antibody or antigen binding fragment of the present disclosure binds to an epitope comprising an amino acid sequence according to SEQ ID NO: 85:
STTSTGPCRTC
or to an amino acid sequence sharing at least 80%, at least 90% or at least 95% sequence identity with SEQ ID NO: 85.

In certain embodiments, an antibody or antigen binding fragment of the present disclosure binds to an epitope comprising an amino acid sequence comprising at least amino acids 145-151 of the S domain of HBsAg:
GNCTCIP
(SEQ ID NO: 81).

In still other embodiments, an antibody or antigen binding fragment of the present disclosure binds to an epitope comprising an amino acid sequence according to SEQ ID NO: 80 and an amino acid sequence according to SEQ ID NO: 81.

In other embodiments, an antibody or antigen binding fragment of the present disclosure binds to an epitope comprising an amino acid sequence according to SEQ ID NO: 85 and/or an amino acid sequence according to SEQ ID NO: 87.

As described above, an epitope to which an antibody or antigen binding fragment of the present disclosure binds may be linear (continuous) or conformational (discontinuous). In some embodiments, an antibody or antigen binding fragment of the disclosure binds to a conformational epitope, and in certain such embodiments, the conformational epitope is present only under non-reducing conditions.

In certain embodiments, an antibody or antigen binding fragment of the present disclosure, binds to a linear epitope. In certain such embodiments, the the linear epitope is present under both, non-reducing conditions and reducing conditions.

In particular embodiments, an antibody or antigen binding fragment of the present disclosure binds to an epitope in the antigenic loop of HBsAg formed by an amino acid sequence according to SEQ ID NO: 1:
X₁ X₂ X₃ TC X₄ X₅ X₆A X₇G
wherein X₁, X₂, X₃, X₄, X₅, X₆ and X₇ may be any amino acid (SEQ ID NO: 1).

In some embodiments, X₁, X₂, X₃, X₄, X₅, X₆ and X₇ are amino acids, which are conservatively substituted in comparison to amino acids 120-130 of SEQ ID NO: 3. In some embodiments, X₁, X₂, X₃, X₄, X₅, X₆ and X₇ are amino acids, which are conservatively substituted in comparison to amino acids 20-30 of any of SEQ ID NOs 5-33.

In specific embodiments, X₁ of SEQ ID NO: 1 X₁ is a small amino acid. A “small” amino acid, as used herein, refers to any amino acid selected from the group consisting of alanine, aspartic acid, asparagine, cysteine, glycine, proline, serine, threonine and valine. In certain such embodiments, X₁ is proline, serine or threonine.

In certain embodiments, X₂ of SEQ ID NO: 1 X₂ is a small amino acid. In certain embodiments, X₂ may be selected from cystein or threonine.

In some embodiments, X₃ of SEQ ID NO: 1 is a charged amino acid or an aliphatic amino acid. A “charged” amino acid, as used herein, refers to any amino acid selected from the group consisting of arginine, lysine, aspartic acid, glutamic acid and histidine. A “aliphatic” amino acid, as used herein, refers to any amino acid selected from the group consisting of alanine, glycine, isoleucine, leucine, and valine. In certain embodiments, X₃ is selected from arginine, lysine, aspartic acid or isoleucine.

In some embodiments, X₄ of SEQ ID NO: 1 is a small amino acid and/or a hydrophobic amino acid. A “hydrophobic” amino acid, as used herein, refers to any amino acid selected from the group consisting of alanine, isoleucine, leucine, phenylalanine, valine, tryptophan, tyrosine, methionine, proline and glycine. In certain embodiments, X₄ is selected from methionine or threonine.

In some embodiments, X₅ of SEQ ID NO: 1 X₅ is a small amino acid and/or a hydrophobic amino acid. In certain embodiments, X₅ is selected from threonine, alanine or isoleucine.

In some embodiments, X₆ of SEQ ID NO: 1 X₆ is a small amino acid and/or a hydrophobic amino acid. In certain embodiments, X₆ is selected from threonine, proline or leucine.

In some embodiments, X₇ of SEQ ID NO: 1 is a polar amino acid or an aliphatic amino acid. A “polar” amino acid, as used herein, refers to any amino acid selected from the group consisting of aspartic acid, asparagine, arginine, glutamic acid, histidine, lysine, glutamine, tryptophan, tyrosine, serine, and threonine. In certain such embodiments, X₇ is glutamine, histidine or leucine.

In some embodiments, abinding protein according to the present disclosure binds to an epitope in the antigenic loop of HBsAg formed by an amino acid sequence according to SEQ ID NO: 2:
X₁ X₂ X₃ TC X₄ X₅ X₆A X₇G
wherein X₁ is P, T or S,
   X₂ is C or S,
   X₃ is R, K, D or I,
   X₄ is M or T,
   X₅ is T, A or I,
   X₆ is T, P or L, and
   X₇ is Q, H or L
(SEQ ID NO: 2).

With regard to the epitopes formed by the amino acid sequences according to SEQ ID NO: 1 or 2, it is noted that the term “formed by” as used herein is not intended to imply that a disclosed binding protein necessarily binds to each and every amino acid of SEQ ID NO: 1 or 2. In particular, a binding protein may bind only to some of the amino acids of SEQ ID NO: 1 or 2, whereby other amino acid residues may act as “spacers”.

In particular embodiments, an antibody or antigen binding fragment according to the present disclosure binds to an epitope in the antigenic loop of HBsAg formed by one or more, two or more, three or more, or four or more amino acids of an amino acid sequence selected from SEQ ID NOs 5-33 shown below in Table 3.

In some embodiments, an antibody or antigen binding fragment according to the present disclosure binds to an antigenic loop region of HBsAg having an amino acid sequence according to any one or more of SEQ ID NOs 5-33 shown below in Table 3, or to a sequence variant thereof. In certain embodiments, an antibody or antigen binding fragment according to the present disclosure binds to all of the antigenic loop variants of HBsAg having an amino acid sequence according to any of SEQ ID NOs 5-33 shown below in Table 3.

TABLE 3
Exemplary amino acid sequences of the antigenic loop region of the
S domain of HBsAg (residues 101-172 of the S domain of HBsAg-
except for SEQ ID NO: 16, which refers to residues 100-172 of the
S domain of HBsAg in order to include the relevant mutation) of
the different genotypes and mutants as used herein.

| Name | SEQ ID NO. | Amino acid sequence |
|---|---|---|
| J02203 | 5 | QGMLPVCPLIPGSSTTSTGPCRTCMTTAQGT |
| (D, ayw3) | | SMYPSCCCTKPSDGNCTCIPIPSSWAFGKFL |
| | | WEWASARFSW |
| FJ899792 | 6 | QGMLPVCPLIPGSSTTGTGPCRTCT |
| (D, adw2) | | TPAQGTSMYPSCCCTKPSDGNCTCI |
| | | PIPSSWAFGKFLWEWASARFSW |
| AM282986 | 7 | QGMLPVCPLIPGTTTTSTGPCKTCTTPAQGN |
| (A) | | SMFPSCCCTKPSDGNCTCIPIPSSWAFAKYL |
| | | WEWASVRFSW |
| D23678 (B1) | 8 | QGMLPVCPLIPGSSTTSTGPCKTCTTPAQGTS |
| | | MFPSCCCTKPTDGNCTCIPIPSSWAFAKYLW |
| | | EWASVRFSW |
| AB117758 (C1) | 9 | QGMLPVCPLLPGTSTTSTGPCKTCTIPAQGTS |
| | | MFPSCCCTKPSDGNCTCIPIPSSWAFARFLW |
| | | EWASVRFSW |
| AB205192 (E) | 10 | QGMLPVCPLIPGSSTTSTGPCRTCTTLAQGTS |
| | | MFPSCCCSKPSDGNCTCIPIPSSWAFGKFLW |
| | | EWASARFSW |
| X69798 (F4) | 11 | QGMLPVCPLLPGSTTTSTGPCKTCTTLAQGT |
| | | SMFPSCCCSKPSDGNCTCIPIPSSWALGKYL |
| | | WEWASARFSW |
| AF160501 (G) | 12 | QGMLPVCPLIPGSSTTSTGPCKTCTTPAQGN |
| | | SMYPSCCCTKPSDGNCTCIPIPSSWAFAKYL |
| | | WEWASVRFSW |
| AY090454 (H) | 13 | QGMLPVCPLLPGSTTTSTGPCKTCTTLAQGT |
| | | SMFPSCCCTKPSDGNCTCIPIPSSWAFGKYL |
| | | WEWASARFSW |
| AF241409 (I) | 14 | QGMLPVCPLIPGSSTTSTGPCKTCTTPAQGN |
| | | SMYPSCCCTKPSDGNCTCIPIPSSWAFAKYL |
| | | WEWASARFSW |
| AB486012 (J) | 15 | QGMLPVCPLLPGSTTTSTGPCRTCTITAQGTS |
| | | MFPSCCCTKPSDGNCTCIPIPSSWAFAKFLW |
| | | EWASVRFSW |
| HBsAg | 16 | CQGMLPVCPLIPGSSTTGTGTCRTCTTPAQG |
| Y100C/P120T | | TSMYPSCCCTKPSDGNCTCIPIPSSWAFGKFL |
| | | WEWASARFSW |
| HBsAg P120T | 17 | QGMLPVCPLIPGSSTTGTGTCRTCTTPAQGT |
| | | SMYPSCCCTKPSDGNCTCIPIPSSWAFGKFL |
| | | WEWASARFSW |
| HBsAg | 18 | QGMLPVCPLIPGSSTTGTGTCRTCTTPAQGT |
| P120T/S143L | | SMYPSCCCTKPLDGNCTCIPIPSSWAFGKFL |
| | | WEWASARFSW |
| HBsAg C121S | 19 | QGMLPVCPLIPGSSTTGTGPSRTCTTPAQGTS |
| | | MYPSCCCTKPSDGNCTCIPIPSSWAFGKFLW |
| | | EWASARFSW |
| HBsAg R122D | 20 | QGMLPVCPLIPGSSTTGTGPCDTCTTPAQGT |
| | | SMYPSCCCTKPSDGNCTCIPIPSSWAFGKFL |
| | | WEWASARFSW |
| HBsAg R122I | 21 | QGMLPVCPLIPGSSTTGTGPCITCTTPAQGTS |
| | | MYPSCCCTKPSDGNCTCIPIPSSWAFGKFLW |
| | | EWASARFSW |
| HBsAg T123N | 22 | QGMLPVCPLIPGSSTTGTGPCRNCTTPAQGT |
| | | SMYPSCCCTKPSDGNCTCIPIPSSWAFGKFL |
| | | WEWASARFSW |
| HBsAg Q129H | 23 | QGMLPVCPLIPGSSTTGTGPCRTCTTPAHGT |
| | | SMYPSCCCTKPSDGNCTCIPIPSSWAFGKFL |
| | | WEWASARFSW |
| HBsAg Q129L | 24 | QGMLPVCPLIPGSSTTGTGPCRTCTTPALGTS |
| | | MYPSCCCTKPSDGNCTCIPIPSSWAFGKFLW |
| | | EWASARFSW |
| HBsAg M133H | 25 | QGMLPVCPLIPGSSTTGTGPCRTCTTPAQGT |
| | | SHYPSCCCTKPSDGNCTCIPIPSSWAFGKFL |
| | | WEWASARFSW |
| HBsAg M133L | 26 | QGMLPVCPLIPGSSTTGTGPCRTCTTPAQGT |
| | | SLYPSCCCTKPSDGNCTCIPIPSSWAFGKFLW |
| | | EWASARFSW |
| HBsAg M133T | 27 | QGMLPVCPLIPGSSTTGTGPCRTCTTPAQGT |
| | | STYPSCCCTKPSDGNCTCIPIPSSWAFGKFLW |
| | | EWASARFSW |
| HBsAg K14IE | 28 | QGMLPVCPLIPGSSTTGTGPCRTCTTPAQGT |
| | | SMYPSCCCTEPSDGNCTCIPIPSSWAFGKFL |
| | | WEWASARFSW |
| HBsAg P142S | 29 | QGMLPVCPLIPGSSTTGTGPCRTCTTPAQGT |
| | | SMYPSCCCTKSSDGNCTCIPIPSSWAFGKFL |
| | | WEWASARFSW |
| HBsAg S143K | 30 | QGMLPVCPLIPGSSTTGTGPCRTCTTPAQGT |
| | | SMYPSCCCTKPKDGNCTCIPIPSSWAFGKFL |
| | | WEWASARFSW |
| HBsAg D144A | 31 | QGMLPVCPLIPGSSTTGTGPCRTCTTPAQGT |
| | | SMYPSCCCTKPSAGNCTCIPIPSSWAFGKFL |
| | | WEWASARFSW |
| HBsAg G145R | 32 | QGMLPVCPLIPGSSTTGTGPCRTCTTPAQGT |
| | | SMYPSCCCTKPSDRNCTCIPIPSSWAFGKFL |
| | | WEWASARFSW |
| HBsAg N146A | 33 | QGMLPVCPLIPGSSTTGTGPCRTCTTPAQGT |
| | | SMYPSCCCTKPSDGACTCIPIPSSWAFGKFL |
| | | WEWASARFSW |

Accordingly, in certain aspects, the present disclosure provides an isolated antibody, or an antigen binding fragment thereof suitable for use in the pharmaceutical compositions and methods of the present disclosure, which comprises: (i) a heavy chain variable region (V_{H}) comprising at least 90% identity to the amino acid sequence according to SEQ ID NO:41 or 67; and (ii) a light chain variable region (V_{L}) comprising at least 90% identity to the amino acid sequence according to any one of SEQ ID NOs:42; 59; 65; 89, 90, or 110-120, provided that the amino acid at position 40 of the VL according to IMGT numbering is not a cysteine, wherein the antibody or antigen binding fragment thereof binds to the antigenic loop region of HBsAg and neutralizes infection with hepatitis B virus and hepatitis delta virus.

In further embodiments, (i) the V_{H} comprises at least 95% identity to the amino acid sequence according to SEQ ID NO:41 or 67; and/or (ii) the V_{L} comprises at least 95% identity to the amino acid sequence according to any one of SEQ ID NOs:42, 59, 65, 89, 90, or 110-120.

In certain embodiments, the amino acid at position 40 of the V_{L} is alanine. In other embodiments, the amino acid at position 40 of the V_{L} is serine. In still other embodiments, the amino acid at position 40 of the V_{L} is glycine.

In any of the embodiments disclosed herein, the antibody or antigen binding fragment suitable for use in the pharmaceutical compositions and methods of the present disclosure can comprise CDRH1, CDRH2, CDRH3, CDRL1, CDRL2, and CDRL3 sequences according to SEQ ID NOs: (i) 34-36, 37, 38, and 40, respectively; (ii) 34, 66, 36, 37, 38, and 40, respectively; (iii) 34-36, 37, 39, and 40, respectively; (iv) 34, 66, 36, 37, 39, and 40, respectively; (v) 34-36, 37, 38, and 58, respectively; (vi) 34, 66, 36, 37, 38, and 58, respectively; (vii) 34-36, 37, 39, and 58, respectively; or (vii) 34, 66, 36, 37, 39, and 58, respectively.

In some embodiments, the V_{L} of the antibody or antigen binding fragment suitable for use in the pharmaceutical compositions and methods of the present disclosure comprises or consists of the amino acid sequence according to SEQ ID NO:89. In some embodiments, the V_{L} of the antibody or antigen binding fragment suitable for use in the pharmaceutical compositions and methods of the present disclosure comprises or consists of the amino acid sequence according to SEQ ID NO:90. In other embodiments, the V_{L} of the antibody or antigen binding fragment suitable for use in the pharmaceutical compositions and methods of the present disclosure comprises or consists of the amino acid sequence according to any one of SEQ ID NOs: 109-120. In certain embodiments, the V_{H} comprises or consists of the amino acid sequence according to SEQ ID NO:41. In other embodiments, the V_{H} comprises or consists of the amino acid sequence according to SEQ ID NO:67.

In particular embodiments, the V_{H} comprises or consists of the amino acid sequence according to SEQ ID NO:41 and the V_{L} comprises or consists of the amino acid sequence according to SEQ ID NO: 89. In other embodiments, the V_{H} comprises or consists of the amino acid sequence according to SEQ ID NO:41 and the V_{L} comprises or consists of the amino acid sequence according to SEQ ID NO:90. In certain embodiments, the the V_{H} comprises or consists of the amino acid sequence according to SEQ ID NO:41 and the V_{L} comprises or consists of the amino acid sequence according to any one of SEQ ID NOs:109-120. In other embodiments, the the V_{H} comprises or consists of the amino acid sequence according to SEQ ID NO:67 and the V_{L} comprises or consists of the amino acid sequence according to any one of SEQ ID NOs:109-120.

In another aspect, the present disclosure provides an isolated antibody, or an antigen binding fragment thereof, suitable for use in the pharmaceutical compositions and methods of the present disclosure, which comprises: (i) a heavy chain variable region (V_{H}) comprising at least 90% identity to the amino acid sequence according to SEQ ID NO:95; and (ii) a light chain variable region (V_{L}) comprising at least 90% identity to the amino acid sequence according to SEQ ID NO:96, wherein the antibody or antigen binding fragment thereof binds to the antigenic loop region of HBsAg and neutralizes infection with hepatitis B virus and hepatitis delta virus.

In further embodiments, (i) the VH comprises at least 95% identity to the amino acid sequence according to SEQ ID NO:95; and/or (ii) the VL comprises at least 95% identity to the amino acid sequence according to SEQ ID NO:96. In certain embodiments, the antibody or antigen binding fragment comprises CDRH1, CDRH2, CDRH3, CDRL1, CDRL2, and CDRL3 sequences according to SEQ ID NOs:97-102, respectively.

In particular embodiments, the VH comprises or consists of the amino acid sequence according to SEQ ID NO:95; and the VL comprises or consists of the amino acid sequence according to SEQ ID NO:96

### Fc Moiety

In some embodiments, an antibody or antigen binding fragment thereof suitable for use in the pharmaceutical compositions and methods of the present disclosure comprises an Fc moiety. In certain embodiments, the Fc moiety may be derived from human origin, e.g., from human IgG1, IgG2, IgG3, and/or IgG4. In specific embodiments, an antibody or antigen binding fragment can comprise an Fc moiety derived from human IgG1.

As used herein, the term “Fc moiety” refers to a sequence comprising or derived from a portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. In certain embodiments, a complete Fc moiety comprises a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Amino acid positions within an Fc moiety herein are numbered according to the EU numbering system of Kabat, see e.g., Kabat et al., “Sequences of Proteins of Immunological Interest”, U.S. Dept. Health and Human Services, 1983 and 1987. Amino acid positions of an Fc moirty can also be numbered according to the IMGT numbering system (including unique numbering for the C-domain and exon numbering) and the Kabat numbering system.

In some embodiments, an Fc moiety comprises at least one of: a hinge (e.g., upper, middle, and/or lower hinge region) domain, a CH2 domain, a CH3 domain, or a variant, portion, or fragment thereof. In some embodiments, an Fc moiety comprises at least a hinge domain, a CH2 domain or a CH3 domain. In further embodiments, the Fc moiety is a complete Fc moiety. The amino acid sequence of an exemplary Fc moiety of human IgG1 isotype is provided in SEQ ID NO:137. The Fc moiety may also comprise one or more amino acid insertions, deletions, or substitutions relative to a naturally occurring Fc moiety. For example, at least one of a hinge domain, CH2 domain, or CH3 domain, or a portion thereof, may be deleted. For example, an Fc moiety may comprise or consist of: (i) hinge domain (or a portion thereof) fused to a CH2 domain (or a portion thereof), (ii) a hinge domain (or a portion thereof) fused to a CH3 domain (or a portion thereof), (iii) a CH2 domain (or a portion thereof) fused to a CH3 domain (or a portion thereof), (iv) a hinge domain (or a portion thereof), (v) a CH2 domain (or a portion thereof), or (vi) a CH3 domain or a portion thereof.

An Fc moiety of the present disclosure may be modified such that it varies in amino acid sequence from the complete Fc moiety of a naturally occurring immunoglobulin molecule, while retaining or enhancing at least one desirable function conferred by the naturally occurring Fc moiety. Such functions include, for example, Fc receptor (FcR) binding, antibody half-life modulation (e.g., by binding to FcRn), ADCC function, protein A binding, protein G binding, and complement binding. Portions of naturally occurring Fc moieties which are involved with such functions have been described in the art.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189:4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189:4981-4988).

In some embodiments the antibodies disclosed herein and the antigent binding fragments thereof comprise an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB seems to function to suppress further immunoglobulin production and isotype switching to, for example, the IgE class. On macrophages, FcγRIIB is thought to inhibit phagocytosis as mediated through FcγRIIA. On eosinophils and mast cells, the b form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 103-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164:5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164:5313-5318).

In some embodiments, an antibody or antigen binding fragment thereof suitable for use in the pharmaceutical compositions and methods of the present disclosure comprises an Fc moiety comprising mutations that increase binding affinity of the Fc moiety to a (i.e., one or more) Fcγ receptor, such as a human FcγRIIa, a human FcγRIIIa, or both (e.g., as compared to a reference Fc moiety or antibody containing the same that does not comprise the mutation(s)). See, e.g., Delillo and Ravetch, Cell 161 (5): 1035-1045 (2015) and Ahmed et al., J. Struc. Biol. 194 (1): 78 (2016), the Fc mutations and techniques of which are incorporated herein by reference. In particular embodiments, an antibody or antigen binding fragment thereof suitable for use in the pharmaceutical compositions and methods of the present disclosure comprises a Fc moiety comprising a mutation selected from G236A; S239D; A330L; and 1332E; or a combination comprising the same; e.g., S239D/1332E; S239D/A330L/1332E; G236A/S239D/1332E; G236A/A330L/1332E (also referred to herein as “GAALIE”); or G236A/S239D/A330L/1332E.

In certain embodiments, the Fc moiety may comprise or consist of at least a portion of an Fc moiety that is involved in binding to FcRn (e.g., to a human FcRn). In certain embodiments, the Fc moiety comprises one or more amino acid modifications that improve binding affinity for FcRn and, in some embodiments, thereby extend in vivo half-life of a molecule comprising the Fc moiety (e.g., as compared to a reference Fc moiety or antibody that does not comprise the modification(s)). In certain embodiments, the Fc moiety comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen binding fragment thereof suitable for use in the pharmaceutical compositions and methods of the present disclosure includes a Fc moiety that comprises the substitution mutations M428L/N434S. In some embodiments, a binding protein includes a Fc moiety that comprises the substitution mutations G236A/A330L/1332E. In certain embodiments, an antibody or antigen binding fragment thereof suitable for use in the pharmaceutical compositions and methods of the present disclosure includes a Fc moiety that comprises a G236A mutation, an A330L mutation, and a 1332E mutation (GAALIE), and does not comprise a S239D mutation. In some embodiments, the Fc moiety comprises a Ser at position 239. In particular embodiments, an antibody or antigen binding fragment thereof suitable for use in the pharmaceutical compositions and methods of the present disclosure includes an Fc moiety that comprises the substitution mutations: M428L/N434S and G236A/A330L/1332E. In certain embodiments, an antibody or antigen binding fragment thereof suitable for use in the pharmaceutical compositions and methods of the present disclosure includes a Fc moiety that comprises the substitution mutations: M428L/N434S and G236A/S239D/A330L/1332E. In certain further embodiments, the Fc moiety does not comprise any substitution mutations except for M428L/N434S and G236A/S239D/A330L/1332E.

In certain embodiments, an antibody or antigen binding fragment thereof suitable for use in the pharmaceutical compositions and methods of the present disclosure comprises: CDRs and/or a variable domain and/or a heavy chain and/or a light chain according to any one of the exemplary anti-HBV antibodies disclosed herein and/or in PCT Publication No. WO 2017/060504 (including antibodies HBC34, HBC34v7, HBC34v23, HBC34v31, HBC34v32, HBC34v33, HBC34v34, HBC34v35, (including herein disclosed variants of HBC antibodies which comprise a substitution mutation at position 40 in the light chain (e.g., a substitution of a native cysteine with an alanine, a serine, or the like)); and a Fc moiety comprising a G236A mutation, an A330L mutation, and a 1332E (GAALIE) mutation, wherein the Fc moiety optionally further comprises a M428L/N434S (MLNS) mutation. In certain embodiments, the Fc moiety does not comprise S239D.

In certain embodiments, an antibody or antigen binding fragment thereof suitable for use in the pharmaceutical compositions and methods of the present disclosure comprises: a CDRH1 amino acid sequence according to SEQ ID NO:34, a CDRH2 amino acid sequence according to SEQ ID NO:35 or 66, a CDRH3 amino acid sequence according to SEQ ID NO:36, a CDRL1 acid sequence according to SEQ ID NO:37, a CDRL2 acid sequence according to SEQ ID NO:38 or 39, and CDRL3 amino acid sequence according to SEQ ID NO:58 or 40; and a Fc moiety comprising a GAALIE mutation. In certain embodiments, the Fc moiety further comprises a MLNS mutation.

In certain embodiments, an antibody or antigen binding fragment thereof suitable for use in the pharmaceutical compositions and methods of the present disclosure comprises: a heavy chain variable domain (VH) amino acid sequence according to any one of SEQ ID NOs:41 or 67 and a light chain variable domain (VL) amino acid sequence according to any one of SEQ ID NOs:42, 59, 65, 89, 90, and 111-120; and a Fc moiety comprising a GAALIE mutation. In certain embodiments, the Fc moiety further comprises a MLNS mutation.

In certain embodiments, an antibody or antigen binding fragment thereof suitable for use in the pharmaceutical compositions and methods of the present disclosure comprises a heavy chain amino acid sequence according to SEQ ID NO:138 or 91.

In certain embodiments, an antibody or antigen binding fragment thereof suitable for use in the pharmaceutical compositions and methods of the present disclosure comprises: a CDRH1 amino acid sequence according to SEQ ID NO:97, a CDRH2 amino acid sequence according to SEQ ID NO:98, a CDRH3 amino acid sequence according to SEQ ID NO:99, a CDRL1 acid sequence according to SEQ ID NO:100, a CDRL2 acid sequence according to SEQ ID NO:100, and CDRL3 amino acid sequence according to SEQ ID NO:102; and a Fc moiety comprising a GAALIE mutation. In certain embodiments, the Fc moiety further comprises a MLNS mutation.

In any of the presently disclosed embodiments, a binding protein of the present disclosure includes a Fc moiety comprising a GAALIE mutation and has enhanced binding to a human FcγRIIa and/or a human FcγRIIIa, as compared to a reference polypeptide (i.e., a polypeptide, which may be a binding protein, that includes a Fc moiety that does not comprise the GAALIE mutation).

In certain embodiments, the reference polypeptide includes a Fc moiety that is a wild-type Fc moiety or is a Fc moiety that comprises one or more substitution mutation (or insertion or deletion), provided that the substitution mutation is not GAALIE. In certain embodiments, an antibody or antigen binding fragment thereof suitable for use in the pharmaceutical compositions and methods of the present disclosure comprises HBC34v35 antibody with a GAALIE and MLNS mutations, and a reference polypeptide is HBC34v35 (including a wild-type Fc moiety of a same isotype as the Fc moiety of the antibody or antigen binding fragment thereof suitable for use in the pharmaceutical compositions and methods of the present disclosure). In certain embodiments, the reference polypeptide does not comprise a substitution mutation that is known or believed to affect binding to a human FcγRIIa and/or a human FcγRIIIa.

Binding between polypeptides, such as binding between a Fc moiety (or a binding protein comprising the same) and a human Fcγ Receptor, such as human FcγRIIA, human FcγRIIIA, or human Fc FcγRIIB, or a complement protein, such as C1q, can be determined or detected using methods known in the art. For example, a biolayer interferometry (BLI) assay can be performed using an Octet® RED96 (ForteBio, Fremont, California USA) instrument according to manufacturer's instructions to determine real-time association and dissociation between a first polypeptide of interest (e.g., HBC34v35 comprising a GAALIE mutation) and a second polypeptide of interest (e.g., a FcγRIIA (H131), a FcγRIIA (R131), a FcγRIIIA (F158), a FcγRIIIA (V158), or a FcγRIIb) that is captured on a sensor substrate.

In certain embodiments, an antibody or antigen binding fragment thereof suitable for use in the pharmaceutical compositions and methods of the present disclosure includes a Fc moiety comprising a GAALIE mutation and has enhanced binding to a human FcγRIIA (H131), a human FcγRIIA (R131), a human FcγRIIIA (F158), a human FcγRIIIA (V158), or any combination thereof, as compared to a reference polypeptide that includes a Fc moiety that does not comprise the GAALIE mutation. In certain embodiments, enhanced binding is determined by an increase (e.g., one or more of: a higher peak signal; a greater rate of association; a slower rate of dissociation; or a greater area under the curve) in signal shift versus the reference binding protein in a BLI assay. In certain embodiments, the BLI assay comprises use of Octet® RED96 (ForteBio, Fremont, California USA) instrument. In further embodiments, the BLI assay comprises a tagged human FcγR captured onto an anti-penta-tag sensor and exposed to the binding protein. In some embodiments, the binding protein comprises a IgG Fab and the BLI assay further comprises exposing the captured human FcγR to the antibody or antigen binding fragment in the presence of an anti-IgG Fab binding fragment to cross-link the binding proteins through the Fab fragment.

In certain embodiments, an an antibody or antigen binding fragment thereof suitable for use in the pharmaceutical compositions and methods of the present disclosure includes a Fc moiety comprising a GAALIE mutation and has enhanced binding to a human FcγRIIA (H131), a human FcγRIIA (R131), a human FcγRIIIA (F158), and/or a human FcγRIIIA (V158) as compared to a reference polypeptide, wherein the enhanced binding comprises to a signal shift (nanometers) in a BLI assay of 1.5, 2, 2.5, 3, or more times greater than the signal shift observed using the reference antibody.

In certain embodiments, an antibody or antigen binding fragment thereof suitable for use in the pharmaceutical compositions and methods of the present disclosure includes a Fc moiety comprising a GAALIE mutation and has enhanced binding to a human FcγRIIA (H131), a human FcγRIIA (R131), a human FcγRIIIA (F158), and a human FcγRIIIA (V158), as compared to a reference polypeptide.

In any of the presently disclosed embodiments, an antibody or antigen binding fragment thereof suitable for use in the pharmaceutical compositions and methods of the present disclosure includes a Fc moiety comprising a GAALIE mutation and has reduced binding to a human FcγRIIB, as compared to a reference polypeptide. In certain embodiments, an antibody or antigen binding fragment thereof suitable for use in the pharmaceutical compositions and methods of the present disclosure includes a Fc moiety comprising a GAALIE mutation and does not bind to a human FcγRIIB, as determined, for example, by the absence of a statistically significant signal shift versus baseline in a BLI assay.

In any of the presently disclosed embodiments, an antibody or antigen binding fragment thereof suitable for use in the pharmaceutical compositions and methods of the present disclosure includes a Fc moiety comprising a GAALIE mutation and has reduced binding to a human C1q (complement protein), as compared to a reference polypeptide. In certain embodiments, a binding protein includes a Fc moiety comprising a GAALIE mutation and does not bind to a human C1q, as determined by the absence of a statistically significant signal shift versus baseline in a BLI assay.

In any of the presently disclosed embodiments, an antibody or antigen binding fragment thereof suitable for use in the pharmaceutical compositions and methods of the present disclosure includes a Fc moiety comprising a GAALIE mutation and activates a human FcγRIIA, a human FcγRIIIA, or both, to a greater degree than does a reference polypeptide. (i.e., a polypeptide, which may be an antibody or antigen binding fragment thereof, that includes a Fc moiety that does not comprise the GAALIE mutation). In certain embodiments, the reference polypeptide includes a Fc moiety that is a wild-type Fc moiety or that comprises one or more substitution mutation, provided that the substitution mutation is not GAALIE. In certain embodiments, an antibody or antigen binding fragment thereof comprises HBC34v35 antibody with a GAALIE mutation (and optionally other substitution mutations, such as, for example, MLNS), and a reference polypeptide is HBC34v35 with a wild-type Fc moiety.

Activation of a human FcγR can be determined or detected using methods known in the art. For example, a well-validated, commercially available bioreporter assay involves incubating a HBsAg-specific binding protein with a recombinant HBsAg (Engerix B, GlaxoSmithKline) in the presence of Jurkat effector cells (Promega; Cat. no: G9798) stably expressing (i) a FcγR of interest and (ii) firefly luciferase reporter under the control of a NFAT response element. Binding of Fc to cell surface-expressed FcγR drives NFAT-mediated expression of luciferase reporter gene. Luminescence is then measured with a luminometer (e.g., Bio-Tek) using the Bio-Glo-™ Luciferase Assay Reagent (Promega) according to the manufacturer's instructions. Activation is expressed as the average of relative luminescence units (RLU) over the background by applying the following formula: (RLU at concentration [x] of binding protein (e.g., mAbs)—RLU of background).

In certain embodiments, an antibody or antigen binding fragment thereof includes a Fc moiety comprising a GAALIE mutation activates a human FcγRIIA (H131), a human FcγRIIIA (F158), and/or a human FcγRIIIA (V158) to a greater degree than does a reference polypeptide. In certain embodiments, a greater degree of activation refers to a higher peak luminescence and/or a greater luminescence area under the curve, as determined using a luminescence bioreporter assay as described herein. In certain embodiments, an antibody or antigen binding fragment thereof includes a Fc moiety comprising a GAALIE mutation and activates a human FcγRIIA (H131), a human FcγRIIA (R131), and a human FcγRIIIA (F158) to a greater degree than does a reference polypeptide, wherein the greater degree of activation can be represented by a peak RLU that is 1.5, 2, 2.5, 3, or more times greater than the peak RLU observed using the reference polypeptide.

In any of the presently disclosed embodiments, an antibody or antigen binding fragment thereof includes a Fc moiety comprising a GAALIE mutation does not activate a human FcγRIIB, as determined by the absence of a statistically significant and/or measurable RLU in a luminescence bioreporter assay as described above.

In any of the presently disclosed embodiments, an antibody or antigen binding fragment thereof includes a Fc moiety comprising a GAALIE mutation and activates a human natural killer (NK) cell in the presence of HBsAg to a greater degree than does a reference polypeptide. In certain embodiments, activation of a NK cell is determined by CD107a expression (e.g., by flow cytometry). In certain embodiments, the NK cell comprises a cell that comprises V158/V158 homozygous, a F158/F158 homozygous, or a V158/F158 heterozygous FcγRIIIa genotype.

It will be appreciated that any an antibody or antigen binding fragment thereof including a Fc moiety comprising a GAALIE mutation according to the present disclosure can perform or possess any one or more of the features described herein; e.g., enhanced binding to a human FcγRIIA and/or a human FcγRIIIA as compared to a reference polypeptide; reduced binding to a human FcγRIIB as compared to a reference polypeptide (and/or no binding to a human FcγRIIB); reduced binding to a human C1q as compared to a reference polypeptide (and/or no binding to a human C1q); activates a FcγRIIA, a human FcγRIIIA, or both, to a greater degree than does a reference polypeptide; does not activate a human FcγRIIB; and/or activates a human natural killer (NK) cell in the presence of HBsAg to a greater degree than does a reference polypeptide (e.g., an antibody that is specific for HBsAg and includes a Fc moiety that does not comprise a GAALIE mutation).

Alternatively or additionally, the Fc moiety of an antibody or antigen binding fragment thereof of the disclosure can comprise at least a portion known in the art to be required for Protein A binding; and/or the Fc moiety of an antibody of the disclosure comprises at least the portion of an Fc molecule known in the art to be required for protein G binding. In some embodiments, a retained function comprises the clearance of HBsAg and HBVg. Accordingly, in certain embodiments, an Fc moiety comprises at least a portion known in the art to be required for FcγR binding. As outlined above, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

In some embodiments, an antibody or antigen binding fragment thereof according to the present disclosure comprises an Fc region. As used herein, the term “Fc region” refers to the portion of an immunoglobulin formed by two or more Fc moieties of antibody heavy chains. For example, an Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2, and are incorporated by reference herein. The Fc region can be or comprise a dimeric Fc region. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties (e.g., one Fc monomer of the dimeric Fc region comprises at least one amino acid modification (e.g., substitution, deletion, insertion, or chemical modification) that is not present in the other Fc monomer, or one Fc monomer may be truncated as compared to the other).

Particular embodiments include those antibodies and antigen binding fragments having a heavy chain (e.g., VH-hinge-CH1-CH2-CH3) according to SEQ ID NO:91 or SEQ ID NO:92, and those having a light chain (i.e., VL-CL) according to SEQ ID NO:93 or SEQ ID NO:94. In certain embodiments, an antibody or antigen binding fragment comprises a heavy chain according to SEQ ID NO:91 and a light chain according to SEQ ID NO:93. In other embodiments, an antibody or antigen binding fragment comprises a heavy chain according to SEQ ID NO:92 and a light chain according to SEQ ID NO:94. In other embodiments, an antibody or antigen binding fragment comprises a heavy chain according to SEQ ID NO:91 and a light chain according to SEQ ID NO:94. In other embodiments, an antibody or antigen binding fragment comprises a heavy chain according to SEQ ID NO:92 and a light chain according to SEQ ID NO:93. In some embodiments, an antibody or antigen binding fragment comprises or consists of a heavy chain according to SEQ ID NO:129. In some embodiments, an antibody or antigen binding fragment comprises or consists of a heavy chain according to SEQ ID NO:138. These sequences are provided in the Sequence Listing.

Presently disclosed Fc moieties may comprise Fc sequences or regions of the same or different class and/or subclass. For example, Fc moieties may be derived from an immunoglobulin (e.g., a human immunoglobulin) of an IgG1, IgG2, IgG3 or IgG4 subclass, or from any combination thereof. In certain embodiments, the Fc moieties of Fc region are of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. In certain embodiments, a dimeric Fc region can comprise sequences from two or more different isotypes or subclasses; e.g., a SEEDbody (“strand-exchange engineered domains”), see Davis et al., Protein Eng. Des. Sel. 23 (4): 195 (2010)

Additionally or alternatively, chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

It will also be appreciated that any antibody, antigen-binding fragment, or Fc region or moiety of the present disclosure can be of any allotype and/or haplotype. For example, human Immunoglobulin G allotypes include those disclosed in Jefferis and LeFranc, mAbs 1(4): 1-7 (2009), which allotypes (including G1m (1(a); 2(x); 3(f); and 17(z)); G2m (23(n)); G3m (21(g1); 28(g5); 11(b0); 5(b2); 13(b3); 14(b4); 10(b5); 15(s); 16(t); 6(c3); 24(c5); 26(u); and 27(v)); A2m (1 and 2); and Km (1; 2; and 3) and haplotypes, and resultant amino acid sequences, and combinations thereof, are incorporated herein by reference. In certain embodiments, an antibody, antigen-binding fragment, or Fc region or moiety of the present disclosure comprises a IgG1 allotype glm17, k1.

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In another embodiment, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP). Further chimeric hinges, which may be used in the Fc moiety of the antibody according to the present disclosure are described in US 2005/0163783 A1.

In some embodiments of the an antibody or antigen binding fragment thereof disclosed herein, the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

### Nucleic Acid Molecule

In another aspect, the disclosure provides a nucleic acid molecule comprising a polynucleotide encoding an antibody or antigen binding fragment thereof according to the present disclosure

TABLE 4
shows exemplary VH-, VL-, CH-, CL-, HC-, and LC-encoding nucleotide sequences
according to the present disclosure

| Antibody nucleotide acid | SEQ ID | |
|---|---|---|
| sequence description | NO: | nuclecotide sequence |
| V_{H} of HBC34-V7, HBC34- | 103 | GAGCTGCAGCTGGTGGAGTCCGGCGGCG |
| V35, and HBC34-V34 | | GCTGGGTGCAGCCTGGCGGCTCCCAGAG |
| (codon optimized) | | GCTGAGCTGTGCCGCTTCTGGCAGGATCT |
| | | TCCGGTCCTTTTACATGTCTTGGGTGCGG |
| | | CAGGCTCCAGGCAAGGGCCTGGAGTGGG |
| | | TGGCTACCATCAACCAGGACGGCTCCGA |
| | | GAAGCTGTATGTGGATAGCGTGAAGGGC |
| | | AGATTCACAATCTCTCGCGACAACGCCAA |
| | | GAACTCCCTGTTTCTGCAGATGAACAATC |
| | | TGAGGGTGGAGGATACCGCCGTGTACTA |
| | | TTGCGCCGCTTGGTCTGGCAATAGCGGCG |
| | | GCATGGACGTGTGGGGACAGGGCACCAC |
| | | CGTGTCCGTGTCCAGC |
| HBC34-V34 V_{L} | 104 | AGCTACGAGCTGACACAGCCCCCTTCCGT |
| (codon optimized) | | GTCCGTGTCCCCTGGACAGACCGTGTCCA |
| | | TCCCATGCAGCGGCGACAAGCTGGGCAA |
| | | CAAGAACGTGTCCTGGTTTCAGCATAAGC |
| | | CTGGCCAGTCCCCCGTGCTGGTCATCTAC |
| | | GAGGTGAAGTATAGGCCCAGCGGCATCC |
| | | CTGAGCGGTTCTCTGGCTCCAACAGCGGC |
| | | AATACAGCCACCCTGACAATCTCTGGCAC |
| | | ACAGGCTATGGACGAGGCCGCTTATTTCT |
| | | GCCAGACCTTTGATTCCACCACAGTGGTG |
| | | TTCGGCGGCGGCACCAGACTGACAGTGC |
| | | TG |
| HBC34-V35 V_{L} | 105 | AGCTACGAGCTGACACAGCCCCCTTCCGT |
| (codon optimized) | | GTCCGTGTCCCCTGGACAGACCGTGTCCA |
| | | TCCCATGCAGCGGCGACAAGCTGGGCAA |
| | | CAAGAACGTGGCCTGGTTTCAGCATAAG |
| | | CCTGGCCAGTCCCCCGTGCTGGTCATCTA |
| | | CGAGGTGAAGTATAGGCCCAGCGGCATC |
| | | CCTGAGCGGTTCTCTGGCTCCAACAGCGG |
| | | CAATACAGCCACCCTGACAATCTCTGGCA |
| | | CACAGGCTATGGACGAGGCCGCTTATTTC |
| | | TGCCAGACCTTTGATTCCACCACAGTGGT |
| | | GTTCGGCGGCGGCACCAGACTGACAGTG |
| | | CTG |
| HBC34-V7 V_{L} | 110 | AGCTACGAGCTGACACAGCCCCCTTCCGT |
| (codon optimized) | | GTCCGTGTCCCCTGGACAGACCGTGTCCA |
| | | TCCCATGCAGCGGCGACAAGCTGGGCAA |
| | | CAAGAACGTGTGCTGGTTTCAGCATAAGC |
| | | CTGGCCAGTCCCCCGTGCTGGTCATCTAC |
| | | GAGGTGAAGTATAGGCCCAGCGGCATCC |
| | | CTGAGCGGTTCTCTGGCTCCAACAGCGGC |
| | | AATACAGCCACCCTGACAATCTCTGGCAC |
| | | ACAGGCTATGGACGAGGCCGCTTATTTCT |
| | | GCCAGACCTTTGATTCCACCACAGTGGTG |
| | | TTCGGCGGCGGCACCAGACTGACAGTGC |
| | | TG |
| HBC24 V_{H} | 106 | gaggtgcagttgttggagtctgggggaggcttggtacagcctggg |
| (wild type) | | gggtccctgagactctcctgtgcagcctctGGATCCACTTT |
| | | TACCAAATATGCCatgagctgggtccgtcaggctccag |
| | | ggaaggggctggagtgggtcgcaagtATTAGTGGAAG |
| | | TgttcctggttttGGTATTGACACAtactacgcagactcc |
| | | gttaagggccggttcaccatctccagagacacttccaagaacaccct |
| | | gtatctgcaaatgaacagcctgagagccgaggacacggccttatatt |
| | | actgtGCGAAAGATGTCGGGGTTATCGGGTC |
| | | ATACTATTACTACGCTATGGACGTCtggggtc |
| | | aa |
| HBC24 V_{L} | 107 | aaattgtgttgacgcagtctccaggcaccctgtctttgtctccagggg |
| (wild type) | | aaagagccaccctctcctgcagggccagtCAGGGTCTTA |
| | | GCAGCAGTTACttagcctggtaccagcagaaacctggcc |
| | | aggctcccaggctcctcatctatAGTGCGTCCaccagggcc |
| | | actggcatcccagacaggttcagtggcagtgggtctgggacagact |
| | | tcactctcaccatcagcagactggagcctgaagattttgcagtgtatt |
| | | actgtCAACAGTATGCTTACTCACCTCGGTG |
| | | GACGttcggccaagggaccaaggtggagatcaaac |
| HBC24 V_{H} | 108 | GAGGTGCAGCTGCTGGAAAGCGGCGGCG |
| (codon optimized) | | GCCTGGTGCAGCCCGGCGGCTCCCTGAG |
| | | GCTGTCTTGCGCCGCCTCTGGCAGCACCT |
| | | TCACAAAGTATGCAATGTCTTGGGTGCGC |
| | | CAGGCACCAGGCAAGGGCCTGGAGTGGG |
| | | TGGCCTCCATCTCTGGCAGCGTGCCTGGC |
| | | TTCGGCATCGACACCTACTATGCCGATTC |
| | | CGTGAAGGGCCGGTTTACAATCAGCAGA |
| | | GACACCTCCAAGAACACACTGTATCTGCA |
| | | GATGAATTCTCTGCGGGCCGAGGACACC |
| | | GCCCTGTACTATTGTGCCAAGGATGTGGG |
| | | CGTGATCGGCAGCTACTATTACTATGCAA |
| | | TGGACGTGTGGGGACAGGGAACAGCAGT |
| | | GACAGTGAGCTCC |
| HBC24 V_{L} | 109 | GAGATCGTGCTGACCCAGTCTCCTGGCAC |
| (codon optimized) | | ACTGTCCCTGTCCCCTGGAGAGAGAGCCA |
| | | CCCTGTCCTGCAGAGCCTCTCAGGGCCTG |
| | | AGCTCCTCTTACCTGGCCTGGTATCAGCA |
| | | GAAGCCTGGACAGGCCCCTCGGCTGCTG |
| | | ATCTACTCTGCCTCCACCAGAGCAACAGG |
| | | CATTCCTGACCGCTTCTCCGGATCTGGAA |
| | | GCGGCACAGACTTCACCCTGACAATCAG |
| | | CCGGCTGGAGCCTGAGGACTTCGCCGTGT |
| | | ACTATTGTCAGCAGTACGCCTATTCCCCA |
| | | AGGTGGACCTTTGGCCAGGGCACAAAGG |
| | | TGGAGATCAAG |
| HBC34-V7, HBC34-V34, | 130 | GCCTCCACAAAGGGCCCAAGCGTGTTTCC |
| HBC34-V35 | | ACTGGCTCCCTCTTCCAAGTCTACCTCCG |
| CH1-hinge-CH2-CH3 | | GCGGCACAGCCGCTCTGGGATGTCTGGTG |
| (codon-optimized) | | AAGGATTACTTCCCAGAGCCCGTGACCGT |
| | | GTCTTGGAACTCCGGCGCCCTGACCAGCG |
| | | GAGTGCATACATTTCCAGCTGTGCTGCAG |
| | | AGCTCTGGCCTGTACTCTCTGTCCAGCGT |
| | | GGTGACCGTGCCCTCTTCCAGCCTGGGCA |
| | | CCCAGACATATATCTGCAACGTGAATCAC |
| | | AAGCCAAGCAATACAAAGGTGGACAAGA |
| | | AGGTGGAGCCCAAGTCTTGTGATAAGAC |
| | | CCATACATGCCCTCCATGTCCAGCTCCAG |
| | | AGCTGCTGGGCGGCCCAAGCGTGTTCCTG |
| | | TTTCCACCCAAGCCTAAGGATACCCTGAT |
| | | GATCTCCAGAACCCCCGAGGTGACATGC |
| | | GTGGTGGTGGACGTGAGCCACGAGGATC |
| | | CTGAGGTGAAGTTCAACTGGTACGTGGA |
| | | CGGCGTGGAGGTGCATAATGCTAAGACC |
| | | AAGCCCAGGGAGGAGCAGTACAACTCTA |
| | | CCTATCGGGTGGTGTCCGTGCTGACAGTG |
| | | CTGCACCAGGATTGGCTGAACGGCAAGG |
| | | AGTATAAGTGCAAGGTGTCTAATAAGGC |
| | | CCTGCCCGCTCCTATCGAGAAGACCATCT |
| | | CCAAGGCCAAGGGCCAGCCTAGAGAGCC |
| | | ACAGGTGTACACACTGCCTCCATCTCGCG |
| | | ATGAGCTGACCAAGAACCAGGTGTCCCT |
| | | GACATGTCTGGTGAAGGGCTTCTATCCTT |
| | | CCGACATCGCTGTGGAGTGGGAGAGCAA |
| | | TGGCCAGCCAGAGAACAATTACAAGACC |
| | | ACACCCCCTGTGCTGGACAGCGATGGCTC |
| | | TTTCTTTCTGTATAGCAAGCTGACCGTGG |
| | | ACAAGTCTCGCTGGCAGCAGGGCAACGT |
| | | GTTTAGCTGTTCTGTGATGCATGAGGCCC |
| | | TGCACAATCATTATACACAGAAGTCCCTG |
| | | AGCCTGTCTCCTGGCAAG |
| HBC34-V7, HBC34-V34, | 131 | GAGCTGCAGCTGGTGGAGTCCGGCGGCG |
| HBC34-V35 | | GCTGGGTGCAGCCTGGCGGCTCCCAGAG |
| HC (VH-CH1-hinge-CH2-CH3) | | GCTGAGCTGTGCCGCTTCTGGCAGGATCT |
| (codon-optimized) | | TCCGGTCCTTTTACATGTCTTGGGTGCGG |
| | | CAGGCTCCAGGCAAGGGCCTGGAGTGGG |
| | | TGGCTACCATCAACCAGGACGGCTCCGA |
| | | GAAGCTGTATGTGGATAGCGTGAAGGGC |
| | | AGATTCACAATCTCTCGCGACAACGCCAA |
| | | GAACTCCCTGTTTCTGCAGATGAACAATC |
| | | TGAGGGTGGAGGATACCGCCGTGTACTA |
| | | TTGCGCCGCTTGGTCTGGCAATAGCGGCG |
| | | GCATGGACGTGTGGGGACAGGGCACCAC |
| | | CGTGTCCGTGTCCAGCGCCTCCACAAAGG |
| | | GCCCAAGCGTGTTTCCACTGGCTCCCTCT |
| | | TCCAAGTCTACCTCCGGCGGCACAGCCGC |
| | | TCTGGGATGTCTGGTGAAGGATTACTTCC |
| | | CAGAGCCCGTGACCGTGTCTTGGAACTCC |
| | | GGCGCCCTGACCAGCGGAGTGCATACAT |
| | | TTCCAGCTGTGCTGCAGAGCTCTGGCCTG |
| | | TACTCTCTGTCCAGCGTGGTGACCGTGCC |
| | | CTCTTCCAGCCTGGGCACCCAGACATATA |
| | | TCTGCAACGTGAATCACAAGCCAAGCAA |
| | | TACAAAGGTGGACAAGAAGGTGGAGCCC |
| | | AAGTCTTGTGATAAGACCCATACATGCCC |
| | | TCCATGTCCAGCTCCAGAGCTGCTGGGCG |
| | | GCCCAAGCGTGTTCCTGTTTCCACCCAAG |
| | | CCTAAGGATACCCTGATGATCTCCAGAAC |
| | | CCCCGAGGTGACATGCGTGGTGGTGGAC |
| | | GTGAGCCACGAGGATCCTGAGGTGAAGT |
| | | TCAACTGGTACGTGGACGGCGTGGAGGT |
| | | GCATAATGCTAAGACCAAGCCCAGGGAG |
| | | GAGCAGTACAACTCTACCTATCGGGTGGT |
| | | GTCCGTGCTGACAGTGCTGCACCAGGATT |
| | | GGCTGAACGGCAAGGAGTATAAGTGCAA |
| | | GGTGTCTAATAAGGCCCTGCCCGCTCCTA |
| | | TCGAGAAGACCATCTCCAAGGCCAAGGG |
| | | CCAGCCTAGAGAGCCACAGGTGTACACA |
| | | CTGCCTCCATCTCGCGATGAGCTGACCAA |
| | | GAACCAGGTGTCCCTGACATGTCTGGTGA |
| | | AGGGCTTCTATCCTTCCGACATCGCTGTG |
| | | GAGTGGGAGAGCAATGGCCAGCCAGAGA |
| | | ACAATTACAAGACCACACCCCCTGTGCTG |
| | | GACAGCGATGGCTCTTTCTTTCTGTATAG |
| | | CAAGCTGACCGTGGACAAGTCTCGCTGG |
| | | CAGCAGGGCAACGTGTTTAGCTGTTCTGT |
| | | GATGCATGAGGCCCTGCACAATCATTATA |
| | | CACAGAAGTCCCTGAGCCTGTCTCCTGGC |
| | | AAGTGATGAGGTACCGTGCGACGGCCGG |
| | | CAAGCCCCCGCTCCCCGGGCTCTCGCGGT |
| | | CGTACGAGGAAAGCTT |
| HBC34-V7 CL | 132 | GGACAGCCAAAGGCTGCTCCATCTGTGA |
| (codon-optimized) | | CCCTGTTTCCACCCTCTTCCGAGGAGCTG |
| | | CAGGCCAACAAGGCCACCCTGGTGTGCC |
| | | TGATCTCTGACTTCTACCCTGGAGCTGTG |
| | | ACAGTGGCTTGGAAGGCTGATAGCTCTCC |
| | | CGTGAAGGCTGGCGTGGAGACAACAACC |
| | | CCTAGCAAGCAGTCTAACAATAAGTACG |
| | | CCGCTTCCAGCTATCTGTCTCTGACACCA |
| | | GAGCAGTGGAAGTCCCACCGCTCTTATTC |
| | | CTGCCAGGTGACCCATGAGGGCAGCACC |
| | | GTGGAGAAGACAGTGGCCCCCACCGAGT |
| | | GTTCT |
| HBC34-V7 LC | 133 | AGCTACGAGCTGACACAGCCCCCTTCCGT |
| (VL-CL) (codon-optimized) | | GTCCGTGTCCCCTGGACAGACCGTGTCCA |
| | | TCCCATGCAGCGGCGACAAGCTGGGCAA |
| | | CAAGAACGTGTGCTGGTTTCAGCATAAGC |
| | | CTGGCCAGTCCCCCGTGCTGGTCATCTAC |
| | | GAGGTGAAGTATAGGCCCAGCGGCATCC |
| | | CTGAGCGGTTCTCTGGCTCCAACAGCGGC |
| | | AATACAGCCACCCTGACAATCTCTGGCAC |
| | | ACAGGCTATGGACGAGGCCGCTTATTTCT |
| | | GCCAGACCTTTGATTCCACCACAGTGGTG |
| | | TTCGGCGGCGGCACCAGACTGACAGTGC |
| | | TGGGACAGCCAAAGGCTGCTCCATCTGTG |
| | | ACCCTGTTTCCACCCTCTTCCGAGGAGCT |
| | | GCAGGCCAACAAGGCCACCCTGGTGTGC |
| | | CTGATCTCTGACTTCTACCCTGGAGCTGT |
| | | GACAGTGGCTTGGAAGGCTGATAGCTCTC |
| | | CCGTGAAGGCTGGCGTGGAGACAACAAC |
| | | CCCTAGCAAGCAGTCTAACAATAAGTAC |
| | | GCCGCTTCCAGCTATCTGTCTCTGACACC |
| | | AGAGCAGTGGAAGTCCCACCGCTCTTATT |
| | | CCTGCCAGGTGACCCATGAGGGCAGCAC |
| | | CGTGGAGAAGACAGTGGCCCCCACCGAG |
| | | TGTTCT |
| HBC34-V34, HBC34-V35 | 134 | GGACAGCCAAAGGCTGCTCCATCTGTGA |
| CL | | CCCTGTTTCCACCCTCTTCCGAGGAGCTG |
| (codon-optimized) | | CAGGCCAACAAGGCCACCCTGGTGTGCC |
| | | TGATCTCTGACTTCTACCCTGGAGCTGTG |
| | | ACAGTGGCTTGGAAGGCTGATAGCTCTCC |
| | | CGTGAAGGCTGGCGTGGAGACAACAACC |
| | | CCTAGCAAGCAGTCTAACAATAAGTACG |
| | | CCGCTTCCAGCTATCTGTCTCTGACACCA |
| | | GAGCAGTGGAAGTCCCACCGCTCTTATTC |
| | | CTGCCAGGTGACCCATGAGGGCAGCACC |
| | | GTGGAGAAGACAGTGGCCCCCACCGAGT |
| | | GTTCT |
| HBC34-V34 LC | 135 | AGCTACGAGCTGACACAGCCCCCTTCCGT |
| (VL-CL) | | GTCCGTGTCCCCTGGACAGACCGTGTCCA |
| (codon-optimized) | | TCCCATGCAGCGGCGACAAGCTGGGCAA |
| | | CAAGAACGTGTCCTGGTTTCAGCATAAGC |
| | | CTGGCCAGTCCCCCGTGCTGGTCATCTAC |
| | | GAGGTGAAGTATAGGCCCAGCGGCATCC |
| | | CTGAGCGGTTCTCTGGCTCCAACAGCGGC |
| | | AATACAGCCACCCTGACAATCTCTGGCAC |
| | | ACAGGCTATGGACGAGGCCGCTTATTTCT |
| | | GCCAGACCTTTGATTCCACCACAGTGGTG |
| | | TTCGGCGGCGGCACCAGACTGACAGTGC |
| | | TGGGACAGCCAAAGGCTGCTCCATCTGTG |
| | | ACCCTGTTTCCACCCTCTTCCGAGGAGCT |
| | | GCAGGCCAACAAGGCCACCCTGGTGTGC |
| | | CTGATCTCTGACTTCTACCCTGGAGCTGT |
| | | GACAGTGGCTTGGAAGGCTGATAGCTCTC |
| | | CCGTGAAGGCTGGCGTGGAGACAACAAC |
| | | CCCTAGCAAGCAGTCTAACAATAAGTAC |
| | | GCCGCTTCCAGCTATCTGTCTCTGACACC |
| | | AGAGCAGTGGAAGTCCCACCGCTCTTATT |
| | | CCTGCCAGGTGACCCATGAGGGCAGCAC |
| | | CGTGGAGAAGACAGTGGCCCCCACCGAG |
| | | TGTTCT |
| HBC34-V35 LC | 136 | AGCTACGAGCTGACACAGCCCCCTTCCGT |
| (VL-CL) (codon-optimized) | | GTCCGTGTCCCCTGGACAGACCGTGTCCA |
| | | TCCCATGCAGCGGCGACAAGCTGGGCAA |
| | | CAAGAACGTGGCCTGGTTTCAGCATAAG |
| | | CCTGGCCAGTCCCCCGTGCTGGTCATCTA |
| | | CGAGGTGAAGTATAGGCCCAGCGGCATC |
| | | CCTGAGCGGTTCTCTGGCTCCAACAGCGG |
| | | CAATACAGCCACCCTGACAATCTCTGGCA |
| | | CACAGGCTATGGACGAGGCCGCTTATTTC |
| | | TGCCAGACCTTTGATTCCACCACAGTGGT |
| | | GTTCGGCGGCGGCACCAGACTGACAGTG |
| | | CTGGGACAGCCAAAGGCTGCTCCATCTGT |
| | | GACCCTGTTTCCACCCTCTTCCGAGGAGC |
| | | TGCAGGCCAACAAGGCCACCCTGGTGTG |
| | | CCTGATCTCTGACTTCTACCCTGGAGCTG |
| | | TGACAGTGGCTTGGAAGGCTGATAGCTCT |
| | | CCCGTGAAGGCTGGCGTGGAGACAACAA |
| | | CCCCTAGCAAGCAGTCTAACAATAAGTA |
| | | CGCCGCTTCCAGCTATCTGTCTCTGACAC |
| | | CAGAGCAGTGGAAGTCCCACCGCTCTTAT |
| | | TCCTGCCAGGTGACCCATGAGGGCAGCA |
| | | CCGTGGAGAAGACAGTGGCCCCCACCGA |
| | | GTGTTCT |

Due to the redundancy of the genetic code, the present disclosure also comprises sequence variants of these nucleic acid sequences and in particular such sequence variants, which encode the same amino acid sequences.

In certain embodiments, a polynucleotide or nucleic acid molecule comprises a nucleotide sequence sharing at least 80% identity to the nucleotide sequence according to any one of SEQ ID NOs: 103-110 and 130-136, wherein the nucleotide sequence is codon optimized for expression by a host cell.

In particular embodiments, a nucleic acid molecule according to the present disclosure comprises or consists of a nucleic acid sequence according to any one of SEQ ID NOs: 103-110 and 130-136.

In certain embodiments, a polynucleotide comprises a V_{H}-encoding nucleotide sequence according to SEQ ID NO:103 and a V_{L}-encoding nucleotide sequence according to SEQ ID NO:105. In other embodiments, a polynucleotide comprises a V_{H}-encoding nucleotide sequence according to SEQ ID NO:103, and a V_{L}-encoding nucleotide sequence according to SEQ ID NO:104. In other embodiments, a polynucleotide comprises a V_{H}-encoding nucleotide sequence according to SEQ ID NO: 108, and a V_{L}-encoding nucleotide sequence according to SEQ ID NO: 109.

Also provided herein are polynucleotides that encode an antibody or antigen binding fragment, wherein the polynucleotide comprises or consists of a VH-encoding nucleotide sequence according to SEQ ID NO:103 and a VL-encoding nucleotide sequence according to SEQ ID NO:110, wherein the encoded antibody or antigen binding fragment binds to the antigenic loop region of HBsAg and neutralizes infection with hepatitis B virus and hepatitis delta virus.

In any of the presently disclosed embodiments, a polynucleotide can comprise a CH1-hinge-CH2-CH3-encoding nucleotide sequence according to SEQ ID NO:130, and/or comprises a HC (VH-CH1-hinge-CH3-CH3)-encoding nucleotide sequence according to SEQ ID NO:131. In some embodiments, a polynucleotide comprises a CL-encoding nucleotide sequence according to SEQ ID NO:132 and/or comprises a LC (VL-CL)-encoding nucleotide sequence according to SEQ ID NO:133. In other embodiments, a polynucleotide comprises a CL-encoding nucleotide sequence according to SEQ ID NO:134 and/or comprises a LC (VL-CL)-encoding nucleotide sequence according to SEQ ID NO:135 or SEQ ID NO:136.

### Vectors

Further included within the scope of the disclosure are vectors, for example, expression vectors, that comprise a nucleic acid molecule according to the present disclosure.

The term “vector” refers to a construct comprising a nucleic acid molecule. A vector in the context of the present disclosure is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired antibody or antibody fragment thereof according to the present description.

As used herein, “expression vector” refers to a DNA construct containing a nucleic acid molecule that is operably linked to a suitable control sequence capable of effecting the expression of the nucleic acid molecule in a suitable host. Such control sequences include a promoter (e.g., a heterologous promoter) to effect transcription, an optional operator sequence to control such transcription, a sequence encoding suitable mRNA ribosome binding sites, and sequences which control termination of transcription and translation. Any of the elements of an expression vector that contribute to transcription of a nucleic acid molecule of interest may be heterologous to the vector. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

A cloning vector is typically a vector that contains a cloning site, which may be used to incorporate nucleic acid sequences into the vector. A cloning vector may be, e.g., a plasmid vector or a bacteriophage vector.

A transfer vector may be a vector which is suitable for transferring nucleic acid molecules into cells or organisms, for example, viral vectors. A vector in the context of the present disclosure may be, e.g., an RNA vector or a DNA vector. A vector may be a DNA molecule. For example, a vector in the sense of the present application comprises a cloning site, a selection marker, such as an antibiotic resistance factor, and a sequence suitable for multiplication of the vector, such as an origin of replication. In some embodiments, a vector in the context of the present application is a plasmid vector. In certain such embodiments, a vector comprises a lentiviral vector or a retroviral vector.

### Cells

In a further aspect, the present disclosure also provides a cell (also referred to as a “host cell”) expressing an antibody, antigen binding fragment, or fusion protein according to the present disclosure; or comprising a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, YO cells, Sp2/0 cells. NSO cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

Insect cells useful expressing an antibody or antigen binding fragment thereof of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153 (3-4): 160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody or antigen binding fragment thereof of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

Any protein expression system compatible with the disclosure may be used to produce a disclosed an antibody or antigen binding fragment thereof. Suitable expression systems include transgenic animals described in Gene Expression Systems, Academic Press, eds. Fernandez et al., 1999. In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine etc. In certain embodiments, the introduction is non-viral.

Moreover, cells of the present disclosure may be transfected stably or transiently with the vector according to the present description, e.g. for expressing an antibody, or an antigen binding fragment thereof, according to the present description. In such embodiments, the cells are stably transfected with the vector as described herein encoding a binding protein. Alternatively, cells may be transiently transfected with a vector according to the present disclosure encoding a binding protein according to the present description. In any of the presently disclosed embodiments, a polynucleotide may be heterologous to the host cell.

In a related aspect, the present disclosure provides methods for producing an antibody or antigen binding fragment thereof, wherein the methods comprise culturing a host cell of the present disclosure under conditions and for a time sufficient to produce the antibody or antigen binding fragment thereof.

Accordingly, the present disclosure also provides recombinant host cells that heterologously express an antibody or antigen binding fragment thereof of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen binding fragment in nature. Moreover, the host cell may impart a post-translational modification (PTM; e.g., glysocylation or fucosylation) on the antibody or antigen binding fragment that is not present in a native state of the antibody or antigen binding fragment (or in a native state of a parent antibody from which the antibody or antigen binding fragment was engineered or derived). Such a PTM may result in a functional difference (e.g., reduced immunogenicity). Accordingly, an antibody or antigen binding fragment of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell,

### Optional Additional Features of the Antibodies or Antigen Binding Fragments

Antibodies and antigen binding fragments of the disclosure may be coupled, for example, to a drug for delivery to a treatment site or coupled to a detectable label to facilitate imaging of a site comprising cells of interest. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody (or antigen binding fragment or fusion protein) of the disclosure and an epitope of interest on HBsAg, in particular on the antigenic loop region of HBsAg, can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include 125I, 131I, 35S, or 3H. Such labeled reagents may be used in a variety of well-known assays, such as radioimmunoassays, enzyme immunoassays, e.g., ELISA, fluorescent immunoassays, and the like. Labeled antibodies and antigen binding fragments according to the present disclosure may be thus be used in such assays for example as described in U.S. Pat. Nos. 3,766,162; 3,791,932; 3,817,837; and 4,233,402.

An antibody or antigen binding fragment thereof according to the present disclosure may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like.

Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. pp. 475-506 (Editrice Kurtis, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, an antibody or antigen binding fragment thereof can be conjugated to a second antibody, or antibody fragment thereof, (or second fusion protein) to form a heteroconjugate as described in U.S. Pat. No. 4,676,980. In addition, linkers may be used between the labels and the antibodies of the description, e.g., as described in U.S. Pat. No. 4,831,175. Antibodies, antigen-binding fragments, and fusion proteins may be directly labeled with radioactive iodine, indium, yttrium, or other radioactive particle known in the art, e.g., as described in U.S. Pat. No. 5,595,721. Treatment may consist of a combination of treatment with conjugated and non-conjugated antibodies and/or antigen binding fragments, administered simultaneously or subsequently e.g., as described in WO00/52031; WO00/52473.

Antibodies and antigen binding fragments as described herein may also be attached to a solid support. Additionally, the antibodies of the present disclosure, or functional antibody fragments thereof, can be chemically modified by covalent conjugation to a polymer to, for example, increase their circulating half-life. Examples of polymers, and methods to attach them to peptides, are shown in U.S. Pat. Nos. 4,766,106; 4,179,337; 4,495,285 and 4,609,546. In some embodiments, the polymers may be selected from polyoxyethylated polyols and polyethylene glycol (PEG). PEG is soluble in water at room temperature and has the general formula: R(O-CH₂—CH₂)ₙO—R, wherein R can be hydrogen, or a protective group such as an alkyl or alkanol group. In certain embodiments, the protective group may have between 1 and 8 carbons. For example, the protective group may be methyl. The symbol n is a positive integer. In one embodiment, n is between 1 and 1,000. In another embodiment n is between 2 and 500. In some embodiments, the PEG has an average molecular weight selected from between 1,000 and 40,000, between 2,000 and 20,000, and between 3,000 and 12,000. Furthermore, PEG may have at least one hydroxy group, for example the PEG may have a terminal hydroxy group. For example, it is the terminal hydroxy group which is activated to react with a free amino group on the inhibitor. However, it will be understood that the type and amount of the reactive groups may be varied to achieve a covalently conjugated PEG/antibody of the present description.

Water-soluble polyoxyethylated polyols may also be utilized in the context of the antibodies and antigen binding fragments described herein. They include polyoxyethylated sorbitol, polyoxyethylated glucose, polyoxyethylated glycerol (POG), and the like. In one embodiment, POG is used. Without being bound by any theory, because the glycerol backbone of polyoxyethylated glycerol is the same backbone occurring naturally in, for example, animals and humans in mono-, di-, triglycerides, this branching would not necessarily be seen as a foreign agent in the body. POG may have a molecular weight in the same range as PEG. Another drug delivery system that can be used for increasing circulatory half-life is the liposome. Methods of preparing liposome delivery systems are known to one of skill in the art. Other drug delivery systems are known in the art and are described in, for example, referenced in Poznansky et al. (1980) and Poznansky (1984).

Typically, the antibody or antigen binding fragment will be present in a composition that is substantially free of other polypeptides e.g., where less than 90% (by weight), usually less than 60% and more usually less than 50% of the composition is made up of other polypeptides.

Antibodies, or antigen binding fragments of the disclosure may be immunogenic in non-human (or heterologous) hosts e.g., in mice. In particular, the antibodies, antigen binding fragments, or fusion proteins may have an idiotope that is immunogenic in non-human hosts, but not in a human host. In particular, such molecules of the disclosure for human use include those that cannot be easily isolated from hosts such as mice, goats, rabbits, rats, non-primate mammals, etc. and cannot generally be obtained by humanization or from xeno-mice.

### Production of Antibodies, Antigen Binding Fragments, and Fusion Proteins

Antibodies and antigen binding fragments according to the disclosure can be made by any method known in the art. For example, the general methodology for making monoclonal antibodies using hybridoma technology is well known (Kohler, G. and Milstein, C., 1975; Kozbar et al. 1983). In one embodiment, the alternative EBV immortalization method described in WO2004/076677 is used.

In one embodiment, antibodies are produced using a method described in WO 2004/076677. In such methods, B cells producing the antibody are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another method for producing antibodies is described in WO 2010/046775. In such a method, plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The V_{H} and V_{L} regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies, antibody fragments, or fusion proteins of the present description. Desired DNA sequences may be synthesized completely or in part using oligonucleotide synthesis techniques.

Site-directed mutagenesis and polymerase chain reaction (PCR) techniques may be used as appropriate.

Any suitable host cell/vector system may be used for expression of the DNA sequences encoding the antibody or fusion protein molecules of the present disclosure or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. Eukaryotic, e.g., mammalian, host cell expression systems may be used for production of larger antibody molecules, including complete antibody molecules. Suitable mammalian host cells include, but are not limited to, CHO, HEK293T, PER.C6, NSO, myeloma or hybridoma cells.

The present disclosure also provides a process for the production of an antibody or antigen binding fragment according to the present disclosure comprising culturing a host cell comprising a vector encoding a nucleic acid of the present disclosure under conditions suitable for expression of protein from DNA encoding the antibody molecule of the present description, and isolating the antibody molecule.

An antibody molecule or antibody fragment may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding a light chain polypeptide and and a heavy chain polypeptide.

Alternatively, antibodies and antigen binding fragments according to the disclosure may be produced by (i) expressing a nucleic acid sequence according to the disclosure in a host cell, e.g. by use of a vector according to the present description, and (ii) isolating the expressed desired product. Additionally, the method may include (iii) purifying the isolated antibody or antigen binding fragment. Transformed B cells and cultured plasma cells may be screened for those producing antibodies and antigen binding fragments of the desired specificity or function.

Screening may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, or the like.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of described herein can be used in various ways e.g., as a source of monoclonal antibodies, as a source of nucleic acid (DNA or mRNA) encoding a monoclonal antibody of interest, for research, etc.

### Pharmaceutical Compositions

The present disclosure provides pharmaceutical compositions comprising an antibody that neutralizes hepatitis B virus and a pharmaceutically acceptable, aqueous vehicle. A vehicle is typically understood to be a material that is suitable for storing, transporting, formulating and/or administering a compound, such as a pharmaceutically active compound, in particular the antibodies according to the present disclosure. For example, the vehicle may be a physiologically acceptable liquid, which is suitable for storing, transporting, and/or administering a pharmaceutically active compound, in particular the antibodies according to the present disclosure.

The pharmaceutical compositions described herein are prepared for injection or infusion into a patient. In some embodiments, the composition may be prepared for intravenous (“IV” or “i.v.”), intra-arterial, or intraventricular infusion. In other embodiments, the composition may be prepared for intravenous, intra-arterial, intraventricular, intramedullary, intraperitoneal, intrathecal, intraventricular, or injection. In particular embodiments, the composition is prepared for subcutaneous (“SC” or “s.c.”) injection. In specific embodiments, the compositions described herein are pharmaceutically acceptable, sterile aqueous solutions exhibiting suitable pH, isotonicity and stability for administration to a human subject. Aqueous vehicles suitable for formulation of the compositions described herein include water (e.g., sterile water, USP water for injection), as well as isotonic vehicles such as Sodium Chloride Injection, Ringer's Injection, Lactated Ringer's Injection.

Pharmaceutical compositions according to the present description include an antibody selected from HBV neutralizing antibodies according to the present description. For example, in some embodiments, pharmaceutical compositions according to the present description include an (isolated) antibody comprising (i) a heavy chain variable region (VH) comprising at least 90% identity to the amino acid sequence according to SEQ ID NO:41; and (ii) a light chain variable region (VL) comprising at least 90% identity to the amino acid sequence according to any one of SEQ ID NOs: 59, 89, or 90, provided that the amino acid at position 40 of the VL according to IMGT numbering is not a cysteine, wherein the antibody or antigen binding fragment thereof binds to the antigenic loop region of HBsAg and neutralizes infection with hepatitis B virus and hepatitis delta virus.

In certain embodiments: (i) the VH comprises at least 95% identity to the amino acid sequence according to SEQ ID NO:41; and/or (ii) the VL comprises at least 95% identity to the amino acid sequence according to any one of SEQ ID NOs: 59, 89, or 90.

In certain embodiments, the amino acid at position 40 of the VL is alanine. In certain embodiments, the amino acid at position 40 of the VL is serine. In certain embodiments, the amino acid at position 40 of the VL is glycine.

In certain embodiments, the antibody comprises CDRH1, CDRH2, CDRH3, CDRL1, CDRL2, and CDRL3 sequences according to SEQ ID NOs: (i) 34-36, 37, 38, and 40, respectively; (ii) 34, 66, 36, 37, 38, and 40, respectively; (iii) 34-36, 37, 39, and 40, respectively; (iv) 34, 66, 36, 37, 39, and 40, respectively; (v) 34-36, 37, 38, and 58, respectively; (vi) 34, 66, 36, 37, 38, and 58, respectively; (vii) 34-36, 37, 39, and 58, respectively; or (viii) 34, 66, 36, 37, 39, and 58, respectively.

In certain embodiments, the VL comprises or consists of the amino acid sequence according to SEQ ID NO:89.

In certain embodiments, the VL comprises or consists of the amino acid sequence according to SEQ ID NO:90.

In certain embodiments, the VH comprises or consists of the amino acid sequence according to SEQ ID NO:41.

In certain embodiments, the VH comprises or consists of the amino acid sequence according to SEQ ID NO:41 and VL comprises or consists of the amino acid sequence according to SEQ ID NO:89.

In certain embodiments, the VH comprises or consists of the amino acid sequence according to SEQ ID NO:41 and VL comprises or consists of the amino acid sequence according to SEQ ID NO:90.

In certain embodiments, the antibody comprises a human antibody and/or a monoclonal antibody.

In certain embodiments, the antibody is a multi specific antibody. In certain embodiments, the antibody is a bispecific antibody.

In certain embodiments, the antibody comprises a Fc moiety.

In certain embodiments, the Fc moiety comprises a mutation that enhances binding to a (e.g., human) FcRn as compared to a reference Fc moiety that does not comprise the mutation.

In certain embodiments, Fc moiety comprises a mutation that enhances binding to a (e.g., human) FcγR (e.g., such as a FcγRIIa, a FcγRIIIa, or both) as compared to a reference Fc moiety that does not comprise the mutation.

In certain embodiments, the Fc moiety is an IgG isotype or is derived from an IgG isotype.

In certain embodiments, the mutation that enhances binding to FcRn comprises: M428L; N434S; N434H; N434A; N434S; M252Y; S254T; T256E; T250Q; P257I; Q311I; D376V; T307A; E380A; or any combination thereof.

In certain embodiments, the mutation that enhances binding to FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

In certain embodiments, the mutation that enhances binding to FcRn comprises M428L/N434S.

In certain embodiments, the mutation that enhances binding to a FcγR comprises S239D; 1332E; A330L; G236A; or any combination thereof.

In certain embodiments, the mutation that enhances binding to a FcγR comprises: (i) S239D/1332E; (ii) S239D/A330L/1332E; (iii) G236A/S239D/1332E; or (iv) G236A/A330L/1332E.

In certain embodiments, the mutation that enhances binding to a FcγR comprises or consists of G236A/A330L/1332E. In some embodiments, the mutation that enhances binding to a FcγR does not comprise S239D. In some embodiments, the Fc moiety comprises a native Ser(S) at position 239.

In certain embodiments, the Fc moiety comprises the amino acid substitution mutations: M428L; N434S; G236A; A330L; and I332E. In certain further embodiments, the Fc moiety does not comprise a further mutation.

In certain embodiments, the antibody comprises the heavy chain (HC) amino acid sequence according to SEQ ID NO: 91.

In certain embodiments, the antibody comprises the heavy chain (HC) amino acid sequence according to SEQ ID NO: 92.

In certain embodiments, the antibody comprises the light chain (LC) amino acid sequence according to SEQ ID NO:93.

In certain embodiments, the antibody comprises the light chain (LC) amino acid sequence according to SEQ ID NO:94.

In certain embodiments, the antibody comprises the HC amino acid sequence according to SEQ ID NO:91 and the LC amino acid sequence according to SEQ ID NO:93.

In certain embodiments, the antibody comprises the HC amino acid sequence according to SEQ ID NO:92 and the LC amino acid sequence according to SEQ ID NO:94.

In certain embodiments, the antibody comprises the HC amino acid sequence according to SEQ ID NO:91 and the LC amino acid sequence according to SEQ ID NO:94.

In certain embodiments, the antibody comprises the HC amino acid sequence according to SEQ ID NO:92 and the LC amino acid sequence according to SEQ ID NO:93

In some embodiments, pharmaceutical compositions according to the present description include an (isolated) antibody comprising: (i) a heavy chain (HC) comprising the amino acid sequence according to SEQ ID NO:91; and (ii) a light chain (LC) comprising the amino acid sequence according to SEQ ID NO:93, wherein the antibody binds to the antigenic loop region of HBsAg and neutralizes infection with hepatitis B virus and hepatitis delta virus.

In some embodiments, the antibody binds an HBsAg of a genotype selected from the HBsAg genotypes A, B, C, D, E, F, G, H, I, and J, or any combination thereof.

In some embodiments, the antibody or pharmaceutical composition reduces a serum concentration of HBV DNA in a mammal having an HBV infection. In some embodiments, the antibody or pharmaceutical composition reduces a serum concentration of HBsAg in a mammal having an HBV infection. In some embodiments, the antibody or pharmaceutical composition reduces a serum concentration of HBeAg in a mammal having an HBV infection. In some embodiments, the antibody or pharmaceutical composition reduces a serum concentration of HBcrAg in a mammal having an HBV infection.

In some embodiments, pharmaceutical compositions according to the present description include an antibody comprising: a heavy chain variable region (V_{H}) comprising a CDRH1 amino acid sequence according to SEQ ID NO:34, a CDRH2 amino acid sequence according to SEQ ID NO:35 or 66, a CDRH3 amino acid sequence according to SEQ ID NO:36; and a light chain variable region (V_{L}) comprising a CDRL1 acid sequence according to SEQ ID NO:37, a CDRL2 acid sequence according to SEQ ID NO:38 or 39, and CDRL3 amino acid sequence according to SEQ ID NO:58 or 40; and a Fc moiety, wherein the Fc moiety comprises G236A/A330L/1332E.

In certain embodiments, the Fc moiety does not comprise S239D. In certain embodiments, the Fc moiety comprises a Ser(S) at position 239.

In certain embodiments, the Fc moiety further comprises M428L/N434S.

In certain embodiments, the V_{H} comprises or consists of the amino acid sequence according to any one of SEQ ID NOs:41 or 67 and the V_{L} comprises or consists of the amino acid sequence according to any one of SEQ ID NOs:42, 59, 65, 89, 90, and 111-120.

In other embodiments, a pharmaceutical composition is provided that comprises an antibody, or an antigen binding fragment thereof, comprising: (i) a heavy chain variable region (V_{H}) comprising a CDRH1 amino acid sequence according to SEQ ID NO:97, a CDRH2 amino acid sequence according to SEQ ID NO:98, a CDRH3 amino acid sequence according to SEQ ID NO:99; (ii) a light chain variable region (V_{L}) comprising a CDRL1 acid sequence according to SEQ ID NO:100, a CDRL2 acid sequence according to SEQ ID NO:100, and CDRL3 amino acid sequence according to SEQ ID NO:102; and (iii) a Fc moiety, wherein the Fc moiety comprises G236A/A330L/1332E.

In particular such embodiments of the antibody of the pharmaceutical composition, V_{H} comprises or consists of the amino acid sequence according to SEQ ID NO:95, and wherein the V_{L} comprises or consists of the amino acid sequence according to SEQ ID NO:96.

In certain embodiments, the Fc moiety does not comprise S239D. In certain embodiments, the Fc moiety further comprises M428L/N434S.

In some embodiments, the antibody of the pharmaceutical composition: has enhanced binding to a human FcγRIIA, a human FcγRIIIA, or both, as compared to a reference polypeptide that includes a Fc moiety that does not comprise G236A/A330L/1332E, wherein the human FcγRIIA is optionally H131 or R131, and/or the human FcγRIIIA is optionally F158 or V158; has reduced binding to a human FcγRIIB, as compared to a reference polypeptide that includes a Fc moiety that does not comprise G236A/A330L/1332E; does not bind to a human FcγRIIB; has reduced binding to a human C1q, as compared to a reference polypeptide that includes a Fc moiety that does not comprise G236A/A330L/1332E; does not bind to a human C1q; activates a FcγRIIA, a human FcγRIIIA, or both, to a greater degree than does a reference polypeptide that includes a Fc moiety that does not comprise G236A/A330L/1332E, wherein the human FcγRIIA is optionally H131 or R131, and/or the human FcγRIIIA is optionally F158 or V158; does not activate a human FcγRIIB; and/or activates a human natural killer (NK) cell in the presence of HBsAg to a greater degree than does a reference polypeptide that includes a Fc moiety that does not comprise G236A/A330L/1332E.

The pharmaceutical compositions include sufficient antibody material to facilitate administration of a therapeutically effective amount of antibody to a patient. In some embodiments, the antibody is included at a concentration selected from 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, and 200 mg/mL. In other embodiments, the antibody is included in the composition at a concentration selected from above 50 mg/mL, above 75 mg/mL, above 100 mg/mL, above 125 mg/mL, above 150 mg/mL, above 175 mg/mL, above 200 mg/mL, above 225 mg/mL, and above 250 mg/mL. In other embodiments, the composition comprises the antibody at a concentration selected from a range of 50 mg/mL to 200 mg/mL, a range of 75 mg/mL to 225 mg/mL, and a range of 100 mg/mL to 200 mg/mL. In some embodiments, composition comprises the antibody at a concentration ranging from 125 mg/ml to 150 mg/ml. In still other embodiments, the composition comprises the antibody at a concentration of 150 mg/mL.

Compositions according to the present description may include one or more of a buffer, a surfactant or a triblock copolymer, a salt (e.g., sodium chloride), and a stabilizer (such as a sugar alcohol, disaccharide, or polysaccharide stabilizer, and/or a stabilizing amino acid, (e.g., arginine and/or glycine)). In addition, where needed or desired, the compositions described herein may be formulated to additionally include one or more antioxidants (e.g., ascorbic acid, methionine, ethylenediaminetetraacetic acid (EDTA)).

Pharmaceutical compositions of the disclosure exhibit and maintain a pH that maintains the viability of the antibody, while also being suitable for injection or infusion. The compositions described herein are generally have a pH in a range from about 5.5 to about 8.5. In certain embodiments, the pharmaceutical composition has a pH in a range from about 5.5 to about 6.5, such as in a range from 5.5 to 6.5. In some embodiments, the pharmaceutical composition has a pH in a range from 5.8 to 6.2, for example, about 6.0. In certain embodiments, the pH may be 5.5, 5.6, 5.7, 5.8, 5.9, 6.0, 6.1, 6.2, 6.3, 6.4, or 6.5. In some embodiments, the composition has a pH in a range from 6 to 8, for example, about 7. In certain such embodiments, the pH may be about 6, such as, for example, 6.

The composition may include a buffering agent to achieve and maintain a desired pH. Buffers suitable for use in the compositions described herein include, e.g., acetate, citrate, histidine, succinate, phosphate, and hydroxymethylaminomethane (Tris) buffers. In particular embodiments, the composition includes a buffer selected from a histidine buffer and a phosphate buffer. In specific embodiments, the composition exhibits of pH of 6 and includes a histidine buffer. In such embodiments, the histidine may be included in the composition at a concentration in a range from 10 mM to 40 mM (e.g., 10 mM, 15 mM, 20 mM, 25 mM, 30 mM, 35 mM, or 40 mM). For example, in specific embodiments, the composition according to the present description exhibits a pH of 6 and includes histidine at a concentration selected from 10 mM, 15 mM, 20 mM, 25 mM, 30 mM, 35 mM, and 40 mM.

The pharmaceutical compositions described herein may also include a surfactant or a triblock copolymer. Surfactants, sometimes referred to as “detergents,” can serve one or more functions. For instance, in aqueous antibody solutions, surfactants and serve to preserve antibody functionality, aid in dissolution of the antibody or other excipients, and/or work to control microbial growth. Surfactants that may be used in the compositions described herein include, e.g., polysorbate 80 (Tween 80), polysorbate 20 (Tween 20). Additionally or alternatively, a triblock copolymer such as poloxamer 188 may be used. In some embodiments, the composition includes a surfactant at a concentration ranging from 0.01% to 0.05% (w/v). In such embodiments, the surfactant may be selected from polysorbate 80 (Tween 80), polysorbate 20 (Tween 20), and poloxamer 188. In specific embodiments, the pharmaceutical composition of the present description includes polysorbate 80 (Tween 80) at a concentration ranging from 0.01% to 0.05% (w/v). In other embodiments, the pharmaceutical composition of the present description includes polysorbate 80 (Tween 80) at a concentration 0.02% (w/v).

Where the compositions according to the present disclosure include a sugar alcohol, disaccharide, or polysaccharide stabilizer, the stabilizer may be selected from, e.g., mannitol, sorbitol, sucrose, trehalose, and dextran 40. In particular embodiments, the stabilizer is a disaccharide. In certain embodiments, the pharmaceutical composition includes a disaccharide at a concentration selected from 4.0% to 10% (w/v). In certain such embodiments, the disaccharide is sucrose. In other embodiments, the pharmaceutical composition includes sucrose at a concentration selected from 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0% (w/v), or is within a range bounded by and including any two of these values. In still other embodiments, the pharmaceutical composition includes sucrose at a concentration of about 7%, such as 7% (w/v).

In some embodiments, the compositions are adapted for administration to mammalian, e.g., human subjects. In such embodiments, the composition is sterile, and may be specifically prepared to be pyrogen free. In addition, the composition may be isotonic with respect to humans.

The compositions described herein may be prepared for direct administration to a subject (i.e., without a reconstitution or mixing step), or they may be prepared as a lyophilized material to be reconstituted in an aqueous vehicle prior to injection or infusion to a patient. For direct administration to a subject, the pharmaceutical composition according to the present disclosure may be provided, e.g., in a pre-filled syringe, or in a vial, such as a glass vial. In some embodiments pharmaceutical compositions of the disclosure are supplied in hermetically-sealed containers. In some embodiments, the composition may may be in kit form, designed such that a combined composition is reconstituted just prior to administration to a subject. For example, a lyophilized antibody may be provided in kit form with sterile water or a sterile buffer.

The administration a pharmaceutical composition according to the present disclosure in the methods and uses according to the disclosure can be carried out alone or in combination with a co-agent (also referred to as “additional active component” herein), which may be useful for preventing and/or treating hepatitis B virus infection.

The disclosure encompasses the administration of a pharmaceutical composition according to the present disclosure, wherein it is administered to a subject prior to, simultaneously with or after a co-agent or another therapeutic regimen useful for treating and/or preventing hepatitis B virus infection. Said pharmaceutical composition administered in combination with said co-agent can be administered in the same or different composition(s) and by the same or different route(s) of administration. As used herein, expressions like “combination therapy”, “combined administration”, “administered in combination” and the like refer to a combined action of the drugs (which are to be administered “in combination”). To this end, the combined drugs are usually present at a site of action at the same time and/or within an overlapping time window. It may also be possible that the effects resulting from one of the drugs are still ongoing (even if the drug itself may no longer be present at a detectable) while the other drug is administered, such that effects of both drugs can interact. However, a drug which was administered long before another drug (e.g., more than one, two, three or more months or a year), such that it is no longer present at a detectable level (or its effects are not ongoing) when the other drug is administered, is typically not considered to be administered “in combination”.

In certain embodiments, a pharmaceutical composition of the present disclosure is used in combination with a PD-1 inhibitor, for example a PD-1-specific antibody or binding fragment thereof, such as pidilizumab, nivolumab, pembrolizumab, MEDI0680 (formerly AMP-514), AMP-224, BMS-936558 or any combination thereof.

In certain embodiments, a pharmaceutical composition of the present disclosure is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a pharmaceutical composition of the present disclosure is used in combination with a LAG3 inhibitor, such as LAG525, IMP321, IMP701, 9H12, BMS-986016, or any combination thereof.

In certain embodiments, a pharmaceutical composition of the present disclosure is used in combination with an inhibitor of CTLA4. In particular embodiments, a pharmaceutical composition of the present disclosure is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a pharmaceutical composition of the present disclosure is used in combination with a B7-H3 specific antibody or binding fragment thereof, such as enoblituzumab (MGA271), 376.96, or both. A B7-H3 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a pharmaceutical composition of the present disclosure is used in combination with an inhibitor of CD244.

In certain embodiments, a pharmaceutical composition of the present disclosure is used in combination with an inhibitor of BLTA, HVEM, CD160, or any combination thereof. Anti CD-160 antibodies are described in, for example, PCT Publication No. WO 2010/084158.

In certain embodiments, a pharmaceutical composition of the present disclosure is used in combination with an inhibitor of TIM3.

In certain embodiments, a pharmaceutical composition of the present disclosure is used in combination with an inhibitor of Gal9.

In certain embodiments, a pharmaceutical composition of the present disclosure is used in combination with an inhibitor of adenosine signaling, such as a decoy adenosine receptor.

In certain embodiments, a pharmaceutical composition of the present disclosure is used in combination with an inhibitor of A2aR.

In certain embodiments, a pharmaceutical composition of the present disclosure is used in combination with an inhibitor of KIR, such as lirilumab (BMS-986015).

In certain embodiments, a pharmaceutical composition of the present disclosure is used in combination with an inhibitor of an inhibitory cytokine (typically, a cytokine other than TGFß) or Treg development or activity.

In certain embodiments, a pharmaceutical composition of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a pharmaceutical composition of the present disclosure is used in combination with an arginase inhibitor, such as N (omega)-Nitro-L-arginine methyl ester (L-NAME), N-omega-hydroxy-nor-l-arginine (nor-NOHA), L-NOHA, 2 (S)-amino-6-boronohexanoic acid (ABH), S-(2-boronoethyl)-L-cysteine (BEC), or any combination thereof.

In certain embodiments, a pharmaceutical composition of the present disclosure is used in combination with an inhibitor of VISTA, such as CA-170 (Curis, Lexington, Mass.).

In certain embodiments, a pharmaceutical composition of the present disclosure is used in combination with an inhibitor of TIGIT such as, for example, COM902 (Compugen, Toronto, Ontario Canada), an inhibitor of CD155, such as, for example, COM701 (Compugen), or both.

In certain embodiments, a pharmaceutical composition of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition of the present disclosure is used in combination with a LAIR1 inhibitor.

In certain embodiments a pharmaceutical composition of the present disclosure is used in combination with an inhibitor of CEACAM-1, CEACAM-3, CEACAM-5, or any combination thereof.

In certain embodiments, a pharmaceutical composition of the present disclosure is used in combination with an agent that increases the activity (i.e., is an agonist) of a stimulatory immune checkpoint molecule. For example, a composition of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a pharmaceutical composition of the present disclosure with one or more agonist of a stimulatory immune checkpoint molecule, including any of the foregoing, singly or in any combination.

In some embodiments, a pharmaceutical composition of this disclosure is used in combination with a nucleos (t) ide reverse transcriptase inhibitor (NRTI), an interferon (e.g., IFNα, IFNβ, or both), or any combination thereof. In some embodiments, the NRTI comprises one or more of: tenofovir; tenofovir disoproxil (e.g., tenofovir disproxil fumarate); tenofovir alafenamide; Entecavir; Lamivudine; Adefovir; and adefovir dipivoxil.

### Medical Treatments and Uses

In a further aspect, the present disclosure provides the use of a pharmaceutical composition according to the present disclosure in treatment of infection with Hepatitis B virus. In particular embodiments, the present disclosure provides methods for treatment of infection with Hepatitis B virus, with the methods comprising: administering to a subject in need thereof, a therapeutically effective amount of a pharmaceutical composition according to the present disclosure.

In therapeutic settings, the subject is infected with Hepatitis B virus infection, diagnosed with Hepatitis B virus infection, and/or showing symptoms of Hepatitis B virus infection. Of note, the terms “treatment” and “therapy”/“therapeutic” of Hepatitis B virus infection include (complete) cure as well as attenuation/reduction of Hepatitis B virus infection and/or related symptoms (e.g., attenuation/reduction of severity of infection and/or symptoms, number of symptoms, duration of infection and/or symptoms, or any combination thereof).

In certain embodiments, the subject is an adult. In certain embodiments, the subject is in a range from 18 years of age to 65 years of age. In certain embodiments, the subject weighs from 40 kg to 125 kg.

In certain embodiments, a subject administered a pharmaceutical composition of the present disclosure has a chronic HBV infection; e.g., defined by positive serum HBsAg, HBV DNA, and/or HBeAg on 2 occasions at least 6 months apart.

In certain embodiments, a subject administered a pharmaceutical composition of the present disclosure does not have cirrhosis. Absence of cirrhosis is determined by: Fibroscan evaluation (e.g., within 6 months prior to administering the single dose of the pharmaceutical composition); or liver biopsy (e.g., within 12 months prior to administering the single dose of the pharmaceutical composition), wherein, preferably, the absence of cirrhosis is determined by the absence of Metavir F3 fibrosis or the absence of F4 cirrhosis.

In certain embodiments, a subject administered a pharmaceutical composition of the present disclosure has received a nucleos (t) ide reverse transcriptase inhibitor (NRTI), optionally within 120 days, further optionally within 60 days, prior to the single dose of the pharmaceutical composition being administered. In other words, the subject has previously received NRTI, such as within 120 days or within 60 days of administration of the pharmaceutical composition.

In certain embodiments, the NRTI comprises one or more of: tenofovir; tenofovir disoproxil (e.g., tenofovir disproxil fumarate); tenofovir alafenamide; Entecavir; Lamivudine; Adefovir; and adefovir dipivoxil.

In certain embodiments, a subject administered a pharmaceutical composition of the present disclosure has a serum HBV DNA concentration of less than 100 IU/mL (e.g., 99, 98, 97, 96, 95, 90, 80, 70, 60, or the like) no more than 28 days prior to the single dose being administered.

In certain embodiments, a subject administered a pharmaceutical composition of the present disclosure has a serum HBsAg concentration of less than 1,000 IU/mL prior to the single dose being administered.

In certain embodiments, a subject administered a pharmaceutical composition of the present disclosure has a serum HB surface antigen (HBsAg) concentration of greater than or equal to 1,000 IU/mL no more than 28 days prior to the single dose being administered. HBsAg concentration can be determined using, for example using an Abbott ARCHITECT assay.

In certain embodiments, a subject administered a pharmaceutical composition of the present disclosure was HB e-antigen (HBeAg)-negative no more than 28 days prior to the single dose being administered.

In certain embodiments, the subject was negative for anti-HB antibodies no more than 28 days prior to the single dose being administered.

In certain embodiments, a subject administered a pharmaceutical composition of the present disclosure: (i) does not have fibrosis and/or does not have cirrhosis; and/or (ii) has (serum) alanine aminotransferase (ALT)<2× Upper Limit of Normal (ULN).

In certain embodiments, a method comprises administering a single dose of a pharmaceutical composition of the present disclosure.

In some embodiments, the single dose of the pharmaceutical composition comprises the antibody in a range from 2 to 18 mg/kg (subject body weight); e.g., 2, 3, 4, 5, 6, 7, 8, 9, 10, 11, 12, 13, 14, 15, 16, 17 or 18 mg/kg.

In certain embodiments, a single dose of the pharmaceutical composition comprises up to 6 mg, up to 18 mg, up to 75 mg, up to 90 mg, up to 300 mg, up to 900 mg, or up to 3000 mg of the antibody. In particular embodiments, the single dose of the pharmaceutical composition comprises about 10, about 25, about 50, about 75, about 90, about 100, about 125, about 150, about 175, about 200, about 250, about 300, about 350, about 400, about 450, about 500, about 550, about 600, about 650, about 700, about 750, about 800, about 850, about 900, about 950, about 1000, about 1250, about 1500, about 1750, about 2000, about 2250, about 2500, about 2750, or about 3000 mg of the antibody.

In particular embodiments, the single dose of the pharmaceutical composition comprises about 75 mg of the antibody. In other embodiments, the single dose of the pharmaceutical composition comprises about 90 mg of the antibody. In still other embodiments, the single dose of the pharmaceutical composition comprises up to 300 mg of the antibody. In yet other embodiments, the single dose of the pharmaceutical composition comprises up to 900 mg of the antibody. In yet other embodiments, the single dose of the pharmaceutical composition comprises up to 3,000 mg of the antibody.

In certain embodiments, a single dose of the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

In any of the methods for treatment of infection with Hepatitis B virus described herein, the pharmaceutical composition can be administered via injection or infusion. When administered by infusion, the pharmaceutical compositions may be administered by, e.g., intravenous, intra-arterial, or intraventricular infusion. When administered by injection, the pharmaceutical compositions may be administered by, e.g., intravenous, intra-arterial, intraventricular, intramedullary, intraperitoneal, intrathecal, intraventricular, or subcutaneous injection. In specific embodiments of the methods described herein, the pharmaceutical composition is administered via subcutaneous (“SC”) injection, or via intravenous (“IV”) injection.

Even where multiple injections or infusions are needed to administer a defined dose, the dose is referred to as a “single dose” and the administration is regarded to be a “single administration.” In general, if multiple injections or infusions are needed to administer a single defined dose, the multiple injections or infusions are administered over a period of about 5 minutes or less, about 15 minutes or less, about 30 minutes or less, about 1 hour or less, about 2 hours or less, about 4 hours or less, about 6 hours or less, about 1 day or less, about 1 week or less, or about 1 month or less.

In certain embodiments, wherein at about 56 days following administration of the single dose, the subject has a >2-fold reduction in serum HBsAg (e.g., concentration of HBsAg in serum, e.g., as determined using an Abbott ARCHITECT assay) as compared to the subject's serum HBsAg at from 0 days to 28 days prior to administration of the single dose.

In certain embodiments, following administration of the single dose of the pharmaceutical composition (e.g., at 56 days following administration of the single dose), the subject has: (i) has reduced or less severe intrahepatic spread of HBV as compared to a reference subject (e.g., a subject having a HBV infection of similar severity and of a same gender, age, body weight, and/or general health as the subject receiving the pharmaceutical composition) over a same time period who received a placebo or did not receive a therapy for HBV.; and/or (ii) comprises an adaptive immune response against HBV, e.g., including a T cell response specific for HBV.

The present disclosure also includes the following exemplary embodiments.

Embodiment 1. An isolated antibody, or an antigen binding fragment thereof, comprising: (i) a heavy chain variable region (V_{H}) comprising at least 90% identity to the amino acid sequence according to SEQ ID NO:41; and (ii) a light chain variable region (V_{L}) comprising at least 90% identity to the amino acid sequence according to any one of SEQ ID NOs: 59, 89, or 90, provided that the amino acid at position 40 of the V_{L} according to IMGT numbering is not a cysteine, wherein the antibody or antigen binding fragment thereof binds to the antigenic loop region of HBsAg and neutralizes infection with hepatitis B virus and hepatitis delta virus.

Embodiment 2. The antibody or antigen binding fragment of Embodiment 1, wherein: (i) the V_{H} comprises at least 95% identity to the amino acid sequence according to SEQ ID NO:41; and/or (ii) the V_{L} comprises at least 95% identity to the amino acid sequence according to any one of SEQ ID NOs: 59, 89, or 90.

Embodiment 3. The antibody or antigen binding fragment of Embodiment 1 or 2, wherein the amino acid at position 40 of the V_{L} is alanine.

Embodiment 4. The antibody or antigen binding fragment of Embodiment 1 or 2, wherein the amino acid at position 40 of the V_{L} is serine.

Embodiment 5. The antibody or antigen binding fragment of Embodiment 1 or 2, wherein the amino acid at position 40 of the V_{L} is glycine.

Embodiment 6. The antibody or antigen binding fragment of any one of Embodiments 1-5, comprising CDRH1, CDRH2, CDRH3, CDRL1, CDRL2, and CDRL3 sequences according to SEQ ID NOs: (i) 34-36, 37, 38, and 40, respectively; (ii) 34, 66, 36, 37, 38, and 40, respectively; (iii) 34-36, 37, 39, and 40, respectively; (iv) 34, 66, 36, 37, 39, and 40, respectively; (v) 34-36, 37, 38, and 58, respectively; (vi) 34, 66, 36, 37, 38, and 58, respectively; (vii) 34-36, 37, 39, and 58, respectively; or (viii) 34, 66, 36, 37, 39, and 58, respectively.

Embodiment 7. The antibody or antigen binding fragment of any one of Embodiments 1-3 or 6, wherein the V_{L} comprises or consists of the amino acid sequence according to SEQ ID NO:89.

Embodiment 8. The antibody or antigen binding fragment of any one of Embodiments 1, 2, 4, or 6, wherein the V_{L} comprises or consists of the amino acid sequence according to SEQ ID NO:90.

Embodiment 9. The isolated antibody of any one of Embodiments 1-8, wherein the V_{H} comprises or consists of the amino acid sequence according to SEQ ID NO:41.

Embodiment 10. The isolated antibody of any one of Embodiments 1-3, 6, 7, or 9, wherein the V_{H} comprises or consists of the amino acid sequence according to SEQ ID NO:41 and V_{L} comprises or consists of the amino acid sequence according to SEQ ID NO:89.

Embodiment 11. The isolated antibody of any one of Embodiments 1, 2, 4, 6, 8, or 9, wherein the V_{H} comprises or consists of the amino acid sequence according to SEQ ID NO:41 and V_{L} comprises or consists of the amino acid sequence according to SEQ ID NO:90.

Embodiment 12. An isolated antibody, or an antigen binding fragment thereof, comprising:
(i) a heavy chain variable region (V_{H}) comprising at least 90% identity to the amino acid sequence according to SEQ ID NO:95; and
(ii) a light chain variable region (V_{L}) comprising at least 90% identity to the amino acid sequence according to SEQ ID NO:96, wherein the antibody or antigen binding fragment thereof binds to the antigenic loop region of HBsAg and neutralizes infection with hepatitis B virus and hepatitis delta virus.

Embodiment 13. The antibody or antigen binding fragment of Embodiment 12, wherein:
(i) the V_{H} comprises at least 95% identity to the amino acid sequence according to SEQ ID NO:95; and/or
(ii) the V_{L} comprises at least 95% identity to the amino acid sequence according to SEQ ID NO:96.

Embodiment 14. The antibody or antigen binding fragment of Embodiment 12 or 13, comprising CDRH1, CDRH2, CDRH3, CDRL1, CDRL2, and CDRL3 sequences according to SEQ ID NOs:97-102, respectively.

Embodiment 15. The antibody or antigen binding fragment of any one of Embodiments 1-14, wherein the antibody, or the antigen binding fragment thereof, comprises a human antibody, a monoclonal antibody, a purified antibody, a single chain antibody, a Fab, a Fab′, a F(ab′)2, a Fv, or a scFv.

Embodiment 16. The antibody or antigen binding fragment of any one of Embodiments 1-15, wherein the antibody or antigen binding fragment is a multi-specific antibody or antigen binding fragment.

Embodiment 17. The antibody or antigen binding fragment of any one of Embodiment 16, wherein the antibody or antigen binding fragment is a bispecific antibody or antigen binding fragment.

Embodiment 18. The antibody of any one of Embodiments 1-17, or an antigen binding fragment thereof, wherein the antibody or the antigen binding fragment comprises a Fc moiety.

Embodiment 19. The antibody or antigen binding fragment of Embodiment 18, wherein the Fc moiety comprises a mutation that enhances binding to (e.g., human) FcRn as compared to a reference Fc moiety that does not comprise the mutation.

Embodiment 20. The antibody or antigen binding fragment of Embodiment 18 or 19, wherein the Fc moiety comprises a mutation that enhances binding to a (e.g., human) FcγR as compared to a reference Fc moiety that does not comprise the mutation.

Embodiment 21. The antibody or antigen binding fragment of any one of Embodiments 18-20, wherein the Fc moiety is an IgG isotype or is derived from an IgG isotype.

Embodiment 22. The antibody or antigen binding fragment of Embodiment 21, wherein the mutation that enhances binding to FcRn comprises: M428L; N434S; N434H; N434A; N434S; M252Y; S254T; T256E; T250Q; P257I; Q311I; D376V; T307A; E380A; or any combination thereof.

Embodiment 23. The antibody or antigen binding fragment of Embodiment 21 or 22, wherein the mutation that enhances binding to FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 24. The antibody or antigen binding fragment of Embodiment 23, wherein the mutation that enhances binding to FcRn comprises M428L/N434S.

Embodiment 25. The antibody or antigen binding fragment of any one of Embodiments 20-24, wherein the mutation that enhances binding to a FcγR comprises S239D; I332E; A330L; G236A; or any combination thereof.

Embodiment 26. The antibody or antigen binding fragment of Embodiment 25, wherein the mutation that enhances binding to a FcγR comprises: (i) S239D/1332E; (ii) S239D/A330L/1332E; (iii) G236A/S239D/1332E; or (iv) G236A/A330L/1332E.

Embodiment 27. The antibody or antigen binding fragment of Embodiment 25 or 26, wherein the mutation that enhances binding to a FcγR comprises or consists of G236A/A330L/1332E.

Embodiment 28. The antibody or antigen binding fragment of any one of Embodiments 18-27, wherein the Fc moiety comprises the amino acid substitution mutations: M428L; N434S; G236A; A330L; and I332E.

Embodiment 29. An isolated antibody, or an antigen binding fragment thereof, comprising:
(i) a heavy chain variable region (V_{H}) comprising the amino acid sequence according to SEQ ID NO:41; and
(ii) a light chain variable region (V_{L}) comprising the amino acid sequence according to SEQ ID NO:89, wherein the antibody or antigen binding fragment thereof binds to the antigenic loop region of HBsAg and neutralizes infection with hepatitis B virus and hepatitis delta virus.

Embodiment 30. The isolated antibody or antigen binding fragment of Embodiment 29, further comprising an Fc moiety.

Embodiment 31. The isolated antibody or antigen binding fragment of Embodiment 30, wherein the Fc moiety is derived from an IgG isotype and comprises M428L and N434S substitution mutations.

Embodiment 32. The isolated antibody or antigen binding fragment of Embodiment 30 or 31, wherein the Fc moiety is derived from an IgG isotype and comprises G236A, A330L, and I332E substitution mutations.

Embodiment 33. The isolated antibody or antigen binding fragment of Embodiment 32, wherein the Fc moiety comprises M428L, N434S, G236A, A330L, and I332E substitution mutations.

Embodiment 34. The antibody or antigen binding fragment of any one of Embodiments 1-10, 15-33, comprising the heavy chain (HC) amino acid sequence according to SEQ ID NO: 91.

Embodiment 35. The antibody or antigen binding fragment of any one of Embodiments 1-10 and 15-32, comprising the heavy chain (HC) amino acid sequence according to SEQ ID NO: 92.

Embodiment 36. The antibody or antigen binding fragment of any one of Embodiments 1-3, 6, 7, 9, 10, and 15-35, comprising the light chain (LC) amino acid sequence according to SEQ ID NO:93.

Embodiment 37. The antibody or antigen binding fragment of any one of Embodiments 1, 2, 4, 6, 8, 11, and 15-28, comprising the light chain (LC) amino acid sequence according to SEQ ID NO:94.

Embodiment 38. The antibody or antigen binding fragment of Embodiment 34 or 36, comprising the HC amino acid sequence according to SEQ ID NO:91 and the LC amino acid sequence according to SEQ ID NO:93.

Embodiment 39. The antibody or antigen binding fragment of Embodiment 35 or 37, comprising the HC amino acid sequence according to SEQ ID NO:92 and the LC amino acid sequence according to SEQ ID NO:94.

Embodiment 40. The antibody or antigen binding fragment of Embodiment 34 or 37, comprising the HC amino acid sequence according to SEQ ID NO:91 and the LC amino acid sequence according to SEQ ID NO:94.

Embodiment 41. The antibody or antigen binding fragment of Embodiment 35 or 36, comprising the HC amino acid sequence according to SEQ ID NO:92 and the LC amino acid sequence according to SEQ ID NO:93

Embodiment 42. An isolated antibody, or an antigen binding fragment thereof, comprising:
(i) a heavy chain (HC) comprising the amino acid sequence according to SEQ ID NO:91; and
(ii) a light chain (LC) comprising the amino acid sequence according to SEQ ID NO:93,
wherein the antibody or antigen binding fragment thereof binds to the antigenic loop region of HBsAg and neutralizes infection with hepatitis B virus and hepatitis delta virus.

Embodiment 43. The antibody or antigen binding fragment of any one of Embodiments 1-42, wherein the antibody or the antigen binding fragment binds an HBsAg of a genotype selected from the HBsAg genotypes A, B, C, D, E, F, G, H, I, and J, or any combination thereof.

Embodiment 44. The antibody or antigen binding fragment of any one of Embodiments 1-43, wherein the antibody or antigen binding fragment reduces a serum concentration of HBV DNA in a mammal having an HBV infection.

Embodiment 45. The antibody or antigen binding fragment of any one of Embodiments 1-44, wherein the antibody or antigen binding fragment reduces a serum concentration of HBsAg in a mammal having an HBV infection.

Embodiment 46. The antibody or antigen binding fragment of any one of Embodiments 1-45, wherein the antibody or antigen binding fragment reduces a serum concentration of HBeAg in a mammal having an HBV infection.

Embodiment 47. The antibody or antigen binding fragment of any one of Embodiments 1-46, wherein the antibody or antigen binding fragment reduces a serum concentration of HBcrAg in a mammal having an HBV infection.

Embodiment 48. A kit comprising:
(i) the antibody or antigen binding fragment of any one of Embodiments 1-47; and
(ii) instructions for using the component to prevent, treat, attenuate, and/or diagnose a hepatitis B infection and/or a hepatitis D infection.

Embodiment 49. The kit of Embodiment 48, further comprising:
(i) a polymerase inhibitor, wherein the polymerase inhibitor optionally comprises Lamivudine, Adefovir, Entecavir, Telbivudine, Tenofovir, or any combination thereof;
(ii) an interferon, wherein the interferon optionally comprises IFNbeta and/or IFNalpha;
(iii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof;
(iv) an agonist of a stimulatory immune checkpoint molecule; or
(v) any combination of (viii)-(xii).

Embodiment 50. The kit of Embodiment 49, wherein the polymerase inhibitor comprises lamivudine.

Embodiment 51. Use of the antibody or antigen binding fragment of any one of Embodiments 1-47 in the manufacture of a medicament to prevent, treat, attenuate, and/or diagnose a hepatitis B infection and/or a hepatitis D infection in a subject.

Embodiment 52. A method of treating, preventing, and/or attenuating a hepatitis B and/or hepatitis D infection in a subject, comprising administering to the subject an effective amount of: (i) the antibody or antigen binding fragment of any one of Embodiments 1-47.

Embodiment 53. The method of Embodiment 52, further comprising administering to the subject one or more of: a polymerase inhibitor, wherein the polymerase inhibitor optionally comprises Lamivudine, Adefovir, Entecavir, Telbivudine, Tenofovir, or any combination thereof; an interferon, wherein the interferon optionally comprises IFNbeta and/or IFNalpha; a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; an agonist of a stimulatory immune checkpoint molecule; or any combination thereof.

Embodiment 54. The method of Embodiment 52 or 53, wherein the hepatitis B infection is a chronic hepatitis B infection.

Embodiment 55. The method of any one of Embodiments 52-54, wherein the subject has received a liver transplant.

Embodiment 56. The method of any one of Embodiments 52-55, wherein the subject is non-immunized against hepatitis B.

Embodiment 57. The method of any one of Embodiments 52-56, wherein the subject is a newborn.

Embodiment 58. The method of any one of Embodiments 52-57, wherein the subject is undergoing or has undergone hemodialysis.

Embodiment 59. An isolated antibody, or an antigen binding fragment thereof, comprising:
a heavy chain variable region (V_{H}) comprising a CDRH1 amino acid sequence according to SEQ ID NO:34, a CDRH2 amino acid sequence according to SEQ ID NO:35 or 66, a CDRH3 amino acid sequence according to SEQ ID NO:36;
a light chain variable region (V_{L}) comprising a CDRL1 acid sequence according to SEQ ID NO:37, a CDRL2 acid sequence according to SEQ ID NO:38 or 39, and CDRL3 amino acid sequence according to SEQ ID NO:58 or 40; and
a Fc moiety, wherein the Fc moiety comprises G236A/A330L/1332E.

Embodiment 60. The antibody or antigen binding fragment of Embodiment 59, wherein the Fc moiety does not comprise S239D.

Embodiment 61. The antibody or antigen binding fragment of Embodiment 59 or 60, wherein the Fc moiety further comprises M428L/N434S.

Embodiment 62. The antibody or antigen binding fragment of any one of Embodiments 59-61, wherein the V_{H} comprises or consists of the amino acid sequence according to any one of SEQ ID NOs:41 or 67 and wherein the V_{L} comprises or consists of the amino acid sequence according to any one of SEQ ID NOs:42, 59, 65, 89, 90, and 111-120.

Embodiment 63. An isolated antibody, or an antigen binding fragment thereof, comprising:
(i) a heavy chain variable region (V_{H}) comprising a CDRH1 amino acid sequence according to SEQ ID NO:97, a CDRH2 amino acid sequence according to SEQ ID NO:98, a CDRH3 amino acid sequence according to SEQ ID NO:99;
(ii) a light chain variable region (V_{L}) comprising a CDRL1 acid sequence according to SEQ ID NO:100, a CDRL2 acid sequence according to SEQ ID NO:100, and CDRL3 amino acid sequence according to SEQ ID NO:102; and
(iii) a Fc moiety, wherein the Fc moiety comprises G236A/A330L/1332E.

Embodiment 64. The antibody or antigen binding fragment of Embodiment 63, wherein the Fc moiety does not comprise S239D.

Embodiment 65. The antibody or antigen binding fragment of Embodiment 63 or 64, wherein the Fc moiety further comprises M428L/N434S.

Embodiment 66. The antibody or antigen binding fragment of any one of Embodiments 63-65, wherein the V_{H} comprises or consists of the amino acid sequence according to SEQ ID NO:95, and wherein the V_{L} comprises or consists of the amino acid sequence according to SEQ ID NO:96.

Embodiment 67. The antibody or antigen binding fragment of any one of Embodiments 63-66, wherein the antibody or antigen binding fragment: (i) has enhanced binding to a human FcγRILA, a human FcγRIIIA, or both, as compared to a reference polypeptide that includes a Fc moiety that does not comprise G236A/A330L/1332E, wherein the human FcγRIIA is optionally H131 or R131, and/or the human FcγRIIIA is optionally F158 or V158; (ii) has reduced binding to a human FcγRIIB, as compared to a reference polypeptide that includes a Fc moiety that does not comprise G236A/A330L/1332E; (iii) does not bind to a human FcγRIIB; has reduced binding to a human C1q, as compared to a reference polypeptide that includes a Fc moiety that does not comprise G236A/A330L/1332E; does not bind to a human C1q; activates a FcγRIIA, a human FcγRIIIA, or both, to a greater degree than does a reference polypeptide that includes a Fc moiety that does not comprise G236A/A330L/1332E, wherein the human FcγRIIA is optionally H131 or R131, and/or the human FcγRIIIA is optionally F158 or V158; does not activate a human FcγRIIB; and/or activates a human natural killer (NK) cell in the presence of HBsAg to a greater degree than does a reference polypeptide that includes a Fc moiety that does not comprise G236A/A330L/1332E.

Embodiment 68. A method of treating a Hepatitis B Virus infection in a subject, the method comprising administering to the subject a single dose of a composition comprising the antibody or antigen binding fragment of any one of Embodiments 1-47 or 59-67 at 2, 3, 4, 5, 6, 7, 8, 9, 10, 11, 12, 13, 14, 15, 16, 17, 18 mg/kg, or more, of the antibody or antigen-binding fragment, or at a dose of up to 75 mg (i.e., including any integer or non-integer dose up to 75 mg), up to 300 mg, or up to 900 mg, of the antibody or antigen-binding fragment.

Embodiment 69. The method of Embodiment 68, wherein the antibody or antigen-binding fragment comprises a heavy chain (HC) amino acid sequence according to SEQ ID NO:91 and a light chain (LC) amino acid sequence according to SEQ ID NO:93.

Embodiment 70. The method of Embodiment 68 or 69, wherein prior to the administering, the composition comprises the antibody or antigen-binding fragment at 150 mg/mL, optionally in sterile water, and further comprises 20 mM Histidine, 7% sucrose, and 0.02% PS80 at pH 6.

Embodiment 71. The method of any one of Embodiments 68-70, wherein the subject: (i) is aged 18 to 65 years, or is older; (ii) weighs ≥40 kg to ≤125 kg; (iii) has a chronic HBV infection, wherein a chronic HBV infection is defined by: positive serum HBsAg, HBV DNA, or HBeAg on 2 occasions at least 6 months apart based on previous or current laboratory documentation (or a positive result based on any combination of these tests performed at least 6 months apart); (iv) does not have cirrhosis; (v) received a nucleoside reverse transcriptase inhibitor (NTRI) therapy for at least 4 months (120 days) prior to the single dose being administered, wherein the NTRI therapy optionally comprises Tenofovir disoproxil/tenofovir alafenamide, Entecavir, Lamivudine, or Adefovir/adefovir dipivoxil; (vi) had HBV DNA at <100 IU/mL no more than 4 weeks (28 days) prior to the single dose being administered; (vii) had HBsAg> the lower limit of detection no more than 4 weeks (28 days) prior to the single dose being administered; (viii) had HBsAg at <1000 IU/mL no more than 4 weeks (28 days) prior to the single dose being administered; (ix) had HBsAg at ≥1000 IU/mL no more than 4 weeks (28 days) prior to the single dose being administered; (x) was HBeAg-negative no more than 4 weeks (28 days) prior to the single dose being administered; (xi) was negative for anti-Hepatitis B antibodies no more than 4 weeks (28 days) prior to the single dose being administered; (xii) have alanine aminotransferase <2×ULN; or (xiii) any combination of (i)-(xii).

Embodiment 72. The method of any one of Embodiments 68-71, wherein the administering comprises subcutaneous injection.

Embodiment 73. The method of any one of Embodiments 68-72, wherein at 8 weeks following the administering of the single dose, the subject has a >2-fold reduction in HBsAg as compared to from 0 days to 4 weeks (28 days) prior to the administering.

Embodiment 74. A method of treating a Hepatitis B virus (HBV) infection in a subject, the method comprising administering to the subject a single dose of a pharmaceutical composition comprising an antibody according to any one of Embodiments 1-47 or 59-67, wherein, optionally, the antibody comprises the heavy chain amino acid sequence of SEQ ID NO.: 91 and the light chain amino acid sequence of SEQ ID NO.: 93.

Embodiment 75. The method of Embodiment 74, wherein the single dose of the pharmaceutical composition comprises the antibody in a range from 2 to 18 mg/kg (subject body weight).

Embodiment 76. The method of Embodiment 74 or 75, wherein the single dose of the pharmaceutical composition comprises up to 6 mg, up to 18 mg, up to 75 mg, up to 90 mg, up to 300 mg, up to 900 mg, or up to 3000 mg of the antibody.

Embodiment 77. The method of any one of Embodiments 74-76, wherein the single dose of the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

Embodiment 78. The method of any one of Embodiments 74-77, wherein the single dose of the pharmaceutical composition comprises about 75 mg of the antibody.

Embodiment 79. The method of any one of Embodiments 74-78, wherein the single dose of the pharmaceutical composition comprises about 90 mg of the antibody.

Embodiment 80. The method of any one of Embodiments 74-78, wherein the single dose of the pharmaceutical composition comprises up to 300 mg of the antibody.

Embodiment 81. The method of any one of Embodiments 74-78, wherein the single dose of the pharmaceutical composition comprises up to 900 mg of the antibody.

Embodiment 82. The method of any one of Embodiments 74-78, wherein the single dose of the pharmaceutical composition comprises up to 3,000 mg of the antibody.

Embodiment 83. The method of any one of Embodiments 74-82, wherein the method comprises administering the single dose by subcutaneous injection.

Embodiment 84. The method of any one of Embodiments 74-83, wherein the method comprises administering the single dose by intravenous injection.

Embodiment 85. The method of any one of Embodiments 74-84, wherein the pharmaceutical composition further comprises water, optionally USP water.

Embodiment 86. The method of any one of Embodiments 74-85, wherein the pharmaceutical composition further comprises histidine, optionally at a concentration in a range from 10 mM to 40 mM, such as 20 mM, in the pharmaceutical composition.

Embodiment 87. The method of any one of Embodiments 74-86, wherein the pharmaceutical composition further comprises a disaccharide, such as sucrose, optionally at 5%, 6%, 7%, 8%, or 9%, preferably about 7% (w/v).

Embodiment 88. The method of any one of Embodiments 74-87, wherein the pharmaceutical composition further comprises a surfactant or a triblock copolymer, optionally a polysorbate or poloxamer-188, preferably polysorbate 80 (PS80), wherein, optionally, the polysorbate or poloxamer-188 is present in a range from 0.01% to 0.05% (w/v), preferably 0.02% (w/v).

Embodiment 89. The method of any one of Embodiments 74-88, wherein the pharmaceutical composition has a pH in a range from 5.8 to 6.2, in a range from 5.9 to 6.1, or of 5.8, of 5.9, of 6.0, of 6.1, or of 6.2.

Embodiment 90. The method of Embodiment 89, wherein the pharmaceutical composition comprises:
(i) the antibody at 150 mg/mL;
(ii) USP water;
(iii) 20 mM histidine;
(iv) 7% sucrose; and
(v) 0.02% PS80,
wherein the pharmaceutical composition comprises a pH of 6.

Embodiment 91. The method of any one of Embodiments 74-90, wherein the subject is an adult.

Embodiment 92. The method of Embodiment 74-91, wherein the subject is in a range from 18 years of age to 65 years of age.

Embodiment 93. The method of any one of Embodiments 74-92, wherein the subject weighs from 40 kg to 125 kg.

Embodiment 94. The method of any one of Embodiments 74-93, wherein the subject has a chronic HBV infection; e.g., defined by positive serum HBsAg, HBV DNA, and/or HBeAg on 2 occasions, wherein the 2 occasions are at least 6 months apart.

Embodiment 95. The method of any one of Embodiments 74-94, wherein the subject does not have cirrhosis.

Embodiment 96. The method of Embodiment 95, wherein absence of cirrhosis is determined by: Fibroscan evaluation (e.g., within 6 months prior to administering the single dose of the pharmaceutical composition); or liver biopsy (e.g., within 12 months prior to administering the single dose of the pharmaceutical composition), wherein, preferably the absence of cirrhosis is determined by the absence of Metavir F3 fibrosis or the absence of F4 cirrhosis.

Embodiment 97. The method of any one of Embodiments 74-96, wherein the subject has received a nucleos (t) ide reverse transcriptase inhibitor (NRTI), optionally within 120 days, further optionally within 60 days, prior to the single dose being administered.

Embodiment 98. The method of Embodiment 97, wherein the NRTI comprises one or more of: tenofovir; tenofovir disoproxil (e.g., tenofovir disproxil fumarate); tenofovir alafenamide; Entecavir; Lamivudine; Adefovir; and adefovir dipivoxil.

Embodiment 99. The method of any one of Embodiments 74-98, wherein the subject has a serum HBV DNA concentration of less than 100 IU/mL no more than 28 days prior to the single dose being administered.

Embodiment 100. The method of any one of Embodiments 74-99, wherein the subject has a serum HBsAg concentration of less than 1,000 IU/mL prior to the single dose being administered.

Embodiment 101. The method of any one of Embodiments 74-99, wherein the subject has a serum HBsAg concentration of greater than or equal to 1,000 IU/mL no more than 28 days prior to the single dose being administered.

Embodiment 102. The method of any one of Embodiments 74-101, wherein the subject was HB e-antigen (HBeAg)-negative no more than 28 days prior to the single dose being administered.

Embodiment 103. The method of any one of Embodiments 74-102, wherein the subject was negative for anti-HB antibodies no more than 28 days prior to the single dose being administered.

Embodiment 105. The method of any one of Embodiments 74-103, wherein the subject, prior to administration of the single dose: (i) does not have fibrosis and/or does not have cirrhosis; and/or (ii) has alanine aminotransferase (ALT)<2× Upper Limit of Normal (ULN).

Embodiment 106. The method of any one of Embodiments 74-105, wherein at 56 days following administration of the single dose, the subject has a >2-fold reduction in serum HBsAg (e.g., concentration of HBsAg in serum, e.g., as determined using an Abbott ARCHITECT assay) as compared to the subject's serum HBsAg at from 0 days to 28 days prior to administration of the single dose.

Embodiment 107. The method of any one of Embodiments 74-106, wherein following administration of the single dose (e.g., at 56 days following administration of the single dose), the subject has: (i) has reduced or less severe intrahepatic spread of HBV as compared to a reference subject; and/or (ii) comprises an adaptive immune response against HBV.

Embodiment 108. The method of any one of Embodiments 74-107, wherein the subject is male.

Embodiment 109. The method of any one of Embodiments 74-107, wherein the subject is female.

Embodiment 110. A pharmaceutical composition comprising an antibody, wherein the antibody comprises the heavy chain amino acid sequence of SEQ ID NO.: 91 and the light chain amino acid sequence of SEQ ID NO.: 93, wherein the pharmaceutical composition comprises the antibody at a concentration ranging from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

Embodiment 111. The pharmaceutical composition of Embodiment 1110, wherein the pharmaceutical composition comprises up to 6 mg, up to 18 mg, up to 75 mg, up to 90 mg, up to 300 mg, up to 900 mg, or up to 3000 mg of the antibody.

Embodiment 112. The pharmaceutical composition of Embodiment 110 or 111, wherein the pharmaceutical composition comprises about 75 mg of the antibody.

Embodiment 113. The pharmaceutical composition of Embodiment 110 or 111, wherein the pharmaceutical composition comprises about 90 mg of the antibody.

Embodiment 114. The pharmaceutical composition of Embodiment 110 or 111, wherein the pharmaceutical composition comprises about 300 mg of the antibody.

Embodiment 115. The pharmaceutical composition of Embodiment 110 or 111, wherein the pharmaceutical composition comprises about 900 mg of the antibody.

Embodiment 116. The pharmaceutical composition of Embodiment 110 or 111, wherein the pharmaceutical composition comprises about 3,000 mg of the antibody.

Embodiment 117. The pharmaceutical composition of any one of Embodiments 110-116, wherein the pharmaceutical composition further comprises water, optionally USP water.

Embodiment 118. The pharmaceutical composition of any one of Embodiments 110-117, wherein the pharmaceutical composition further comprises histidine, optionally at a concentration from 10 mM to 40 mM, such as 20 mM, in the pharmaceutical composition.

Embodiment 119. The pharmaceutical composition of any one of Embodiments 110-118, wherein the pharmaceutical composition further comprises a disaccharide, such as sucrose, optionally at 5%, 6%, 7%, 8%, or 9%, preferably about 7% (w/v).

Embodiment 120. The pharmaceutical composition of any one of Embodiments 110-119, wherein the pharmaceutical composition further comprises a surfactant, optionally a polysorbate, preferably polysorbate 80 (PS80), wherein, optionally, the polysorbate is present in a range from 0.01% to 0.05% (w/v), preferably 0.02% (w/v).

Embodiment 121. The pharmaceutical composition of any one of Embodiments 110-120, wherein the pharmaceutical composition has a pH ranging from 5.8 to 6.2, ranging from 5.9 to 6.1, or of 5.8, of 5.9, of 6.0, of 6.1, or of 6.2.

Embodiment 122. The pharmaceutical composition of any one of Embodiments 110-121, wherein the pharmaceutical composition comprises:
(i) the antibody at 150 mg/mL;
(ii) USP water;
(iii) 20 mM histidine;
(iv) 7% sucrose; and
(v) 0.02% PS80,
wherein the pharmaceutical composition comprises a pH of 6.

### EXAMPLES

In the following, particular examples illustrating embodiments and aspects of the disclosure are presented. However, the present disclosure shall not to be limited in scope by the specific embodiments described herein. The following preparations and examples are given to enable those skilled in the art to more clearly understand and to practice the present disclosure. The present disclosure, however, is not limited in scope by the exemplified embodiments. Indeed, various modifications of the disclosure in addition to those described herein will become readily apparent to those skilled in the art from the foregoing description, accompanying figures and the examples below. All such modifications fall within the appended claims.

### Example 1: Generation and Testing of Engineered Antibodies

Analysis of some HBC34 antibody variants from PCT Publication No. WO 2017/060504 revealed a cysteine amino acid at position 40 (IMGT numbering) in the light chain variable region that is unpaired and represents a potential liability. Without wishing to be bound by theory, unpaired cysteine residues are potentially reactive and can potentially trigger aggregation through intramolecular scrambling or intermolecular disulfide formation. Variants of HBC34-V7 (WO 2017/060504) were engineered in which the cysteine amino acid at position 40 was substituted with a serine (thereby generating “HBC34-V34”) or with an alanine (thereby generating “HBC34-V35”). The nucleotide sequences encoding these additional variant antibodies were codon-optimized, and antibodies were expressed as IgG1 (glm17, 1 allotype) in ExpiCHO™ cells (ThermoFisher). Codon-optimized nucleotide sequences encoding the V_{H} and V_{L} domains of HBC34-V35 are provided in SEQ ID NOS: 103 and 104, respectively.

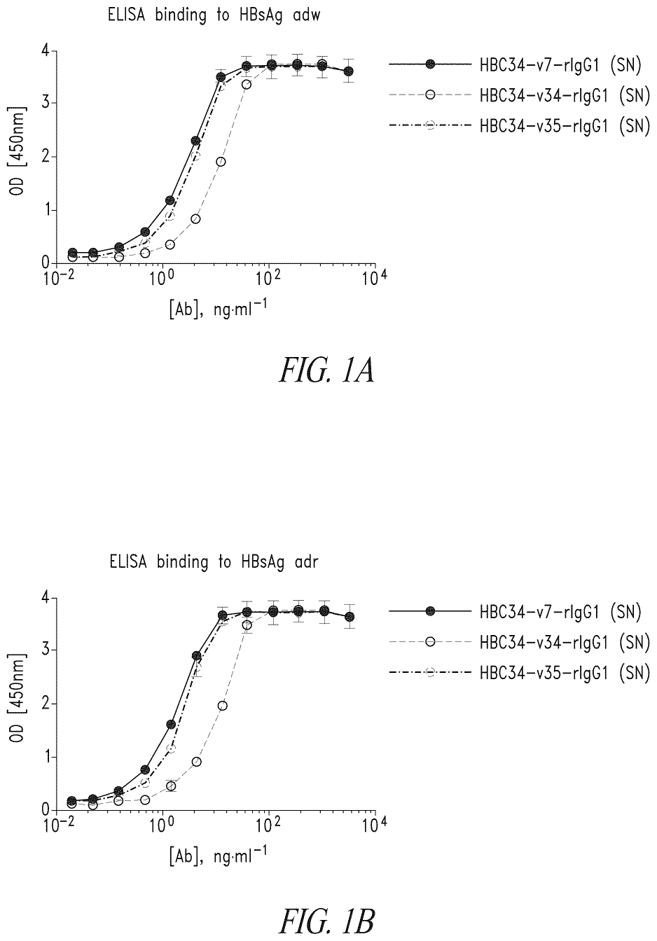

The ability of HBC34-V34 and HBC34-V35 to bind antigen was investigated using a direct antigen-binding ELISA. HBC34-V7 was used as a comparator. As shown in FIG. 1, both HBC34-V34 and HBC34-V35 bound effectively to two recombinant HBsAg antigens (“adw”, top panel; “adr”, bottom panel), and HBC34-V35 had very similar binding as the parent HBC34-V7.

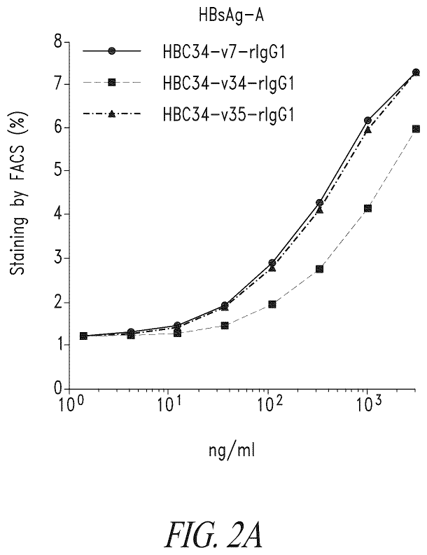

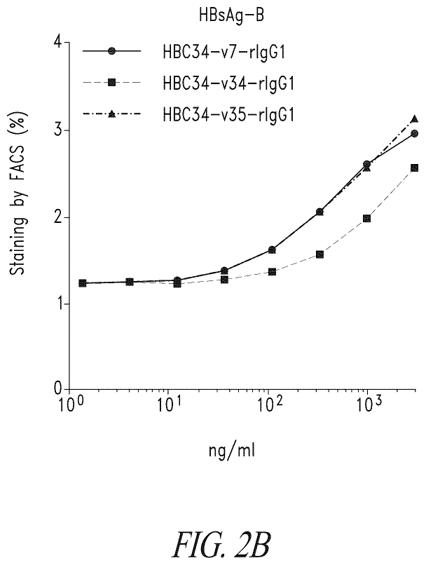

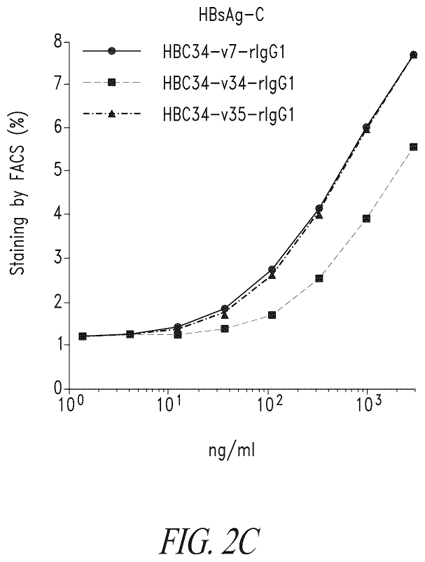

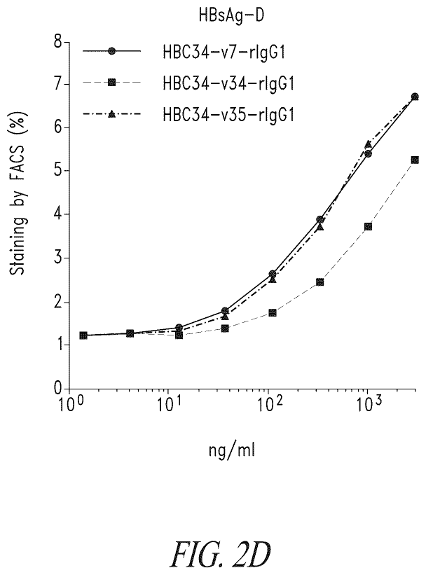

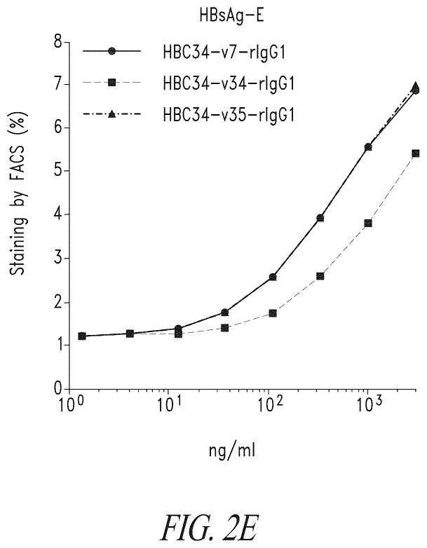

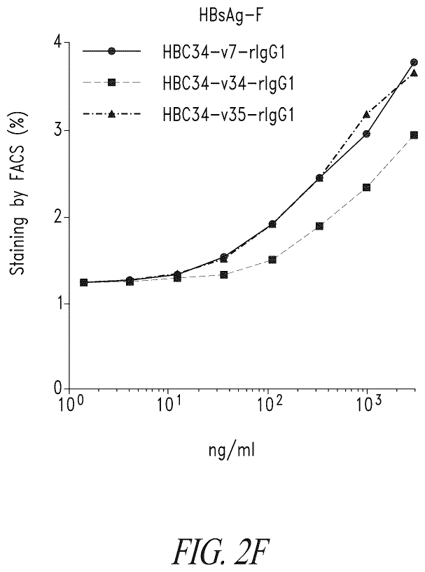

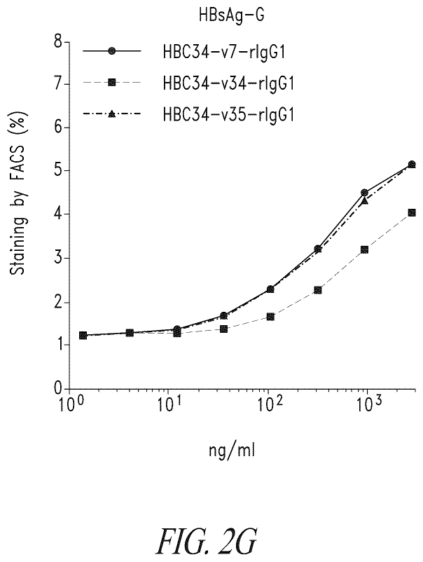

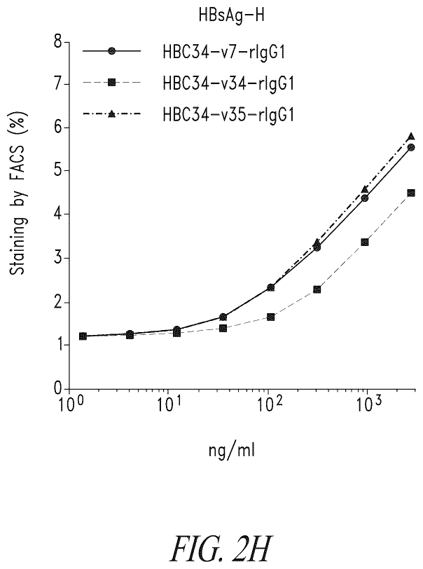

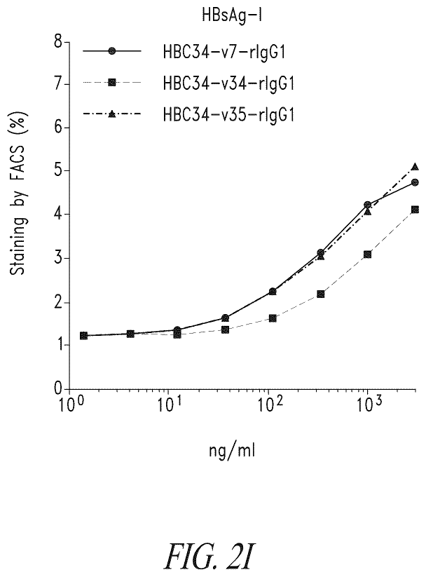

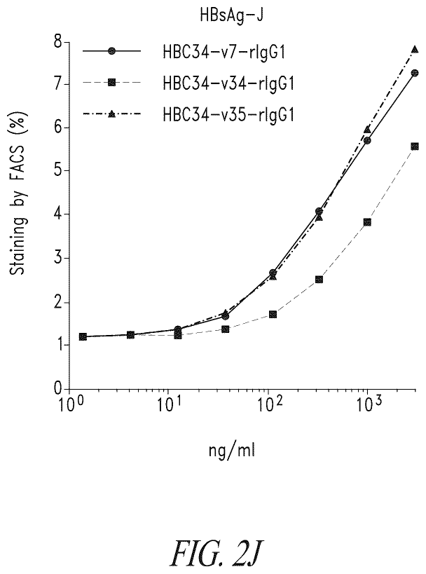

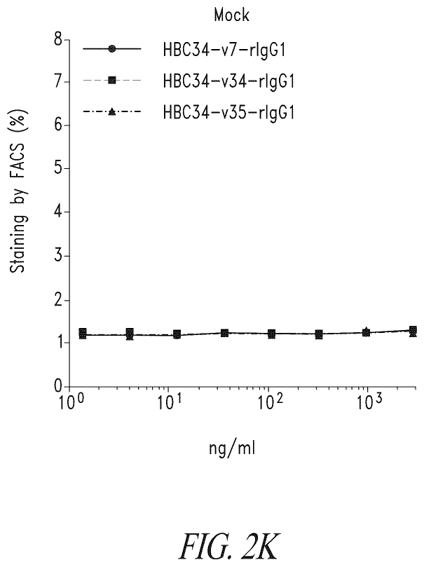

The variant antibodies were examined for binding to all known HBsAg genotypes ((A)-(J)). Briefly, human epithelial cells (Hep2 cells) were transfected with plasmids expressing each of the HBsAg of the 10 HBV genotypes A, B, C, D, E, F, G, H, I, and J. All antibodies were tested at multiple concentrations for staining of transiently transfected permeabilized cells. Two days after transfection, Hep2 cells were collected, fixed and permeabilized with saponin for immunostaining with HBC34 and the five selected variants. HBC34-V7 was included as a comparator. Binding of antibodies to transfected cells was analysed using a Becton Dickinson FACSCanto2 (BD Biosciences) with FlowJo software (TreeStar). As shown in FIGS. 2A-2J, HBC34-V34 and HBC34-V35 recognized all 10 HBV HBsAg genotypes. HBC34-V35 showed somewhat stronger staining than HBC34-V34.

These data show that the antibody variants HBC34-V34 and HBC34-V35 broadly recognize and bind to HBsAG at levels comparable to HBC34-V7.

### Example 2: HBC Antibodies Having Modified Fc Regions Efficiently Bind to Antigen

Modifications in the Fc region may provide advantages to a therapeutic antibody. HBC34-V35 was expressed as IgG1 with wild-type Fc, or with Fc containing a “MLNS” mutation (M428L/N434S) or with MLNS in combination with “GAALIE” (G239A/A330L/1332E). Each construct was tested for binding to recombinant HBsAg (adw) in two separate antigen-binding ELISA experiments. Three (3) lots of HBC34-v35 (wild-type Fc) were tested. Two (2) lots of HBC34-V35-MLNS and two (2) lots of HBC34-V35-MLNS-GAALIE were tested. HBC34v7 (one lot) was tested as a comparator.

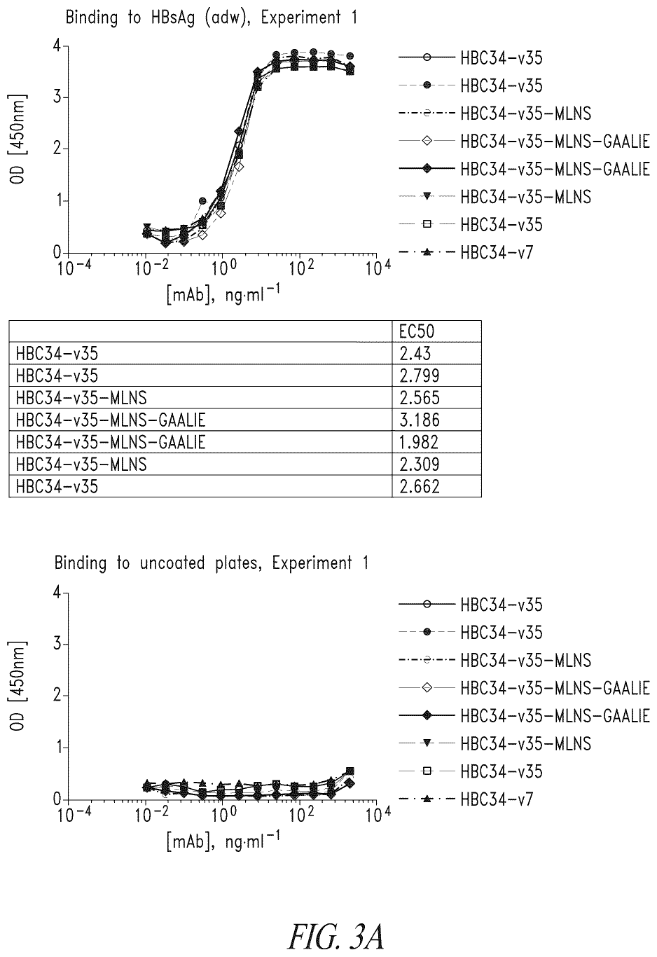

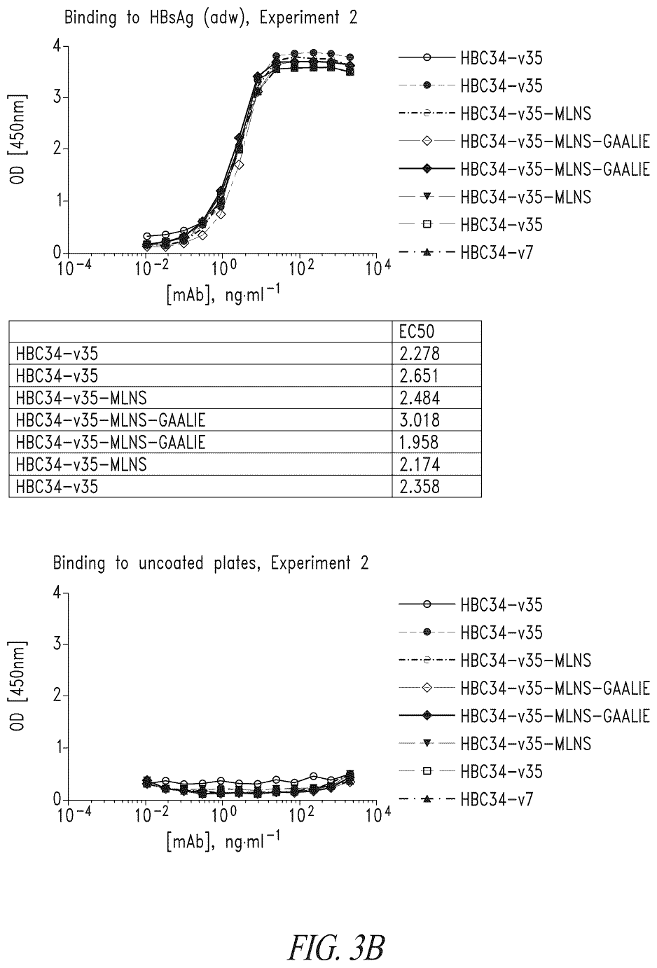

As shown in FIGS. 3A and 3B, the introduced Fc mutations did not affect antigen-binding activity of HBC34-V35. EC50 values varied somewhat between the various constructs and the two experiments, but were generally low.

### Example 3: Additional Functional Studies

In vitro and in vivo neutralization studies are performed using HBC34-V35, HBC34-V35-MLNS, and HBC34-V35-MLNS-GAALIE. In one study, antibodies are tested for neutralizing activity using HBV-infected mouse PXB cells. In another study, antibodies are tested using human hepatocyte cells infected with HBV of the C genotype.

For both studies, Hebsbulin (Human Hepatitis B Immunoglobulin) is used as a positive control. The following data are captured at multiple timepoints: HBV DNA quantification; HBsAg quantification; HBeAg quantification; and hAlb quantification.

### Example 4: Identification and Characterization of Human Monoclonal Antibody HBC24

A human monoclonal antibody was isolated in a similar manner as described in Traggiai E. et al., 2004, Nat Med 10 (8): 871-5 from a human patient. The antibody was characterized by determining the nucleotide and amino acid sequences of its variable regions and the complementarity determining regions (CDRs) therein and termed “HBC24”. Accordingly, HBC24 is an IgG1-type fully human monoclonal antibody having the CDR, V_{H} and V_{L} sequences as shown above in Table 3. Exemplary nucleotide sequences encoding the V_{H} and V_{L} of HBC24 are provided in Table 4.

### Example 5: Clearance of HB Antigens and Viral Entry Inhibition in a Mouse Model

An immune-deficient mouse having transplanted human hepatocytes was used to test the effectiveness of anti-HBV antibodies of the present disclosure in clearing HBsAg. Briefly, primary human hepatocytes were transplanted into SCID mice for which mouse hepatocytes had previously been destroyed enzymatically. The mice were T- and B-cell deficient. This model is useful for studying HBV infection including entry, spreading, cccDNA regulation, hepatocyte-intrinsic immune responses, and viral integration into host genome.

Mice were inoculated via tail vein injection with rAAV8-1.3HBV strain ayw, D type, at 1.0×10⁷ viral genomes per mouse at Day −28. Treatments at Day 0. AAV/HBV-infected mice (n=4 per treatment group) were administered PBS (control) or HBC34-v35 (1, 5, or 15 mg/kg i.p., 2×/week). Antibodies were murinized with the exception of the antigen-binding Fab regions.

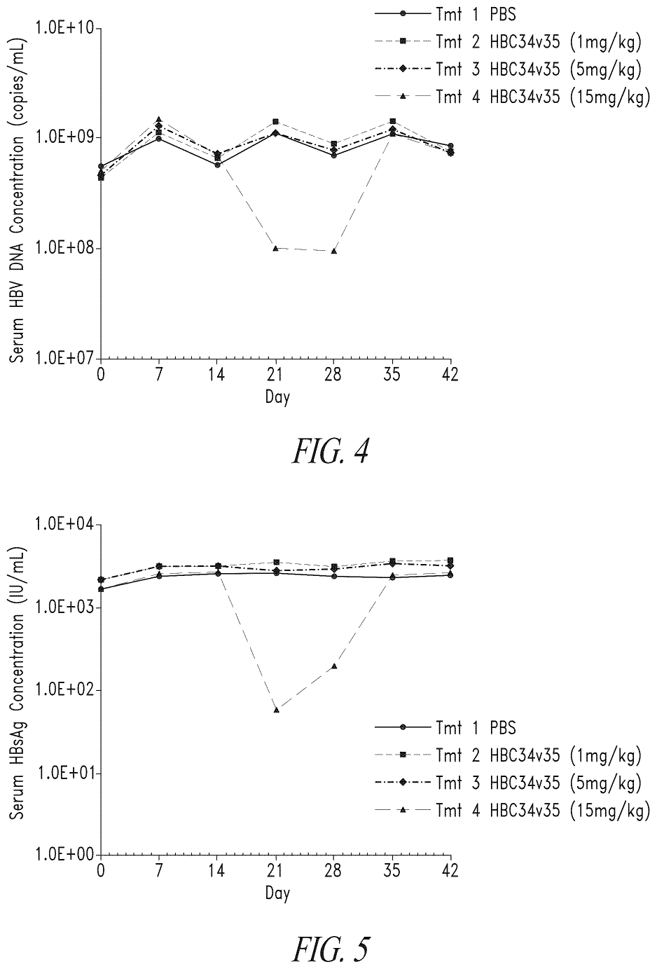

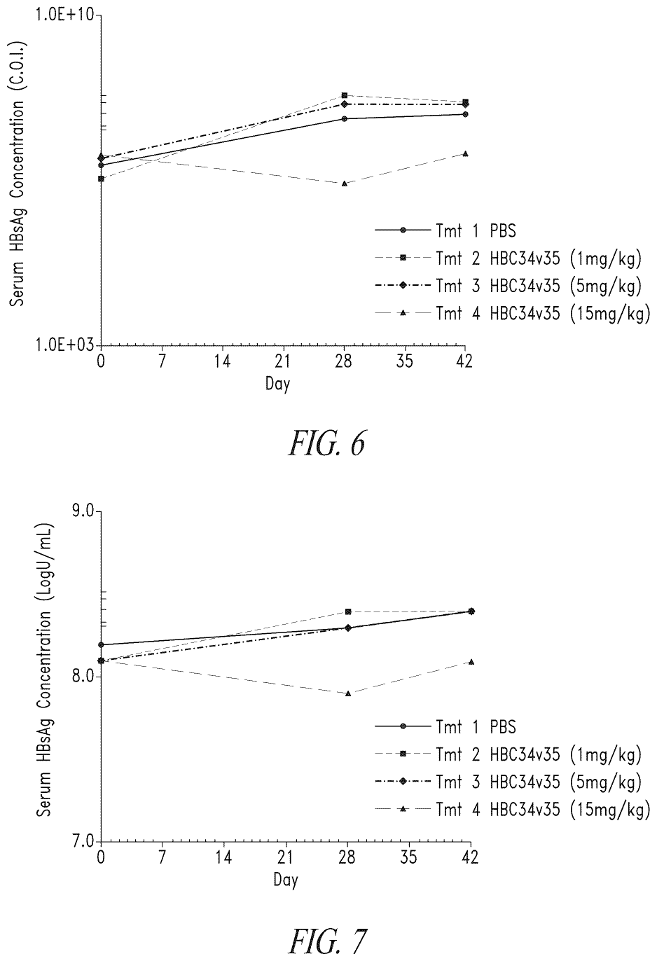

Plasma and serum samples were collected periodically throughout the study, and viral loads, HBV DNA (by PCR), and HB Ag (HBsAg, HBeAg, HBcrAg). Mice were sacrificed at week 6. As shown in FIGS. 4-7, treatment with the highest dose of HBC34-v35 reduced viral load and viral entry into hepatocytes.

### Example 6: Generation of Germlined Variants of HBC24 and Functional Testing

HBC24 is analyzed for the presence of somatic mutations in the variable regions relative to germline sequence. Identified somatic mutations are reverted to germline sequence to produce HBC24 variants. HBC24 and variants are tested for binding (in vitro) and neutralization (in vitro; in vivo) of HBV and HBD serotypes using assays as described in Examples 1 and 3.

### Example 7: Introduction of Fc Modifications to HBC24 and Variants

Further HBC24 variants are produced that contain the MLNS and GAALIE mutations in both Fc monomers. The HC amino acid sequences of selected variants are provided in SEQ ID NOs: 120 and 121. Variants are examined for: (1) in vitro binding to antigen; (2) in vitro neutralization of HBV serotypes using assays as described in Examples 1 and 3.

### Example 8: In Vitro Effector Function Studies

In vitro studies were performed to examine the ability of HBC34 antibodies with modified Fc to: (1) bind to human FcγRs and to complement; (2) activate FcγRIIa, FcγRIIb, and FcγRIIIa; and (3) promote ADCC and activate human Natural Killer (NK) cells. Test articles, cell lines, and reagents used were as described in Tables 5-7, below. The following abbreviations are used in this Example: GLP=Good Laboratory Practice; ADCC=Antibody-dependent cellular cytotoxicity; ADCP=Antibody-dependent cellular phagocytosis; Fc=Fragment crystallizable; HBsAg=Hepatitis B surface Antigen; mAb=Monoclonal antibody; PBS=Phosphate-buffered saline; UHPL-SEC=Ultra-high performance liquid size-exclusion chromatography; ATCC=American Type Culture Collection; FcγRs=Fc gamma receptor(s); CHO cells=Chinese hamster ovary cells; RLU=Relative luminescence units; BLI=Bio-layer interferometry.

TABLE 5
Test Articles.

| Test Article | HBC34v35-MLNS |
|---|---|
| [symbol] Isotype | [symbol] IgG1k |
| [symbol] Relative molecular weight | [symbol] ≈150 kDa |
| [symbol] Concentration | [symbol] 3.47 mg/ml |
| [symbol] Source | [symbol] In-house |
| [symbol] Handling and storage | [symbol] 4° C. short term, −80° C. long |
| conditions | term storage |
| [symbol] Formulation buffer | [symbol] PBS, pH 7.2 |
| Test Article | HBC34-V35-MLNS-GAALIE |
| [symbol] Isotype | [symbol] IgG1k |
| [symbol] Relative molecular weight | [symbol] ≈150 kDa |
| [symbol] Concentration | [symbol] 2.1 mg/ml/0.86 mg/ml |
| [symbol] Source | [symbol] In-house |
| [symbol] Handling and storage | [symbol] 4° C. short term, −80° C. long |
| conditions | term storage |
| [symbol] Formulation buffer | [symbol] PBS, pH 7.2 |
| Test Article | HBC34v35-LALA |
| [symbol] Isotype | [symbol] IgG1k |
| [symbol] Relative molecular weight | [symbol] ≈150 kDa |
| [symbol] Concentration | [symbol] 1.2 mg/ml |
| [symbol] Source | [symbol] In-house |
| [symbol] Handling and storage | [symbol] 4° C. short term, −80° C. long |
| conditions | term storage |
| [symbol] Formulation buffer | [symbol] PBS, pH 7.2 |
| Test Article | mAb 17.1.41 |
| [symbol] Isotype | [symbol] IgG1k |
| [symbol] Relative molecular weight | [symbol] ≈150 kDa |
| [symbol] Concentration | [symbol] 4.4 mg/ml |
| [symbol] Source | [symbol] In-house |
| [symbol] Handling and storage | [symbol] 4° C. short term, −80° C. long |
| conditions | term storage |
| [symbol] Formulation buffer | [symbol] PBS, pH 7.2 |

TABLE 6
Cell Lines

| Cell Line | PLC/PRF/5 |
|---|---|
| [symbol] Catalogue number | [symbol] #4325-FC-050 |
| [symbol] Concentration | [symbol] 100 μg/ml |
| [symbol] Source | [symbol] R&D Systems, mouse myeloma |
| | cell line, NS0-derived, with a |
| | C-terminal 6-His tag |
| [symbol] Stability | [symbol] Stable at −20 to 80° C. |
| [symbol] Handling and storage | [symbol] Store at −80° C. until use, 1 |
| conditions | month, 2 to 8° C. under sterile |
| | conditions after reconstitution |
| [symbol] Formulation buffer | [symbol] PBS |
| Cell Line | Jurkat-FcγRIIIA (F158) |
| Tissue origin | Immortalized line of human T |
| | lymphocyte cells; Jurkat cells |
| | stably expressing the FcγRIIIa |
| | receptor, F158 (low affinity) |
| | variant, and an NFAT response |
| | element driving expression of |
| | firefly luciferase as effector cells |
| Source | Promega (Cat. Nr.: G9798) |
| Assay media | RPMI1640 supplemented with 4% |
| | low IgG serum |
| Cell line | Jurkat-FcγRIIIA (V158) |
| Tissue origin | Immortalized line of human T |
| | lymphocyte cells; Jurkat cells |
| | stably expressing the FcγRIIIa |
| | receptor, V158 (high affinity) |
| | variant, and an NFAT response |
| | element driving expression of |
| | firefly luciferase as effector cells |
| Source | Promega (Cat. Nr.: G7018) |
| Assay media | RPMI1640 supplemented with 4% |
| | low IgG serum |
| Cell line | Jurkat-FcγRIIA (H131) |
| [symbol] Tissue origin | [symbol] Immortalized line of human T |
| | lymphocyte cells; Jurkat cells |
| | stably expressing the FcγRIIa |
| | receptor, H131 (high affinity) |
| | variant, and an NFAT response |
| | element driving expression of |
| | firefly luciferase as effector cells |
| [symbol] Source | [symbol] Promega (Cat. Nr.: G9995) |
| [symbol] Assay media | [symbol] RPMI1640 supplemented with 4% |
| | low IgG fetal bovine serum |
| Cell line | Jurkat-FcγRIIB |
| [symbol] Tissue origin | [symbol] Immortalized line of human T |
| | lymphocyte cells; Jurkat cells |
| | stably expressing the FcγRIIb |
| | receptor, and an NFAT response |
| | element driving expression of |
| | firefly luciferase as effector cells |
| [symbol] Source | [symbol] Promega (Cat. Nr.: CS1781E02) |
| [symbol] Assay media | [symbol] RPMI1640 supplemented with 4% |
| | low IgG fetal bovine serum |
| Cell line | Freshly isolated human NK cells |
| [symbol] Tissue origin | [symbol] Whole blood (EDTA) from donor |
| | HM_WB019 (genotyped for |
| | FcgRIIIa F/V), purified with |
| | MACSxpress NK Isolation Kit |
| | from Miltenyi Biotec (#130-098- |
| | 185) |
| [symbol] Source | [symbol] In-house |
| [symbol] Assay media | [symbol] AIM-V |
| [symbol] Growth media | [symbol] RPMI1640 supplemented with |
| | 10% low IgG fetal bovine serum, |
| | Glutamax |
| [symbol] Growth conditions | [symbol] 37° C., 5% CO₂ |
| Cell line | Freshly isolated human NK cells |
| [symbol] Tissue origin | [symbol] Whole blood (EDTA) from donor |
| | HM_WB002 (genotyped for |
| | FcgRIIIa V/V). purified with |
| | MACSxpress NK Isolation Kit |
| | from Miltenyi Biotec (#130-098- |
| | 185) |
| [symbol] Source | [symbol] In-house |
| [symbol] Assay media | [symbol] AIM-V |
| [symbol] Growth media | [symbol] RPMI1640 supplemented with |
| | 10% low IgG fetal bovine serum, |
| | Glutamax |
| [symbol] Growth conditions | [symbol] 37° C., 5% CO₂ |
| Cell line | Freshly isolated human NK cells |
| [symbol] Tissue origin | [symbol] Whole blood (EDTA) from donor |
| | HM_WB018 (genotyped for |
| | FcgRIIIa F/F), purified with |
| | MACSxpress NK Isolation Kit |
| | from Miltenyi Biotec (#130-098- |
| | 185) |
| [symbol] Source | [symbol] In-house |
| [symbol] Assay media | [symbol] AIM-V |
| [symbol] Growth media | [symbol] RPMI1640 supplemented with |
| | 10% low IgG fetal bovine serum, |
| | Glutamax |
| [symbol] Growth conditions | [symbol] 37° C., 5% CO₂ |

TABLE 7
Other Reagents

| | Recombinant human |
|---|---|
| Reagent | FcγRIIIa (V158) |
| [symbol] Catalogue number | [symbol] #4325-FC-050 |
| [symbol] Concentration | [symbol] 100 μg/ml |
| [symbol] Source | [symbol] R&D Systems, mouse myeloma |
| | cell line, NS0-derived, with a |
| | C-terminal 6-His tag |
| [symbol] Stability | [symbol] Stable at −20 to 80° C. |
| [symbol] Handling and storage conditions | [symbol] Store at −80° C. until use, 1 |
| | month, 2 to 8° C. under sterile |
| | conditions after reconstitution |
| [symbol] Formulation buffer | [symbol] PBS |
| | Recombinant human |
| Reagent | FcγRIIIa (F158) |
| [symbol] Catalogue number | [symbol] 10389-H08H |
| [symbol] Concentration | [symbol] 200 μg/ml (when reconstituted) |
| [symbol] Source | [symbol] Sino Biological, HEK293- |
| | derived, with a C-terminal |
| | 6-His tag |
| [symbol] Stability | [symbol] Stable at −20 to 80° C. |
| [symbol] Handling and storage conditions | [symbol] Store at −80° C. until use |
| [symbol] Formulation buffer | [symbol] PBS |
| | Recombinant human |
| Reagent | FcγRIIa (H131) |
| [symbol] Catalogue number | [symbol] 10374-H08C1 |
| [symbol] Concentration | [symbol] 200 μg/ml (when reconstituted) |
| [symbol] Source | [symbol] Sino Biological, CHO-derived, |
| | with a C-terminal 6-His tag |
| [symbol] Stability | [symbol] Stable at −20 to 80° C. |
| [symbol] Handling and storage conditions | [symbol] Store at −80° C. until use |
| [symbol] Formulation buffer | [symbol] PBS |
| | Recombinant human |
| Reagent | FcγRIIa (H131) |
| [symbol] Catalogue number | [symbol] 10374-H08B |
| [symbol] Concentration | [symbol] 200 μg/ml (when reconstituted) |
| [symbol] Source | [symbol] Sino Biological, insect |
| | cells-derived, with a C- |
| | terminal 6-His tag |
| [symbol] Stability | [symbol] Stable at −20 to 80° C. |
| [symbol] Handling and storage conditions | [symbol] Store at −80° C. until use |
| [symbol] Formulation buffer | [symbol] PBS |
| Reagent | Recombinant human FcγRIIb |
| [symbol] Catalogue number | [symbol] 10259-H08C |
| [symbol] Concentration | [symbol] 200 μg/ml (when reconstituted) |
| [symbol] Source | [symbol] Sino Biological, CHO-derived, |
| | with a C-terminal 6-His tag |
| [symbol] Stability | [symbol] Stable at −20 to 80° C. |
| [symbol] Handling and storage conditions | [symbol] Store at −80° C. until use |
| Reagent | Human complement component C1q |
| Catalogue number | 204873 |
| Concentration | 1.17 mg/ml |
| Source | Sigma-Aldrich, prepared from |
| | human serum |
| Stability | Stable at −80° C. |
| Handling and storage conditions | Store at −80° C. until use |
| Formulation buffer | 10 mM HEPES with 0.3M NaCl, |
| | pH 7.2 |
| Reagent | Source |
| PBS | Sigma-Aldrich Chemie GmbH, |
| | Switzerland |
| AIM-V media | Gibco |
| Ham’s F-12K Medium | Gibco |
| MACSxpress ® NK Isolation Kit | Miltenyi Biotec GmbH, Germany |
| Cytotoxicity Detection Kit (LDH) | Roche Diagnostics GmbH, |
| | Switzerland |
| 96-well round bottom plates | Corning |
| white flat bottom 96-well plate | PerkinElmer |
| 384-well round bottom plates | Corning |
| 384-well flat bottom plates | Corning |
| Spectrophotometer | Bio-Tek |
| RMPI medium | Gibco |
| DMEM High Glucose with stable | Bioconcept |
| Glutamine | |
| FBS | GE Healthcare |
| Glutamax | Gibco |
| Trypsin-EDTA (0.05%), phenol | Gibco |
| red | |
| Prism7 Software Graph | Pad Software, Inc., La Jolla, CA |
| Triton X-100 | Sigma |
| ADCC Assay buffer | Promega |
| Bio-Glo-TM Luciferase Assay | Promega |
| Reagent | |
| ADCC Bioassay | Promega |
| Wash buffer | PBS, 1% FBS |
| Formaldehyde solution, conc. | Sigma (Cat. Nr.: F1635-500ML) |
| 37% | |
| Saponin | Sigma (Cat. Nr.: S7900-100G) |
| Permeabilization buffer | 0.5% saponin, PBS, 1% FBS |
| Alexa Fluor ® 647 secondary Ab | AffiniPure F(ab′)2 Fragment Goat |
| | Anti-Human IgG, Fcγ Fragment |
| | Specific (Jackson ImmunoResearch, |
| | Cat. Nr.: 109-606-098) |
| Anti-CD107 PE secondary Ab | Anti-CD107 PE (BioLegend, Cat. |
| | Nr.: 328608, Clone H4A3, Mouse |
| | IgG1, kappa) |

### EXPERIMENTAL PROCEDURES

### Measurement of Binding to Human Fcγ-Receptors

Binding of HBC34v35-MLNS and HBC34-V35-MLNS-GAALIE to human FcγRs was measured on an Octet instrument (BLI, biolayer interferometry). Briefly, His-tagged human FcγRs (FcγRIla allele H131, FcγRIIa allele R131, FcγRIIAa allele F158, FcγRIIIa allele V158 and FcγRIIb) at 2 μg/ml were captured onto anti-penta-His sensors for 6 minutes. FcγR-loaded sensors were then exposed for 4 minutes to a solution of kinetics buffer (pH 7.1) containing 2 μg/ml of each mAb in the presence 1 μg/ml of affiniPure F(ab′)₂ Fragment Goat Anti-Human IgG, F(ab′)₂ fragment-specific (to cross-link human mAbs through the Fab fragment), followed by a dissociation step in the same buffer for 4 additional minutes (right part of the plot). Association and dissociation profiles were measured in real time as change in the interference pattern using an Octet RED96 (FortéBio).

### Measurement of Binding to Human Complement Protein C1q

Binding of HBC34v35-MLNS and HBC34-V35-MLNS-GAALIE to human complement was measured on an Octet instrument (BLI, biolayer interferometry). Briefly, anti-human Fab (CH1-specific) sensors were used to capture, through the Fab fragment, the full IgG1 of HBC34v35 MLNS and HBC34-V35-MLNS-GAALIE mAbs at 10 μg/ml for 10 minutes. IgG-loaded sensors were then exposed for 4 minutes to a solution of kinetics buffer (pH 7.1) containing 3 μg/ml of purified human C1q (left part of the plot), followed by a dissociation step in the same buffer for 4 additional minutes (right part of the plot). Association and dissociation profiles were measured in real time as change in the interference pattern using an Octet RED96 (FortéBio).

Preparation of Human NK Cells from Whole Blood

NK cells were freshly isolated from whole EDTA blood using the MACSxpress® NK isolation Kit following the manufacturer's instruction. Briefly, anticoagulated blood was mixed in a 50 ml tube with 15 ml of the NK isolation cocktail and incubated for 5 minutes at room temperature using a rotator at approximately 12 rounds per minute. The tube was then placed in the magnetic field of the MACSxpress® Separator for 15 minutes. The magnetically labeled cells adhere to the wall of the tube while the aggregated erythrocytes sediment to the bottom. The target NK cells were then collected from the supernatant while the tube was still inside the MACSxpress® Separator. NK cells were centrifuged, treated with distilled water to remove residual erythrocytes, centrifuged again and finally resuspended in AIM-V medium.

### Determination of Antibody-Dependent NK Cell Killing

MAbs were serially diluted 10-fold in AIM-V medium from 100 μg/ml to 0.001 μg/ml. Target cells (PLC/PRF/5; MacNab, et al., British Journal of Cancer, 34(5), 1976) were added in a round bottom 384-well plate at 7.5×10³ cells/well in 23 μl, then serially diluted antibodies were added to each well (23 μl per well), and the antibody/cell mixture was incubated for 10 minutes at room temperature. Following incubation, human NK cells were added at a cell density of 7.5×10⁴/well in 23 μl, yielding an effector to target ratio of 10:1. Control wells were also included that were used to measure maximal lysis (containing target cells with 23 μl of 3% Triton x-100) and spontaneous lysis (containing target cells and effector cells without antibody). Plates were incubated for 4 hours at 37° C. with 5% CO₂. Cell death was determined by measuring lactate dehydrogenase (LDH) release using a LDH detection kit according to the manufacturer's instructions. In brief, plates were centrifuged for 4 minutes at 400×g, and 35 μl of supernatant was transferred to a flat 384-well plate. LDH reagent was prepared and 35 μl were added to each well. Using a kinetic protocol, the absorbance at 490 nm and 650 nm was measured once every 2 minutes for 8 minutes. The percent specific lysis was determined by applying the following formula: (specific release−spontaneous release)/(maximum release−spontaneous release)×100.

### Determination of Antibody-Dependent NK Cell Activation

Activation of primary NK cells was tested using freshly isolated cells from two donors that had been previously genotyped for expressing homozygous high (V158 allele) or low (F158 allele) affinity FcγRIIIa. Serial dilutions of mAbs (serially diluted 10-fold in AIM-V medium from 100 μg/ml to 0.0001 μg/ml) were incubated with NK cells for 4 hours. Activation of NK cell was measured by flow cytometry by staining NK cells with anti-CD107a mAb (anti-CD107 PE, BioLegend, used diluted 1/35) as a functional marker for NK cell activity.

### Determination of Antibody-Dependent Activation of Human FcγRIIIa

HBC34v35-MLNS and HBC34-V35-MLNS-GAALIE were serially diluted 4-fold in ADCC Assay buffer from 5 μg/ml to 0.076 μg/ml. Target antigen (HBsAg from Engerix B, Glaxo SmithKline) was added in a white flat bottom 96-well plate at 0.6 μg/ml in 25 μl, then serially diluted antibodies were added to each well (25 μl per well), and the antibody/cell mixture was incubated for 10 minutes at room temperature. Effector cells for the ADCC Bioassay were thawed and added at a cell density of 7.5×10⁴/well in 25 μl (final HBsAg concentration was 0.2 μg/ml). Control wells were also included that were used to measure antibody-independent activation (containing HBsAg and effector cells but no antibody) and spontaneous luminescence of the plate (wells containing the ADCC Assay buffer only). Plates were incubated for 24 hours at 37° C. with 5% CO₂. Activation of human FcγRIIIa (V158 or F158 variants) in this bioassay results in NFAT-mediated expression of the luciferase reporter gene. Luminescence was measured with a luminometer using the Bio-Glo-™ Luciferase Assay Reagent according to the manufacturer's instructions. The data (i.e., specific FcγRIIIa activation) are expressed as the average of relative luminescence units (RLU) over the background by applying the following formula: (RLU at concentration x of mAbs-RLU of background).

### Determination of Antibody-Dependent Activation of Human FcγRIIa

HBC34v35-MLNS and HBC34-V35-MLNS-GAALIE were serially diluted 5-fold in ADCP Assay buffer from 50 μg/ml to 0.00013 μg/ml. Target antigen (HBsAg from Engerix B) was added in a white flat bottom 96-well plate at 0.6 or 6 μg/ml in 25 μl, then serially diluted antibodies were added to each well (25 μl per well), and the antigen/antibody was incubated for 25 minutes at room temperature. Effector cells for the FcγRIIa activation bioassay were thawed and added at a cell density of 50.0×10⁴/well in 25 μl (final HBsAg concentration was 0.2 or 2 μg/ml, respectively). Control wells were also included that were used to measure antibody-independent activation (containing HBsAg and effector cells but no antibody) and spontaneous luminescence of the plate (wells containing the ADCP Assay buffer only). Plates were incubated for 23 hours at 37° C. with 5% CO₂. Activation of human FcγRIIa (H131 variants) in this bioassay results in NFAT-mediated expression of the luciferase reporter gene. Luminescence was measured with a luminometer using the Bio-Glo-™ Luciferase Assay Reagent according to the manufacturer's instructions. The data (i.e., specific FcγRIIa activation) are expressed as the average of relative luminescence units (RLU) over the background by applying the following formula: (RLU at concentration [x] of mAbs—RLU of background).

### Determination of Antibody-Dependent Activation of Human FcγRIIb

HBC34v35-MLNS and HBC34-V35-MLNS-GAALIE were serially diluted 5-fold in ADCP Assay buffer from 100 μg/ml to 0.00026 μg/ml. Target antigen (HBsAg from Engerix B) was added in a white flat bottom 96-well plate at 3 μg/ml in 25 μl, then serially diluted antibodies were added to each well (25 μl per well), and the antigen/antibody was incubated for 15 minutes at room temperature. Effector cells for the FcγRIIb activation bioassay were thawed and added at a cell density of 75.0×10⁴/well in 25 μl (the final HBsAg concentration was 1 μg/ml). Control wells were also included that were used to measure antibody-independent activation (containing HBsAg and effector cells but no antibody) and spontaneous luminescence of the plate (wells containing the ADCP Assay buffer only). Plates were incubated for 20 hours at 37° C. with 5% CO₂. Activation of human FcγRIIb in this bioassay results in NFAT-mediated expression of the luciferase reporter gene. Luminescence was measured with a luminometer using the Bio-Glo-™ Luciferase Assay Reagent according to the manufacturer's instructions. The data (i.e., specific FcγRIIb activation) are expressed as the average of relative luminescence units (RLU) over the background by applying the following formula: (RLU at concentration [x] of mAbs—RLU of background).

### Determination of Antibody Binding to Human Hepatoma Cell Line PLC/PRF/5

PLC/PRF/5 cells were trypsinized for 5 min at 37° C., transferred in 7 ml growing medium, centrifugated at 400×g, 4 min, 4° C., and extensively washed at 4° C. in PBS. Some cells were fixed with 4% formaldehyde (20 minutes at 4° C.); others were fixed and then permeabilized with permeabilization buffer (20 minutes at 4° C.). The cellular pellet was resuspended in 2.64 ml of wash buffer (fixed cells) or permeabilization buffer (fix&perm cells) (Table 7) and dispensed at 200 μl/well into 96-well round bottom plates (corresponding to 100′000 cells/well). The plate was centrifugated at 400 g, 4 min, 4° C. Serial 1:5 5-points dilutions of the test antibodies starting from a final concentration of 10 μg/ml were added to cell-containing wells and incubated 30 minutes on ice. After 2 washes at 4° C., 400×g, 4 min in wash buffer (fix cells) or permeabilization buffer (fix&perm cells), 50 μl/well of Alexa Fluor® 647-labelled secondary antibody (Table 7) was added to cells and incubated for 20 min on ice. Cells were washed 2 more times with wash buffer (fix cells) or permeabilization buffer (fix&perm cells), resuspended in 200 μl/well of wash buffer (fix cells) or permeabilization buffer (fix&perm cells) and signal (MFI, mean fluorescence intensity) was quantified with a cytofluorimeter (BD FACSCanto II).

### Results

Direct antiviral mechanisms are important for neutralizing HBV in vivo. Indirect, Fc-dependent mechanisms of action mediated by the interaction of the Fc region with Fc gamma receptors (FcγRs) on immune cells may also have important contributions to in vivo efficacy and to mediate endogenous immune responses. FcγR-dependent mechanisms can be assessed in vitro by measuring binding to FcγRs as well as in antibody-dependent activation of human FcγRs (Hsieh, Y.-T., et al., Journal of Immunological Methods, 441 (C), 56-66. doi.org/10.1016/j.jim.2016.12.002).

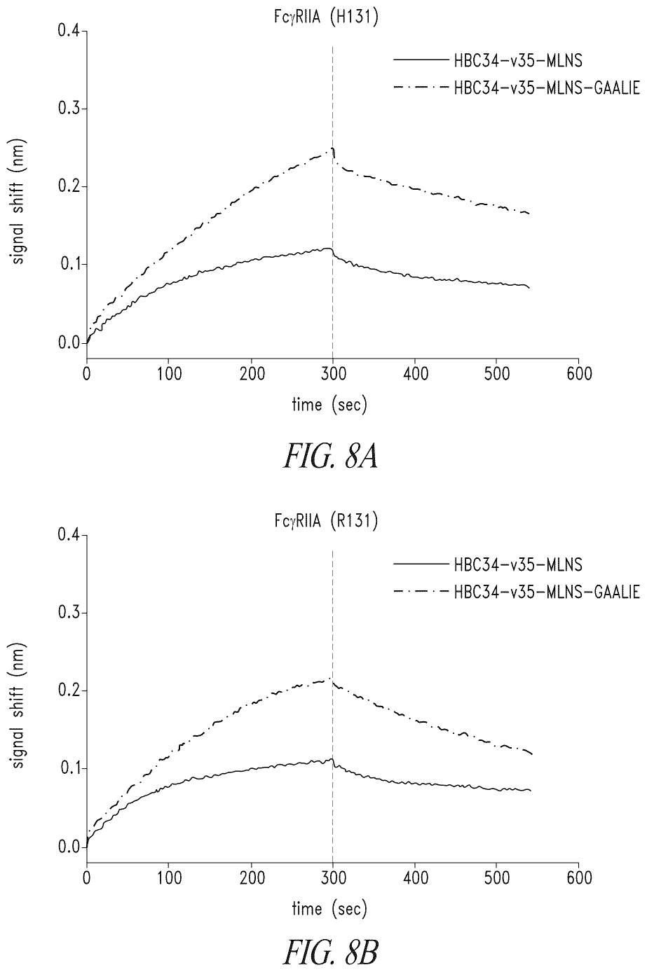

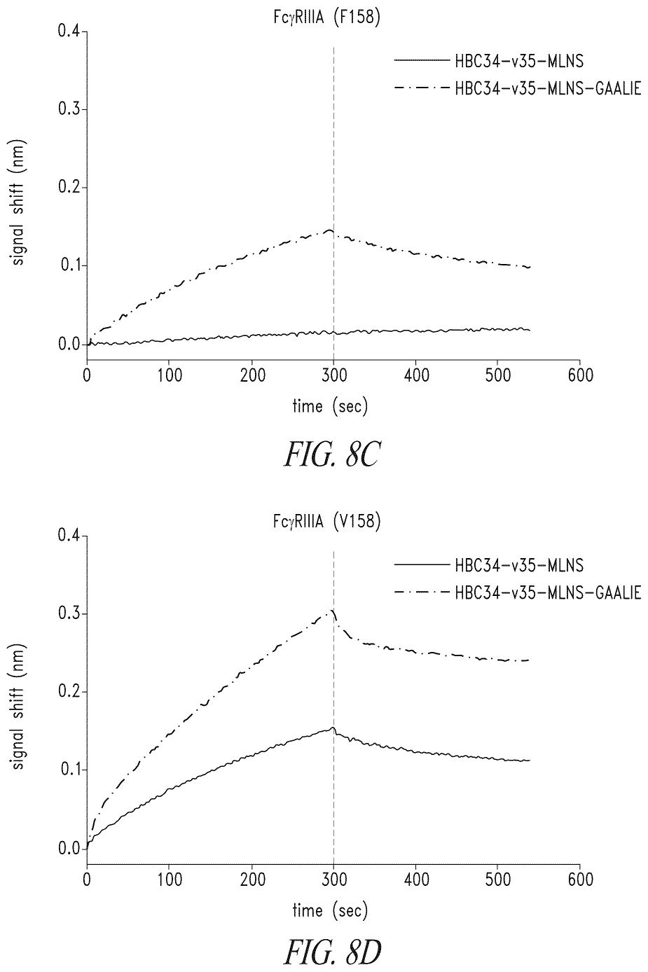

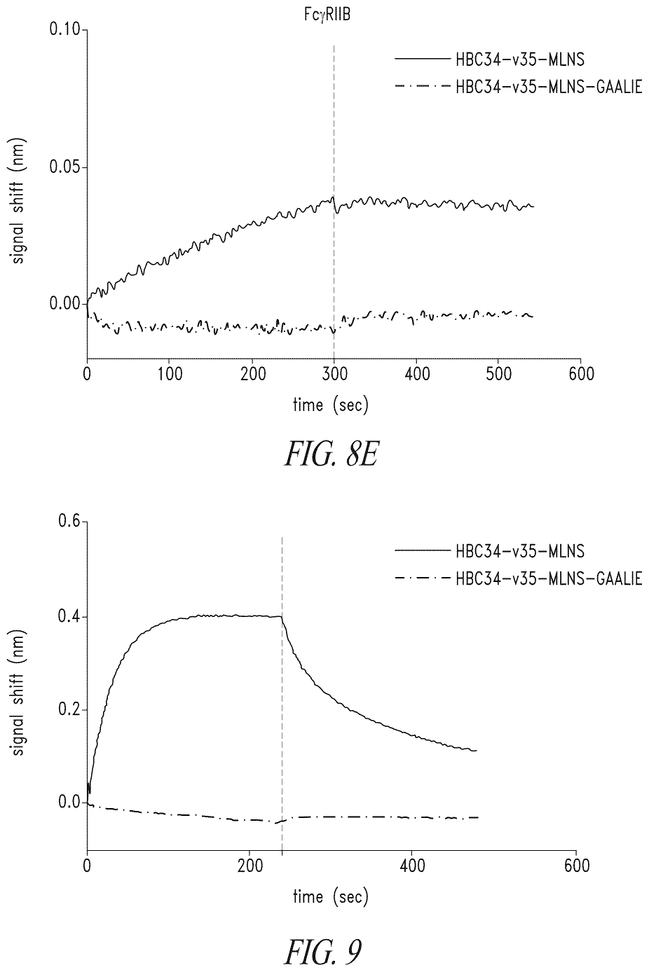

In this study, HBC34v35-MLNS and HBC34-V35-MLNS-GAALIE were compared side-by-side for their ability to bind to the full set of human FcγRs (FcγRIIIa V158 and F158 alleles, FcγRIla H131 and R131 alleles and FcγRIIb) using biolayer interferometry (BLI Octet System, ForteBio). As shown in FIGS. 8A-8E, Fc bearing MLNS-GAALIE mutations have altered interactions with FcγRs; specifically, Fc bearing these mutations have enhanced binding to FcγRIIIa and FcγRIIa, and reduced binding to FcγRIIb. Of note, binding of HBC34-V35-MLNS-GAALIE to C1q was abolished as measured by biolayer interferometry (FIG. 9).

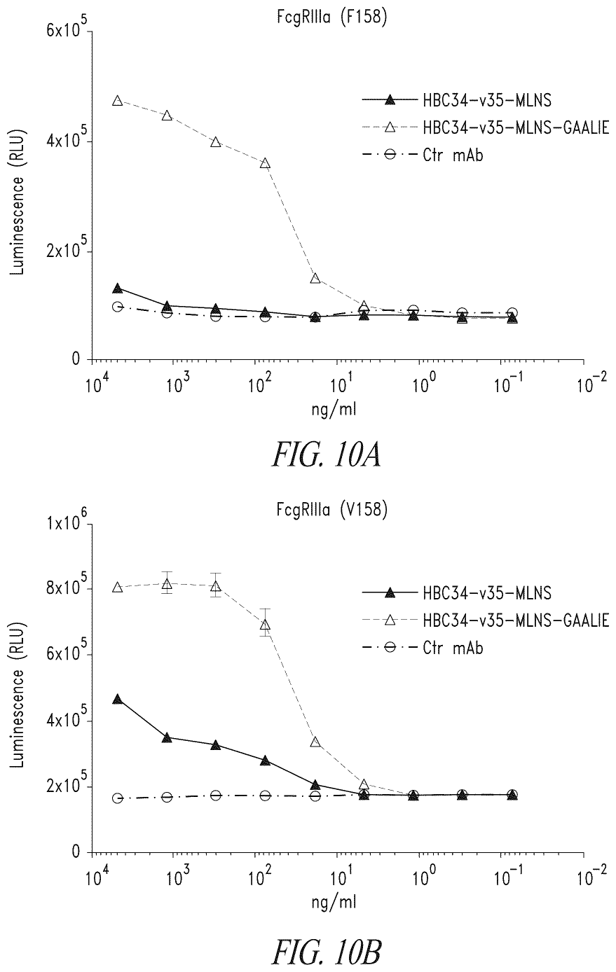

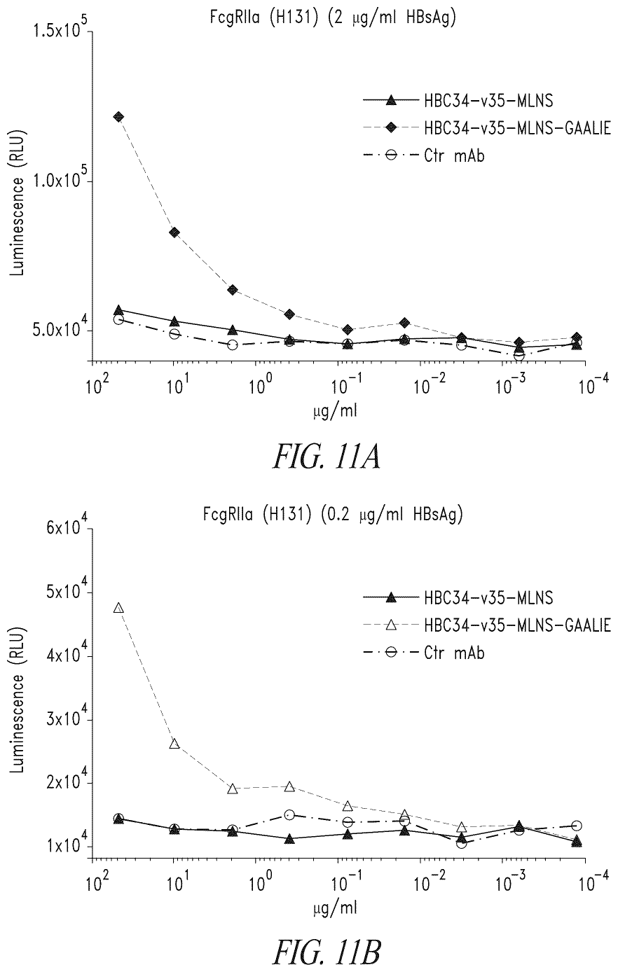

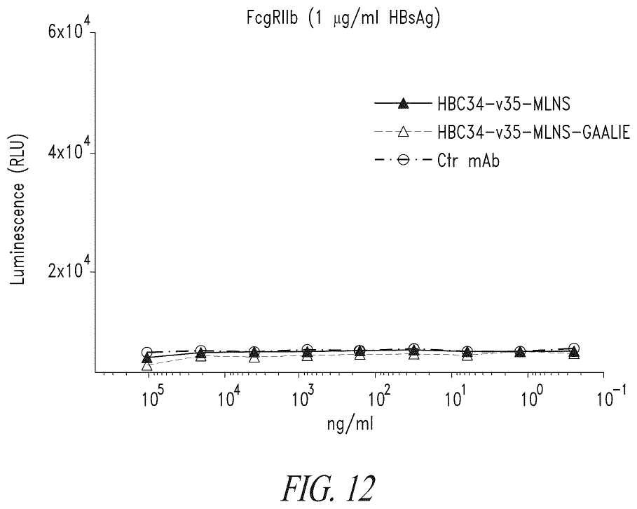

HBC34-V35-MLNS and HBC34-V35-MLNS-GAALIE were also tested for their ability to activate human FcγRIIIa and FcγRIIa using cell-based reporter bioassays. These assays utilize Jurkat cells engineered with a NFAT-mediated luciferase reporter to reflect activation of human FcγRs. While HBC34v35-MLNS poorly activated or did not activate human FcγRIIla and FcγRIla in the presence of HBsAg, HBC34-V35-MLNS-GAALIE showed a dose-dependent activation of all tested FcγRs (FIGS. 10A, 10B, 11A, and 11B). Conversely, HBC34-V35-MLNS-GAALIE did not activate FcγRIIb, even when tested at 100 μg/ml (FIG. 12).

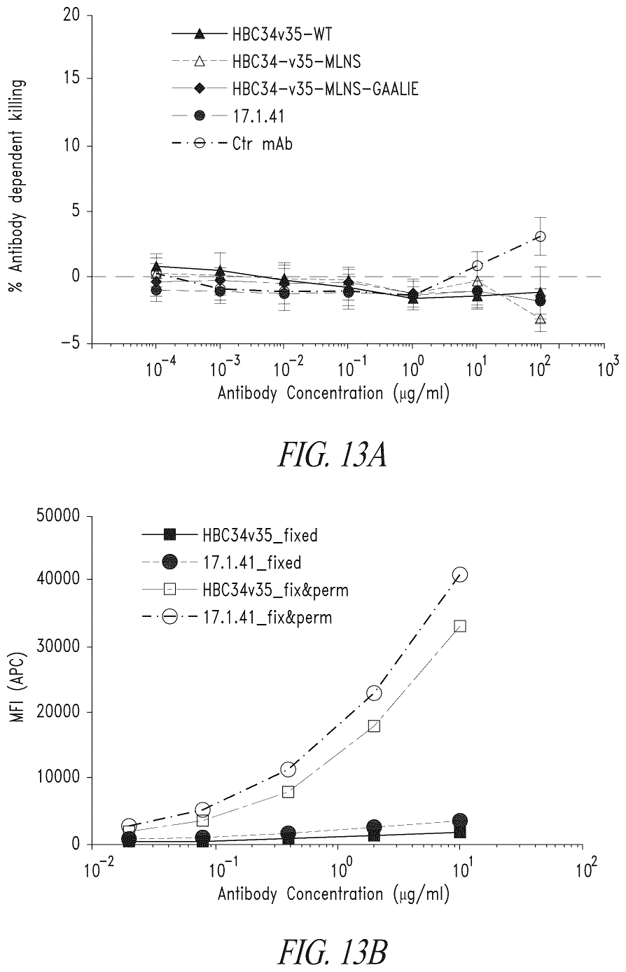

ADCC activity was also measured using natural killer cells (NK) isolated from human peripheral blood mononuclear cells of one donor who was previously genotyped for expressing heterozygous high (V158) and low (F158) affinity FcγRIIIa (F/V). Isolated NK cells were used to measure the killing of the hepatoma cell line PLC/PR/5 upon exposure to HBC34v35; HBC34v35-MLNS; HBC34-V35-MLNS-GAALIE; or another mAb (17.1.41, targeting another epitope on the antigenic loop of the HBsAg; see Eren, R., et al., Hepatology, doi.org/10.1053/jhep.2000.9632; Galun, E., et al., Hepatology, doi.org/10.1053/jhep.2002.31867). Killing in the presence of the HBsAg-specific mAbs HBC34v35, HBC34v35-MLNS, HBC34-V35-MLNS-GAALIE and 17.1.41 was not observed (FIG. 13A). The observed lack of antibody-dependent killing of PLC/PR/5 cells might be related to the poor expression of HBsAg on the surface of these cells (FIG. 13B), which, without wishing to be bound by theory, may not be sufficient to trigger killing by NK cells. Conversely, high levels of HBsAg were detected with HBC34v35 and 17.1.41 when PLC/PR/5 cells were fixed and permeabilized, indicating that most of the HBsAg is found either intracellularly or in secreted forms (i.e. subviral particles) (FIG. 13B).

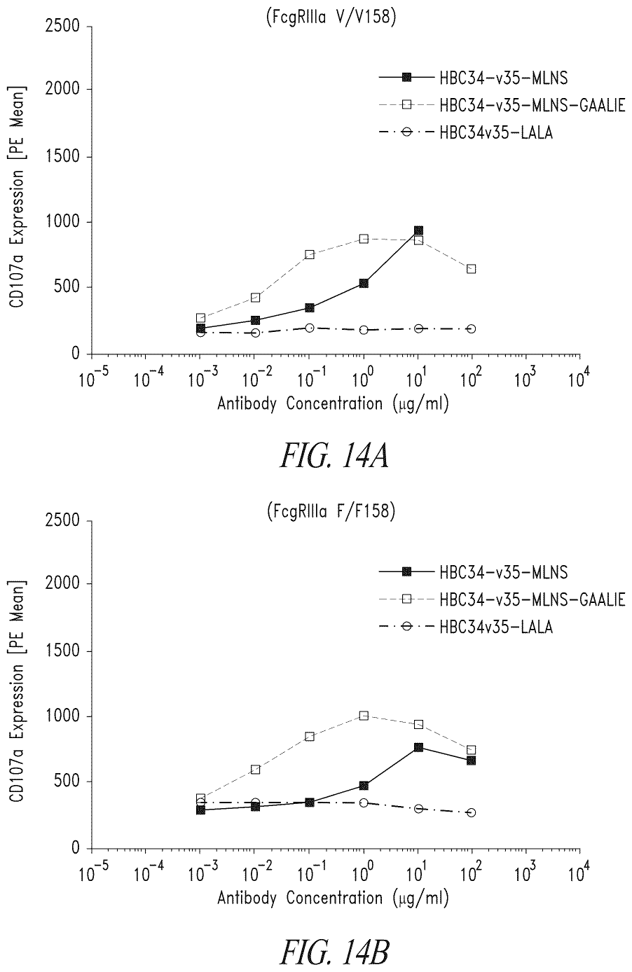

Activation of primary human NK cells (V/F) in the presence of HBC34v35-MLNS or HBC34-V35-MLNS-GAALIE and HBsAg was also examined using anti-CD107a mAb. Data are shown in FIGS. 14A and 14B.

These in vitro data show that HBV-specific binding proteins of the present disclosure bearing the GAALIE Fc mutation bind to and activate low affinity activating FcγRIIa and FcγRIIIa more effectively than the non-GAALIE Fc parental antibody. GAALIE-bearing binding proteins also do not bind to and or activate low affinity inhibitory FcγRIIb. GAALIE-bearing binding proteins also do not bind to C1q. Furthermore, GAALIE-bearing binding proteins do not promote ADCC on hepatoma cells, but activate human NK cells in the presence of soluble HBsAg.

### Example 9: Phase 1 Clinical Study of HBC34-v35-MLNS-GAALIE

A multi-center phase 1, randomized, placebo-controlled study is performed to evaluate the safety, tolerability, pharmacokinetics, and antiviral activity of HBC34-v35-MLNS-GAALIE (comprising the heavy chain amino acid sequence shown in SEQ ID NO.: 91 and the light chain amino acid sequence shown in SEQ ID NO.: 93). The study sites are as follows: Part A (single-center) and Parts B/C (multi-center).

In Part A (up to 40 subjects), the primary objective is to evaluate the safety and tolerability of HBC34-v35-MLNS-GAALIE in healthy adult subjects. The secondary objectives are to characterize the serum pharmacokinetics (PK) of HBC34-v35-MLNS-GAALIE in healthy adult subjects, and to evaluate the immunogenicity (induction of anti-drug antibody [ADA]) of HBC34-v35-MLNS-GAALIE in healthy adult subjects,

In Parts B (up to 56 subjects) and C (up to 24 subjects), the primary objective is to evaluate the safety and tolerability of HBC34-v35-MLNS-GAALIE in adult subjects with chronic HBV infection without cirrhosis. The secondary objectives are: to characterize the serum PK of HBC34-v35-MLNS-GAALIE in adult subjects with chronic HBV infection without cirrhosis; to assess the antiviral activity of HBC34-v35-MLNS-GAALIE in adult subjects with chronic HBV infection without cirrhosis; and to evaluate the immunogenicity (induction of ADA) of HBC34-v35-MLNS-GAALIE in adult subjects with chronic HBV infection without cirrhosis. The exploratory objectives include: to evaluate the effect of HBC34-v35-MLNS-GAALIE on additional viral parameters; to evaluate the effect of HBC34-v35-MLNS-GAALIE on immune responses (or exploratory biomarkers) in adult subjects with chronic HBV infection without cirrhosis; and to evaluate the impact of host polymorphisms (or exploratory biomarkers) on response to HBC34-v35-MLNS-GAALIE in adult subjects with chronic HBV infection without cirrhosis.

### Details of Criteria for Evaluation

For Part A, the primary endpoints of this study are as follows:
Incidence of treatment-emergent adverse events (TEAEs).
Clinical assessments including but not limited to laboratory test results

The secondary endpoints of this study are as follows:
HBC34-v35-MLNS-GAALIE serum free PK parameters, for example: Cₘₐₓ, Clast, Tₘₐₓ, Tₗₐₛₜ, AUC_{inf}, AUCₗₐₛₜ, % AUCₑₓₚ, t_{1/2}, λ_{z}, V_{z} (IV only), CL (IV only), V_{z}/F (SC only), and CL/F (SC only).
Incidence and titers (if applicable) of ADA to HBC34v-35-MLNS-GAALIE For Parts B/C, the primary endpoints of this study are as follows:
Incidence of TEAEs
Clinical assessments including but not limited to laboratory test results

The secondary endpoints of this study are as follows:
HBC34-v35-MLNS-GAALIE serum free and total PK parameters, for example: Cₘₐₓ, Cₗₐₛₜ, Tₘₐₓ, Tₗₐₛₜ, AUC_{inf}, AUCₗₐₛₜ, % AUCₑₓₚ, t_{1/2}, λz, V_{z}/F, and CL/F.
Incidence and titers (if applicable) of ADA to HBC34-v35-MLNS-GAALIE·Maximum reduction of serum HBsAg from baseline (Day 1 predose)

The exploratory endpoints of this study may include:
Assessment of additional viral parameters (for example: HBV RNA and HBcrAg).
Analysis of host immune responses.
Analysis of host factors as determined by RNA-sequencing.
Fc gamma receptor (FcγR) polymorphisms as determined by genotyping
IgG allotypes as determined by genotyping

### Number of Subjects Planned

Part A: Up to 40 healthy adult subjects.
Part B: Up to 56 adult subjects with chronic HBV infection without cirrhosis on nucleos (t) ide reverse transcriptase inhibitor (NRTI) therapy who are HBeAg-negative and who have HBsAg<1000 IU/mL.
Part C: Up to 24 adult subjects with chronic HBV infection without cirrhosis on NRTI therapy who have HBsAg ≥1000 IU/mL.

### Diagnosis and Main Criteria for Inclusion

### Part A Inclusion Criteria Include:

Healthy adult subjects age 18 (or age of legal consent, whichever is older) to 55 years who weigh ≥40 kg to ≤125 kg. Patients are in good health, determined from medical history (e.g. chronic conditions such as hypertension, hyperlipidemia, gastroesophageal reflux disease, asthma, anxiety and depression must be well controlled), and no clinically significant findings from physical examination, 12-lead ECG, vital signs, and laboratory values. Female subjects must have a negative pregnancy test or confirmation of postmenopausal status. Post-menopausal status is defined as 12 months with no menses without an alternative medical cause. Women of child-bearing potential (WOCBP) must have a negative blood pregnancy test at screening and a negative urine pregnancy test on Day 1, cannot be breast feeding, and must be willing to use highly effective methods of contraception, as disclosed herein, 14 days before study drug administration through 40 weeks after study drug administration.

Male subjects with female partners of child-bearing potential must agree to meet 1 of the following contraception requirements from the time of study drug administration until 40 weeks post-dose of study drug: vasectomy with documentation of azoospermia, or male condom use plus partner use of a highly effective contraception often. Male subjects must also agree not to donate sperm from the time of study drug administration through 40 weeks after study drug administration. Patients agree not to donate blood during the duration of the study

Patients are willing to comply with the study requirements and able to provide written informed consent.

### Part B/C Inclusion Critera Include:

1. Aged 18 (or age of legal consent, whichever is older) to 65 years
2. Weigh ≥40 kg to ≤125 kg with chronic HBV infection (defined by: Positive serum HBsAg, HBV DNA, or HBeAg on 2 occasions at least 6 months apart based on previous or current laboratory documentation (any combination of these tests performed 6 months apart is acceptable))
3. Without cirrhosis
4. On NRTI therapy for at least 2 months at the time of screening, are HBeAg-negative. Examples of NRTI therapy include, but are not limited to: Tenofovir disoproxil/tenofovir alafenamide; Entecavir; Lamivudine; Adefovir/adefovir dipivoxil.
5. HBV DNA<100 IU/mL at screening
6. HBsAg>the lower limit of detection
7. HBsAg<1000 IU/mL (Part B only) at screening
8. HBsAg≥1000 IU/mL (Part C only) at screening
9. HBeAg-negative at screening (Part B only)
10. Negative anti-HBs at screening
11. Besides chronic infection with HBV, must be in good health, determined from medical history (e.g. chronic conditions such as hypertension, hyperlipidemia, gastroesophageal reflux disease, asthma, anxiety and depression must be well controlled), and no clinically significant findings from physical examination, 12-lead ECG, vital signs, and laboratory values.
12. Female subjects must have a negative pregnancy test or confirmation of postmenopausal status. Post-menopausal status is defined as 12 months with no menses without an alternative medical cause. Women of childbearing potential must have a negative blood pregnancy test at screening and a negative urine pregnancy test on Day 1, cannot be breast feeding, and must be willing to use highly effective methods of contraception 14 days before study drug administration through 40 weeks after the dose of study drug.
13. Male subjects with female partners of child-bearing potential must agree to meet 1 of the following contraception requirements from the time of study drug administration through 40 weeks after the dose of study drug: vasectomy with documentation of azoospermia, or male condom use plus partner use of 1 of the contraceptive options listed for contraception for WOCBP (see herein). Male subjects must also agree to not donate sperm from the time of first study drug administration through 40 weeks after the dose of study drug.
14. Willing to comply with the study requirements and able to provide written informed consent.

Birth control methods which are considered highly effective include:
Established use of combined (estrogen and progestogen containing) oral, intravaginal, or transdermal hormonal methods of contraception associated with inhibition of ovulation OR established use of progestogen-only oral, injectable, or implantable hormonal methods of contraception associated with inhibition of ovulation. It is not currently known whether HBC34-v35-MLNS_GAALIE will impact the effectiveness of hormonal contraceptive methods; therefore, it is recommended to use an additional form of contraception (ie, barrier method) throughout the study and for 40 weeks after study drug administration.
Placement of an intrauterine device.
Placement of an intrauterine hormone-releasing system.
Surgical sterilization of male partner (with the appropriate post-vasectomy documentation of the absence of sperm in the ejaculate; for female subjects on the study, the vasectomized male partner should be the sole partner for that subject).
True sexual abstinence from heterosexual contact, when in line with the preferred and usual lifestyle of the subject. Periodic abstinence (eg, calendar, ovulation, symptothermal, post ovulation methods) and withdrawal are not acceptable methods of contraception. Abstinent subjects have to agree to use 1 of the above-mentioned contraceptive methods, if they start sexual relationships during the study and for up to 40 weeks after study drug administration, or for as long as the subject is followed on study, whichever is longer.
Barrier method in combination with hormonal contraceptive, as described above

Post-menopausal status is defined as 12 months with no menses without an alternative medical cause.

Male subjects with female partners of child-bearing potential must agree to meet 1 of the following contraception requirements from the time of study treatment administration until 40 weeks after study drug administration.
Vasectomy with documentation of azoospermia.
Male condom plus partner use of 1 of the contraceptive options listed above for contraception for WOCBP (hormonal contraceptive, intrauterine device)

Male subjects must also agree not to donate sperm for the 40 weeks following last study drug administration.

### Duration of Study Participation

Part A: The duration of study drug treatment is a single dose. The estimated total time on study, inclusive of screening and follow-up, for each subject is up to 28 weeks.
Parts B/C: The duration of study drug treatment is a single dose. The estimated total time on study, inclusive of screening and follow-up, for each subject is up to 44 weeks.

### Duration of Follow-Up

Part A: All subjects are followed for 24 weeks after study drug administration.
Parts B/C: All subjects are followed for 8 weeks after study drug administration. Subjects with >2-fold HBsAg reduction at Week 8 undergo extended follow-up for up to 40 weeks total or until the reduction in HBsAg is <2-fold relative to baseline at 2 consecutive collections, whichever occurs first. The extended follow-up may be discontinued based on emerging data.

### Study Design

A Safety Review Committee (SRC) performs ongoing reviews of safety, tolerability, and antiviral activity data (Parts B and C only) at specified timepoints based on available data collected throughout the study. While the primary data that will be reviewed by the SRC for dose escalations and enrollment of optional cohorts is listed throughout the protocol, additional relevant data from other cohorts is also reviewed by the SRC as indicated to inform decisions.

The study is conducted in 3 Parts:
Part A: Randomized, double-blind, placebo-controlled, single ascending dose (SAD) study of HBC34-v35-MLNS-GAALIE administered via subcutaneous (SC) injection or intravenous (IV) infusion to healthy adult subjects.
Part B: Randomized, double-blind, placebo-controlled, SAD study of HBC34-v35-MLNS-GAALIE administered via SC injection to adult subjects with chronic HBV infection without cirrhosis who are on NRTI therapy, are HBeAg-negative, and have HBsAg<1000 IU/mL.
Part C: Optional, randomized, double-blind, placebo-controlled, SAD study of HBC34v35-MLNS-GAALIE administered via SC injection to adult subjects with chronic HBV infection without cirrhosis who are on NRTI therapy and who have HBsAg≥1000 IU/mL

### Overall Risk/Benefit Assessment

The potential risks for healthy adult subjects are based on the common safety risks observed with the mAb class of therapeutics and are not specific to HBC34-v35-MLNS-GAALIE: anaphylaxis and other serious allergic reactions and injection/infusion-related reactions. The risk of developing such conditions after dosing with HBC34v35-MLNS-GAALIE specifically is unknown.

Part A of the study gathers information on the safety and tolerability of HBC34v35-MLNS-GAALIE as well as relevant data on the PK profile and the generation of anti-drug antibodies (ADAs). HBC34-v35-MLNS-GAALIE is not expected to offer benefit to healthy subjects enrolled in Part A of this study. Subjects will be monitored for important potential risks, and routine pharmacovigilance and risk minimization activities will be performed.

The potential benefits of HBC34-v35-MLNS-GAALIE in subjects with Chronic HBV infection over the current standard of care are:
Reduction in serum HBsAg, inhibition of intrahepatic spread of HBV, elimination of infected hepatocytes, and stimulation of adaptive immune responses against HBV.
A pangenotypic therapy for HBV infection that is well-tolerated and administered SC for a finite duration of time

In addition to anaphylaxis, other serious allergic reactions, and injection/infusion-related reactions, potential risks associated with the administration of HBC34-v35-MLNS-GAALIE to subjects with chronic HBV infection include immune complex disease and hepatotoxicity due to the elimination of infected hepatocytes via ADCC/ADCP and/or cytotoxic T-cells induced via a vaccinal effect. The study design of Parts B/C includes several elements to mitigate these risks:
Part B enrolls subjects with serum HBsAg<1000 IU/mL, to mitigate the risk for immune complex disease and hepatotoxicity. Additionally, Part B safety data is reviewed by the SRC prior to enrolling subjects with potentially higher baseline HBsAg values in the optional Part C of the study.
Parts B and C enroll subjects who are on NRTIs and have HBV DNA<100 IU/mL at screening and have good hepatic reserve and a low level of hepatic inflammation at baseline as determined by the following attributes: ALT or AST≤2×ULN, no history of hepatic decompensation, and lack of significant fibrosis and cirrhosis.
Two sentinel subjects are randomized 1:1 to HBC34-v35-MLNS-GAALIE or placebo and dosed. These sentinel subjects are monitored through at least 72 hours post-dose and if the investigator(s) have no safety concerns, the remaining 6 subjects in the same cohort is dosed (5 active and 1 placebo).
Dose escalation occurs after SRC review of available safety data up to 4 weeks after dose administration to account for the anticipated timing of potential immune complex disease and hepatotoxicity due to the elimination of infected hepatocytes via ADCC/ADCP and/or cytotoxic T-cells induced via a vaccinal effect
Safety monitoring, including liver function tests, urinalysis, renal function, vital signs, and physical examination findings, is designed to detect evidence of HBC34-v35-MLNS-GAALIE-associated immune adverse events.

### Part A

Three sequential cohorts for Part A evaluate 90 mg, up to 300 mg, and up to 900 mg administered by SC injection. The SRC reviews available clinical and laboratory safety data up to 2 weeks post-dose for all available subjects within a cohort prior to dose escalation. Two optional cohorts in Part A can be added evaluating up to 900 mg and 3000 mg administered by IV infusion. Enrollment of these optional cohorts can occur following SRC review of available Week 2 data from all available subjects in Cohort 3a (up to 900 mg SC).

While all of the SC cohorts (Cohort 1a, 2a, and 3a) in Part A are enrolled sequentially, cohorts may be enrolled in parallel if the additional cohort(s) is examining a dose level which is at or below a dose level that has previously been found to have an acceptable safety and tolerability profile in a prior cohort in Part A.

In each cohort, 2 sentinel subjects are randomized 1:1 to receive HBC34-v35-MLNS-GAALIE or placebo. These subjects are dosed and monitored for at least 24 hours in an inpatient setting; if the investigator has no safety concerns, the remainder of the subjects in the same cohort are dosed. The remaining subjects are randomized 5:1 to receive HBC34-v35-MLNS-GAALIE or placebo.

The maximum dose escalation factor in Part A does not exceed 5-fold.

### Part B

The first cohort in Part B (Cohort 1b) is enrolled after SRC review of available Week 2 data from all available subjects in Cohort 1a (90 mg SC).

Five cohorts are planned for Part B evaluating 6 mg (Cohort 1b), 18 mg (Cohort 2b), up to 75 mg (Cohort 3b), up to 300 mg (Cohort 4b), and up to 900 mg (Cohort 5b) administered by SC injection. The SRC reviews available clinical and laboratory safety data and antiviral activity data up to 4 weeks post-dose for all available subjects within the prior cohort prior to dose escalation. Two optional cohorts in Part B may be added following the same dosing schedule. The optional cohorts may be dosed at a lower, equivalent, or intermediate dose level relative to the dose levels explored in the planned Part B cohorts, or after cohort 5b at a dose level not exceeding 900 mg. The maximum dose level for the optional cohorts in Part B does not exceed the highest single dose found to have an acceptable safety and tolerability profile in Part A. The optional cohorts are enrolled at any time within the Part B planned cohorts based on the approval of the SRC.

While all of the cohorts in Part B are to be enrolled sequentially, cohorts may be enrolled in parallel if the additional cohort(s) is examining a dose level which is at or below a dose level that has previously been found to have an acceptable safety and tolerability profile in a prior cohort in Part A and Part B.

In each cohort, 2 sentinel subjects are randomized 1:1 to receive HBC34-v35-MLNS-GAALIE or placebo by SC injection. These subjects are dosed and monitored through at least 72 hours post-dose (including inpatient monitoring over at least the first 24 hours); if the investigator(s) have no safety concerns, the remainder of the subjects in the same cohort are dosed. The remaining subjects are randomized 5:1 to receive HBC34-v35-MLNS-GAALIE or placebo by SC injection.

The maximum dose escalation factor in Part B does not exceed 5-fold.

### Part C

Part C is optional and may be conducted based on an acceptable safety and tolerability profile of HBC34-v35-MLNS-GAALIE in HBeAg-negative subjects with HBsAg levels <1000 IU/mL in Part B. The first cohort in Part C is enrolled after SRC review of available data for all subjects in Part A and Part B through the Week 4 visit for the cohort of subjects in Part B who are receiving a matching or higher dose relative to the proposed starting dose level in Part C.

Three optional cohorts can be enrolled in Part C. Each cohort may evaluate up to 900 mg administered by SC injection and the dose utilized in Part C cohorts does not exceed the highest dose level in Part B that was found to have an acceptable safety and tolerability profile by the SRC. Cohorts may be enrolled in parallel.

In each cohort, 2 sentinel subjects are randomized 1:1 to receive HBC34-v35-MLNS-GAALIE or placebo by SC injection. These subjects are dosed and monitored through at least 72 hours post-dose (including inpatient monitoring over at least the first 24 hours); if the investigator(s) have no safety concerns, the remainder of the subjects in the same cohort are dosed. The remaining subjects are randomized 5:1 to receive HBC34-v35-MLNS-GAALIE or placebo by SC injection.

### Study Procedures

### Part A

### Screening

Healthy adult subjects will be enrolled in 1 of 5 cohorts (3 planned, 2 optional) in Part A. Screening is performed no more than 4 weeks prior to the Day 1 visit and includes written informed consent, determination of eligibility, collection of demographics and medical history, physical examination, vital signs, laboratory tests, 12-lead electrocardiogram (ECG) and other assessments per the schedule of assessments (SoA).
Eligible subjects are admitted into the clinical investigative site on Day −1 or 1. On Day 1, eligibility criteria related to vital signs, pregnancy testing, drugs of abuse, blood donation, presence of any clinically significant acute condition, and use of prescription, OTC, herbal, or investigational agents are evaluated to ensure ongoing eligibility for the study. Any changes to medical history are also evaluated and recorded. Eligible subjects in each cohort are randomized to receive HBC34-v35-MLNS-GAALIE or placebo within 48 hours prior to study drug administration. Subjects receive a single dose of study drug on Day 1 (HBC34-v35-MLNS-GAALIE or placebo).
Adverse events (AEs) related to screening activities are collected from the time of consent onwards; any other events occurring during the screening period are reported as medical history. All serious adverse events (SAEs) are collected from the time of consent onwards.
Screening viral serology parameters are as follows: active infection with HIV, HCV, and HBV

### Dosing Day (Day 1)

Eligible subjects are randomized to receive HBC34-v35-MLNS-GAALIE or placebo within 48 hours prior to study drug administration on Day 1.
Eligible subjects receive a single dose of study drug and applicable assessments are performed on Day 1.
At the start of each cohort, 2 sentinel subjects are randomized 1:1 to HBC34v-35-MLNS-GAALIE or placebo. These subjects are dosed and monitored for at least 24 hours in an inpatient setting. Vital signs, ECG, symptom-directed physical examination(s), and AEs are reviewed by the investigator; if the investigator has no safety concerns, the remainder of the subjects in the same cohort are dosed. The remaining subjects in the cohort are randomized 5:1 to receive a single dose of HBC34-v35-MLNS-GAALIE or placebo. All subjects are closely monitored following dose administration.

### Follow-Up Period

Subjects are discharged after all study assessments are performed on Day 2. All subsequent study visits are outpatient.
Subjects return to the clinical investigative site for in-person assessments per the SoA including but not limited to physical examination, vital signs, laboratory testing, PK assessments, and review of AEs and concomitant medications through Week 24.

### Parts B/C

### Screening

Screening is performed no more than 4 weeks prior to the Day 1 visit and includes written informed consent, determination of eligibility, collection of demographics and medical history, physical examination, vital signs, laboratory tests, 12-lead ECG and other assessments per the SoA. Adverse events related to screening activities are collected from the time of consent onwards; any other events occurring during the screening period are reported as medical history. All SAEs are collected from the time of consent onwards.
Adult subjects with HBeAg-negative chronic HBV infection without cirrhosis and with HBsAg<1000 IU/mL on NRTI therapy for ≥2 months are enrolled in 1 of 7 cohorts (5 planned, 2 optional) in Part B. Subject screening occurs no more than 4 weeks prior to the Day 1 visit. Subjects are admitted into the clinical investigative site on Day-1 or 1. On Day 1, eligibility criteria related to NRTI adherence, vital signs, pregnancy testing, presence of any clinically significant acute condition, hepatic decompensation, and use of prescription, OTC, herbal, or investigational agents will be evaluated to ensure ongoing eligibility. Any changes to medical history are also evaluated and recorded. Eligible subjects in each cohort are randomized to receive HBC34-v35-MLNS-GAALIE or placebo within 48 hours prior to study drug administration on Day 1.
To exclude the presence of cirrhosis, subjects in Parts B and C have a FibroScan evaluation. This is not required to be performed if the subject has had a FibroScan in the 6 months prior to screening or liver biopsy in the year prior to screening that confirmed the absence of Metavir F3 fibrosis or F4 cirrhosis.
Screening viral serology parameters are as follows: active infection with HIV, HCV, and hepatitis Delta virus. Subjects who have positive HCV serology result may have HCV-RT PCR reflex testing to determine eligibility.
Chronic HBV infection will be determined at screening and is defined as the following: Positive serum HBsAg, HBV DNA, or HBeAg on 2 occasions at least 6 months apart based on previous or current laboratory documentation (any combination of these tests performed 6 months apart is acceptable).

### Dosing Day (Day 1)

Eligible subjects are randomized to receive HBC34-v35-MLNS-GAALIE or placebo within 48 hours prior to study drug administration on Day 1.
Subjects are admitted into the clinical investigative site on Day 1.
Eligible subjects receive a single dose of study drug and applicable assessments will be performed on Day 1.
At the start of each cohort, 2 sentinel subjects are randomized 1:1 to HBC34-v35-MLNS-GAALIE or placebo. These subjects are dosed and monitored through at least 72 hours post-dose (including inpatient monitoring over at least the first 24 hours); if the investigator(s) haves no safety concerns, the remainder of the subjects in the same cohort are dosed. Vital signs, symptom-directed physical examination(s), and AEs are reviewed by the investigator prior to dosing any additional subjects. The remaining subjects in the cohort are randomized 5:1 to receive a single dose of antibody composition or placebo. All subjects are closely monitored following dose administration.

### Follow-Up Period

Subjects are discharged after all study assessments are performed on Day 2. All subsequent study visits are outpatient.
Subjects return to the clinical investigative site for assessments per the SoA including but not limited to physical examination, vital signs, laboratory testing, PK assessments, efficacy assessments and review of AEs and concomitant medications through Week 8.

### Extended Follow-Up Period

Subjects with >2-fold HBsAg reduction at Week 8 return to the clinical investigative site for in-person assessments per the SoA through Week 40 or until the reduction in HBsAg is <2-fold relative to baseline at 2 consecutive collections, whichever occurs first. The extended follow-up may be discontinued based on emerging data.

### Product, Dosage, and Mode of Administration

HBC34v35-MLNS-GAALIE is supplied as a lyophilized solid to be reconstituted with Sterile Water for Injection (USP) at a concentration of 150 mg/mL and administered as a SC injection or IV infusion. The unit dose is based on volume and administration method. Upon reconstitution to 150 mg/mL with sterile water for injection, USP, the drug product, as administered, contains 20 mM Histidine, 7% sucrose, 0.02% PS80 at pH 6. Placebo is a sterile, preservative-free normal saline 0.9% solution for IV infusion or SC injection
Cohort 1a: HBC34v35-MLNS-GAALIE, single dose of 90 mg administered by SC injection
Cohort 2a: HBC34v35-MLNS-GAALIE, single dose of up to 300 mg administered by SC injection
Cohort 3a: HBC34v35-MLNS-GAALIE, single dose of up to 900 mg administered by SC injection
Cohort 4a (optional): HBC34v35-MLNS-GAALIE, single dose of up to 900 mg administered by IV infusion
Cohort 5a (optional): HBC34v35-MLNS-GAALIE, single dose of up to 3000 mg administered by IV infusion
Cohort 1b: HBC34v35-MLNS-GAALIE, single dose of 6 mg administered by SC injection
Cohort 2b: HBC34v35-MLNS-GAALIE, single dose of 18 mg administered by SC injection
Cohort 3b: HBC34v35-MLNS-GAALIE, single dose of up to 75 mg administered by SC injection
Cohort 4b: HBC34v35-MLNS-GAALIE, single dose of up to 300 mg administered by SC injection
Cohort 5b: HBC34v35-MLNS-GAALIE, single dose of up to 900 mg administered by SC injection
Cohort 6b (optional): HBC34v35-MLNS-GAALIE, single dose of up to 900 mg administered by SC injection
Cohort 7b (optional): HBC34v35-MLNS-GAALIE, single dose of up to 900 mg administered by SC injection
Cohort 1c (optional): HBC34v35-MLNS-GAALIE, single dose of up to 900 mg administered by SC injection
Cohort 2c (optional): HBC34v35-MLNS-GAALIE, single dose of up to 900 mg administered by SC injection
Cohort 3c (optional): HBC34v35-MLNS-GAALIE, single dose of up to 900 mg administered by SC injection

TABLE 8
Part A Dose Escalation Plan

| Study | | | | Frequency of |
|---|---|---|---|---|
| Part | Cohort | Active:Placebo | Dose (Route) | Administration |
| A | 1a | 6:2 | 90 mg (SC) | Once |
| | 2a | 6:2 | Up to 300 mg (SC) | Once |
| | 3a | 6:2 | Up to 900 mg (SC) | Once |
| | 4a | 6:2 | Up to 900 mg (IV) | Once |
| | (Optional) | | | |
| | 5a | 6:2 | Up to 3000 mg (IV) | Once |
| | (Optional) | | | |

IV = intravenous;
SC = subcutaneous.

Part B: Single Ascending Dose Study in Subjects with Chronic HBV Infection

In Part B, subjects with chronic HBV infection receive a single dose of study drug. The presence of the therapeutic target of HBC34-v35-MLNS-GAALIE, HBsAg, in subjects with chronic HBV infection alters the potential risks of HBC34-v35-MLNS-GAALIE administration. Potential risks include immune complex disease due to the formation of antigen-antibody complexes and hepatotoxicity due to elimination of infected hepatocytes via ADCC/ADCP and/or a “vaccinal effect”. To minimize risks to subjects, Part B will be conducted in subjects who are on NRTIs and have HBV DNA<100 IU/mL at screening and have good hepatic reserve and low levels of hepatic inflammation, as determined by lack of fibrosis/cirrhosis and ALT <2×ULN. Five dose level cohorts are used for Part B. Doses increase stepwise by a factor of approximately 3 to 4-fold to a maximum planned dose of 900 mg administered by SC injection:

Two optional cohorts are enrolled up to a maximum dose of 900 mg administered by SC injection. Cohort 7b may be enrolled for the purpose of, but not limited to, collection and evaluation of immune response samples and hepatic fine needle aspirate samples at select sites when and where available. These dose levels are based on preclinical animal models and translational PK/PD modeling that predict a significant HBsAg decline for doses in the range of 2 to 15 mg/kg. Details on the dose escalation plan for Part B can be found in Table 9.

TABLE 9
Part B Dose Escalation Plan

| Study | | | | Frequency of |
|---|---|---|---|---|
| Part | Cohort | Active:Placebo | Dose (Route) | Administration |
| B | 1b | 6:2 | 6 mg (SC) | Once |
| | 2b | 6:2 | 18 mg (SC) | Once |
| | 3b | 6:2 | Up to 75 mg | Once |
| | | | (SC) | |
| | 4b | 6:2 | Up to 300 mg | Once |
| | | | (SC) | |
| | 5b | 6:2 | Up to 900 mg | Once |
| | | | (SC) | |
| | 6b | 6:2 | Up to 900 mg | Once |
| | (Optional) | | (SC) | |
| | 7b | 6:2 | Up to 900 mg | Once |
| | (Optional) | | (SC) | |

SC = subcutaneous

Optional Part C: Single Ascending Dose Study in Subjects with Chronic HBV Infection

To evaluate the safety, tolerability and anti-viral activity of HBC34-v35-MLNS-GAALIE in subjects with a baseline HBsAg level ≥1000 IU/mL, an optional Part C is conducted after the safety, tolerability, and antiviral activity of HBC34-v35-MLNS-GAALIE has been established in HBeAg-negative subjects with HBsAg<1000 IU/mL in Part B. Part C consists of three optional dose level cohorts, with each evaluating a dose of up to 900 mg administered by SC injection. Table 10. One or more of the optional cohorts in Part C may be enrolled for the purpose of, but not limited to, collection and evaluation of immune response samples and hepatic fine needle aspirate samples at select sites when and where available.

TABLE 10
Part C Cohort Overview

| Study Part | Cohort | Route | Dose Level | Active:Placebo |
|---|---|---|---|---|
| C | 1c (optional) | SC | up to 900 mg | 6:2 |
| C | 2c (optional) | SC | up to 900 mg | 6:2 |
| C | 3c (optional) | SC | up to 900 mg | 6:2 |

### Reference Therapy, Dosage, and Mode of Administration:

Subjects randomized to placebo are administered sterile, preservative-free normal saline 0.9% solution by SC injection (Parts A, B, and C) or IV infusion (Part A only).

### Local Tolerability

For all study parts, a local tolerability assessment is performed per the Schedule of Assessments (Figure for subjects receiving study drug by SC injection. Injection site(s) will be marked and mapped for later observation and should be documented. Injection site(s) should be monitored for pain/tenderness, swelling, redness, bruising, and pruritus.

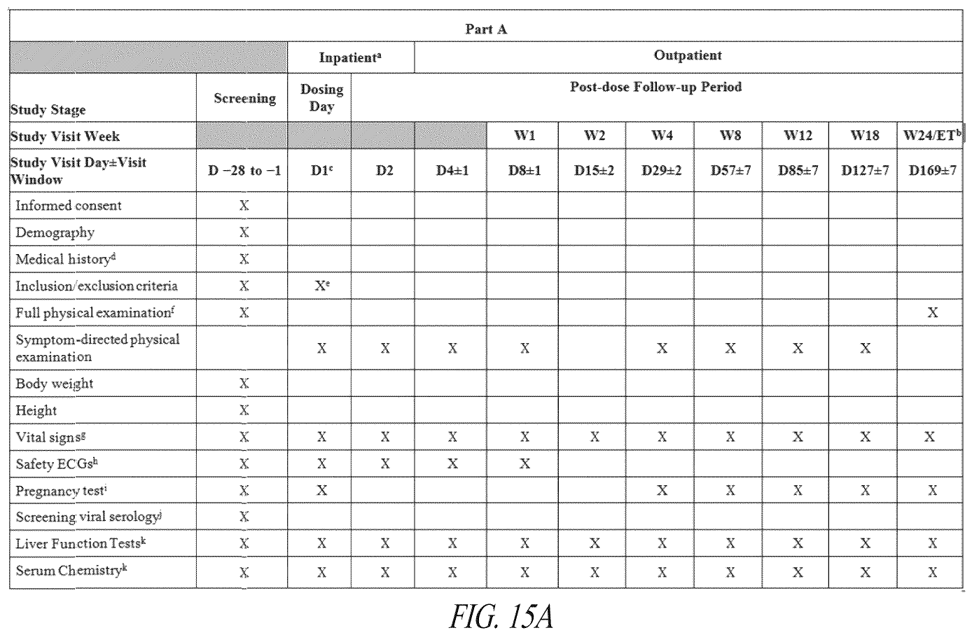

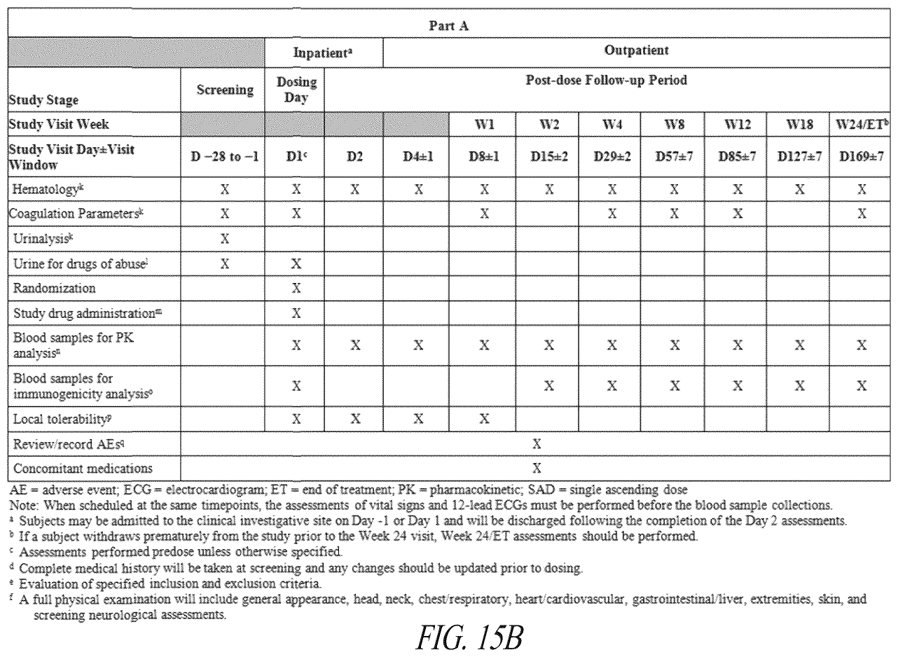

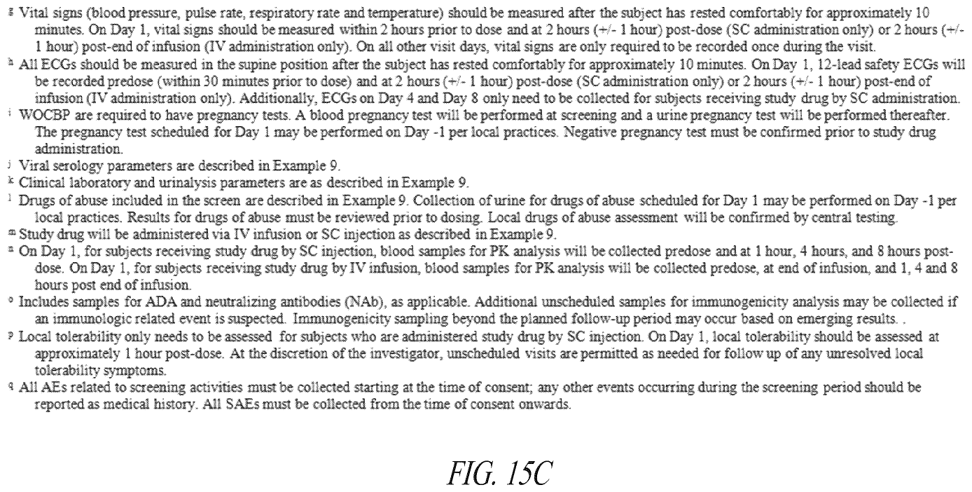

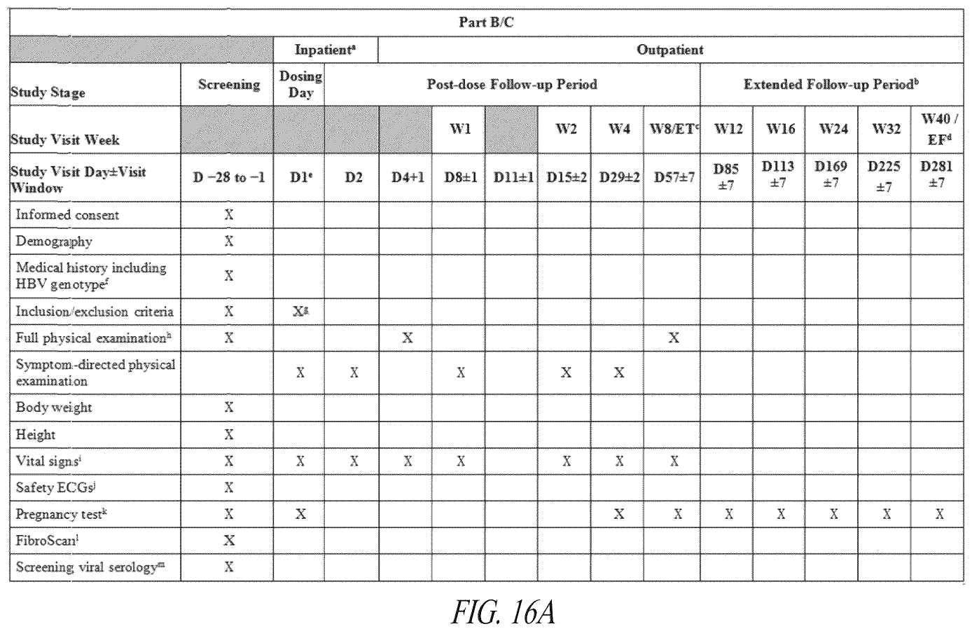

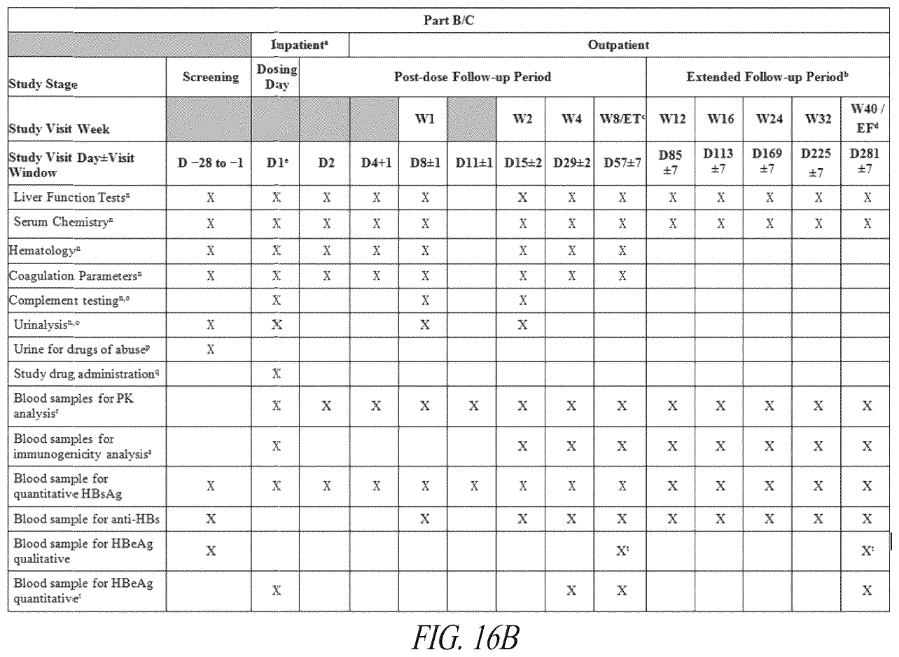

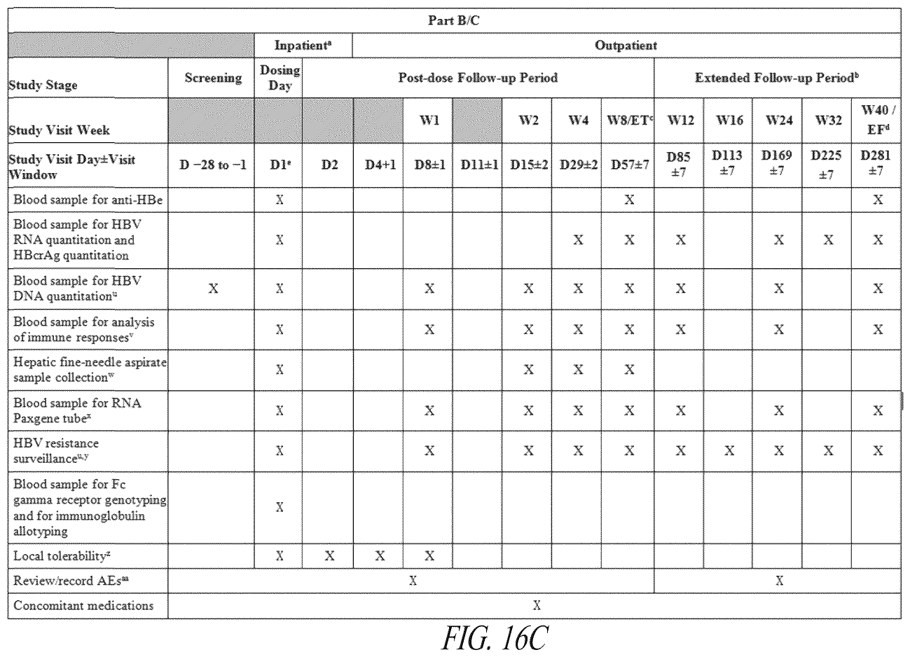

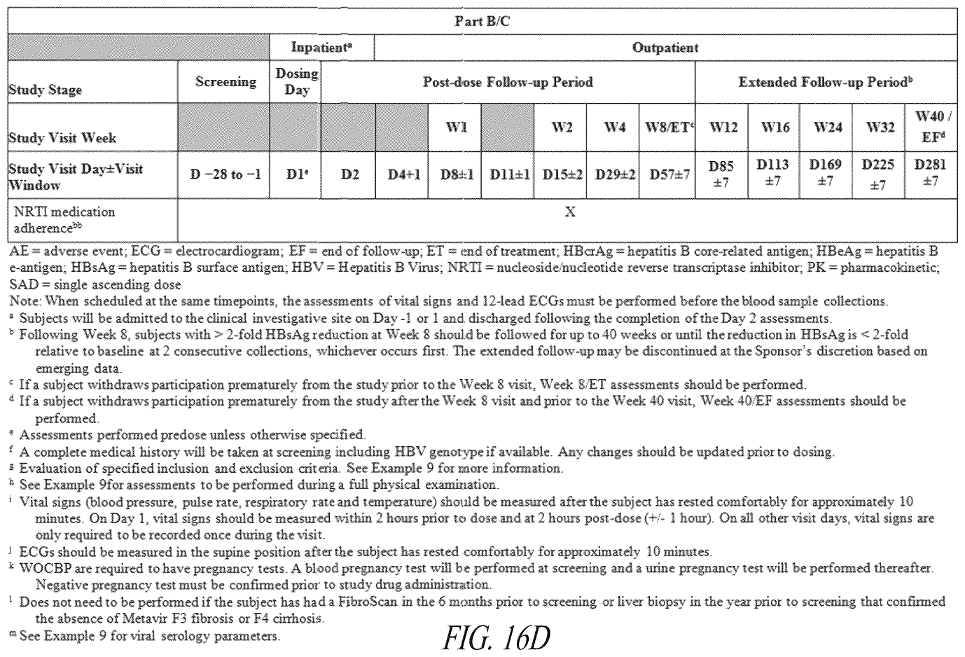

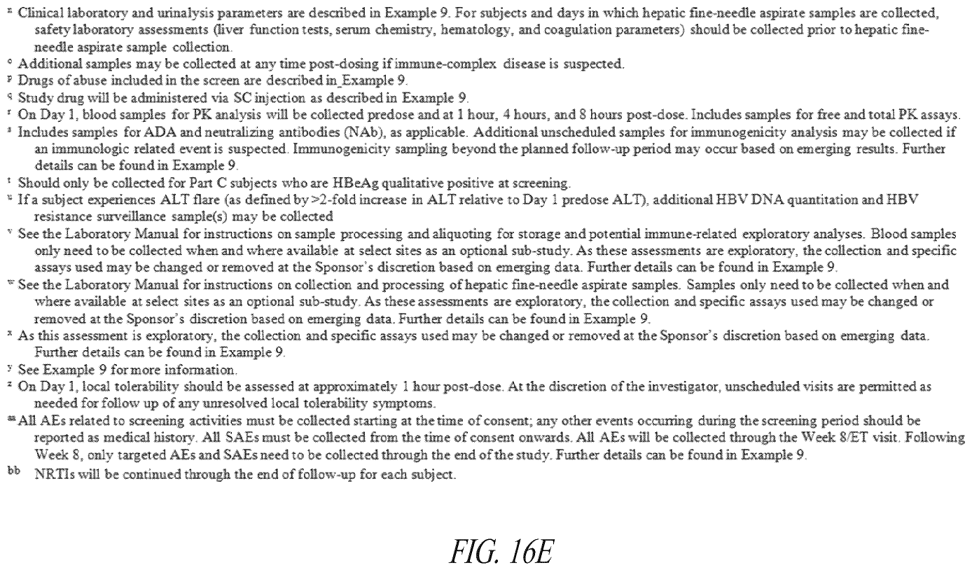

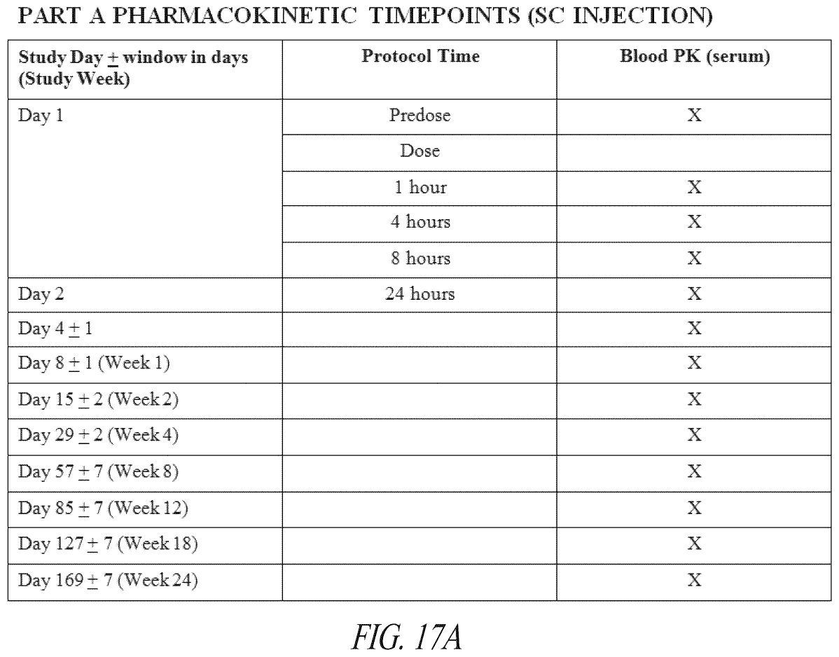

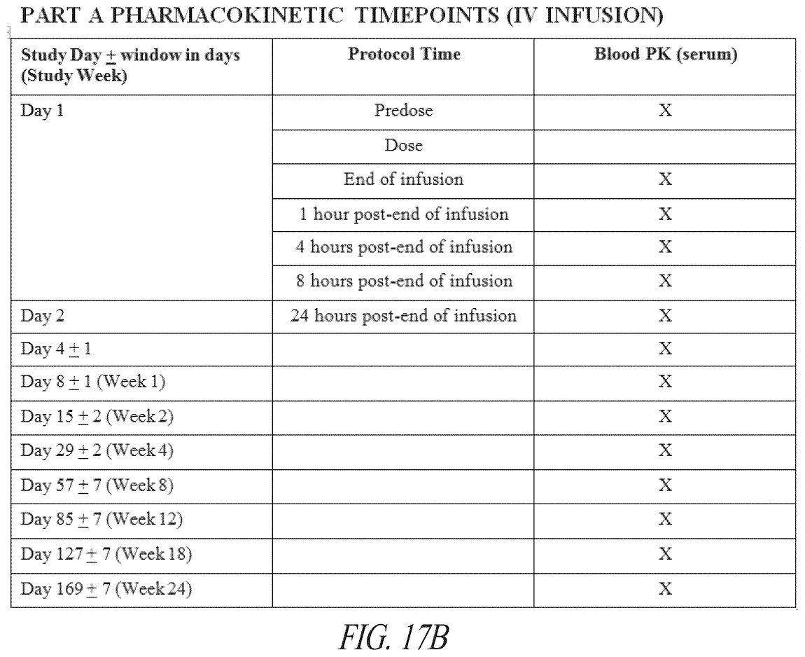

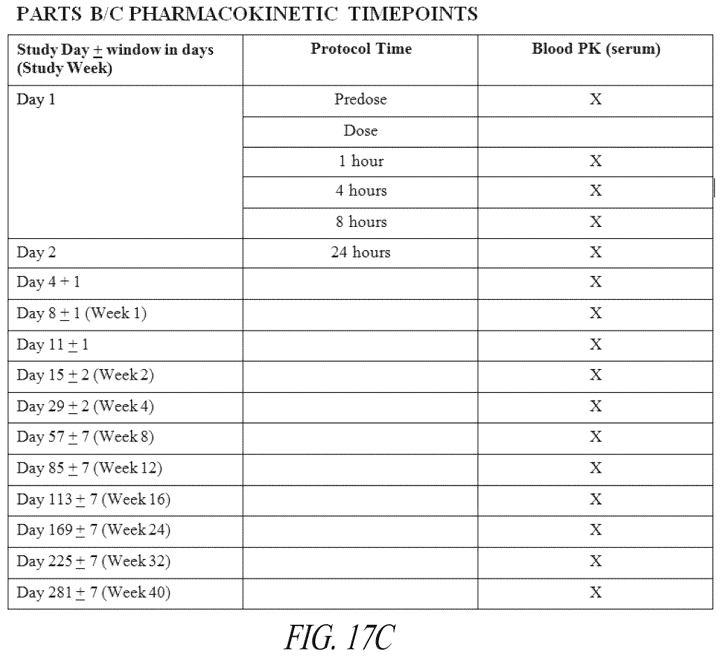

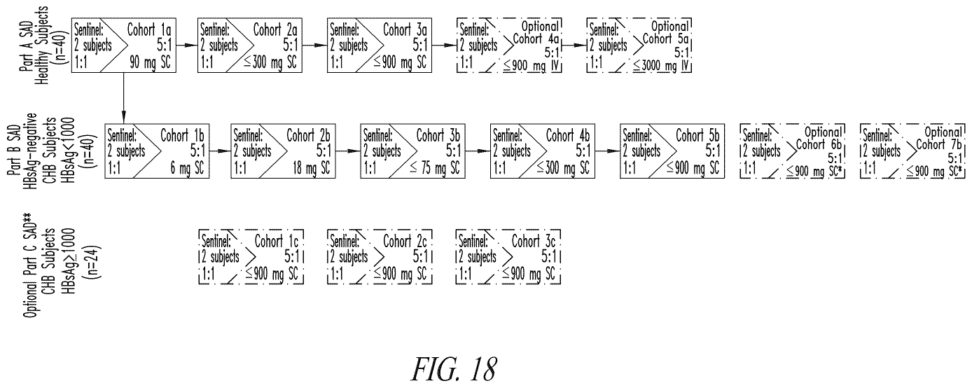

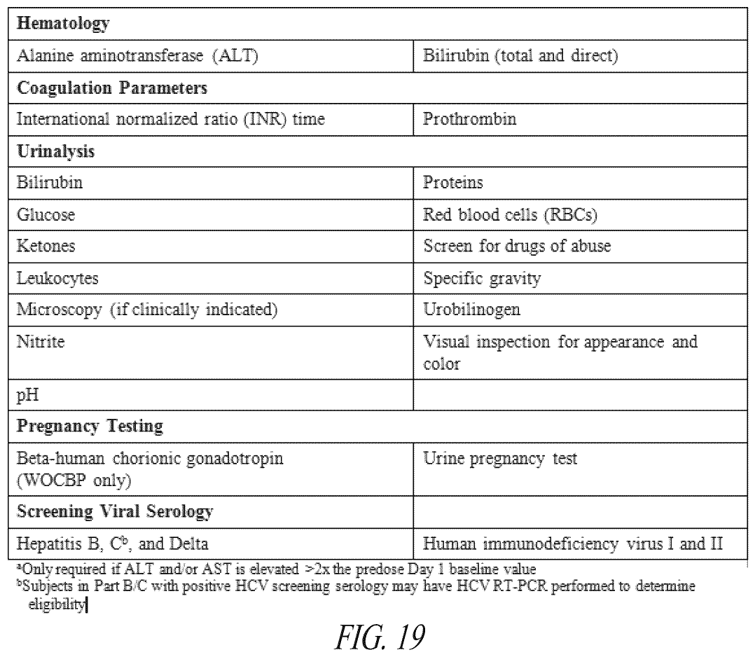

The timing of local tolerability assessments for Part A is shown in FIGS. 15A-15C. The timing of the local tolerability assessments for Parts B/C is shown in FIGS. 16A-16E.

At the discretion of the investigator, unscheduled visits are permitted as needed for follow up of any unresolved local tolerability symptoms.

### Screening for Drugs of Abuse

For Parts A, B, and C of the study, urine is collected for drugs of abuse screening. The panel includes amphetamines, cocaine, methadone, and opiates.

### Pharmacokinetic Assessments

Blood samples will be collected to assess concentrations of HBC34-v35-MLNS-GAALIE. Timepoints for the collection of samples for HBC34-v35-MLNS-GAALIE PK analysis for Part A of the study are provided herein. Timepoints for the collection of samples for HBC34-v35-MLNS-GAALIE PK analysis for Parts B and C of the study are provided herein.

### Pharmacokinetic Analysis

### Part A

Free PK parameters of HBC34-v35-MLNS-GAALIE are computed using standard noncompartmental methods. Parameters include, but not be limited to, serum: Cₘₐₓ, Cₗₐₛₜ, Tₘₐₓ, Tₗₐₛₜ, AUC_{inf}, AUCₗₐₛₜ, % AUCₑₓₚ, t_{1/2}, λ_{z}, V_{z} (IV only), CL (IV only), V_{z}/F (SC only), and CL/F (SC only). Other parameters are calculated as necessary.

### Parts B/C

Free and total PK parameters of HBC34-v35-MLNS-GAALIE are computed using standard noncompartmental methods. Parameters include, but are not limited to, serum: Cₘₐₓ, Cₗₐₛₜ, Tₘₐₓ, Tₗₐₛₜ, AUC_{inf}, AUCₗₐₛₜ, % AUCₑₓₚ, t_{1/2}, λz, V_{z}/F, and CL/F. Other parameters are calculated as necessary.

PK/pharmacodynamic analyses are conducted to explore exposure-response relationships between PK parameters and selected antiviral variables.

### Antiviral Activity Analysis

For Parts B and C, selected data relating to the antiviral activity of HBC34-v35-MLNS-GAALIE, such as HBsAg, anti-HBs, HBeAg, anti-HBe, HBV RNA, HBcrAg, and HBV DNA levels, are summarized (n, mean, SD, median, Q1, Q3, minimum, and maximum) by cohort and study visit along with corresponding change from baseline. Summaries (number and percentage of subjects) of HBsAg loss (defined as undetectable HBsAg measured on 2 separate, consecutive occasions, at least 2 weeks apart) are provided by cohort and study visit.

### Immunogenicity

Blood samples are collected for analysis of immunogenic responses to determine presence/absence and titers of anti-drug antibodies (ADA) as applicable, according to the time points defined in the Schedule of Assessments (FIGS. 15A-16E). Samples are characterized for neutralizing potential of HBC34-v35-MLNS-GAALIE (NAb), as appropriate.

### Assessment of Screening Viral Parameters, Antiviral Activity, and Resistance Surveillance

During Parts B and C, assessment of screening viral parameters include: HBsAg, anti-HBS, HBeAg (qualitative), and HBV DNA.

Assessments of antiviral activity performed after screening include: HBsAg, anti-HBS, HBeAg (qualitative; should only be collected for Part C subjects who are HBeAg qualitative positive at screening), HBeAg (quantitative; should only be collected for Part C subjects who are HBeAg qualitative positive at screening), anti-HBe, HBV RNA, hepatitis B core-related antigen (HBcrAg), and HBV DNA.

Resistance surveillance to monitor for the potential development of resistance to NRTIs or HBC34-v35-MLNS-GAALIE is conducted for all subjects who receive study drug. HBV genome sequencing is attempted in subjects with confirmed HBV DNA breakthrough as defined by HBV DNA≥500 IU/mL measured at 2 consecutive study visits, or subjects who discontinue early from the study with HBV DNA≥500 IU/mL. As it will not be known at the time of visit if a subject has virologic breakthrough, samples for resistance surveillance is collected at all study visits noted in the SOA. Samples collected for resistance surveillance may be used to perform additional viral analyses, including viral sequencing.

### Assessment of Immune Responses

To examine the host immune response and potential biomarkers of infection, subjects may consent to optional sub-studies in which peripheral immune samples with or without hepatic immune samples (via fine-needle aspiration) will be collected at the timepoints outlined in FIGS. 15A-16E. These optional sub-studies and associated assessments are done when and where available at select sites.

### Fc Gamma Receptor (FcγR) Genotyping and Immunoglobulin Allotyping

Blood samples for FcγR genotyping and immunoglobulin allotyping are collected at baseline for all subjects in Parts B and C to evaluate a possible association between Fc-gamma receptor polymorphisms or immunoglobulin allotype with antiviral activity of HBC34-v35-MLNS-GAALIE.

### Statistical Methods

Statistical analyses are primarily descriptive. All study data are presented by subject data listings. For all Study Parts, summary tables present results by cohort for HBC34-v35-MLNS-GAALIE and placebo, where the placebo subjects are combined across dose cohorts by route of administration for each Part.

This study is conducted in accordance with the ethical principles that have their origin in the Declaration of Helsinki, and that are consistent with Good Clinical Practice (GCP) and the applicable regulatory requirement, including archiving of essential documents.

### LIST OF ABBREVIATIONS AND DEFINITIONS OF TERMS IN THIS EXAMPLE

ADA anti-drug antibodies
AE adverse event
ALT alanine aminotransferase
ANC absolute neutrophil count
AP alkaline phosphatase
AST aspartate aminotransferase
AUC area under the curve
BLQ below the limit of quantitation
BMI body mass index
BUN blood urea nitrogen
CLcr creatinine clearance
CRF case report form
CTCAE Common Terminology Criteria for Adverse Events
DNA deoxyribonucleic acid
ECG electrocardiogram
eCRF electronic case report form
EF end of follow-up
ET end of treatment
FDA Food and Drug Administration
GCP Good Clinical Practice
GGT gamma glutamyl transferase
GLP Good Laboratory Practice
GNA glycol nucleic acid
HBcrAg hepatitis B core-related antigen
HBeAg hepatitis B e-antigen
HBIG hepatitis B immune globulin
HBsAg hepatitis B surface antigen
HBV hepatitis B virus
HCC hepatocellular carcinoma
HED human equivalent dose
Hgb hemoglobin
ICF informed consent form
ICH International Conference on Harmonisation
IgG immunoglobulin G
IgM immunoglobulin M
IEC Independent Ethics Committee
INR international normalized ratio
IRB Institutional Review Board
IV intravenous
IWRS interactive web response system
LDH lactate dehydrogenase
LLN lower limit of normal
LLOQ lower limit of quantitation
LLT Lower-Level Term
mAb monoclonal antibody
MedDRA Medical Dictionary for Regulatory Activities
Nab Neutralizing antibodies
NOAEL no observed adverse effect level
OTC over-the-counter
PK pharmacokinetics
PT Preferred Term
Q1 first quartile
Q3 third quartile
RBC red blood cell (count)
RNA ribonucleic acid
SAD single ascending dose
SAE serious adverse event
SC subcutaneous
SD standard deviation
SoA schedule of assessments
SOC System Organ Class
SRC Safety Review Committee
SUSAR suspected unexpected serious adverse reaction
TCR tissue cross reactivity
TEAE treatment-emergent adverse event
US United States
ULN upper limit of normal
WBC white blood cell (count)
WHO World Health Organization
WOCBP women of child-bearing potential

### Example 10: Activation of Dendritic Cells by HBsAg: HBC34-v35 Antibody Immune Complexes

Activation of monocyte-derived (mo)DCs in the presence of immune complexes (ICs), formed by HBC34-V35-MLNS_GAALIE (HC SEQ ID NO.: 91, LC SEQ ID NO.: 93) or HBC34-V35_MLNS (HC SEQ ID NO.: 92, LC SEQ ID NO.: 93) and HBsAg in the serum of HBV+ patients (supplier: BioIVT), was tested.Material and methods:

CD14+ monocytes were isolated from human PBMCs from healthy donors (n=2) and cultured in RPMI 1640, 10% FBS (Hyclone), 1% non-essential amino acids, 1% Glutamine, 1% Pen/Strep, 1% Sodium Pyruvate, 50 μM β-mercaptoethanol, 50 ng/mL GM-CSF (Miltenyi) and 1000U/mL

IL-4 (R&D) for 6 days. The then differentiated immature monocyte-derived DCs (moDCs) were stimulated for 22 hours with HBsAg alone (diluted to final 250 IU/ml of two patient sera at 1890 and 4460 IU/ml), with ICs of HBsAg and HBC34-v35-MLNS or HBC34-v35-MLNS_GAALIE (mAbs at 20-100 μg/ml) or mAb alone. Reagents were tested to be endotoxin free. Surface expression of co-stimulatory markers CD83 and CD86 and HLA-DR was measured via flow cytometry. The levels of ten (10) human proinflammatory cytokines (IFNγ, IL-1β, IL-2, IL-4, IL-6, IL-8, IL-10, IL-12p70, IL-13, and TNFα) were measured using the Meso Scale Diagnostics (MSD) V-PLEX Proinflammatory Panel 1 Human Kit. Culture medium was used as a negative control. LPS (Sigma, 100 ng/ml) served as positive control.

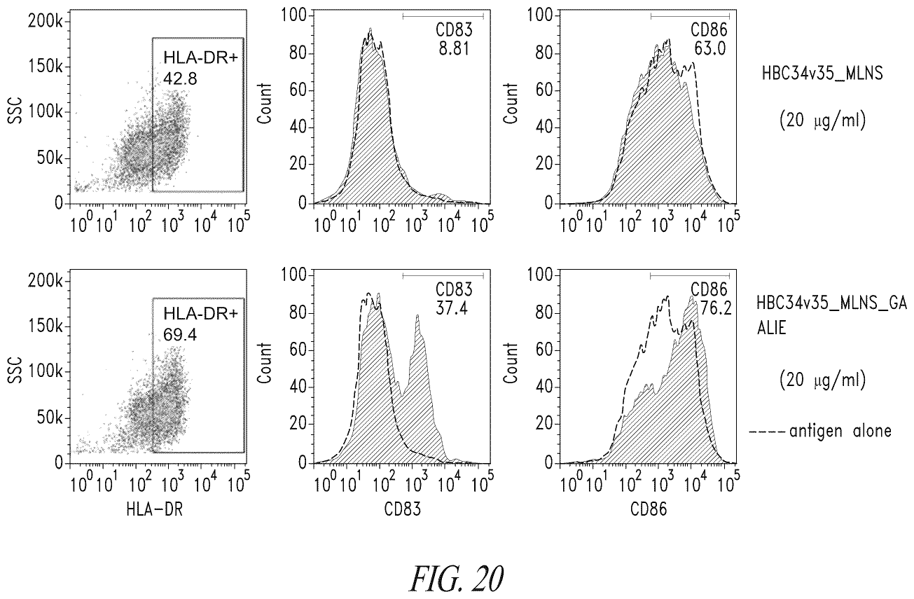

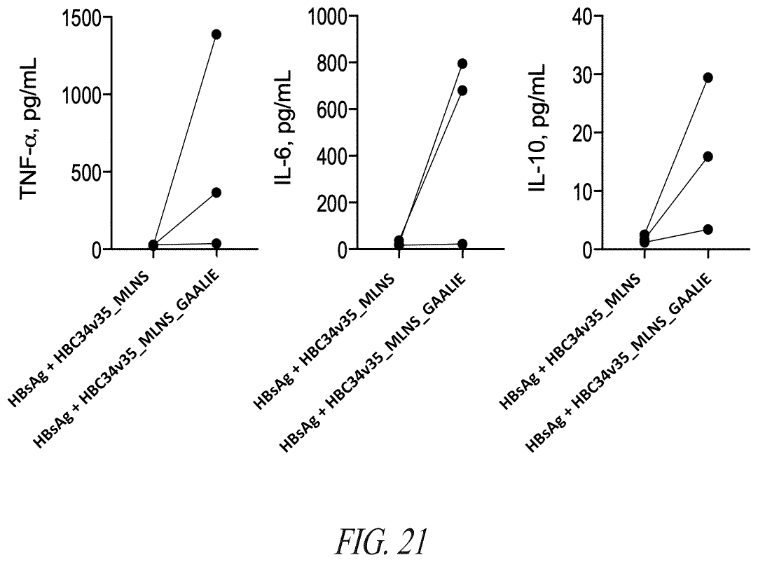

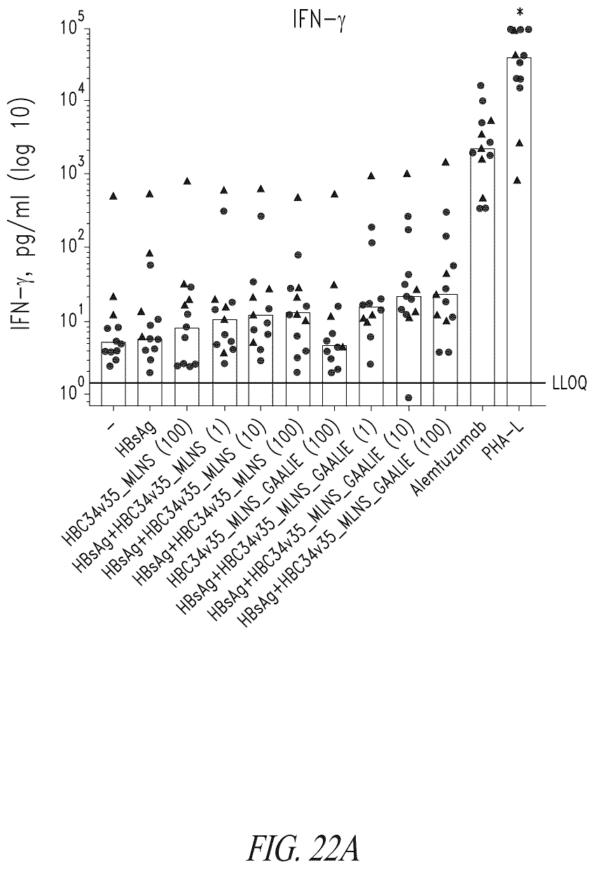

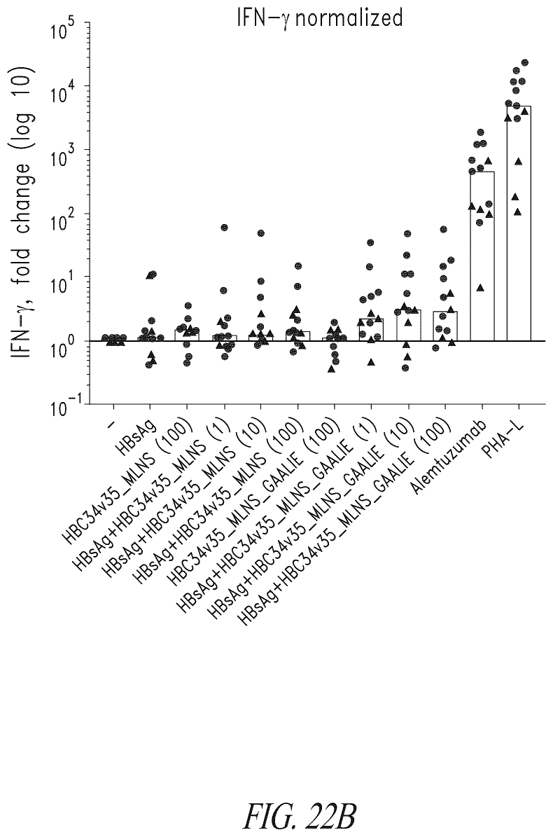

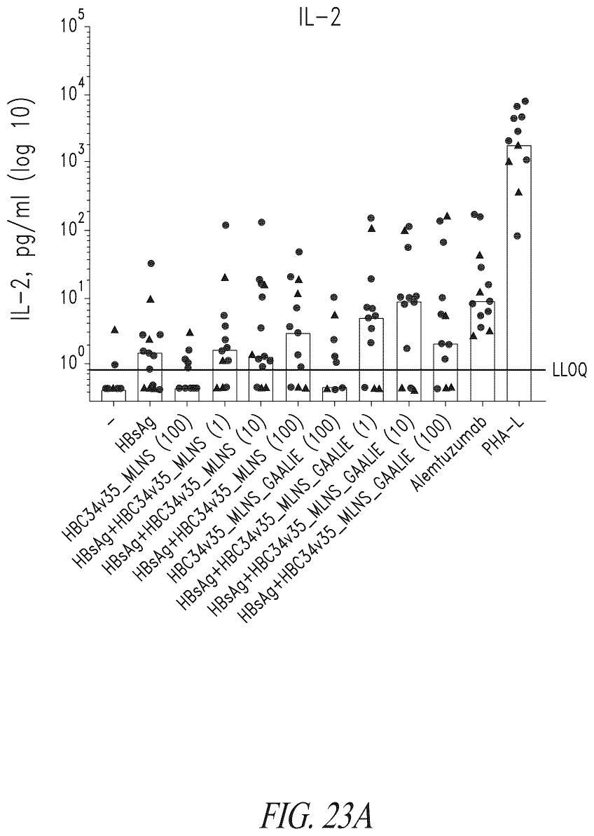

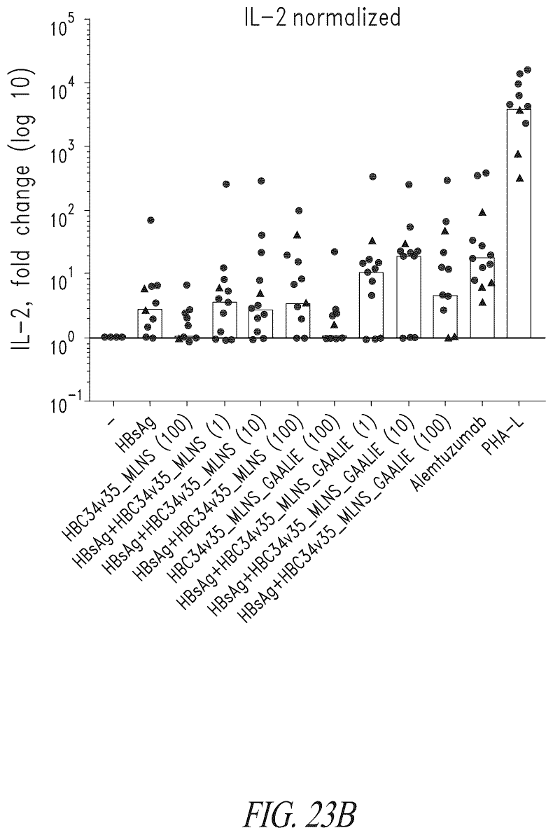

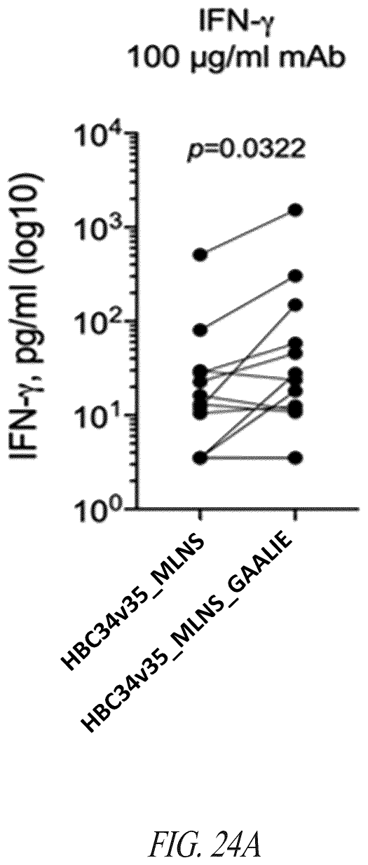

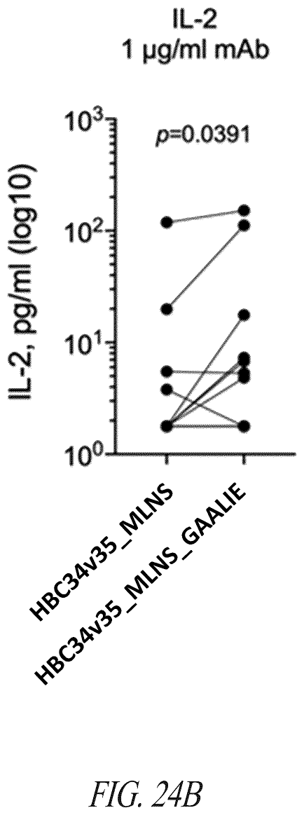

Data are shown in FIGS. 20-24B. Immune complexes (ICs) of HBsAg with HBC34-v35-MLNS-GAALIE induced upregulation of co-stimulatory markers CD83 and CD86, as well as HLA-DR, on the surface of moDCs. In addition, ICs of HBsAg with HBC34-v35-MLNS-GAALIE induced moDCs to secrete cytokines TNFα, IL-6 and IL-10.

TABLE OF SEQUENCES AND SEQ ID NUMBERS (SEQUENCE LISTING):

| SEQ | | |
|---|---|---|
| ID | | |
| NO | Sequence | Remarks |
| 1 | X₁ X₂ X₃ TC X₄ X₅ X₆A X₇G | epitope |
| | wherein X₁, X₂, X₃, X₄, X₅, X₆ and X₇ may be any amino acid | |
| 2 | X₁ X₂ X₃ TC X₄ X₅ X₆A X₇G | |
| | wherein X₁ is P, T or S, | |
| | X₂ is C or S, | |
| | X₃ is R, K, D or I, | |
| | X₄ is M or T, | |
| | X₅ is T, A or I, | |
| | [symbol] | |
| 3 | MENITSGFLGPLLVLQAGFFLLTRILTIPQSLDSWWTSLNF | S domain of HBsAg |
| | LGGTTVCLGQNSQSPTSNHSPTSCPPTCPGYRWMCLRRFI | (GenBank acc. no. |
| | IFLFILLLCLIFLLVLLDYQGMLPVCPLIPGSSTTSTGPCRT | J02203) |
| | CMTTAQGTSMYPSCCCTKPSDGNCTCIPIPSSWAFGKFL | |
| | WEWASARFSWLSLLVPFVQWFVGLSPTVWLSVIWMMW | |
| | YWGPSLYSILSPFLPLLPIFFCLWVYI | |
| 4 | MENVTSGFLGPLLVLQAGFFLLTRILTIPQSLDSWWTSLN | S domain of HBsAg |
| | FLGGTTVCLGQNSQSPTSNHSPTSCPPTCPGYRWMCLRR | (GenBank acc. no. |
| | FIIFLFILLLCLIFLLVLLDYQGMLPVCPLIPGSSTTGTGPCR | FJ899792) |
| | TCTTPAQGTSMYPSCCCTKPSDGNCTCIPIPSSWAFGKFL | |
| | WEWASARFSWLSLLVPFVQWFVGLSPTVWLSVIWMMW | |
| | YWGPSLYSTLSPFLPLLPIFFCLWVYI | |
| 5 | QGMLPVCPLIPGSSTTSTGPCRTCMTTAQGTSMYPSCCCT | J02203 (D, ayw3) |
| | KPSDGNCTCIPIPSSWAFGKFLWEWASARFSW | |
| 6 | QGMLPVCPLIPGSSTTGTGPCRTCTTPAQGTSMYPSCCCT | FJ899792 (D, adw2) |
| | KPSDGNCTCIPIPSSWAFGKFLWEWASARFSW | |
| 7 | QGMLPVCPLIPGTTTTSTGPCKTCTTPAQGNSMFPSCCCT | AM282986 (A) |
| | KPSDGNCTCIPIPSSWAFAKYLWEWASVRFSW | |
| 8 | QGMLPVCPLIPGSSTTSTGPCKTCTTPAQGTSMFPSCCCT | D23678 (B1) |
| | KPTDGNCTCIPIPSSWAFAKYLWEWASVRFSW | |
| 9 | QGMLPVCPLLPGTSTTSTGPCKTCTIPAQGTSMFPSCCCT | AB117758 (C1) |
| | KPSDGNCTCIPIPSSWAFARFLWEWASVRFSW | |
| 10 | QGMLPVCPLIPGSSTTSTGPCRTCTTLAQGTSMFPSCCCS | AB205192 (E) |
| | KPSDGNCTCIPIPSSWAFGKFLWEWASARFSW | |
| 11 | QGMLPVCPLLPGSTTTSTGPCKTCTTLAQGTSMFPSCCCS | X69798 (F4) |
| | KPSDGNCTCIPIPSSWALGKYLWEWASARFSW | |
| 12 | QGMLPVCPLIPGSSTTSTGPCKTCTTPAQGNSMYPSCCCT | AFI60501(G) |
| | KPSDGNCTCIPIPSSWAFAKYLWEWASVRFSW | |
| 13 | QGMLPVCPLLPGSTTTSTGPCKTCTTLAQGTSMFPSCCCT | AY090454 (H) |
| | KPSDGNCTCIPIPSSWAFGKYLWEWASARFSW | |
| 14 | QGMLPVCPLIPGSSTTSTGPCKTCTTPAQGNSMYPSCCCT | AF241409 (I) |
| | KPSDGNCTCIPIPSSWAFAKYLWEWASARFSW | |
| 15 | QGMLPVCPLLPGSTTTSTGPCRTCTITAQGTSMFPSCCCT | AB486012 (J) |
| | KPSDGNCTCIPIPSSWAFAKFLWEWASVRFSW | |
| 16 | CQGMLPVCPLIPGSSTTGTGTCRTCTTPAQGTSMYPSCCC | HBsAg Y100C/P120T |
| | TKPSDGNCTCIPIPSSWAFGKFLWEWASARFSW | |
| 17 | QGMLPVCPLIPGSSTTGTGTCRTCTTPAQGTSMYPSCCCT | HBsAg P120T |
| | KPSDGNCTCIPIPSSWAFGKFLWEWASARFSW | |
| 18 | QGMLPVCPLIPGSSTTGTGTCRTCTTPAQGTSMYPSCCCT | HBsAg P120T/S143L |
| | KPLDGNCTCIPIPSSWAFGKFLWEWASARFSW | |
| 19 | QGMLPVCPLIPGSSTTGTGPSRTCTTPAQGTSMYPSCCCT | HBsAg C121S |
| | KPSDGNCTCIPIPSSWAFGKFLWEWASARFSW | |
| 20 | QGMLPVCPLIPGSSTTGTGPCDTCTTPAQGTSMYPSCCCT | HBsAg R122D |
| | KPSDGNCTCIPIPSSWAFGKFLWEWASARFSW | |
| 21 | QGMLPVCPLIPGSSTTGTGPCITCTTPAQGTSMYPSCCCT | HBsAg R122I |
| | KPSDGNCTCIPIPSSWAFGKFLWEWASARFSW | |
| 22 | QGMLPVCPLIPGSSTTGTGPCRNCTTPAQGTSMYPSCCCT | HBsAg T123N |
| | KPSDGNCTCIPIPSSWAFGKFLWEWASARFSW | |
| 23 | QGMLPVCPLIPGSSTTGTGPCRTCTTPAHGTSMYPSCCCT | HBsAg Q129H |
| | KPSDGNCTCIPIPSSWAFGKFLWEWASARFSW | |
| 24 | QGMLPVCPLIPGSSTTGTGPCRTCTTPALGTSMYPSCCCT | HBsAg Q129L |
| | KPSDGNCTCIPIPSSWAFGKFLWEWASARFSW | |
| 25 | QGMLPVCPLIPGSSTTGTGPCRTCTTPAQGTSHYPSCCCT | HBsAg M133H |
| | KPSDGNCTCIPIPSSWAFGKFLWEWASARFSW | |
| 26 | QGMLPVCPLIPGSSTTGTGPCRTCTTPAQGTSLYPSCCCT | HBsAg M133L |
| | KPSDGNCTCIPIPSSWAFGKFLWEWASARFSW | |
| 27 | QGMLPVCPLIPGSSTTGTGPCRTCTTPAQGTSTYPSCCCT | HBsAg M133T |
| | KPSDGNCTCIPIPSSWAFGKFLWEWASARFSW | |
| 28 | QGMLPVCPLIPGSSTTGTGPCRTCTTPAQGTSMYPSCCCT | HBsAg K141E |
| | EPSDGNCTCIPIPSSWAFGKFLWEWASARFSW | |
| 29 | QGMLPVCPLIPGSSTTGTGPCRTCTTPAQGTSMYPSCCCT | HBsAg P142S |
| | KSSDGNCTCIPIPSSWAFGKFLWEWASARFSW | |
| 30 | QGMLPVCPLIPGSSTTGTGPCRTCTTPAQGTSMYPSCCCT | HBsAg S143K |
| | KPKDGNCTCIPIPSSWAFGKFLWEWASARFSW | |
| 31 | QGMLPVCPLIPGSSTTGTGPCRTCTTPAQGTSMYPSCCCT | HBsAg D144A |
| | KPSAGNCTCIPIPSSWAFGKFLWEWASARFSW | |
| 32 | QGMLPVCPLIPGSSTTGTGPCRTCTTPAQGTSMYPSCCCT | HBsAg G145R |
| | KPSDRNCTCIPIPSSWAFGKFLWEWASARFSW | |
| 33 | QGMLPVCPLIPGSSTTGTGPCRTCTTPAQGTSMYPSCCCT | HBsAg N146A |
| | KPSDGACTCIPIPSSWAFGKFLWEWASARFSW | |
| 34 | GRIFRSFY | HBC34 CDRH1 aa |
| 35 | NQDGSEK | HBC34 CDRH2 aa |
| 36 | AAWSGNSGGMDV | HBC34 CDRH3 aa |
| 37 | KLGNKN | HBC34 CDRL1 aa |
| 38 | EVK | HBC34 CDRL2 aa |
| 39 | VIYEVKYRP | HBC34 CDRL2 long |
| | | aa |
| 40 | QTWDSTTVV | HBC34 CDRL3 aa |
| 41 | ELQLVESGGGWVQPGGSQRLSCAASGRIFRSFYMSWVR | HBC34, HBC34-V7, |
| | QAPGKGLEWVATINQDGSEKLYVDSVKGRFTISRDNAK | HBC34-V34, HBC34- |
| | NSLFLQMNNLRVEDTAVYYCAAWSGNSGGMDVWGQG | V35 VH aa |
| | TTVSVSS | |
| 42 | SYELTQPPSVSVSPGQTVSIPCSGDKLGNKNVCWFQHKP | HBC34 VL aa |
| | GQSPVLVIYEVKYRPSGIPERFSGSNSGNTATLTISGTQAM | |
| | DEAAYFCQTWDSTTVVFGGGTRLTVL | |
| 43 | GGACGCATCTTTAGAAGTTTTTAC | HBC34 CDRH1 nuc |
| 44 | ATAAACCAAGATGGAAGTGAGAAA | HBC34 CDRH2 nuc |
| 45 | GCGGCTTGGAGCGGCAATAGTGGGGGTATGGACGTC | HBC34 CDRH3 nuc |
| 46 | AAATTGGGGAATAAAAAT | HBC34 CDRL1 nuc |
| 47 | GAGGTTAAA | HBC34 CDRL2 nuc |
| 48 | gtcatctatGAGGTTAAAtaccgcccc | HBC34 CDRL2 long |
| | | nuc |
| 49 | CAGACGTGGGACAGCACCACTGTGGTG | HBC34 CDRL3 nuc |
| 50 | GAACTGCAGCTGGTGGAGTCTGGGGGAGGCTGGGTCC | HBC34 VH nuc |
| | AGCCGGGGGGGTCCCAGAGACTGTCCTGTGCAGCCTC | |
| | TGGACGCATCTTTAGAAGTTTTTACATGAGCTGGGTC | |
| | CGCCAGGCCCCAGGGAAGGGGCTGGAGTGGGTGGCCA | |
| | CTATAAACCAAGATGGAAGTGAGAAATTATATGTGG | |
| | ACTCTGTGAAGGGCCGATTCACCATCTCCAGAGACAA | |
| | CGCCAAGAACTCACTATTTCTGCAAATGAACAACCTGA | |
| | GAGTCGAGGACACGGCCGTTTATTACTGCGCGGCTTG | |
| | GAGCGGCAATAGTGGGGGTATGGACGTCTGGGGCC | |
| | AGGGGACCACGGTCTCCGTCTCCTCA | |
| 51 | TCCTATGAGCTGACTCAGCCACCCTCAGTGTCCGTGTC | HBC34 VL nuc |
| | CCCAGGACAGACAGTCAGCATCCCCTGCTCTGGAGAT | |
| | AAATTGGGGAATAAAAATGTTTGCTGGTTTCAGCATA | |
| | AGCCAGGCCAGTCCCCTGTGTTGGTCATCTATGAGGTT | |
| | AAATACCGCCCCTCGGGGATTCCTGAGCGATTCTCTGG | |
| | CTCCAACTCTGGGAACACAGCCACTCTGACCATCAGCG | |
| | GGACCCAGGCTATGGATGAGGCTGCCTATTTCTGTCAG | |
| | ACGTGGGACAGCACCACTGTGGTGTTCGGCGGAGGG | |
| | ACCAGGCTGACCGTCCTA | |
| 52 | XGSSTTSTGPCRTCMTXPSDGNATAIPIPSSWX | peptide |
| | wherein the residues coded as X were substituted with Cysteines | |
| 53 | TSTGPCRTCMTTAQG | peptide |
| 54 | GMLPVCPLIPGSSTTSTGPCRTCMTT | peptide |
| 55 | XSMYPSASATKPSDGNXTGPCRTCMTTAQGTSX | peptide |
| | wherein the residues coded as X were substituted with Cysteines | |
| 56 | PCRTCMTTAQG | amino acids 120-130 |
| | | of the S domain of |
| | | HBsAg (HBV-D |
| | | J02203 |
| 57 | PCX₁TCX₂X₃X₄AQG, | epitope |
| | wherein X₁ is R or K, | |
| | X₂ is M or T, | |
| | X₃ is T or I, and | |
| 58 | QTFDSTTVV | HBC34-V7 CDRL3 |
| | | and HBC34-V23 |
| | | CDRL3 (aa) |
| 59 | SYELTQPPSVSVSPGQTVSIPCSGDKLGNKNVCWFQHKPG | HBC34-V7 VL |
| | QSPVLVIYEVKYRPSGIPERFSGSNSGNTATLTISGTQAMD | |
| | EAAYFCQTFDSTTVVFGGGTRLTVL | |
| 60 | AAGCTGGGGAACAAAAAT | HBC34v7 CDRL1 |
| | | and HBC-V23 |
| | | CDRL1 (nuc) |
| 61 | GAGGTGAAA | HBC34-V7 CDRL2 |
| | | and HBC34v23 |
| | | CDRL2 nuc |
| 62 | GTCATCTACGAGGTGAAATATCGGCCT | HBC34-V7 CDRL2 |
| | | long and CDRL2 |
| | | HBC34-V23 long nuc |
| 63 | CAGACATTCGATTCCACCACAGTGGTC | CDRL3 HBC34-V7 |
| | | and CDRL3 HBC34- |
| | | V23 nuc |
| 64 | TCTTACGAGCTGACACAGCCACCTAGCGTGTCCGTCTCT | HBC34-V7, HBC34- |
| | CCAGGACAGACCGTGTCCATCCCTTGCTCTGGCGACAA | V34, and HBC34-V35 |
| | GCTGGGGAACAAAAATGTCTGTTGGTTCCAGCACAAG | VL nuc |
| | CCAGGGCAGAGTCCCGTGCTGGTCATCTACGAGGTGA | |
| | AATATCGGCCTTCAGGAATTCCAGAACGGTTCAGCGGA | |
| | TCAAACAGCGGCAATACTGCAACCCTGACAATTAGCGG | |
| | GACCCAGGCCATGGACGAAGCCGCTTATTTCTGCCAGA | |
| | CATTCGATTCCACCACAGTGGTCTTTGGCGGGGGAAC | |
| | TAGGCTGACCGTGCTG | |
| 65 | SYELTQPPSVSVSPGQTASITCSGDKLGNKNACWYQQKP | HBC34-V23 VL aa |
| | GQSPVLVIYEVKYRPSGIPERFSGSNSGNTATLTISGTQAM | |
| | DEADYYCQTFDSTTVVFGGGTKLTVL | |
| 66 | INQDGSEK | HBC34 wt CDRH2 aa |
| 67 | EVQLVESGGGLVQPGGSLRLSCAASGRIFRSFYMSWVRQ | HBC34-V31, HBC34- |
| | APGKGLEWVANINQDGSEKLYVDSVKGRFTISRDNAKNS | V32 and HBC34-V33 |
| | LFLQMNNLRVEDTAVYYCAAWSGNSGGMDVWGQGTTV | VH |
| 68 | GAGGTGCAGCTGGTGGAATCCGGCGGGGGACTGGTGC | HBC34-V31, HBC34- |
| | AGCCTGGCGGCTCACTGAGACTGAGCTGTGCAGCTTCT | V32 and HBC34-V33 |
| | GGAAGAATCTTCAGATCTTTTTACATGAGTTGGGTGAG | VH (nuc) |
| | ACAGGCTCCTGGGAAGGGACTGGAGTGGGTCGCAAAC | |
| | ATCAATCAGGACGGATCAGAAAAGCTGTATGTGGATAG | |
| | CGTCAAAGGCAGGTTCACTATTTCCCGCGACAACGCCA | |
| | AAAATTCTCTGTTTCTGCAGATGAACAATCTGCGGGTG | |
| | GAGGATACCGCTGTCTACTATTGTGCAGCCTGGTCTGG | |
| | CAACAGTGGAGGCATGGACGTGTGGGGACAGGGAACC | |
| | ACAGTGACAGTCAGCTCC | |
| 69 | TCTTACGAGCTGACACAGCCCCCTAGCGTGTCCGTCTCT | HBC34v23 VL nuc |
| | CCAGGCCAGACAGCATCCATCACTTGCTCTGGCGACAA | |
| | GCTGGGGAACAAAAATGCCTGTTGGTATCAGCAGAAG | |
| | CCAGGGCAGAGTCCCGTGCTGGTCATCTACGAGGTGA | |
| | AATATCGGCCTTCAGGAATTCCAGAAAGATTCAGTGGA | |
| | TCAAACAGCGGCAATACTGCTACCCTGACAATTAGCGG | |
| | GACCCAGGCCATGGACGAAGCTGATTACTATTGCCAGA | |
| | CATTCGATTCCACCACAGTGGTCTTTGGCGGGGGAAC | |
| | TAAGCTGACCGTGCTG | |
| 70 | GAACTGCAGCTGGTCGAATCAGGAGGAGGGTGGGTCC | HBC34 wt |
| | AGCCCGGAGGGAGCCAGAGACTGTCTTGTGCCGCATCA | VH codon optimized |
| | GGGAGGATCTTCAGGAGCTTCTACATGTCCTGGGTGC | |
| | GCCAGGCACCAGGCAAGGGACTGGAGTGGGTCGCCAC | |
| | CATCAACCAGGACGGATCTGAAAAGCTGTATGTGGAT | |
| | AGTGTCAAAGGCCGGTTCACAATTAGCAGAGACAACGC | |
| | TAAAAATTCTCTGTTTCTGCAGATGAACAATCTGCGAG | |
| | TGGAGGATACCGCCGTCTACTATTGCGCCGCTTGGTCT | |
| | GGCAACAGCGGCGGGATGGATGTCTGGGGGCAGGGC | |
| | ACAACAGTGAGCGTCTCTTCC | |
| 71 | TCATACGAACTGACTCAGCCTCCCTCCGTCTCCGTCTCA | HBC34 wt VL codon |
| | CCTGGACAGACCGTCTCAATCCCCTGCTCCGGCGAT | optimized |
| | AAACTGGGCAACAAGAACGTGTGCTGGTTCCAGCACA | |
| | AACCCGGACAGAGTCCTGTGCTGGTCATCTACGAGGTC | |
| | AAGTATCGGCCAAGCGGCATTCCCGAAAGATTCAGCGG | |
| | CTCCAACTCTGGGAATACCGCAACACTGACTATCTCTG | |
| | GAACCCAGGCAATGGACGAGGCAGCTTACTTTTGCCAG | |
| | ACTTGGGATTCAACTACTGTCGTGTTCGGCGGCGGAA | |
| | CTAGACTGACTGTCCTG | |
| 72 | GGGAGGATCTTCAGGAGCTTCTAC | HBC34 wt CDRH1 |
| | | codon optimized |
| 73 | ATCAACCAGGACGGATCTGAAAAG | HBC34 wt CDRH2 |
| | | codon optimized |
| 74 | GCCGCTTGGTCTGGCAACAGCGGCGGGATGGATGTC | HBC34 wt CDRH3 |
| | | codon optimized |
| 75 | AAACTGGGCAACAAGAAC | HBC34 wt CDRL1 |
| | | codon optimized |
| 76 | GAGGTCAAG | HBC34 wt CDRL2 |
| | | codon optimized |
| 77 | GTCATCTACGAGGTCAAGTATCGGCCA | HBC34 wt CDRL2 |
| | | long codon optimized |
| 78 | CAGACTTGGGATTCAACTACTGTCGTG | HBC34 wt CDRL3 |
| | | codon optimized |
| 79 | GGSGG | linker |
| 80 | TGPCRTC | epitope |
| 81 | GNCTCIP | epitope |
| 82 | CCIPIPSSWAFGCSTTSTGPCRTCC | discontinuous epitope |
| | wherein in particular thy cysteines at positions 2, 21, and 24 are | mimic |
| | coupled to acetamidomethyl. | |
| 83 | CGNCTCIPIPSSWAFCSTTSTGPCRTCC | discontinuous epitope |
| | wherein in particular thy cysteines at positions 4, 6, 24, and 27 | mimic |
| | are coupled to acetamidomethyl. | |
| 84 | CGGGCSTTSTGPCRTCC | looped epitope mimic |
| | wherein in particular thy cysteines at positions 13 and 16 are | |
| | coupled to acetamidomethyl. | |
| 85 | STTSTGPCRTC | epitope |
| 86 | GNCTCIPIPSSWAFC | epitope |
| 87 | GNCTCIPIPSSWAF | epitope |
| 88 | PCRXC | epitope |
| 89 | SYELTQPPSVSVSPGQTVSIPCSGDKLGNKNVAWFQHKPG | HBC34-V35 VL |
| | QSPVLVIYEVKYRPSGIPERFSGSNSGNTATLTISGTQAMD | |
| | EAAYFCQTFDSTTVVFGGGTRLTVL | |
| 90 | SYELTQPPSVSVSPGQTVSIPCSGDKLGNKNVSWFQHKPG | HBC34-V34 VL |
| | QSPVLVIYEVKYRPSGIPERFSGSNSGNTATLTISGTQAMD | |
| | EAAYFCQTFDSTTVVFGGGTRLTVL | |
| 91 | ELQLVESGGGWVQPGGSQRLSCAASGRIFRSFYMSWVRQ | HC of HBC34-V35- |
| | APGKGLEWVATINQDGSEKLYVDSVKGRFTISRDNAKNS | MLNS-GAALIE and |
| | LFLQMNNLRVEDTAVYYCAAWSGNSGGMDVWGQGTTV | HBC34-V34-MLNS- |
| | SVSSASTKGPSVFPLAPSSKSTSGGTAALGCLVKDYFPEPV | GAALIE (g1M17, 1) |
| | TVSWNSGALTSGVHTFPAVLQSSGLYSLSSVVTVPSSSLG | |
| | TQTYICNVNHKPSNTKVDKKVEPKSCDKTHTCPPCPAPEL | |
| | LAGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPLPEEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVLHEA | |
| | LHSHYTQKSLSLSPGK | |
| 92 | ELQLVESGGGWVQPGGSQRLSCAASGRIFRSFYMSWVRQ | HC of HBC34-V35- |
| | APGKGLEWVATINQDGSEKLYVDSVKGRFTISRDNAKNS | MLNS and HBC34- |
| | LFLQMNNLRVEDTAVYYCAAWSGNSGGMDVWGQGTTV | V34-MLNS (g1M17, |
| | SVSSASTKGPSVFPLAPSSKSTSGGTAALGCLVKDYFPEPV | 1) |
| | TVSWNSGALTSGVHTFPAVLQSSGLYSLSSVVTVPSSSLG | |
| | TQTYICNVNHKPSNTKVDKKVEPKSCDKTHTCPPCPAPEL | |
| | LGGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVLHEAL | |
| | HSHYTQKSLSLSPGK | |
| 93 | SYELTQPPSVSVSPGQTVSIPCSGDKLGNKNVAWFQHKPG | LC of HBC34-V35 |
| | QSPVLVIYEVKYRPSGIPERFSGSNSGNTATLTISGTQAMD | |
| | EAAYFCQTFDSTTVVFGGGTRLTVLGQPKAAPSVTLFPPS | |
| | SEELQANKATLVCLISDFYPGAVTVAWKADSSPVKAGVE | |
| | TTTPSKQSNNKYAASSYLSLTPEQWKSHRSYSCQVTHEGS | |
| | TVEKTVAPTECS | |
| 94 | SYELTQPPSVSVSPGQTVSIPCSGDKLGNKNVSWFQHKPG | LC of HBC34-V34 |
| | QSPVLVIYEVKYRPSGIPERFSGSNSGNTATLTISGTQAMD | |
| | EAAYFCQTFDSTTVVFGGGTRLTVLGQPKAAPSVTLFPPS | |
| | SEELQANKATLVCLISDFYPGAVTVAWKADSSPVKAGVE | |
| | TTTPSKQSNNKYAASSYLSLTPEQWKSHRSYSCQVTHEGS | |
| | TVEKTVAPTECS | |
| 95 | EVQLLESGGGLVQPGGSLRLSCAASGSTFTKYAMSWVRQ | HBC24 VH |
| | APGKGLEWVASISGSVPGFGIDTYYADSVKGRFTISRDTS | |
| | KNTLYLQMNSLRAEDTALYYCAKDVGVIGSYYYYAMDV | |
| | WGQGTAVTVSS | |
| 96 | EIVLTQSPGTLSLSPGERATLSCRASQGLSSSYLAWYQQKP | HBC24 VL |
| | GQAPRLLIYSASTRATGIPDRFSGSGSGTDFTLTISRLEPED | |
| | FAVYYCQQYAYSPRWTFGQGTKVEIK | |
| 97 | GSTFTKYA | CDRH1 of HBC24 |
| 98 | ISGSVPGF | CDRH2 of HBC24 |
| 99 | LYYCAKDVGVIGSYYYYAMDV | CDRH3 of HBC24 |
| 100 | QGLSSSY | CDRL1 of HBC24 |
| 101 | SAS | CDRL2 of HBC24 |
| 102 | QQYAYSPRWT | CDRL3 of HBC24 |
| 103 | gagctgcagctggtggagtccggcggcggctgggtgcagcctggcggctcccagaggct | VH of HBC34-V7, |
| | gagctgtgccgcttctggcaggatcttccggtccttttacatgtcttgggtgcggcaggctcc | HBC34-V35, and |
| | aggcaagggcctggagtgggtggctaccatcaaccaggacggctccgagaagctgtat | HBC34-V34 (codon |
| | gtggatagcgtgaagggcagattcacaatctctcgcgacaacgccaagaactccctgtttct | optimized) |
| | gcagatgaacaatctgagggtggaggataccgccgtgtactattgcgccgcttggtctggc | |
| | aatagcggcggcatggacgtgtggggacagggcaccaccgtgtccgtgtccagc | |
| 104 | agctacgagctgacacagcccccttccgtgtccgtgtcccctggacagaccgtgtccatccc | HBC34-V34 VL |
| | atgcagcggcgacaagctgggcaacaagaacgtgtcctggtttcagcataagcctggcca | (codon optimized) |
| | gtcccccgtgctggtcatctacgaggtgaagtataggcccagcggcatccctgagcggttct | |
| | ctggctccaacagcggcaatacagccaccctgacaatctctggcacacaggctatggacg | |
| | aggccgcttatttctgccagacctttgattccaccacagtggtgttcggcggcggcaccaga | |
| | ctgacagtgctg | |
| 105 | agctacgagctgacacagcccccttccgtgtccgtgtcccctggacagaccgtgtccatccc | HBC34-V35 VL |
| | atgcagcggcgacaagctgggcaacaagaacgtggcctggtttcagcataagcctggcca | (codon optimized) |
| | gtcccccgtgctggtcatctacgaggtgaagtataggcccagcggcatccctgagcggttct | |
| | ctggctccaacagcggcaatacagccaccctgacaatctctggcacacaggctatggacg | |
| | aggccgcttatttctgccagacctttgattccaccacagtggtgttcggcggcggcaccaga | |
| | ctgacagtgctg | |
| 106 | gaggtgcagttgttggagtctgggggaggcttggtacagcctggggggtccctgagactct | HBC24 VH |
| | cctgtgcagcctctGGATCCACTTTTACCAAATATGCCatgagctggg | (wild type) |
| | tccgtcaggctccagggaaggggctggagtgggtcgcaagtATTAGTGGAAGT | |
| | gttcctggttttGGTATTGACACAtactacgcagactccgttaagggccggttcac | |
| | catctccagagacacttccaagaacaccctgtatctgcaaatgaacagcctgagagccgag | |
| | gacacggccttatattactgtGCGAAAGATGTCGGGGTTATCGGGTC | |
| | ATACTATTACTACGCTATGGACGTCtggggtcaa | |
| 107 | aaattgtgttgacgcagtctccaggcaccctgtctttgtctccaggggaaagagccaccctct | HBC24 VL |
| | cctgcagggccagtCAGGGTCTTAGCAGCAGTTACttagcctggtacca | (wild type) |
| | gcagaaacctggccaggctcccaggctcctcatctatAGTGCGTCCaccagggcc | |
| | actggcatcccagacaggttcagtggcagtgggtctgggacagacttcactctcaccatcag | |
| | cagactggagcctgaagattttgcagtgtattactgtCAACAGTATGCTTACT | |
| | CACCTCGGTGGACGttcggccaagggaccaaggtggagatcaaac | |
| 108 | GAGGTGCAGCTGCTGGAAAGCGGCGGCGGCCTGGTGC | HBC24 VH |
| | AGCCCGGCGGCTCCCTGAGGCTGTCTTGCGCCGCCTCT | (codon optimized) |
| | GGCAGCACCTTCACAAAGTATGCAATGTCTTGGGTGCG | |
| | CCAGGCACCAGGCAAGGGCCTGGAGTGGGTGGCCTCC | |
| | ATCTCTGGCAGCGTGCCTGGCTTCGGCATCGACACCTA | |
| | CTATGCCGATTCCGTGAAGGGCCGGTTTACAATCAGCA | |
| | GAGACACCTCCAAGAACACACTGTATCTGCAGATGAAT | |
| | TCTCTGCGGGCCGAGGACACCGCCCTGTACTATTGTGC | |
| | CAAGGATGTGGGCGTGATCGGCAGCTACTATTACTATG | |
| | CAATGGACGTGTGGGGACAGGGAACAGCAGTGACAGT | |
| | GAGCTCC | |
| 109 | GAGATCGTGCTGACCCAGTCTCCTGGCACACTGTCCCT | HBC24 VL |
| | GTCCCCTGGAGAGAGAGCCACCCTGTCCTGCAGAGCCT | (codon optimized) |
| | CTCAGGGCCTGAGCTCCTCTTACCTGGCCTGGTATCAGC | |
| | AGAAGCCTGGACAGGCCCCTCGGCTGCTGATCTACTCT | |
| | GCCTCCACCAGAGCAACAGGCATTCCTGACCGCTTCTC | |
| | CGGATCTGGAAGCGGCACAGACTTCACCCTGACAATCA | |
| | GCCGGCTGGAGCCTGAGGACTTCGCCGTGTACTATTGT | |
| | CAGCAGTACGCCTATTCCCCAAGGTGGACCTTTGGCCA | |
| | GGGCACAAAGGTGGAGATCAAG | |
| 110 | agctacgagctgacacagcccccttccgtgtccgtgtcccctggacagaccgtgtccatccc | HBC34-V7 VL |
| | atgcagcggcgacaagctgggcaacaagaacgtgtgctggtttcagcataagcctggcca | (codon optimized) |
| | gtcccccgtgctggtcatctacgaggtgaagtataggcccagcggcatccctgagcggttct | |
| | ctggctccaacagcggcaatacagccaccctgacaatctctggcacacaggctatggacg | |
| | aggccgcttatttctgccagacctttgattccaccacagtggtgttcggcggcggcaccaga | |
| | ctgacagtgctg | |
| 111 | SYELTQPPSVSVSPGQTASITCSGDKLGNKNASWYQQKPG | HBC34v23-L_C40S |
| | QSPVLVIYEVKYRPSGIPERFSGSNSGNTATLTISGTQAMD | |
| | EADYYCQTFDSTTVVFGGGTKLTVL | |
| 112 | SYELTQPPSVSVSPGQTASITCSGDKLGNKNAAWYQQKP | HBC34v23-L_C40A |
| | GQSPVLVIYEVKYRPSGIPERFSGSNSGNTATLTISGTQAM | |
| | DEADYYCQTFDSTTVVFGGGTKLTVL | |
| 113 | SYELTQPPSVSVSPGQTVSIPCSGDKLGNKNVSWFQHKPG | HBC34v31-L_C40S |
| | QSPVLVIYEVKYRPSGIPERFSGSNSGNTATLTISGTQAMD | |
| | EAAYFCQTWDSTTVVFGGGTRLTVL | |
| 114 | SYELTQPPSVSVSPGQTVSIPCSGDKLGNKNVAWFQHKPG | HBC34v31-L_C40A |
| | QSPVLVIYEVKYRPSGIPERFSGSNSGNTATLTISGTQAMD | |
| | EAAYFCQTWDSTTVVFGGGTRLTVL | |
| 115 | SYELTQPPSVSVSPGQTVSIPCSGDKLGNKNVSWFQHKPG | HBC34v32-L_C40S |
| | QSPVLVIYEVKYRPSGIPERFSGSNSGNTATLTISGTQAMD | |
| | EAAYFCQTFDSTTVVFGGGTRLTVL | |
| 116 | SYELTQPPSVSVSPGQTVSIPCSGDKLGNKNVAWFQHKPG | HBC34v32-L_C40A |
| | QSPVLVIYEVKYRPSGIPERFSGSNSGNTATLTISGTQAMD | |
| | EAAYFCQTWDSTTVVFGGGTRLTVL | |
| 117 | SYELTQPPSVSVSPGQTASITCSGDKLGNKNASWYQQKPG | HBC34v33-L_C40S |
| | QSPVLVIYEVKYRPSGIPERFSGSNSGNTATLTISGTQAMD | |
| | EADYYCQTFDSTTVVFGGGTKLTVL | |
| 118 | SYELTQPPSVSVSPGQTASITCSGDKLGNKNAAWYQQKP | HBC34v33-L_C40A |
| | GQSPVLVIYEVKYRPSGIPERFSGSNSGNTATLTISGTQAM | |
| | DEADYYCQTFDSTTVVFGGGTKLTVL | |
| 119 | SYELTQPPSVSVSPGQTVSIPCSGDKLGNKNVSWFQHKPG | HBC34-LC40S |
| | QSPVLVIYEVKYRPSGIPERFSGSNSGNTATLTISGTQAMD | |
| | EAAYFCQTWDSTTVVFGGGTRLTVL | |
| 120 | SYELTQPPSVSVSPGQTVSIPCSGDKLGNKNVAWFQHKPG | HBC34-LC40A |
| | QSPVLVIYEVKYRPSGIPERFSGSNSGNTATLTISGTQAMD | |
| | EAAYFCQTWDSTTVVFGGGTRLTVL | |
| 121 | EVQLLESGGGLVQPGGSLRLSCAASGSTFTKYAMSWVRQ | HBC24-MLNS |
| | APGKGLEWVASISGSVPGFGIDTYYADSVKGRFTISRDTS | |
| | KNTLYLQMNSLRAEDTALYYCAKDVGVIGSYYYYAMDV | |
| | WGQGTAVTVSSASTKGPSVFPLAPSSKSTSGGTAALGCLV | |
| | KDYFPEPVTVSWNSGALTSGVHTFPAVLQSSGLYSLSSVV | |
| | TVPSSSLGTQTYICNVNHKPSNTKVDKKVEPKSCDKTHTC | |
| | PPCPAPELLGGPSVFLFPPKPKDTLMISRTPEVTCVVVDVS | |
| | HEDPEVKFNWYVDGVEVHNAKTKPREEQYNSTYRVVSV | |
| | LTVLHQDWLNGKEYKCKVSNKALPAPIEKTISKAKGQPR | |
| | EPQVYTLPPSRDELTKNQVSLTCLVKGFYPSDIAVEWESN | |
| | GQPENNYKTTPPVLDSDGSFFLYSKLTVDKSRWQQGNVF | |
| | SCSVLHEALHSHYTQKSLSLSPGK | |
| 122 | EVQLLESGGGLVQPGGSLRLSCAASGSTFTKYAMSWVRQ | HBC24-MLNS- |
| | APGKGLEWVASISGSVPGFGIDTYYADSVKGRFTISRDTS | GAALIE |
| | KNTLYLQMNSLRAEDTALYYCAKDVGVIGSYYYYAMDV | |
| | WGQGTAVTVSSASTKGPSVFPLAPSSKSTSGGTAALGCLV | |
| | KDYFPEPVTVSWNSGALTSGVHTFPAVLQSSGLYSLSSVV | |
| | TVPSSSLGTQTYICNVNHKPSNTKVDKKVEPKSCDKTHTC | |
| | PPCPAPELLAGPSVFLFPPKPKDTLMISRTPEVTCVVVDVS | |
| | HEDPEVKFNWYVDGVEVHNAKTKPREEQYNSTYRVVSV | |
| | LTVLHQDWLNGKEYKCKVSNKALPLPEEKTISKAKGQPR | |
| | EPQVYTLPPSRDELTKNQVSLTCLVKGFYPSDIAVEWESN | |
| | GQPENNYKTTPPVLDSDGSFFLYSKLTVDKSRWQQGNVF | |
| | SCSVLHEALHSHYTQKSLSLSPGK | |
| 123 | ELQLVESGGGWVQPGGSQRLSCAASGRIFRSFYMSWVRQ | HBC34-V7-mu |
| | APGKGLEWVATINQDGSEKLYVDSVKGRFTISRDNAKNS | (IgG2a) HC |
| | LFLQMNNLRVEDTAVYYCAAWSGNSGGMDVWGQGTTV | |
| | SVSSAKTTAPSVYPLAPVCGDTTGSSVTLGCLVKGYFPEP | |
| | VTLTWNSGSLSSGVHTFPAVLQSDLYTLSSSVTVTSSTWP | |
| | SQSITCNVAHPASSTKVDKKIEPRGPTIKPCPPCKCPAPNLL | |
| | GGPSVFIFPPKIKDVLMISLSPIVTCVVVDVSEDDPDVQISW | |
| | FVNNVEVHTAQTQTHREDYNSTLRVVSALPIQHQDWMS | |
| | GKEFKCKVNNKDLPAPIERTISKPKGSVRAPQVYVLPPPEE | |
| | EMTKKQVTLTCMVTDFMPEDIYVEWTNNGKTELNYKNT | |
| | EPVLDSDGSYFMYSKLRVEKKNWVERNSYSCSVVHEGLH | |
| | NHHTTKSFSRTPGK | |
| 124 | SYELTQPPSVSVSPGQTVSIPCSGDKLGNKNVCWFQHKPG | HBC34-V7-mu |
| | QSPVLVIYEVKYRPSGIPERFSGSNSGNTATLTISGTQAMD | (IgG2a) LC |
| | EAAYFCQTFDSTTVVFGGGTRLTVLGQPKSSPSVTLFPPSS | |
| | EELETNKATLVCTITDFYPGVVTVDWKVDGTPVTQGMET | |
| | TQPSKQSNNKYMASSYLTLTARAWERHSSYSCQVTHEGH | |
| | TVEKSLSRADCS | |
| 125 | ELQLVESGGGWVQPGGSQRLSCAASGRIFRSFYMSWVRQ | HBC34-V35-mu |
| | APGKGLEWVATINQDGSEKLYVDSVKGRFTISRDNAKNS | (IgG2a) HC |
| | LFLQMNNLRVEDTAVYYCAAWSGNSGGMDVWGQGTTV | |
| | SVSSAKTTAPSVYPLAPVCGDTTGSSVTLGCLVKGYFPEP | |
| | VTLTWNSGSLSSGVHTFPAVLQSDLYTLSSSVTVTSSTWP | |
| | SQSITCNVAHPASSTKVDKKIEPRGPTIKPCPPCKCPAPNLL | |
| | GGPSVFIFPPKIKDVLMISLSPIVTCVVVDVSEDDPDVQISW | |
| | FVNNVEVHTAQTQTHREDYNSTLRVVSALPIQHQDWMS | |
| | GKEFKCKVNNKDLPAPIERTISKPKGSVRAPQVYVLPPPEE | |
| | EMTKKQVTLTCMVTDFMPEDIYVEWTNNGKTELNYKNT | |
| | EPVLDSDGSYFMYSKLRVEKKNWVERNSYSCSVVHEGLH | |
| | NHHTTKSFSRTPGK | |
| 126 | SYELTQPPSVSVSPGQTVSIPCSGDKLGNKNVAWFQHKPG | HBC34-V35-mu |
| | QSPVLVIYEVKYRPSGIPERFSGSNSGNTATLTISGTQAMD | (IgG2a) LC |
| | EAAYFCQTFDSTTVVFGGGTRLTVLGQPKSSPSVTLFPPSS | |
| | EELETNKATLVCTITDFYPGVVTVDWKVDGTPVTQGMET | |
| | TQPSKQSNNKYMASSYLTLTARAWERHSSYSCQVTHEGH | |
| | TVEKSLSRADCS | |
| 127 | EVQLLESGGGLVQPGGSLRLSCAASGSTFTKYAMSWVRQ | HBC24-mu (IgG2a) |
| | APGKGLEWVASISGSVPGFGIDTYYADSVKGRFTISRDTS | HC |
| | KNTLYLQMNSLRAEDTALYYCAKDVGVIGSYYYYAMDV | |
| | WGQGTAVTVSSAKTTAPSVYPLAPVCGDTTGSSVTLGCL | |
| | VKGYFPEPVTLTWNSGSLSSGVHTFPAVLQSDLYTLSSSV | |
| | TVTSSTWPSQSITCNVAHPASSTKVDKKIEPRGPTIKPCPPC | |
| | KCPAPNLLGGPSVFIFPPKIKDVLMISLSPIVTCVVVDVSED | |
| | DPDVQISWFVNNVEVHTAQTQTHREDYNSTLRVVSALPI | |
| | QHQDWMSGKEFKCKVNNKDLPAPIERTISKPKGSVRAPQ | |
| | VYVLPPPEEEMTKKQVTLTCMVTDFMPEDIYVEWTNNGK | |
| | TELNYKNTEPVLDSDGSYFMYSKLRVEKKNWVERNSYSC | |
| | SVVHEGLHNHHTTKSFSRTPGK | |
| 128 | EIVLTQSPGTLSLSPGERATLSCRASQGLSSSYLAWYQQKP | HBC24-mu (IgG2a) |
| | GQAPRLLIYSASTRATGIPDRFSGSGSGTDFTLTISRLEPED | LC |
| | FAVYYCQQYAYSPRWTFGQGTKVEIKADAAPTVSIFPPSS | |
| | EQLTSGGASVVCFLNNFYPKDINVKWKIDGSERQNGVLN | |
| | SWTDQDSKDSTYSMSSTLTLTKDEYERHNSYTCEATHKT | |
| | STSPIVKSFNRNEC | |
| 129 | ELQLVESGGGWVQPGGSQRLSCAASGRIFRSFYMSWVRQ | HBC34-V7, HBC34- |
| | APGKGLEWVATINQDGSEKLYVDSVKGRFTISRDNAKNS | V34, HBC34-V35 |
| | LFLQMNNLRVEDTAVYYCAAWSGNSGGMDVWGQGTTV | HC (wild-type) |
| | SVSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKKVEPKSCDKTHTCPPCPAPEL | |
| | LGGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPGK | |
| 130 | GCCTCCACAAAGGGCCCAAGCGTGTTTCCACTGGCTCC | HBC34-V7, HBC34- |
| | CTCTTCCAAGTCTACCTCCGGCGGCACAGCCGCTCTGG | V34, HBC34-V35 |
| | GATGTCTGGTGAAGGATTACTTCCCAGAGCCCGTGACC | CH1-hinge-CH2-CH3 |
| | GTGTCTTGGAACTCCGGCGCCCTGACCAGCGGAGTGCA | (codon-optimized) |
| | TACATTTCCAGCTGTGCTGCAGAGCTCTGGCCTGTACTC | |
| | TCTGTCCAGCGTGGTGACCGTGCCCTCTTCCAGCCTGGG | |
| | CACCCAGACATATATCTGCAACGTGAATCACAAGCCAA | |
| | GCAATACAAAGGTGGACAAGAAGGTGGAGCCCAAGTC | |
| | TTGTGATAAGACCCATACATGCCCTCCATGTCCAGCTCC | |
| | AGAGCTGCTGGGCGGCCCAAGCGTGTTCCTGTTTCCAC | |
| | CCAAGCCTAAGGATACCCTGATGATCTCCAGAACCCCC | |
| | GAGGTGACATGCGTGGTGGTGGACGTGAGCCACGAGG | |
| | ATCCTGAGGTGAAGTTCAACTGGTACGTGGACGGCGTG | |
| | GAGGTGCATAATGCTAAGACCAAGCCCAGGGAGGAGC | |
| | AGTACAACTCTACCTATCGGGTGGTGTCCGTGCTGACA | |
| | GTGCTGCACCAGGATTGGCTGAACGGCAAGGAGTATAA | |
| | GTGCAAGGTGTCTAATAAGGCCCTGCCCGCTCCTATCG | |
| | AGAAGACCATCTCCAAGGCCAAGGGCCAGCCTAGAGA | |
| | GCCACAGGTGTACACACTGCCTCCATCTCGCGATGAGC | |
| | TGACCAAGAACCAGGTGTCCCTGACATGTCTGGTGAAG | |
| | GGCTTCTATCCTTCCGACATCGCTGTGGAGTGGGAGAG | |
| | CAATGGCCAGCCAGAGAACAATTACAAGACCACACCC | |
| | CCTGTGCTGGACAGCGATGGCTCTTTCTTTCTGTATAGC | |
| | AAGCTGACCGTGGACAAGTCTCGCTGGCAGCAGGGCA | |
| | ACGTGTTTAGCTGTTCTGTGATGCATGAGGCCCTGCAC | |
| | AATCATTATACACAGAAGTCCCTGAGCCTGTCTCCTGG | |
| | CAAG | |
| 131 | GAGCTGCAGCTGGTGGAGTCCGGCGGCGGCTGGGTGCA | HBC34-V7, HBC34- |
| | GCCTGGCGGCTCCCAGAGGCTGAGCTGTGCCGCTTCTG | V34, HBC34-V35 |
| | GCAGGATCTTCCGGTCCTTTTACATGTCTTGGGTGCGGC | VH-CH1-hinge-CH2- |
| | AGGCTCCAGGCAAGGGCCTGGAGTGGGTGGCTACCATC | CH3 (codon- |
| | AACCAGGACGGCTCCGAGAAGCTGTATGTGGATAGCGT | optimized) |
| | GAAGGGCAGATTCACAATCTCTCGCGACAACGCCAAGA | |
| | ACTCCCTGTTTCTGCAGATGAACAATCTGAGGGTGGAG | |
| | GATACCGCCGTGTACTATTGCGCCGCTTGGTCTGGCAA | |
| | TAGCGGCGGCATGGACGTGTGGGGACAGGGCACCACC | |
| | GTGTCCGTGTCCAGCGCCTCCACAAAGGGCCCAAGCGT | |
| | GTTTCCACTGGCTCCCTCTTCCAAGTCTACCTCCGGCGG | |
| | CACAGCCGCTCTGGGATGTCTGGTGAAGGATTACTTCC | |
| | CAGAGCCCGTGACCGTGTCTTGGAACTCCGGCGCCCTG | |
| | ACCAGCGGAGTGCATACATTTCCAGCTGTGCTGCAGAG | |
| | CTCTGGCCTGTACTCTCTGTCCAGCGTGGTGACCGTGCC | |
| | CTCTTCCAGCCTGGGCACCCAGACATATATCTGCAACG | |
| | TGAATCACAAGCCAAGCAATACAAAGGTGGACAAGAA | |
| | GGTGGAGCCCAAGTCTTGTGATAAGACCCATACATGCC | |
| | CTCCATGTCCAGCTCCAGAGCTGCTGGGCGGCCCAAGC | |
| | GTGTTCCTGTTTCCACCCAAGCCTAAGGATACCCTGATG | |
| | ATCTCCAGAACCCCCGAGGTGACATGCGTGGTGGTGGA | |
| | CGTGAGCCACGAGGATCCTGAGGTGAAGTTCAACTGGT | |
| | ACGTGGACGGCGTGGAGGTGCATAATGCTAAGACCAA | |
| | GCCCAGGGAGGAGCAGTACAACTCTACCTATCGGGTGG | |
| | TGTCCGTGCTGACAGTGCTGCACCAGGATTGGCTGAAC | |
| | GGCAAGGAGTATAAGTGCAAGGTGTCTAATAAGGCCCT | |
| | GCCCGCTCCTATCGAGAAGACCATCTCCAAGGCCAAGG | |
| | GCCAGCCTAGAGAGCCACAGGTGTACACACTGCCTCCA | |
| | TCTCGCGATGAGCTGACCAAGAACCAGGTGTCCCTGAC | |
| | ATGTCTGGTGAAGGGCTTCTATCCTTCCGACATCGCTGT | |
| | GGAGTGGGAGAGCAATGGCCAGCCAGAGAACAATTAC | |
| | AAGACCACACCCCCTGTGCTGGACAGCGATGGCTCTTT | |
| | CTTTCTGTATAGCAAGCTGACCGTGGACAAGTCTCGCT | |
| | GGCAGCAGGGCAACGTGTTTAGCTGTTCTGTGATGCAT | |
| | GAGGCCCTGCACAATCATTATACACAGAAGTCCCTGAG | |
| | CCTGTCTCCTGGCAAGTGATGAGGTACCGTGCGACGGC | |
| | CGGCAAGCCCCCGCTCCCCGGGCTCTCGCGGTCGTACG | |
| | AGGAAAGCTT | |
| 132 | GGACAGCCAAAGGCTGCTCCATCTGTGACCCTGTTTCC | HBC34-V7 CL |
| | ACCCTCTTCCGAGGAGCTGCAGGCCAACAAGGCCACCC | (codon-optimized) |
| | TGGTGTGCCTGATCTCTGACTTCTACCCTGGAGCTGTGA | |
| | CAGTGGCTTGGAAGGCTGATAGCTCTCCCGTGAAGGCT | |
| | GGCGTGGAGACAACAACCCCTAGCAAGCAGTCTAACA | |
| | ATAAGTACGCCGCTTCCAGCTATCTGTCTCTGACACCA | |
| | GAGCAGTGGAAGTCCCACCGCTCTTATTCCTGCCAGGT | |
| | GACCCATGAGGGCAGCACCGTGGAGAAGACAGTGGCC | |
| | CCCACCGAGTGTTCT | |
| 133 | AGCTACGAGCTGACACAGCCCCCTTCCGTGTCCGTGTC | HBC34-V7 LC (VL- |
| | CCCTGGACAGACCGTGTCCATCCCATGCAGCGGCGACA | CL) (codon- |
| | AGCTGGGCAACAAGAACGTGTGCTGGTTTCAGCATAAG | optimized) |
| | CCTGGCCAGTCCCCCGTGCTGGTCATCTACGAGGTGAA | |
| | GTATAGGCCCAGCGGCATCCCTGAGCGGTTCTCTGGCT | |
| | CCAACAGCGGCAATACAGCCACCCTGACAATCTCTGGC | |
| | ACACAGGCTATGGACGAGGCCGCTTATTTCTGCCAGAC | |
| | CTTTGATTCCACCACAGTGGTGTTCGGCGGCGGCACCA | |
| | GACTGACAGTGCTGGGACAGCCAAAGGCTGCTCCATCT | |
| | GTGACCCTGTTTCCACCCTCTTCCGAGGAGCTGCAGGC | |
| | CAACAAGGCCACCCTGGTGTGCCTGATCTCTGACTTCT | |
| | ACCCTGGAGCTGTGACAGTGGCTTGGAAGGCTGATAGC | |
| | TCTCCCGTGAAGGCTGGCGTGGAGACAACAACCCCTAG | |
| | CAAGCAGTCTAACAATAAGTACGCCGCTTCCAGCTATC | |
| | TGTCTCTGACACCAGAGCAGTGGAAGTCCCACCGCTCT | |
| | TATTCCTGCCAGGTGACCCATGAGGGCAGCACCGTGGA | |
| | GAAGACAGTGGCCCCCACCGAGTGTTCT | |
| 134 | GGACAGCCAAAGGCTGCTCCATCTGTGACCCTGTTTCC | HBC34-V34, |
| | ACCCTCTTCCGAGGAGCTGCAGGCCAACAAGGCCACCC | HBC34-V35 |
| | TGGTGTGCCTGATCTCTGACTTCTACCCTGGAGCTGTGA | CL (codon-optimized) |
| | CAGTGGCTTGGAAGGCTGATAGCTCTCCCGTGAAGGCT | |
| | GGCGTGGAGACAACAACCCCTAGCAAGCAGTCTAACA | |
| | ATAAGTACGCCGCTTCCAGCTATCTGTCTCTGACACCA | |
| | GAGCAGTGGAAGTCCCACCGCTCTTATTCCTGCCAGGT | |
| | GACCCATGAGGGCAGCACCGTGGAGAAGACAGTGGCC | |
| | CCCACCGAGTGTTCT | |
| 135 | AGCTACGAGCTGACACAGCCCCCTTCCGTGTCCGTGTC | HBC34-V34 LC (VL- |
| | CCCTGGACAGACCGTGTCCATCCCATGCAGCGGCGACA | CL) (codon- |
| | AGCTGGGCAACAAGAACGTGTCCTGGTTTCAGCATAAG | optimized) |
| | CCTGGCCAGTCCCCCGTGCTGGTCATCTACGAGGTGAA | |
| | GTATAGGCCCAGCGGCATCCCTGAGCGGTTCTCTGGCT | |
| | CCAACAGCGGCAATACAGCCACCCTGACAATCTCTGGC | |
| | ACACAGGCTATGGACGAGGCCGCTTATTTCTGCCAGAC | |
| | CTTTGATTCCACCACAGTGGTGTTCGGCGGCGGCACCA | |
| | GACTGACAGTGCTGGGACAGCCAAAGGCTGCTCCATCT | |
| | GTGACCCTGTTTCCACCCTCTTCCGAGGAGCTGCAGGC | |
| | CAACAAGGCCACCCTGGTGTGCCTGATCTCTGACTTCT | |
| | ACCCTGGAGCTGTGACAGTGGCTTGGAAGGCTGATAGC | |
| | TCTCCCGTGAAGGCTGGCGTGGAGACAACAACCCCTAG | |
| | CAAGCAGTCTAACAATAAGTACGCCGCTTCCAGCTATC | |
| | TGTCTCTGACACCAGAGCAGTGGAAGTCCCACCGCTCT | |
| | TATTCCTGCCAGGTGACCCATGAGGGCAGCACCGTGGA | |
| | GAAGACAGTGGCCCCCACCGAGTGTTCT | |
| 136 | AGCTACGAGCTGACACAGCCCCCTTCCGTGTCCGTGTC | HBC34-V35 LC (VL- |
| | CCCTGGACAGACCGTGTCCATCCCATGCAGCGGCGACA | CL) (codon- |
| | AGCTGGGCAACAAGAACGTGGCCTGGTTTCAGCATAAG | optimized) |
| | CCTGGCCAGTCCCCCGTGCTGGTCATCTACGAGGTGAA | |
| | GTATAGGCCCAGCGGCATCCCTGAGCGGTTCTCTGGCT | |
| | CCAACAGCGGCAATACAGCCACCCTGACAATCTCTGGC | |
| | ACACAGGCTATGGACGAGGCCGCTTATTTCTGCCAGAC | |
| | CTTTGATTCCACCACAGTGGTGTTCGGCGGCGGCACCA | |
| | GACTGACAGTGCTGGGACAGCCAAAGGCTGCTCCATCT | |
| | GTGACCCTGTTTCCACCCTCTTCCGAGGAGCTGCAGGC | |
| | CAACAAGGCCACCCTGGTGTGCCTGATCTCTGACTTCT | |
| | ACCCTGGAGCTGTGACAGTGGCTTGGAAGGCTGATAGC | |
| | TCTCCCGTGAAGGCTGGCGTGGAGACAACAACCCCTAG | |
| | CAAGCAGTCTAACAATAAGTACGCCGCTTCCAGCTATC | |
| | TGTCTCTGACACCAGAGCAGTGGAAGTCCCACCGCTCT | |
| | TATTCCTGCCAGGTGACCCATGAGGGCAGCACCGTGGA | |
| | GAAGACAGTGGCCCCCACCGAGTGTTCT | |
| 137 | APELLGGPSVFLFPPKPKDTLMISRTPEVTCVVVDVSHEDP | WT hIgG1 Fc |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPGK | |
| 138 | ELQLVESGGGWVQPGGSQRLSCAASGRIFRSFYMSWVRQ | HBC34, |
| | APGKGLEWVATINQDGSEKLYVDSVKGRFTISRDNAKNS | HBC34v7, |
| | LFLQMNNLRVEDTAVYYCAAWSGNSGGMDVWGQGTTV | HBC34v23, |
| | SVSSASTKGPSVFPLAPSSKSTSGGTAALGCLVKDYFPEPV | HBC34v34, |
| | TVSWNSGALTSGVHTFPAVLQSSGLYSLSSVVTVPSSSLG | HBC34v35, |
| | TQTYICNVNHKPSNTKVDKKVEPKSCDKTHTCPPCPAPEL | HBC34_C40S, |
| | LAGPSVFLFPPKPKDTLMISRTPEVTCVVVDVSHEDPEVKF | HBC34_C40A, |
| | NWYVDGVEVHNAKTKPREEQYNSTYRVVSVLTVLHQD | HBC34v23_C40S, |
| | WLNGKEYKCKVSNKALPLPEEKTISKAKGQPREPQVYTL | HBC34v23_C40A |
| | PPSRDELTKNQVSLTCLVKGFYPSDIAVEWESNGQPENNY | HC with GAALIE |
| | KTTPPVLDSDGSFFLYSKLTVDKSRWQQGNVFSCSVMHE | mutation in hIgG1 Fc |
| | ALHNHYTQKSLSLSPGK | |

[symbol]

indicates data missing or illegible when filed

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description.

U.S. Provisional Application 62/893,742, filed Aug. 29, 2019, and U.S. patent application Ser. No. 17/637,605, filed Feb. 23, 2022, are each incorporated herein by reference, in their entireties.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating a Hepatitis B virus (HBV) infection in a subject, the method comprising administering to the subject a single dose of a pharmaceutical composition comprising an antibody, wherein the antibody comprises the heavy chain amino acid sequence of SEQ ID NO.:91 and the light chain amino acid sequence of SEQ ID NO.:93.

2.-35. (canceled)

36. A pharmaceutical composition comprising an antibody, wherein the antibody comprises the heavy chain amino acid sequence of SEQ ID NO.:91 and the light chain amino acid sequence of SEQ ID NO.:93, wherein the pharmaceutical composition comprises the antibody at a concentration ranging from 100 mg/mL to 200 mg/mL.

37.-48. (canceled)

49. The pharmaceutical composition of claim 36, wherein the pharmaceutical composition comprises the antibody at a concentration of 140 mg/mL.

50. The pharmaceutical composition of claim 36, wherein the pharmaceutical composition comprises up to 300 mg, up to 900 mg, or up to 3000 mg of the antibody.

51. The pharmaceutical composition of claim 36, wherein the pharmaceutical composition comprises about 300 mg of the antibody.

52. The pharmaceutical composition of claim 36, further comprising histidine.

53. The pharmaceutical composition of claim 52, wherein histidine is present in the pharmaceutical composition at a concentration from 10 mM to 40 mM.

54. The pharmaceutical composition of claim 36, further comprising a disaccharide.

55. The pharmaceutical composition of claim 54, wherein the disaccharide is sucrose.

56. The pharmaceutical composition of claim 55, wherein sucrose is present in the pharmaceutical composition at a concentration in a range of 4.0% to 10.0% (w/v).

57. The pharmaceutical composition of claim 36, further comprising a surfactant or a triblock copolymer.

58. The pharmaceutical composition of claim 57, wherein the surfactant is a polysorbate.

59. The pharmaceutical composition of claim 57, wherein the surfactant is polysorbate 80 (PS80).

60. The pharmaceutical composition of claim 58, wherein the polysorbate is present in the pharmaceutical composition in a range from 0.01% to 0.05% (w/v).

61. The pharmaceutical composition of claim 36, wherein the pharmaceutical composition has a pH ranging from 5.8 to 6.2.

62. The pharmaceutical composition of claim 36, wherein the pharmaceutical composition comprises:
(i) the antibody at 100 mg/mL to 200 mg/mL;
(ii) water;
(iii) histidine; and
(iv) a surfactant or triblock copolymer,
wherein the pharmaceutical composition has a pH in a range from 5.8 to 6.2.

63. The pharmaceutical composition of claim 36, wherein the pharmaceutical composition comprises:
(i) the antibody at 100 mg/mL to 200 mg/mL;
(ii) water;
(iii) histidine at 10 mM to 40 mM; and
(iv) sucrose at 4.0-10.0% (w/v).

64. The pharmaceutical composition of claim 63, wherein the pharmaceutical composition has a pH in a range of from 5.8 to 6.2.

65. The pharmaceutical composition of claim 36, wherein the pharmaceutical composition comprises:
(i) the antibody at 150 mg/mL;
(ii) United States Pharmacopeia (USP) water;
(iii) 20 mM histidine;
(iv) 7% sucrose; and
(v) 0.02% PS80,
wherein the pharmaceutical composition comprises a pH of 6.

66. A method of treating a Hepatitis B virus (HBV) infection or Hepatitis D virus (HDV) infection in a subject, comprising administering to the subject the pharmaceutical composition of claim 36.
